(19) United States
(12) Patent Application Publication
(10) Pub. No.: US 2024/0343802 A1
(43) Pub. Date: Oct. 17, 2024

(54) Bispecific Asymmetric Heterodimers Comprising Anti-CD3 Constructs

(71) Applicant: ZYMEWORKS BC INC., Vancouver (CA)

(72) Inventors: Gordon Yiu Kon Ng, Vancouver (CA); Surjit Bhimarao Dixit, Richmond (CA); Thomas Spreter von Kreudenstein, Vancouver (CA); Nina E. Weisser, Delta (CA)

(21) Appl. No.: 18/612,960

(22) Filed: Mar. 21, 2024

Related U.S. Application Data
(63) Continuation of application No. 17/207,526, filed on Mar. 19, 2021, now abandoned.
Application No. 17/207,526 is a continuation of application No. 16/256,824, filed on Jan. 24, 2019, now abandoned.
Application No. 16/256,824 is a continuation of application No. 13/941,449, filed on Jul. 13, 2013, now abandoned.
(60) Provisional application No. 61/845,948, filed on Jul. 12, 2013.
(60) Provisional application No. 61/671,640, filed on Jul. 13, 2012.

(51) Int. Cl. C07K 16/28; C07K 16/32

(52) U.S. Cl. CPC C07K 16/2809; C07K 16/2803; C07K 16/2887; C07K 16/32; C07K 2317/31; C07K 2317/35; C07K 2317/52; C07K 2317/524; C07K 2317/526; C07K 2317/528; C07K 2317/622; C07K 2317/64; C07K 2317/71; C07K 2317/72; C07K 2317/73; C07K 2317/732; C07K 2317/74; C07K 2317/92; C07K 2317/94; C07K 2319/31

(57) ABSTRACT

Disclosed herein are isolated multi-specific heteromultimer constructs that bind to CD3 expressed on T-cells and to an antigen expressed on B-cells. The multi-specific heteromultimer constructs are capable of bridging T- and B-cells and mediating killing of B-cells. The multi-specific heteromultimer constructs are based on a heterodimeric Fc scaffold or on a segmented albumin scaffold. Also disclosed herein are multi-specific heteromultimer constructs that bind to HER2 and HER3.

### CROSS-REFERENCE TO RELATED APPLICATIONS

This application is a continuation of U.S. application Ser. No. 17/207,526, filed on Mar. 19, 2021, which is a continuation of U.S. application Ser. No. 16/256,824, filed on Jan. 24, 2019, now abandoned, which is a continuation of U.S. application Ser. No. 13/941,449, filed Jul. 13, 2013, now abandoned, which claims the benefit of U.S. Application No. 61/671,640, filed Jul. 13, 2012; and U.S. Application No. 61/845,948, filed Jul. 12, 2013, which are hereby incorporated by reference in their entirety.

### SEQUENCE LISTING

The instant application contains a Sequence Listing which has been submitted electronically in XML format and is hereby incorporated by reference in its entirety. Said XML file, created on Jun. 14, 2024, is named ZWI-007C3_SL.xml, and is 529,253 bytes in size.

### FIELD OF THE INVENTION

The field of the invention is the rational design of multispecific scaffolds comprising a CD3 binding domain for custom development of biotherapeutics.

### BACKGROUND OF THE INVENTION

In the realm of therapeutic proteins, antibodies with their multivalent target binding features are excellent scaffolds for the design of drug candidates. Advancing these features further, designed bispecific antibodies and other fused multispecific therapeutics exhibit dual or multiple target specificities and an opportunity to create drugs with novel modes of action. The development of such multivalent and multispecific therapeutic proteins with favorable pharmacokinetics and functional activity has been a challenge.

The immune system of both humans and animals include two principal classes of lymphocytes: the thymus derived cells (T cells), and the bone marrow derived cells (B cells). T cells exhibit immunological specificity and are directly involved in cell-mediated immune responses (such as graft rejection). T cells act against or in response to a variety of foreign structures (antigens). In many instances these foreign antigens are expressed on host cells as a result of infection. However, foreign antigens can also come from the host having been altered by neoplasia or infection.

T cell activation is a complex phenomenon that depends on the participation of a variety of cell surface molecules expressed on the responding T cell population. For example, the antigen-specific T cell receptor (TcR) is composed of a disulfide-linked heterodimer, containing two clonally distributed, integral membrane glycoprotein chains, alpha and beta (α and β), or gamma and delta (γ and δ), non-covalently associated with a complex of low molecular weight invariant proteins, commonly designated as CD3.

### SUMMARY OF THE INVENTION

Provided herein are multispecific heteromultimers comprising a CD3 binding domain. In an embodiment is provided a bispecific asymmetric heterodimer comprising anti-CD3 constructs.

Provided herein are isolated multispecific heteromultimer constructs comprising: a first polypeptide construct comprising a first heavy chain polypeptide and a CD3 binding polypeptide construct that binds to a CD3 complex on at least one CD3 expressing cell; a second polypeptide construct comprising a second heavy chain polypeptide which is different from said first heavy chain polypeptide, and an antigen binding polypeptide construct that binds to a target antigen on at least one B cell; wherein: at least one of said CD3 binding polypeptide construct and said antigen binding polypeptide construct comprises a single chain Fv region; wherein said multispecific heteromultimer construct simultaneously engages said at least one B cell and said at least one CD3 expressing cell such that the CD3 expressing cell is activated, thereby inducing killing of the B cell; and said first and second heavy chain polypeptides form a heterodimeric Fc region comprising a variant immunoglobulin CH3 region comprising at least one amino acid mutation that promotes the formation of said heterodimeric Fc with stability at least comparable to a native homodimeric Fc, and with purity such that when said multispecific heteromultimer construct is coexpressed from a stable mammalian cell in an expression product, said expression product comprises at least about 70% of said multispecific heteromultimer, and less than 10% monomers or homodimers of said first or second polypeptide constructs. In certain embodiments, said stable mammalian cell is transfected at least a first DNA sequence encoding said first polypeptide construct and at least a second DNA sequence encoding said second polypeptide construct in a pre-determined ratio of 1:1. In certain embodiments, the first or second polypeptide construct is devoid of at least one of immunoglobulin light chain, and immunoglobulin first constant (CH1) region.

In certain embodiments are the isolated heteromultimer constructs described herein, wherein the heterodimer Fc region comprises a variant CH2 domain or hinge comprising amino acid modifications that prevents functionally effective binding to all the Fcgamma receptors. In some embodiments is provided the isolated multispecific heteromultimer described herein, wherein wherein said variant CH2 domain or hinge comprising amino acid modification also prevents functionally effective binding to complement proteins (C1q complex). In an embodiment is the isolated multispecific heteromultimer described herein, wherein the heterodimer Fc region comprises a variant CH2 domain or hinge comprising amino acid modifications that enhance binding to the FcγRIIb receptor.

In an embodiment is provided an isolated multispecific heteromultimer construct comprising: a first polypeptide construct comprising a first heavy chain polypeptide and a CD3 binding polypeptide construct that binds to a CD3 complex on at least one CD3 expressing cell; a second polypeptide construct comprising a second heavy chain polypeptide which is different from said first heavy chain polypeptide, and an antigen binding polypeptide construct that binds to a target antigen on at least one B cell; wherein: at least one of said CD3 binding polypeptide construct and said antigen binding polypeptide construct optionally comprises a single chain Fv region; said first and second heavy chain polypeptides form a heterodimeric Fc region comprising a variant immunoglobulin CH3 region comprising at least one amino acid mutation that promotes the formation of said heterodimeric Fc, wherein: said heterodimeric Fc is formed with stability at least comparable to a native homodimeric Fc, and said heterodimeric Fc is formed with purity such that when said multispecific heteromultimer construct is coexpressed from a mammalian cell in an expression product, said expression product comprises greater than 70% of said multispecific heteromultimer, and less than 10% monomers or homodimers of said first or second polypeptide constructs; and said multispecific heteromultimer construct binds said at least one B cell with a valency greater than one, and said multispecific heteromultimer simultaneously engages said at least one B cell and said at least one CD3 expressing cell such that the CD3 expressing cell is activated, thereby inducing killing of the B cell. In certain embodiments, said multispecific heteromultimer construct binds said at least one B cell with a valency of two.

Provided herein is an isolated multispecific heteromultimer construct comprising: a first polypeptide construct comprising a first heavy chain polypeptide and a CD3 binding polypeptide construct that binds to a CD3 complex on at least one CD3 expressing cell; a second polypeptide construct comprising a second heavy chain polypeptide which is different from said first heavy chain polypeptide, and a steric modulator construct which exhibits negligible receptor binding; wherein: said multispecific heteromultimer construct simultaneously engages at least one B cell and said at least one CD3 expressing cell such that the CD3 expressing cell is activated, thereby inducing killing of the B cell; and said first and second heavy chain polypeptides form a heterodimeric Fc region comprising a variant immunoglobulin CH3 region comprising at least one amino acid mutation that promotes the formation of said heterodimeric Fc, wherein: said heterodimeric Fc is formed with stability at least comparable to a native homodimeric Fc, and said heterodimeric Fc is formed with purity such that when said multispecific heteromultimer construct is coexpressed from a stable mammalian cell in an expression product, said expression product comprises at least about 75% of said multispecific heteromultimer, and less than 10% monomers or homodimers of said first or second polypeptide constructs.

Provided is an isolated multispecific heteromultimer construct comprising: a first polypeptide construct comprising a first heavy chain polypeptide and a CD3 binding polypeptide construct that binds to a CD3 complex on at least one CD3 expressing cell; a second polypeptide construct comprising a second heavy chain polypeptide which is different from said first heavy chain polypeptide, and wherein said second polypeptide construct does not comprise an antigen binding polypeptide construct; wherein: said multispecific heteromultimer construct simultaneously engages at least one B cell and said at least one CD3 expressing cell such that the CD3 expressing cell is activated, thereby inducing killing of the B cell; and said first and second heavy chain polypeptides form a heterodimeric Fc region comprising a variant immunoglobulin CH3 region comprising at least one amino acid mutation that promotes the formation of said heterodimeric Fc, wherein: said heterodimeric Fc is formed with stability at least comparable to a native homodimeric Fc, and said heterodimeric Fc is formed with purity such that when said multispecific heteromultimer construct is coexpressed from a stable mammalian cell in an expression product, said expression product comprises at least about 75% of said multispecific heteromultimer, and less than 10% monomers or homodimers of said first or second polypeptide constructs.

In certain embodiments is the isolated multispecific heteromultimer described herein wherein the heterodimer Fc region comprises a variant CH2 domain comprising amino acid modifications to promote selective binding of a Fcgamma receptor. In some embodiments, the variant CH2 domain selectively binds FcgammaIIb receptor greater than to wild-type CH2 domain. In certain embodiments, the variant CH2 domain selectively binds at least one of FcgammaIIIa and FcgammaIIa receptor greater than a wild-type CH2 domain.

In certain embodiments is an isolated multispecific heteromultimer construct described herein, wherein the variant CH3 domain has a melting temperature (Tm) of about 73° C. or greater. In some embodiments, the heterodimer Fc region is formed with a purity greater than about 90%. In certain embodiments, the heterodimer Fc region is formed with a purity of about 95% or greater and the Tm is at least about 75° C. In some further embodiments, the heterodimer Fc region is formed with a purity of at least about 90% and the Tm is about 75° C. In an embodiment, the variant CH3 sequence of the first heavy chain polypeptide comprises the amino acid modifications L351Y, F405A, and Y407V, and the variant CH3 sequence of the second transporter polypeptide comprises the amino acid modifications T366L, K392M, and T394W. In another embodiment, the variant CH3 sequence of the first heavy chain polypeptide comprises the amino acid modifications L351Y, F405A, and Y407V, and the variant CH3 sequence of the second heavy chain polypeptide comprises the amino acid modifications T366L, K392L, and T394W. In a further embodiment, the variant CH3 sequence of the first heavy chain polypeptide comprises the amino acid modifications T350V, L351Y, F405A, and Y407V, and the variant CH3 sequence of the second heavy chain polypeptide comprises the amino acid modifications T350V, T366L, K392M, and T394W. In some embodiments, the variant CH3 sequence of the first heavy chain polypeptide comprises the amino acid modifications T350V, L351Y, F405A, and Y407V, and the variant CH3 sequence of the second heavy chain polypeptide comprises the amino acid modifications T350V, T366L, K392L, and T394W. In yet another embodiment, the variant CH3 sequence of the first heavy chain polypeptide comprises the amino acid modifications T366L, N390R, K392R, and T394W, and the variant CH3 sequence of the second heavy chain polypeptide comprises the amino acid modifications L351Y, S400E, F405A, and Y407V. In so, e embodiments, the variant CH3 sequence of the first heavy chain polypeptide comprises the amino acid modifications T350V, T366L, N390R, K392R, and T394W, and the variant CH3 sequence of the second heavy chain polypeptide comprises the amino acid modifications T350V, L351Y, S400E, F405A, and Y407V.

In certain embodiments is provided an isolated multispecific heteromultimer described herein, wherein the heterodimer Fc is glycosylated. In some embodiments is provided an isolated multispecific heteromultimer described herein, wherein the heterodimer Fc is afucosylated. In another embodiment is provided an isolated multispecific heteromultimer described herein, wherein the heterodimer Fc is aglycosylated.

In some embodiments is the isolated multispecific heteromultimer described herein wherein the antigen binding polypeptide construct that binds to a target antigen on at least one B cell comprises at least one target antigen binding domain derived from an antibody, a fibronectin, an affibody, anticalin, cysteine knot protein, DARPin, avimer, Kunitz domain or variant or derivative thereof. In an embodiment is the isolated multispecific heteromultimer described herein, wherein said antibody is a heavy chain antibody devoid of light chains. In further embodiments is the isolated multispecific heteromultimer described herein, wherein said antigen binding polypeptide construct comprises at least one CD19 binding domain. In certain embodiments is the isolated multispecific heteromultimer described herein, wherein said antigen binding polypeptide construct comprises at least one CD20 binding domain.

Provided herein is an isolated multispecific heteromultimer construct comprising: a first polypeptide construct comprising a first transporter polypeptide fused to at least one CD3 binding polypeptide construct that binds to a CD3 complex on at least one CD3 expressing cell; a second polypeptide construct comprising a second transporter polypeptide which is different from said first transporter polypeptide, fused to at least one antigen binding polypeptide construct that binds to a target antigen on at least one B cell; wherein said first and second transporter polypeptides are derived from a protein by segmentation of said protein, each transporter polypeptide comprising an amino acid sequence with at least 90% identity to a segment of said protein, and wherein said transporter polypeptides self-assemble to form a quasi-native structure of said monomeric protein.

In certain embodiments, said transporter polypeptides are not derived from an antibody. In certain embodiment, each transporter polypeptide is an albumin derivative. In some embodiments, said albumin is human serum albumin. In seome embodiments, least one transporter polypeptide is an allo-albumin derivative. In certain embodiments, each transporter polypeptide is derived from a different alloalbumin.

Provided herein are isolated multispecific heteromultimer constructs comprising: a first polypeptide construct comprising a first transporter polypeptide fused to at least one CD3 binding polypeptide construct that binds to a CD3 complex on at least one CD3 expressing cell; a second polypeptide construct comprising a second transporter polypeptide which is different from said first transporter polypeptide, fused to at least one antigen binding polypeptide construct that binds to a target antigen on at least one B cell; wherein said first and second transporter polypeptides are obtained by segmentation of albumin, and each transporter polypeptide comprising an amino acid sequence with at least 90% identity to a segment of albumin such that said transporter polypeptides self-assemble to form quasi-native albumin, and wherein said first cargo polypeptide does not have any binding domain present in said second cargo polypeptide.

In certain embodiments is provided a heteromultimer described herein, wherein said multispecific heteromultimer construct simultaneously engages said at least one B cell and said at least one CD3 expressing cell such that the CD3 expressing cell is activated, thereby inducing killing of the B cell. In some embodiments, said antigen binding polypeptide construct that binds to a target antigen on at least one B cell comprises at least one target antigen binding domain derived from an antibody, a fibronectin, an affibody, anticalin, cysteine knot protein, DARPin, avimer, Kunitz domain or variant or derivative thereof. In certain embodiments, the antigen binding polypeptide construct comprises at least one CD19 binding domain.

In certain embodiments is provided a multispecific heteromultimer described herein, wherein said antigen binding polypeptide construct comprises at least one CD20 binding domain.

In certain embodiments is provided a multispecific heteromultimer described herein, wherein the at least one CD3 binding polypeptide construct comprises at least one CD3 binding domain derived from a CD3 specific antibody, a nanobody, fibronectin, affibody, anticalin, cysteine knot protein, DARPin, avimer, Kunitz domain or variant or derivative thereof. In some embodiments, the at least one CD3 binding domain comprises at least one amino acid modification that reduces immunogenicity as compared to a corresponding CD3 binding domain not comprising said modification. In some embodiments is the isolated multispecific heteromultimer described herein, wherein said at least one CD3 binding domain comprises at least one amino acid modification that increases its stability as measured by Tm, as compared to a corresponding CD3 binding domain not comprising said modification. In some embodiments, the at least one CD3 binding polypeptide construct comprises at least one CD3 binding domain derived from a CD3 specific antibody is a heavy chain antibody devoid of light chains. In certain embodiments, the at least one CD3 binding polypeptide construct comprises at least one CD3 binding domain derived from a non-antibody protein scaffold domain.

In an embodiment is provided the isolated heteromutlimer construct described herein, wherein at least one of said first and second polypeptide constructs further comprises a single-chain Fv polypeptide. In certain embodiments, is provided the isolated heteromutlimer construct described herein, wherein at least one of said first and second polypeptide constructs further comprises a single-chain Fab polypeptide.

In some embodiments is the isolated heteromutlimer construct described herein, where in the CD3 expressing cell is a T-cell. In certain embodiments is the isolated heteromultimer described herein, wherein said heteromultimer binds to the T-cell with sufficient affinity and decorates the T cell at sufficient capacity that induces the T-cell to display B cell killing activity when the T cell and the B cell are bridged.

Provided is an isolated heteromutlimer construct described herein, where in the CD3 expressing cell is a human cell. In certain embodiments, the CD3 expressing cell is a non-human, mammalian cell. In some embodiments, the mammalian cell is a primate cell. In certain embodiments, the primate is a monkey. In some embodiments, the at least one CD3 binding polypeptide construct binds to CD3 constructs across multiple species. In certain embodiments the CD3 binding polypeptide binds to CD3 constructs across multiple species which include at least one or more of human, rat, mouse and monkey.

Provided is an isolated heteromutlimer construct described herein wherein the at least one B cell is associated with a disease. In certain embodiments, the disease is a cancer selected from a carcinoma, a sarcoma, leukaemia, lymphoma and glioma. In some embodiments, the cancer is at least one of squamous cell carcinoma, adenocarcinoma, transition cell carcinoma, osteosarcoma and soft tissue sarcoma. In an embodiment, the at least one B cell is an autoimmune reactive cell that is a lymphoid or myeloid cell.

Provided is an isolated heteromutlimer construct described herein wherein said heteromultimer further comprises at least one binding domain that binds at least one of: EpCAM, EGFR, IGFR, HER-2 neu, HER-3, HER-4, PSMA, CEA, MUC-1 (mucin), MUC2, MUC3, MUC4, MUC5, MUC7, CCR4, CCR5, CD19, CD20, CD33, CD30, ganglioside GD3, 9-O-Acetyl-GD3, GM2, Poly SA, GD2, Carboanhydrase IX (MN/CA IX), CD44v6, Sonic Hedgehog (Shh), Wue-1, Plasma Cell Antigen, (membrane-bound), Melanoma Chondroitin Sulfate Proteoglycan (MCSP), CCR8, TNF-alpha precursor, STEAP, mesothelin, A33 Antigen, Prostate Stem Cell Antigen (PSCA), Ly-6; desmoglein 4, E-cadherin neoepitope, Fetal Acetylcholine Receptor, CD25, CA19-9 marker, CA-125 marker and Muellerian Inhibitory Substance (MIS) Receptor type II, sTn (sialylated Tn antigen; TAG-72), FAP (fibroblast activation antigen), endosialin, LG, SAS, EPHA4 CD63, CD3 BsAb immunocytokines TNF, IFNγ, IL-2, and TRAIL.

Provided is an isolated heteromutlimer construct described herein wherein said heteromultimer optionally comprises at least one linker. In some embodiments, said at least one linker is a polypeptide comprising from about 1 to about 100 amino acids.

Provided is a set of expression vectors for expressing a multispecific heteromultimer described herein, comprising at least a first DNA sequence encoding said first polypeptide construct and at least a second DNA sequence encoding said second polypeptide construct.

Provided is a method of producing an expression product containing a multispecific heteromultimer described herein, in stable mammalian cells, the method comprising: transfecting at least one mammalian cell with: at least a first DNA sequence encoding said first polypeptide construct and at least a second DNA sequence encoding said second polypeptide construct, such that said at least one first DNA sequence, said at least one second DNA sequence are transfected in said at least one mammalian cell in a pre-determined ratio to generate stable mammalian cells; culturing said stable mammalian cells to produce said expression product comprising said multispecific heteromultimer. In some embodiments is the method of producing an expressin product containing a multispecific heteromultimer construct described herien, wherein said predetermined ratio of the at least one first DNA sequence: at least one second DNA sequence is about 1:1. In some embodiments, said mammalian cell is selected from the group consisting of a VERO, HeLa, HEK, NS0, Chinese Hamster Ovary (CHO), W138, BHK, COS-7, Caco-2 and MDCK cell, and subclasses and variants thereof.

Provided is a pharmaceutical composition comprising a multispecific heteromultimer described herein, and a suitable excipient. Also provided is a process for the production of said pharmaceutical composition, said process comprising: culturing a host cell under conditions allowing the expression of a heteromultimer as defined herein; recovering the produced heteromultimer from the culture; and producing the pharmaceutical composition.

Provided is a method for the prevention, treatment or amelioration of at least one of: a proliferative disease, a minimal residual cancer, a tumorous disease, an inflammatory disease, an immunological disorder, an autoimmune disease, an infectious disease, viral disease, allergic reactions, parasitic reactions, graft-versus-host diseases or host-versus-graft diseases or cell malignancies, said method comprising administering to a subject in need of such a prevention, treatment or amelioration a pharmaceutical composition described herein. Provided is a method of treating cancer in a mammal in need thereof, comprising administering to the mammal a composition comprising an effective amount of the pharmaceutical composition described herein, optionally in combination with other pharmaceutically active molecules. In certain embodiments said cancer is a solid tumor. In some other embodiments, said solid tumor is one or more of sarcoma, carcinoma, and lymphoma. In certain other embodiments, the cancer is a hematological cancer. In a further embodiment, the cancer is one or more of B-cell lymphoma, non-Hodgkin's lymphoma, and leukemia.

Provided is a method of treating cancer cells comprising providing to said cell a composition comprising a heteromultimer described herein. In certain embodiments, said heteromultimer is provided in conjugation with another therapeutic agent.

Provided is a method of treating a cancer non-responsive to at least one of a CD19 lytic antibody, a CD20 lytic antibody and blinatumomab, in a mammal in need thereof, comprising administering to the mammal a composition comprising an effective amount of the pharmaceutical composition described herein.

Provided is a method of treating a cancer cell regressive after treatment with blinatumomab, comprising providing to said cancer cell a composition comprising an effective amount of the pharmaceutical composition described herein.

Provided is a method of treating an individual suffering from a disease characterized by expression of B cells, said method comprising providing to said individual an effective amount of a composition comprising an effective amount of the pharmaceutical composition described herein. In certain embodiments, said disease is not responsive to treatment with at least one of an anti-CD19 antibody and an anti-CD20 antibody.

Provided is a method of treating an autoimmune condition in a mammal in need thereof, comprising administering to said mammal a composition comprising an effective amount of the pharmaceutical composition provided herein. In certain embodiments, said autoimmune condition is one or more of multiple sclerosis, rheumatoid arthritis, lupus erytematosus, psoriatic arthritis, psoriasis, vasculitis, uveitis, Crohn's disease, and type 1 diabetes.

Provided is a method of treating an inflammatory condition in a mammal in need thereof, comprising administering to said mammal a composition comprising an effective amount of the pharmaceutical composition comprising an heteromultimer provided herein.

Provided is a kit comprising a heteromultimer as defined herein, and instructions for use thereof.

Provided herein is a heteromultimer construct comprising: a first monomer comprising a first transporter polypeptide fused to a first cargo polypeptide that comprises at least one HER2 binding domain; a second monomer comprising a second transporter polypeptide which is different from said first transporter polypeptide, fused to a second cargo polypeptide that comprises at least one HER3 binding domain; wherein said first cargo polypeptide does not have any binding domain present in said second cargo polypeptide; wherein said first and second transporter polypeptide form a heterodimeric Fc region comprising a variant immunoglobulin CH3 region comprising at least one amino acid mutation that promotes the formation of said heterodimer with stability comparable to a native homodimeric Fc.

Provided herein is a heteromultimer construct comprising: a first monomer comprising a first transporter polypeptide fused to a first cargo polypeptide that comprises at least one HER2 binding domain; a second monomer comprising a second transporter polypeptide which is different from said first transporter polypeptide, fused to a second cargo polypeptide polypeptide that comprises at least one HER3 binding domain; wherein said first and second transporter polypeptides are obtained by segmentation of albumin such that said transporter polypeptides self-assemble to form quasi-native albumin.

Provided herein is a pharmaceutical composition comprising an insolated multispecific heteromultimer as defined herein, and a suitable excipient. Also provided is a process for the production of such a pharmaceutical composition, said process comprising: culturing a host cell under conditions allowing the expression of a heteromultimer as defined herein; recovering the produced heteromultimer from the culture; and producing the pharmaceutical composition.

Provided herein are host cells comprising nucleic acid encoding a heteromultimer described herein. In certain embodiments, the nucleic acid encoding the first monomeric protein and the nucleic acid encoding the second monomeric protein are present in a single vector. In certain embodiments, the nucleic acid encoding the first monomeric protein and the nucleic acid encoding the second monomeric protein are present in separate vectors.

Also provided is a kit comprising a heteromultimer as defined herein, and instructions for use thereof.

### BRIEF DESCRIPTION OF THE FIGURES

The patent or application file contains at least one drawing executed in color. Copies of this patent or patent application publication with color drawings will be provided by the U.S. Patent and Trademark Office upon request and payment of the necessary fee.

FIG. 1A depicts exemplary schematic representation of heteromultimer constructs provided herein. For instance the Immunoglobulin based Anti-CD3×CD19 constructs demonstrate different aspects of the heteromultimers for instance the cartoon shows the first and second polypeptide constructs wherein the first polypeptide construct comprises a CH3 binding construct (black) and the second polypeptide construct comprises an antigen binding construct (blue). In some embodiments, the antigen binding construct is absent or replaced by a steric modulating construct. Also shown is the Fc heteromultimer formed by the variant CH3 regions of the first and second polypeptide constructs. FIG. 1B shows the ability of a heteromultimer construct described herein (v873) and a construct that does not comprise a heteromultimer Fc (blinatumomab CD19-CD3 BiTE (v891, MT-103)) to bridge Jurkat CD3 T cells (Top left quadrant) with Raji CD19 B cells (bottom right quadrant) by FACS

FIG. 2 demonstrates that a heteromultimer described herein (v873) is able to selectively bind and bridge to CD3-expressing Jurkat T cells (lower right panel) and to CD19-expressing Raji B cells (upper right panel). FIG. 2 also demonstrates that the one-armed anti-CD3 antibody specifically binds to Jurkat T cells (lower middle panels) and does not cross-react to CD19 expressing B cells, (upper middle panels) and that the one-armed anti-CD19 antibody specifically binds to Raji B cells (upper left panel) and does not cross-react to Jurkat T cells (lower left panel).

FIG. 3A depict the ability of a heteromultimer described herein (v873) to redirect IL-2 activated PBMC to kill target Raji B cells from 3 donors. FIG. 3B demonstrates that a heteromultimer described herein is able to mediate higher redirected T-cell cytotoxicity than a construct lacking the heterodimeric Fc in one of the donors.

FIG. 4 shows that heteromultimers described herein are able to bind to CD3-expressing Jurkat T-cells and to CD19-expressing Raji B-cells.

FIG. 5A indicates that at the concentration tested, v1092, which is a heteromultimer described herein, was able to bridge 31% of total cells, and v873 another heteromultimer construct described herein was able to bridge 25% of total cells. FIG. 5B demonstrates that v1092 is able to bridge Jurkat T cells and Raji B cells to a greater extent than v221 and similar to v891 and v873.

FIG. 6 shows antibody therapeutics that can be provided along with a heteromultimer described herein for treatment of certain indications.

FIG. 7 shows an SDS-PAGE demonstrating that examplary heteromultimer constructs described herein are expressed transiently in CHO3E7 cells with a cell viability of >80%.

FIG. 8 shows that a heteromultimer described herein (v873) induces are higher % cytotoxicity to target B cells when compared to negative control human IgG1 (G1) when comparing across individual donors.

FIG. 9A shows that human IgG (hIgG) does not bind to Jurkat T-cells and has low level binding to Raji B-cells. FIG. 9A also shows that anti-CD19 one arm constructs bind selectively to the Raji B-cells and does not cross-react to Jurkat T cells. FIG. 9B demonstrates FACS assay shows that v873-a heteromultimer described herein, binds selectively to Jurkat T-cells and to Raji B-cells.

FIG. 10 shows that v873-a heteromultimer described herein does not bind to the K562 cell line, which does not express CD19 or CD3.

FIG. 11 shows that v873-a heteromultimer described herein does not bind to mouse lymphoid cells which does not express CD19 or CD3.

FIG. 12A and FIG. 12B show FACS binding curves of heteromultimer constructs described herein (v873, v875) and the construct lacking a heterodimeric Fc (v891), to CD3 expressing HPB-ALL and CD3 expressing Jurkat T cells, and to CD19 expressing Raji B cells.

FIG. 13A illustrates the FACS binding curves for heteromultimer constructs v875, v1379, v1380, v1381, and control v891 binding to CD19 expressing Raji cells tested in 0.1 to 300 nM range. FIG. 13B illustrates the FACS binding curve for heteromultimer constructs v875, v1379, v1380, binding to HBP-ALL T cells tested in 0.1 to 300 nM range.

FIG. 14 indicates that heteomultimer constructs described herein (v875 and v891) facilitate comparable bridging between Raji B-cells and Jurkat T-cells. Use of the control human IgG resulted in 2.5% bridging between Raji and Jurkat cells, while v875 facilitated bridging of 22.9% of total cells, and v891 facilitated bridging of 14.5% of total cells.

FIG. 15A shows the amount of bridging using a 1:1 ratio of T-cells to B-cells, with heteromultimer concentrations ranging from 0.3 nM to 3 nM. FIG. 15B shows the amount of bridging using a 15:1 ratio of T-cells to B-cells, with heteromultimer concentrations ranging from 0.3 nM to 3 nM. Both E:T ratios (1:1 and 15:1) tested with v875 resulted in similar total T cell-B cell bridging when expressed as fold over background.

FIG. 16 depicts the percent cytotoxicity induced by test items usind CD4 and CD8 effectors with and without IL-2 induction.

FIG. 17A illustrates that Fc blocking of IL-2 activated PBMC results in a minor (v875) or no (v873) reduction in the % cytotoxicity of target Raji B cells. FIG. 17B illustrates that Fc blocking of resting PBMC results in a reduction in the % cytotoxicity of target Raji B cells for v875 and v873.

FIG. 18A illustrates that Fc blocking of IL-2 activated PBMC results in a reduction in the % cytotoxicity of target Raji B cells at all antibody concentrations tested for v875 and v873. FIG. 18B illustrates that Fc blocking of resting PBMC results in a reduction in the % cytotoxicity of target Raji B cells at all antibody concentrations tested for v875 and v873.

FIG. 19A shows that v875 and v873 elicit >30% cytotoxicity to target Raji B cells with IL-2 activated CD8+ T cells as effectors, and maximal target cell killing is seen at the 3 nM concentration. FIG. 19B shows that v875 and v873 elicit dose dependent (>20%) cytotoxicity to target Raji B cells with resting CD8+ T cells as effectors.

FIG. 20A shows the target Raji B cell cytotoxicty of v875 with IL-2 activated CD4+ and CD8+ T cells. FIG. 20B shows the target Raji B cell cytotoxicty of v875 with resting CD4+ and CD8+ T cells.

FIG. 21 shows, relative to untreated media and human IgG controls, that v875 and v873 (300 nM) mediate autologous B cell killing in total resting PBMC and total IL-2 activated PBMC.

FIG. 22 shows, relative to untreated media and human IgG controls, v875 has a more selective B cell killing by sparing more autologous T cells compared to v873 and v891.

FIG. 23A shows the effects of v875 on the viability of CD20+, CD4+, CD8+ subsets in IL-2 activated cell cultures. FIG. 23B shows the effects of v875 on the viability of CD20+, CD4+, CD8+ subsets resting cell cultures. FIG. 23C shows the effects of v1379 and v1380 on the viability of CD20+, CD4+, CD8+ subsets in IL-2 activated cell cultures. FIG. 23D shows the effects of v1379 and v1380 on the viability of CD20+, CD4+, CD8+ subsets resting cell cultures.

FIG. 24A shows the results of antibody mediated LDH release in resting effector and Raji B cells. Results of antibody mediated LDH release in activated effector are shown in FIG. 24B.

FIG. 25A illustrates the mediation of ADCC by rituximab and by a heteromultimer described herein with a WT Fc (v875) (ca. 40% max cell lysis). FIG. 25B shows that v1379 which is a heteromultimer described herein with a WT Fc can mediate ADCC while v1380, with a L234A_L235A knock Fc mutation, is impaired in ADCC to target Daudi B cells. FIG. 25C and FIG. 25D show the results of the CDC assay with v1380 and v1379 (FIG. 25C) and v875 (FIG. 25D) of target Daudi B cells with comparisons to positive control Rituximab.

FIG. 26 shows that at 0.3 nM, v875 and v1380 do not induce PBMC proliferation compared to human IgG. The lower panel of FIG. 26 shows the results of the 100 nM antibody concentrations, and shows that v875, v1380 and v891 induce higher cell proliferation relative to human IgG. FIG. 26 (lower panel) also shows that at 100 nM, v875 has a similar proliferative index compared to anti-CD3 OKT3 in all four PBMC populations.

FIG. 27A, FIG. 27B, FIG. 27C, FIG. 27D, and FIG. 27E show that v1380 (L234A_L235A Fc knockout) induces less cytokine release of TNFα, INFγ, IL-2, IL-4, and IL-10 when compared to v875 (WT Fc) and OKT3. The results from the cytokine release assay as shown in FIG. 27A-27E include summary plots of PBMC supernatant TNFα (FIG. 27A) INFγ (FIG. 27B), IL-2 (FIG. 27C), IL-4 (FIG. 27D), and IL-10 (FIG. 27E) levels following incubation with test items at 0.3 nM concentrations for 4 days (graph y-axis represents log cytokine levels in pg per mL from 4 donors).

FIG. 28A and FIG. 28B show the results from the average stimulation index induced by v875 at 0.3 nM (FIG. 28A) and 100 nM (FIG. 28B) concentrations on purified CD8+ T cells in the absence or presence of purified CD19+ B cells at 4 days incubation time-point.

FIG. 29A and FIG. 29B show the results from the average stimulation index induced by v1380 at 0.3 nM (FIG. 29A) and 100 nM (FIG. 29B) concentrations on purified CD8+ T cells in the absence or presence of purified CD19+ B cells at 4 days incubation time-point.

FIG. 30A, FIG. 30B, and FIG. 30C show the results from the T:B cell bridging microscopy comparing v875 and human IgG (3 nM) at 200× and 400× magnification. FIG. 30A shows a direct comparison of human IgG and v875 at 200× magnification and illustrates a higher amount of bridging visible between Raji B cell and Jurkat T cells compared to human IgG. FIG. 30B and FIG. 30C show two fields of view for v875 (FIG. 30B) and human IgG (FIG. 30C) at 400× magnification.

FIG. 31A shows the SDS-PAGE analysis and relative purity of v875, v1380, v1379 and v891 following protein A and SEC purification, and following 47 day storage at 4° C. FIG. 31B shows the SDS-PAGE analysis and relative purity of additional exemplary hetermultimers including v875, v1653, v1654, v1655, v1656, v1660, v1800, and v1802 following protein A and SEC purification.

FIG. 32 shows the LC-MS results of the Max Ent. molecular weight profiles for v875.

FIG. 33A, FIG. 33B, and FIG. 33C show DSC results for heteromultimer constructs described herein, showing that v875 has an estimated CH3 Tm >76° C. (FIG. 33A), v1380 has an estimated CH3 Tm >82.3° C. (FIG. 33B), and v1379 has an estimated CH3 Tm >82.5° C. (FIG. 33C).

FIG. 34A, FIG. 34B, and FIG. 34C show ability of heteromultimers described herein to bridge Raji B and Jurkat T cells (B:T), as well Raji:Raji B cell bridging (B:B) and Jurkat:Jurkat T cell bridging (T:T) assessed by FACS. FIG. 34A shows the amount of T:B, B:B and T:T bridging of v875, v1379, v1380, v891, v1381, commercial OKT3 and human IgG over three experimental replicates. FIG. 34B shows the amount of T:B, B:B and T:T bridging of variants with engineered anti-CD3 warheads for stability enhancement (v1653, v1654, v1655, v1656, v1660, v1800, v1802) and v875, and human IgG. FIG. 34C shows the amount of T:B, B:B and T:T bridging of Fc knock-out variants that have either engineered anti-CD3 warheads for stability enhancement (v1666), or have human/cynomolgous monkey cross-reactive anti-CD3 and anti-CD19 scFvs (v4541, v4543, v4545, v4548) commercial OKT3 anti-CD3 control, v2176 anti-CD19 control and human IgG negative control, and all variants mediate low T:T bridging.

FIG. 35 illustrates binding of heteromultimers described herein to human CD3 (top panel) and binding to the cynomologous CD3 receptor (bottom panel) as determined by ELISA.

FIG. 36A and FIG. 36B illustrates affinity on linear and log scales respectively of HER2/HER3 het-Fc construct demonstrating both bi-specificity and avidity.

FIG. 37 depicts the binding of variant 1090 compared to the control 1087 in MALME-3M cells and indicates that v1090 has similar binding as v1087 to target MALME-3M cells.

### DETAILED DESCRIPTION OF THE INVENTION

Unless defined otherwise, all technical and scientific terms used herein have the same meaning as is commonly understood by one of skill in the art to which the claimed subject matter belongs. In the event that there are a plurality of definitions for terms herein, those in this section prevail. Where reference is made to a URL or other such identifier or address, it is understood that such identifiers can change and particular information on the internet can come and go, but equivalent information can be found by searching the internet. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise.

Terms understood by those in the art of antibody technology are each given the meaning acquired in the art, unless expressly defined differently herein. Antibodies are known to have variable regions, a hinge region, and constant domains. Immunoglobulin structure and function are reviewed, for example, in Harlow et al, Eds., Antibodies: A Laboratory Manual, Chapter 14 (Cold Spring Harbor Laboratory, Cold Spring Harbor, 1988).

In the present description, any concentration range, percentage range, ratio range, or integer range is to be understood to include the value of any integer within the recited range and, when appropriate, fractions thereof (such as one tenth and one hundredth of an integer), unless otherwise indicated. As used herein, “about” means±10% of the indicated range, value, sequence, or structure, unless otherwise indicated. It should be understood that the terms “a” and “an” as used herein refer to “one or more” of the enumerated components unless otherwise indicated or dictated by its context. The use of the alternative (e.g., “or”) should be understood to mean either one, both, or any combination thereof of the alternatives. As used herein, the terms “include” and “comprise” are used synonymously. In addition, it should be understood that the individual single chain polypeptides or immunoglobulin constructs derived from various combinations of the structures and substituents described herein are disclosed by the present application to the same extent as if each single chain polypeptide or heterodimer were set forth individually. Thus, selection of particular components to form individual single chain polypeptides or heterodimers is within the scope of the present disclosure

The section headings used herein are for organizational purposes only and are not to be construed as limiting the subject matter described. All documents, or portions of documents, cited in the application including, but not limited to, patents, patent applications, articles, books, manuals, and treatises are hereby expressly incorporated by reference in their entirety for any purpose.

It is to be understood that the methods and compositions described herein are not limited to the particular methodology, protocols, cell lines, constructs, and reagents described herein and as such may vary. It is also to be understood that the terminology used herein is for the purpose of describing particular embodiments only, and is not intended to limit the scope of the methods and compositions described herein, which will be limited only by the appended claims.

All publications and patents mentioned herein are incorporated herein by reference in their entirety for the purpose of describing and disclosing, for example, the constructs and methodologies that are described in the publications, which might be used in connection with the methods, compositions and compounds described herein. The publications discussed herein are provided solely for their disclosure prior to the filing date of the present application. Nothing herein is to be construed as an admission that the inventors described herein are not entitled to antedate such disclosure by virtue of prior invention or for any other reason.

In the present application, amino acid names and atom names (e.g. N, O, C, etc.) are used as defined by the Protein DataBank (PDB) (www.pdb.org), which is based on the IUPAC nomenclature (IUPAC Nomenclature and Symbolism for Amino Acids and Peptides (residue names, atom names etc.), Eur. J. Biochem., 138, 9-37 (1984) together with their corrections in Eur. J. Biochem., 152, 1 (1985).

The terms “polypeptide,” “peptide” and “protein” are used interchangeably herein to refer to a polymer of amino acid residues. That is, a description directed to a polypeptide applies equally to a description of a peptide and a description of a protein, and vice versa. The terms apply to naturally occurring amino acid polymers as well as amino acid polymers in which one or more amino acid residues is a non-naturally encoded amino acid. As used herein, the terms encompass amino acid chains of any length, including full length proteins, wherein the amino acid residues are linked by covalent peptide bonds.

The term “nucleotide sequence” is intended to indicate a consecutive stretch of two or more nucleotide molecules. The nucleotide sequence may be of genomic, cDNA, RNA, semisynthetic or synthetic origin, or any combination thereof.

The term “polymerase chain reaction” or “PCR” generally refers to a method for amplification of a desired nucleotide sequence in vitro, as described, for example, in U.S. Pat. No. 4,683,195. In general, the PCR method involves repeated cycles of primer extension synthesis, using oligonucleotide primers capable of hybridising preferentially to a template nucleic acid.

“Cell”, “host cell”, “cell line” and “cell culture” are used interchangeably herein and all such terms should be understood to include progeny resulting from growth or culturing of a cell. “Transformation” and “transfection” are used interchangeably to refer to the process of introducing DNA into a cell.

The term “amino acid” refers to naturally occurring and non-naturally occurring amino acids, as well as amino acid analogs and amino acid mimetics that function in a manner similar to the naturally occurring amino acids. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, praline, serine, threonine, tryptophan, tyrosine, and valine) and pyrrolysine and selenocysteine. Amino acid analogs refers to compounds that have the same basic chemical structure as a naturally occurring amino acid, i.e., an a carbon that is bound to a hydrogen, a carboxyl group, an amino group, and an R group, such as, homoserine, norleucine, methionine sulfoxide, methionine methyl sulfonium. Such analogs have modified R groups (such as, norleucine) or modified peptide backbones, but retain the same basic chemical structure as a naturally occurring amino acid. Reference to an amino acid includes, for example, naturally occurring proteogenic L-amino acids; D-amino acids, chemically modified amino acids such as amino acid variants and derivatives; naturally occurring non-proteogenic amino acids such as β-alanine, ornithine, etc.; and chemically synthesized compounds having properties known in the art to be characteristic of amino acids. Examples of non-naturally occurring amino acids include, but are not limited to, α-methyl amino acids (e.g. α-methyl alanine), D-amino acids, histidine-like amino acids (e.g., 2-amino-histidine, β-hydroxy-histidine, homohistidine), amino acids having an extra methylene in the side chain (“homo” amino acids), and amino acids in which a carboxylic acid functional group in the side chain is replaced with a sulfonic acid group (e.g., cysteic acid). The incorporation of non-natural amino acids, including synthetic non-native amino acids, substituted amino acids, or one or more D-amino acids into the proteins of the present invention may be advantageous in a number of different ways. D-amino acid-containing peptides, etc., exhibit increased stability in vitro or in vivo compared to L-amino acid-containing counterparts. Thus, the construction of peptides, etc., incorporating D-amino acids can be particularly useful when greater intracellular stability is desired or required. More specifically, D-peptides, etc., are resistant to endogenous peptidases and proteases, thereby providing improved bioavailability of the molecule, and prolonged lifetimes in vivo when such properties are desirable. Additionally, D-peptides, etc., cannot be processed efficiently for major histocompatibility complex class II-restricted presentation to T helper cells, and are therefore, less likely to induce humoral immune responses in the whole organism.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, “conservatively modified variants” refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of ordinary skill in the art will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of ordinary skill in the art will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the deletion of an amino acid, addition of an amino acid, or substitution of an amino acid with a chemically similar amino acid. Conservative substitution tables providing functionally similar amino acids are known to those of ordinary skill in the art. Such conservatively modified variants are in addition to and do not exclude polymorphic variants, interspecies homologs, and alleles of the invention.

Conservative substitution tables providing functionally similar amino acids are known to those of ordinary skill in the art. The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine(S), Threonine (T); and 8) Cysteine (C), Methionine (M)
(see, e.g., Creighton, Proteins: Structures and Molecular Properties (W H Freeman & Co.; 2nd edition (December 1993)

The terms “identical” or percent “identity,” in the context of two or more nucleic acids or polypeptide sequences, refer to two or more sequences or subsequences that are the same. Sequences are “substantially identical” if they have a percentage of amino acid residues or nucleotides that are the same (i.e., about 50% identity, about 55% identity, 60% identity, about 65%, about 70%, about 75%, about 80%, about 85%, about 90%, or about 95% identity over a specified region), when compared and aligned for maximum correspondence over a comparison window, or designated region as measured using one of the following sequence comparison algorithms (or other algorithms available to persons of ordinary skill in the art) or by manual alignment and visual inspection. This definition also refers to the complement of a test sequence. The identity can exist over a region that is at least about 50 amino acids or nucleotides in length, or over a region that is 75-100 amino acids or nucleotides in length, or, where not specified, across the entire sequence of a polynucleotide or polypeptide. A polynucleotide encoding a polypeptide of the present invention, including homologs from species other than human, may be obtained by a process comprising the steps of screening a library under stringent hybridization conditions with a labeled probe having a polynucleotide sequence of the invention or a fragment thereof, and isolating full-length cDNA and genomic clones containing said polynucleotide sequence. Such hybridization techniques are well known to the skilled artisan.

A derivative, or a variant of a polypeptide is said to share “homology” or be “homologous” with the peptide if the amino acid sequences of the derivative or variant has at least 50% identity with a 100 amino acid sequence from the original peptide. In certain embodiments, the derivative or variant is at least 75% the same as that of either the peptide or a fragment of the peptide having the same number of amino acid residues as the derivative. In certain embodiments, the derivative or variant is at least 85% the same as that of either the peptide or a fragment of the peptide having the same number of amino acid residues as the derivative. In certain embodiments, the amino acid sequence of the derivative is at least 90% the same as the peptide or a fragment of the peptide having the same number of amino acid residues as the derivative. In some embodiments, the amino acid sequence of the derivative is at least 95% the same as the peptide or a fragment of the peptide having the same number of amino acid residues as the derivative. In certain embodiments, the derivative or variant is at least 99% the same as that of either the peptide or a fragment of the peptide having the same number of amino acid residues as the derivative.

The term “bispecific” is intended to include any agent, e.g., heteromultimer, monomer, protein, peptide, or protein or peptide complex, which has two different binding specificities. For example, in some embodiments, the molecule may bind to, or interact with, (a) a cell surface target molecule and (b) an Fc receptor on the surface of an effector cell. In certain embodiments of a heteromultimer described herein, at least one monomer is bispecific formed by attaching to the same transporter polypeptide, two cargo molecules with different binding specificities. In certain embodiments of a heteromultimer described herein, the heteromultimer is itself bispecific formed by attaching to the transporter polypeptides, at least two cargo molecules with different specificities.

The term “multispecific” or “heterospecific” is intended to include any agent, e.g., a protein, peptide, or protein or peptide complex, which has more than two different binding specificities. For example, the molecule may bind to, or interact with, (a) a cell surface target molecule such as but not limited to cell surface antigens, (b) an Fc receptor on the surface of an effector cell, and optionally (c) at least one other component. Accordingly, embodiments of the heteromultimers described herein, are inclusive of, but not limited to, bispecific, trispecific, tetraspecific, and other multispecific molecules. In certain embodiments, these molecules are directed to cell surface antigens, such as CD30, and to other targets, such as Fc receptors on effector cells.

As used herein, “isolated” heteromultimer means a heteromultimer that has been identified and separated and/or recovered from a component of its natural cell culture environment. Contaminant components of its natural environment are materials that would interfere with diagnostic or therapeutic uses for the heteromultimer, and may include enzymes, hormones, and other proteinaceous or non-proteinaceous solutes.

The phrase “selectively (or specifically) hybridizes to” refers to the binding, duplexing, or hybridizing of a molecule only to a particular nucleotide sequence under stringent hybridization conditions when that sequence is present in a complex mixture (including but not limited to, total cellular or library DNA or RNA).

The phrase “stringent hybridization conditions” refers to hybridization of sequences of DNA, RNA, or other nucleic acids, or combinations thereof under conditions of low ionic strength and high temperature as is known in the art. Typically, under stringent conditions a probe will hybridize to its target subsequence in a complex mixture of nucleic acid (including but not limited to, total cellular or library DNA or RNA) but does not hybridize to other sequences in the complex mixture. Stringent conditions are sequence-dependent and will be different in different circumstances. Longer sequences hybridize specifically at higher temperatures. An extensive guide to the hybridization of nucleic acids is found in Tijssen, Laboratory Techniques in Biochemistry and Molecular Biology—Hybridization with Nucleic Probes, “Overview of principles of hybridization and the strategy of nucleic acid assays” (1993).

As used herein, an “antibody” or “immunoglobulin” refers to a polypeptide substantially encoded by an immunoglobulin gene or immunoglobulin genes, or fragments thereof, which specifically bind and recognize an analyte (antigen). The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD, and IgE, respectively.

An exemplary immunoglobulin (antibody) structural unit is composed of two pairs of polypeptide chains, each pair having one “light” (about 25 kD) and one “heavy” chain (about 50-70 kD). The N-terminal domain of each chain defines a variable region of about 100 to 110 or more amino acids primarily responsible for antigen recognition. The terms variable light chain (VL) and variable heavy chain (VH) refer to these light and heavy chain domains respectively. The IgG1 heavy chain comprises of the VH, CH1, CH2 and CH3 domains respectively from the N to C-terminus. The light chain comprises of the VL and CL domains from N to C terminus. The IgG1 heavy chain comprises a hinge between the CH1 and CH2 domains. In certain embodiments, the immunoglobulin constructs comprise at least one immunoglobulin domain from IgG, IgM, IgA, IgD, or IgE connected to a therapeutic polypeptide. In some embodiments, the immunoglobulin domain comprised in an immunoglobulin construct provided herein, is from an immunoglobulin based construct such as a diabody, or a nanobody. In certain embodiments, the immunoglobulin constructs described herein comprise at least one immunoglobulin domain from a heavy chain antibody such as a camelid antibody. In certain embodiments, the immunoglobulin constructs provided herein comprise at least one immunoglobulin domain from a mammalian antibody such as a bovine antibody, a human antibody, a camelid antibody, a mouse antibody or any chimeric antibody.

As used herein, the term “antigenic determinant” is synonymous with “antigen” and “epitope,” and refers to a site (e.g. a contiguous stretch of amino acids or a conformational configuration made up of different regions of non-contiguous amino acids) on a polypeptide macromolecule to which an antigen binding moiety binds, forming an antigen binding moiety-antigen complex. Useful antigenic determinants can be found, for example, on the surfaces of tumor cells, on the surfaces of virus-infected cells, on the surfaces of other diseased cells, on the surface of immune cells, free in blood serum, and/or in the extracellular matrix (ECM). The proteins referred to as antigens herein (e.g. MCSP, FAP, CEA, EGFR, CD33, CD3) can be any native form the proteins from any vertebrate source, including mammals such as primates (e.g. humans) and rodents (e.g. mice and rats), unless otherwise indicated. In a particular embodiment the antigen is a human protein. Where reference is made to a specific protein herein, the term encompasses the “full-length”, unprocessed protein as well as any form of the protein that results from processing in the cell. The term also encompasses naturally occurring variants of the protein, e.g. splice variants or allelic variants. Exemplary human proteins useful as antigens include, but are not limited to: Melanoma-associated Chondroitin Sulfate Proteoglycan (MCSP), also known as Chondroitin Sulfate Proteoglycan 4 (UniProt no. Q6UVK1 (version 70), NCBI RefSeq no. NP 001888.2); Fibroblast Activation Protein (FAP), also known as Seprase (Uni Prot nos. Q12884, Q86Z29, Q99998, NCBI Accession no. NP 004451); Carcinoembroynic antigen (CEA), also known as Carcinoembryonic antigen-related cell adhesion molecule 5 (UniProt no. P06731 (version 119), NCBI RefSeq no. NP 004354.2); CD33, also known as gp67 or Siglec-3 (UniProt no. P20138, NCBI Accession nos. NP 001076087, NP 001171079); Epidermal Growth Factor Receptor (EGFR), also known as ErbB-1 or HerI (UniProt no. P0053, NCBI Accession nos. NP 958439, NP 958440), and CD3, particularly the epsilon subunit of CD3 (see UniProt no. P07766 (version 130), NCBI RefSeq no. NP 000724.1, for the human sequence; or UniProt no. Q95LI5 (version 49), NCBI GenBank no. BAB71849.1, for the cynomolgus [Macaca fascicularis] sequence). In certain embodiments the T cell activating bispecific antigen binding molecule of the invention binds to an epitope of an activating T cell antigen or a target cell antigen that is conserved among the activating T cell antigen or target antigen from different species.

By “specific binding” or “selective binding” is meant that the binding is selective for the antigen and can be discriminated from unwanted or non-specific interactions. The ability of an antigen binding moiety to bind to a specific antigenic determinant can be measured either through an enzyme-linked immunosorbent assay (ELISA) or other techniques familiar to one of skill in the art, e.g. surface plasmon resonance (SPR) technique (analyzed on a BIAcore instrument) (Liljeblad et al, Glyco J 17, 323-329 (2000)), and traditional binding assays (Heeley, Endocr Res 28, 217-229 (2002)). In one embodiment, the extent of binding of an antigen binding moiety to an unrelated protein is less than about 10% of the binding of the antigen binding moiety to the antigen as measured, e.g., by SPR. In certain embodiments, an antigen binding moiety that binds to the antigen, or an antigen binding molecule comprising that antigen binding moiety, has a dissociation constant (K_{D}) of <1 μM, <100 nM, <10 nM, <1 nM, <0.1 nM, <0.01 nM, or <0.001 nM (e.g. 10^{˜8} M or less, e.g. from 10^{˜8} M to 10^{″13} M, e.g., from 10^{″9} M to 10^{″13} M).

“Affinity” refers to the strength of the sum total of non-covalent interactions between a single binding site of a molecule (e.g., a receptor) and its binding partner (e.g., a ligand). Unless indicated otherwise, as used herein, “binding affinity” refers to intrinsic binding affinity which reflects a 1:1 interaction between members of a binding pair (e.g., an antigen binding moiety and an antigen, or a receptor and its ligand). The affinity of a molecule X for its partner Y can generally be represented by the dissociation constant (K_{D}), which is the ratio of dissociation and association rate constants (k_{off} and kₒₙ, respectively). Thus, equivalent affinities may comprise different rate constants, as long as the ratio of the rate constants remains the same. Affinity can be measured by well established methods known in the art, including those described herein. A particular method for measuring affinity is Surface Plasmon Resonance (SPR).

“Reduced binding”, for example reduced binding to an Fc receptor, refers to a decrease in affinity for the respective interaction, as measured for example by SPR. For clarity the term includes also reduction of the affinity to zero (or below the detection limit of the analytic method), i.e. complete abolishment of the interaction. Conversely, “increased binding” refers to an increase in binding affinity for the respective interaction.

An “activating T cell antigen” as used herein refers to an antigenic determinant expressed on the surface of a T lymphocyte, particularly a cytotoxic T lymphocyte, which is capable of inducing T cell activation upon interaction with an antigen binding molecule. Specifically, interaction of an antigen binding molecule with an activating T cell antigen may induce T cell activation by triggering the signaling cascade of the T cell receptor complex. In a particular embodiment the activating T cell antigen is CD3.

“T cell activation” as used herein refers to one or more cellular response of a T lymphocyte, particularly a cytotoxic T lymphocyte, selected from: proliferation, differentiation, cytokine secretion, cytotoxic effector molecule release, cytotoxic activity, and expression of activation markers. The T cell activating bispecific antigen binding molecules of the invention are capable of inducing T cell activation. Suitable assays to measure T cell activation are known in the art described herein.

A “target cell antigen” as used herein refers to an antigenic determinant presented on the surface of a target cell, for example a B cell in a tumor such as a cancer cell or a cell of the tumor stroma. As used herein, the terms “first” and “second” with respect to antigen binding moieties etc., are used for convenience of distinguishing when there is more than one of each type of moiety. Use of these terms is not intended to confer a specific order or orientation of the T cell activating bispecific antigen binding molecule unless explicitly so stated.

A “Fab molecule” refers to a protein consisting of the VH and CH1 domain of the heavy chain (the “Fab heavy chain”) and the VL and CL domain of the light chain (the “Fab light chain”) of an immunoglobulin.

By “fused” is meant that the components (e.g. a Fab molecule and an Fc domain subunit) are linked by peptide bonds, either directly or via one or more peptide linkers.

As used herein, the term “single-chain” refers to a molecule comprising amino acid monomers linearly linked by peptide bonds. In certain embodiments, one of the antigen binding moieties is a single-chain Fab molecule, i.e. a Fab molecule wherein the Fab light chain and the Fab heavy chain are connected by a peptide linker to form a single peptide chain. In a particular such embodiment, the C-terminus of the Fab light chain is connected to the N-terminus of the Fab heavy chain in the single-chain Fab molecule. In certain other embodiments, one of the antigen binding moieties is a single-chain Fv molecule

By a “crossover” Fab molecule (also termed “Crossfab”) is meant a Fab molecule wherein either the variable regions or the constant regions of the Fab heavy and light chain are exchanged, i.e. the crossover Fab molecule comprises a peptide chain composed of the light chain variable region and the heavy chain constant region, and a peptide chain composed of the heavy chain variable region and the light chain constant region. For clarity, in a crossover Fab molecule wherein the variable regions of the Fab light chain and the Fab heavy chain are exchanged, the peptide chain comprising the heavy chain constant region is referred to herein as the “heavy chain” of the crossover Fab molecule. Conversely, in a crossover Fab molecule wherein the constant regions of the Fab light chain and the Fab heavy chain are exchanged, the peptide chain comprising the heavy chain variable region is referred to herein as the “heavy chain” of the crossover Fab molecule.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1 (L1)-FR2-H2 (L2)-FR3-H3 (L3)-FR4.

The “class” of an antibody or immunoglobulin refers to the type of constant domain or constant region possessed by its heavy chain. There are five major classes of antibodies: IgA, IgD, IgE,

IgG, and IgM, and several of these may be further divided into subclasses (isotypes), e.g., IgGi, IgG2, IgG3, IgG4, IgAi, and IgA2. The heavy chain constant domains that correspond to the different classes of immunoglobulins are called a, 0, E, γ, and μ, respectively.

The term “Fc domain” or “Fc region” herein is used to define a C-terminal region of an immunoglobulin heavy chain that contains at least a portion of the constant region. The term includes native sequence Fc regions and variant Fc regions. Although the boundaries of the Fc region of an IgG heavy chain might vary slightly, the human IgG heavy chain Fc region is usually defined to extend from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al, Sequences of Proteins of Immunological Interest, 5th Ed. Public Health Service, National Institutes of Health, Bethesda, M D, 1991. A “subunit” of an Fc domain as used herein refers to one of the two polypeptides forming the dimeric Fc domain, i.e. a polypeptide comprising C-terminal constant regions of an immunoglobulin heavy chain, capable of stable self-association. For example, a subunit of an IgG Fc domain comprises an IgG CH2 and an IgG CH3 constant domain.

A “modification promoting the association of the first and the second subunit of the Fc domain” is a manipulation of the peptide backbone or the post-translational modifications of an Fc domain subunit that reduces or prevents the association of a polypeptide comprising the Fc domain subunit with an identical polypeptide to form a homodimer. A modification promoting association as used herein particularly includes separate modifications made to each of the two Fc domain subunits desired to associate (i.e. the first and the second subunit of the Fc domain), wherein the promote association of the two Fc domain subunits and the formation of heterodimers. For example in certain embodiments, a modification promoting association may alter the structure or charge of one or both of the Fc domain subunits so as to make their association favorable.

The term “effector functions” refers to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (AD CP), cytokine secretion, immune complex-mediated antigen uptake by antigen presenting cells, down regulation of cell surface receptors (e.g. B cell receptor), and B cell activation.

As used herein, “albumin” refers collectively to albumin protein or amino acid sequence, or an albumin segment or variant, having one or more functional activities (e.g., biological activities) of albumin. In particular “albumin” refers to human albumin or segments thereof (see for example, EP 201 239, EP 322 094 WO 97/24445, WO95/23857) especially the mature form of human albumin, or albumin from other vertebrates, or segments thereof, or analogs or variants of these molecules or fragments thereof. Inc ertain embodiments, albumin refers to a truncated version of albumin.

The term “quasi-albumin” refers to a heteromultimer molecule that has structure and/or function similar to the whole albumin, and wherein said heteromultimer molecule is formed by the assembly of two or more monomeric polypeptides designed based on the sequence of the whole albumin. In certain embodiments, the monomeric polypeptides are “segments” that preferentially associate as heteromultimeric pairs to form a quasi-protein. In some embodiments, the quasi-albumin has 90% of the activity of the whole albumin. In some embodiments, the quasi-albumin has 75% of the activity of whole-albumin. In an embodiment, the quasi-albumin has 50% of the activity of whole albumin. In some embodiments, the quasi-albumin has 50-75% of the activity of whole albumin. In an embodiment, quasi-albumin has 80% of the activity of whole albumin. In some embodiments, the quasi-albumin has 90% of the structure of whole albumin as determined by molecular modeling. In some embodiments, the quasi-albumin has 80% of the structure of whole albumin as determined by molecular modeling. In some embodiments, the quasi-albumin has 70% of the structure of whole albumin as determined by molecular modeling. In some embodiments, the quasi-albumin has 50% of the structure of whole albumin as determined by molecular modeling. In some embodiments, the quasi-albumin has 50%-75% of the structure of whole albumin as determined by molecular modeling.

The terms, human serum albumin (HSA) and human albumin (HA) are used interchangeably herein. The terms, “albumin and serum albumin” are broader, and encompass human serum albumin (and fragments and variants thereof) as well as albumin from other species (and fragments and variants thereof).

In certain embodiments, each albumin-based construct described herein is based on a variant of normal HA. The term “variants” includes insertions, deletions and substitutions, either conservative or non conservative, where such changes do not substantially alter one or more of the oncotic, useful ligand-binding and non-immunogenic properties of albumin, or the active site, or active domain which confers the therapeutic activities of therapeutic proteins.

In certain embodiments, the isolated heteromultimeric constructs described herein include naturally occurring polymorphic variants of human albumin and fragments of human albumin, for example those fragments disclosed in EP 322 094 (namely HA (Pn), where n is 369 to 419).

In certain embodiments, the albumin is derived from any vertebrate, especially any mammal that includes but is not limited to human, cow, sheep, rat, mouse, rabbit, horse, dog or pig. In certain embodiments, the albumin is derived from non-mammalian albumins including, but are not limited to hen and salmon.

The sequence of human albumin is as shown: SEQ ID NO: 271

MKWVTFISLLFLFSSAYSRGVFRRDAHKSEVAHRFKDLGEENFKALVLI
AFAQYLQQCPFEDHVKLVNEVTEFAKTCVADESAENCDKSLHTLFGDKL
CTVATLRETYGEMADCCAKQEPERNECFLQHKDDNPNLPRLVRPEVDVM
CTAFHDNEETFLKKYLYEIARRHPYFYAPELLFFAKRYKAAFTECCQAA
DKAACLLPKLDELRDEGKASSAKQRLKCASLQKFGERAFKAWAVARLSQ
RFPKAEFAEVSKLVTDLTKVHTECCHGDLLECADDRADLAKYICENQDS
ISSKLKECCEKPLLEKSHCIAEVENDEMPADLPSLAADFVESKDVCKNY
AEAKDVFLGMFLYEYARRHPDYSVVLLLRLAKTYETTLEKCCAAADPHE
CYAKVFDEFKPLVEEPQNLIKQNCELFEQLGEYKFQNALLVRYTKKVPQ
VSTPTLVEVSRNLGKVGSKCCKHPEAKRMPCAEDYLSVVLNQLCVLHEK
TPVSDRVTKCCTESLVNRRPCFSALEVDETYVPKEFNAETFTFHADICT
LSEKERQIKKQTALVELVKHKPKATKEQLKAVMDDFAAFVEKCCKADDK
ETCFAEEGKKLVAASQAALGL

An “alloalbumin” is a genetic variant of albumin. In certain embodiments the alloalbumin is human alloalbumin (HAA). Alloalbumins that differ in electrophoretic mobility from albumin have been identified through population genetics surveys in the course of clinical electrophoresis, or in blood donor surveys. As markers of mutation and migration, alloalbumins are of interest to geneticists, biochemists, and anthropologists, but most of these alloalbumin are not associated with disease (Minchioti et al. Human Mutations 29 (8), 1007-1016 (2008)).

TABLE 1
List of substitutions comprised by various alloalbumins as compared
to HA of SEQ ID NO: 271. Thermostability, half-life information and
other HAAs are provided in Krogh-hansen et al. Biochim Biophys Acta
1747, 81-88(2005); and WO2011051489 incorporated by reference herein.

| | Thermostability (C.) | Effect on |
|---|---|---|
| | (positive = stabilizing, | half-life |
| Mutation | negative = destabilizing) | (% change) |
| H3Y | N/A | N/A |
| H3Q | N/A | N/A |
| Q32Stop | N/A | N/A |
| E60K | N/A | N/A |
| D63N | 6.07 | N/A |
| L66P | N/A | N/A |
| E82K | 2.03 | N/A |
| R114G | N/A | N/A |
| R114Stop | N/A | N/A |
| E119K | N/A | N/A |
| V122E | 0.57 | N/A |
| H128R | N/A | N/A |
| Y140C | N/A | N/A |
| A175Stop | N/A | N/A |
| C177F | −1.59 | N/A |
| R218H | N/A | N/A |
| R218P | N/A | N/A |
| K225Q* | −4.86 | N/A |
| K240E | N/A | N/A |
| E244Stop | N/A | N/A |
| Q268R | N/A | N/A |
| D269G | 3.67 | N/A |
| K276N | 4.87 | N/A |
| K313N | −7.16 | N/A |
| D314G | −0.38 | N/A |
| D314V | N/A | N/A |
| N318K | N/A | N/A |
| A320T, & -1R | N/A | 6.16 |
| E321K | 1.42 | N/A |
| E333K | −2.56 | N/A |
| E354K | N/A | N/A |
| E358K | N/A | N/A |
| K359K | −6.56 | N/A |
| D365H | 0.89 | N/A |
| D365V | N/A | N/A |
| E368G | N/A | N/A |
| K372E | N/A | N/A |
| D375N | N/A | N/A |
| D375H | −0.09 | N/A |
| E376K | N/A | N/A |
| E376Q | N/A | N/A |
| E382K | N/A | N/A |
| Q385Stop | N/A | N/A |
| Y401Stop | N/A | N/A |
| R410C | N/A | N/A |
| E479K | N/A | N/A |
| D494N | N/A | 0.84 |
| E501K | 0.13 | N/A |
| E505K | 1.87 | N/A |
| I513N | N/A | N/A |
| V533M | N/A | N/A |
| K536E | N/A | N/A |
| K541E | 6.12 | N/A |
| D550G | N/A | N/A |
| D550A | N/A | N/A |
| K560E | 0.70 | N/A |
| D563N | 4.17 | N/A |
| E565K | N/A | N/A |
| E570K | −6.53 | N/A |
| K573E | 2.08 | 2.7 |
| K574N | N/A | N/A |
| L575inser- | −5.30 | N/A |
| tion(TCCCKSSCLRLITSHLKASQPTMRIRERK) | | |
| (SEQ ID NO: 272)) | | |
| Frameshift after | N/A | −5.7% |
| 567; Stop at 582 | | |
| Frameshift after | N/A | −8.9% |
| 572; Stop at 578 | | |

The term “segmentation” refers to a precise internal splice of the original protein sequence which results in “segments” of the protein sequence that preferentially associate as heteromultimers to form a quasi-protein.

### Quasi-Native Structure:

With reference to a native protein or its structure, quasi-native proteins and/or ‘quasi-native structures’ present the native protein like functional and structural characteristics. Proteins are naturally dynamics molecules and display an ensemble of structural configurations although we ascribe a native structure to it, such as the one obtained by X-ray crystallography. The alternate structural configurations observed in the ensemble of geometries of that protein can be deemed to be quasi-native structures relative to each other or relative to the structure observed in the crystal. On a different front, homologous proteins sequences or proteins belonging to common structural families tend to fold into similar structural geometries. The member proteins belonging to this family can be deemed to achieve a quasi-native structure relative to each other. Some of the unique sequences in the protein family could also exhibit similar functional attributes and hence can be referred to as quasi-native proteins relative to each other. In the case of heteromultimers described here comprising of two or more protein constructs each of which have a transporter polypeptide component, the transporter polypeptides assemble to form a quasi-native structure. The reference native protein in this case is the protein from which the transporter polypeptide is derived and the reference native structure is the structure of the monomeric protein from which the transporter polypeptide is derived. We describe a case where two or more different polypeptides self-assemble to form a heteromultimeric structural and exhibit functional characteristics like a native protein which itself is a monomeric entity. In certain embodiments, are provided heteromultimer constructs comprising transporter polypeptides derived from albumin that self-assemble to form a heteromultimer that exhibits native albumin like functional characteristics such as FcRn binding and structural characteristics. These heteromultimers are referred to as being quasi-native.

“CD3 complex” as described herein is a complex of at least five membrane-bound polypeptides in mature T-lymphocytes that are non-covalently associated with one another and with the T-cell receptor. The CD3 complex includes the gamma, delta, epsilon, zeta, and eta chains (also referred to as subunits). Non-human monoclonal antibodies have been developed against some of these chains, as exemplified by the murine antibodies OKT3, SP34, UCHT1 or 64.1. (See e.g., June, et al., J. Immunol. 136:3945-3952 (1986); Yang, et al., J. Immunol. 137:1097-1100 (1986); and Hayward, et al., Immunol. 64:87-92 (1988)). The expression of certain CD antigens is highly restricted to specific lineage. lymphohematopoietic cells and over the past several years, antibodies directed against lymphoid-specific antigens have been used to develop treatments that were effective either in vitro or in animal models (5-13). In this respect CD19 has proved to be a very useful target. CD19 is expressed in the whole B lineage from the pro B cell to the mature B cell, it is not shed, is uniformly expressed on all lymphoma cells, and is absent from stem cells.

The term “effective amount” as used herein refers to that amount of multispecific heteromultimer construct being administered, which will relieve to some extent one or more of the symptoms of the disease, condition or disorder being treated. Compositions containing a multispecific heteromultimeric construct described herein can be administered for prophylactic, enhancing, and/or therapeutic treatments.

As used herein, the terms “engineer, engineered, engineering”, are considered to include any manipulation of the peptide backbone or the post-translational modifications of a naturally occurring or recombinant polypeptide or fragment thereof. Engineering includes modifications of the amino acid sequence, of the glycosylation pattern, or of the side chain group of individual amino acids, as well as combinations of these approaches. The engineered proteins are expressed and produced by standard molecular biology techniques.

By “isolated nucleic acid molecule or polynucleotide” is intended a nucleic acid molecule, DNA or RNA, which has been removed from its native environment. For example, a recombinant polynucleotide encoding a polypeptide contained in a vector is considered isolated. Further examples of an isolated polynucleotide include recombinant polynucleotides maintained in heterologous host cells or purified (partially or substantially) polynucleotides in solution. An isolated polynucleotide includes a polynucleotide molecule contained in cells that ordinarily contain the polynucleotide molecule, but the polynucleotide molecule is present extrachromosomally or at a chromosomal location that is different from its natural chromosomal location. Isolated RNA molecules include in vivo or in vitro RNA transcripts, as well as positive and negative strand forms, and double-stranded forms. Isolated polynucleotides or nucleic acids described herein, further include such molecules produced synthetically. In addition, a polynucleotide or a nucleic acid, in certain embodiments, include a regulatory element such as a promoter, ribosome binding site, or a transcription terminator.

By a nucleic acid or polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These alterations of the reference sequence may occur at the 5′ or 3′ terminal positions of the reference nucleotide sequence or anywhere between those terminal positions, interspersed either individually among residues in the reference sequence or in one or more contiguous groups within the reference sequence. As a practical matter, whether any particular polynucleotide sequence is at least 80%, 85%, 90%, 95%, 96%, 97%, 98% or 99% identical to a nucleotide sequence of the present invention can be determined conventionally using known computer programs, such as the ones discussed above for polypeptides (e.g. ALIGN-2).

The term “expression cassette” refers to a polynucleotide generated recombinantly or synthetically, with a series of specified nucleic acid elements that permit transcription of a particular nucleic acid in a target cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. In certain embodiments, the expression cassette of the invention comprises polynucleotide sequences that encode bispecific antigen binding molecules of the invention or fragments thereof.

The term “vector” or “expression vector” is synonymous with “expression construct” and refers to a DNA molecule that is used to introduce and direct the expression of a specific gene to which it is operably associated in a target cell. The term includes the vector as a self-replicating nucleic acid structure as well as the vector incorporated into the genome of a host cell into which it has been introduced. The expression vector of the present invention comprises an expression cassette. Expression vectors allow transcription of large amounts of stable mRNA. Once the expression vector is inside the target cell, the ribonucleic acid molecule or protein that is encoded by the gene is produced by the cellular transcription and/or translation machinery. In one embodiment, the expression vector of the invention comprises an expression cassette that comprises polynucleotide sequences that encode bispecific antigen binding molecules of the invention or fragments thereof.

The terms “host cell”, “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. In certain embodiments, progeny are not completely identical in nucleic acid content to a parent cell, but may contain mutations. Mutant progeny that have the same function or biological activity as screened or selected for in the originally transformed cell are included herein. A host cell is any type of cellular system that can be used to generate the bispecific antigen binding molecules of the present invention. Host cells include cultured cells, e.g. mammalian cultured cells, such as CHO cells, BHK cells, NS0 cells, SP2/0 cells, YO myeloma cells, P3X63 mouse myeloma cells, PER cells, PER.C6 cells or hybridoma cells, yeast cells, insect cells, and plant cells, to name only a few, but also cells comprised within a transgenic animal, transgenic plant or cultured plant or animal tissue.

An “activating Fc receptor” is an Fc receptor that following engagement by an Fc domain of an antibody elicits signaling events that stimulate the receptor-bearing cell to perform effector functions. Human activating Fc receptors include FcγRIIIa (CD 16a), FcγRI (CD64), and FcγRIIa (CD32).

Antibody-dependent cell-mediated cytotoxicity (ADCC) is an immune mechanism leading to the lysis of antibody-coated target cells by immune effector cells. The target cells are cells to which antibodies or derivatives thereof comprising an Fc region specifically bind, generally via the protein part that is N-terminal to the Fc region. As used herein, the term “reduced ADCC” is defined as either a reduction in the number of target cells that are lysed in a given time, at a given concentration of antibody in the medium surrounding the target cells, by the mechanism of ADCC defined above, and/or an increase in the concentration of antibody in the medium surrounding the target cells, required to achieve the lysis of a given number of target cells in a given time, by the mechanism of ADCC. The reduction in ADCC is relative to the ADCC mediated by the same antibody produced by the same type of host cells, using the same standard production, purification, formulation and storage methods (which are known to those skilled in the art), but that has not been engineered. For example the reduction in ADCC mediated by an antibody comprising in its Fc domain an amino acid substitution that reduces ADCC, is relative to the ADCC mediated by the same antibody without this amino acid substitution in the Fc domain.

An “effective amount” of an agent such as a multispecific heteromultimer described herein, refers to the amount that is necessary to result in a physiological change in the cell or tissue to which it is administered.

A “therapeutically effective amount” of an agent, e.g. a pharmaceutical composition comprising a multispecific heteromultimer described herein, refers to an amount effective, at dosages and for periods of time necessary, to achieve the desired therapeutic or prophylactic result. A therapeutically effective amount of an agent for example eliminates, decreases, delays, minimizes or prevents adverse effects of a disease.

An “individual” or “subject” is a mammal. Mammals include, but are not limited to, domesticated animals (e.g. cows, sheep, cats, dogs, and horses), primates (e.g. humans and non-human primates such as monkeys), rabbits, and rodents (e.g. mice and rats). Particularly, the individual or subject is a human.

The term “pharmaceutical composition” refers to a preparation which is in such form as to permit the biological activity of a multispecific heteromultimer construct contained therein to be effective, and which contains no additional components which are unacceptably toxic to a subject to which the formulation would be administered.

A “pharmaceutically acceptable carrier” refers to an ingredient in a pharmaceutical composition, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, multispecific heteromultimer constructs described herein are used to delay development of a disease or to slow the progression of a disease. The term “instructions” is used to refer to instructions customarily included in commercial packages of therapeutic products that contain information about the indications, usage, dosage, administration, combination therapy, contraindications and/or warnings concerning the use of such therapeutic products.

The term “cross-species binding” or “interspecies binding” as used herein means binding of a binding domain described herein to the same target molecule in humans and other organisims for instance, but not restricted to non-chimpanzee primates. Thus, “cross-species binding” or “interspecies binding” is to be understood as an interspecies reactivity to the same molecule “X” (i.e. the homolog) expressed in different species, but not to a molecule other than “X”. Cross-species specificity of a monoclonal antibody recognizing e.g. human CD3 epsilon, to a non-chimpanzee primate CD3 epsilon, e.g. macaque CD3 epsilon, can be determined, for instance, by FACS analysis. The FACS analysis is carried out in a way that the respective monoclonal antibody is tested for binding to human and non-chimpanzee primate cells, e.g. macaque cells, expressing said human and non-chimpanzee primate CD3 epsilon antigens, respectively. An appropriate assay is shown in the following examples. The above-mentioned subject matter applies mutatis mutandis for the PSCA, CD19, C-MET, Endosialin, EpCAM, IGF-1R and FAPa antigen: Cross-species specificity of a monoclonal antibody recognizing e.g. human PSCA, CD19, C-MET, Endosialin, EpCAM, IGF-1R or FAPa, to a non-chimpanzee primate PSCA, CD19, C-MET, Endosialin, EpCAM, IGF-1R or FAPa, e.g. macaque PSCA, CD19, C-MET, Endosialin, EpCAM, IGF-1R or FAPa, can be determined, for instance, by FACS analysis. The FACS analysis is carried out in a way that the respective monoclonal antibody is tested for binding to human and non-chimpanzee primate cells, e.g. macaque cells, expressing said human and non-chimpanzee primate PSCA, CD19, C-MET, Endosialin, EpCAM, IGF-1R or FAPa antigens, respectively.

### Detailed Description of Certain Embodiments

### Immunoglobulin Based Multispecific Heteromultimer Constructs:

Provided herein are isolated multispecific heteromultimer constructs comprising a first polypeptide construct comprising a first heavy chain polypeptide and a CD3 binding polypeptide construct that binds to a CD3 complex on at least one CD3 expressing cell; and a second polypeptide construct comprising a second heavy chain polypeptide which is different from said first heavy chain polypeptide, and an antigen binding polypeptide construct that binds to a target antigen on at least one B cell, wherein: at least one of said CD3 binding polypeptide construct and said antigen binding polypeptide construct comprises a single chain Fv region; said multispecific heteromultimer construct simultaneously engages said at least one B cell and said at least one CD3 expressing cell such that the CD3 expressing cell is activated, thereby inducing killing of the B cell; and said first and second heavy chain polypeptides form a heterodimeric Fc region comprising a variant immunoglobulin CH3 region comprising at least one amino acid mutation that promotes the formation of said heterodimeric Fc which is formed with stability at least comparable to a native homodimeric Fc, and with purity such that when said multispecific heteromultimer construct is expressed from a mammalian cell in an expression product, said expression product comprises at least about 70% of said multispecific heteromultimer, and less than 10% monomers or homodimers of said first or second polypeptide constructs. In some embodiments, the expression product comprises at least about 75% of said multispecific heteromultimer, and less than 15% monomers or homodimers of said first or second polypeptide constructs. In certain embodiments, the expression product comprises at least about 80% of said multispecific heteromultimer, and less than 10% monomers or homodimers of said first or second polypeptide constructs. In additional embodiments, the expression product comprises at least about 85% of said multispecific heteromultimer, and less than 10% monomers or homodimers of said first or second polypeptide constructs. In a further embodiment expression product comprises at least about 90% of said multispecific heteromultimer, and less than 10% monomers or homodimers of said first or second polypeptide constructs.

In certain embodiments is the isolated multispecific heteromultimer construct, wherein said first or second polypeptide construct is devoid of at least one of immunoglobulin light chain, and immunoglobulin first constant (CH1) region.

In certain embodiments is provided an isolated multispecific heteromultimer construct comprising: a first polypeptide construct comprising a first heavy chain polypeptide and a CD3 binding polypeptide construct that binds to a CD3 complex on at least one CD3 expressing cell; a second polypeptide construct comprising a second heavy chain polypeptide which is different from said first heavy chain polypeptide, and wherein said second polypeptide construct does not comprise an antigen binding polypeptide construct that binds to a target antigen on a B cell; wherein: said multispecific heteromultimer construct simultaneously engages at least one B cell and said at least one CD3 expressing cell such that the CD3 expressing cell is activated, thereby inducing killing of the B cell; and said first and second heavy chain polypeptides form a heterodimeric Fc region comprising a variant immunoglobulin CH3 region comprising at least one amino acid mutation that promotes the formation of said heterodimeric Fc, wherein: said heterodimeric Fc is formed with stability at least comparable to a native homodimeric Fc, and said heterodimeric Fc is formed with purity such that when said multispecific heteromultimer construct is expressed from a stable mammalian cell in an expression product, said expression product comprises at least about 75% of said multispecific heteromultimer, and less than 10% monomers or homodimers of said first or second polypeptide constructs. In some embodiments, the heterodimeric Fc interacts with cell surface receptors such as FcgRIIb on the B-cell. In certain embodiment, the heterodimeric Fc is engineered to interact preferentially with the FcgRIIb receptor relative to the normal antibody.

Provided herein is an isolated multispecific heteromultimer construct comprising: a first polypeptide construct comprising a first heavy chain polypeptide and a CD3 binding polypeptide construct that binds to a CD3 complex on at least one CD3 expressing cell; a second polypeptide construct comprising a second heavy chain polypeptide which is different from said first heavy chain polypeptide, and a steric modulator construct which exhibits negligible receptor binding; wherein: said multispecific heteromultimer construct simultaneously engages at least one B cell and said at least one CD3 expressing cell such that the CD3 expressing cell is activated, thereby inducing killing of the B cell; and said first and second heavy chain polypeptides form a heterodimeric Fc region comprising a variant immunoglobulin CH3 region comprising at least one amino acid mutation that promotes the formation of said heterodimeric Fc, wherein: said heterodimeric Fc is formed with stability at least comparable to a native homodimeric Fc, and said heterodimeric Fc is formed with purity such that when said multispecific heteromultimer construct is coexpressed from a stable mammalian cell in an expression product, said expression product comprises at least about 75% of said multispecific heteromultimer, and less than 10% monomers or homodimers of said first or second polypeptide constructs. In certain embodiment the steric modulator construct is actually incapable of binding any known target tissue or cell surface and thus functions as a dummy polypeptide arm that only plays a steric modulation role in the interactions of the multispecific heteromultimer construct. In some embodiments, the steric modulator construct is a polypeptide sequence that helps modulate sterical features of the multispecific heteromultimer as the multimer binds to T and/or B cells. In certain embodiments the steric modulator construct comprises a polypeptide domain that is designed de-novo. In certain embodiments, the steric modulator construct comprises polypeptide domains obtained by engineering a known polypeptide domain to remove its binding properties. For instance, in certain embodiments, the steric modulator construct comprises an engineered Fab region or fragment thereof which is engineered to remove binding properties.

Provided is an isolated multispecific heteromultimer construct comprising: a first polypeptide construct comprising a first heavy chain polypeptide and a CD3 binding polypeptide construct that binds to a CD3 complex on at least one CD3 expressing cell; a second polypeptide construct comprising a second heavy chain polypeptide which is different from said first heavy chain polypeptide, and wherein said second polypeptide construct does not comprise an antigen binding polypeptide construct; wherein: said multispecific heteromultimer construct simultaneously engages at least one B cell and said at least one CD3 expressing cell such that the CD3 expressing cell is activated, thereby inducing killing of the B cell; and said first and second heavy chain polypeptides form a heterodimeric Fc region comprising a variant immunoglobulin CH3 region comprising at least one amino acid mutation that promotes the formation of said heterodimeric Fc, wherein: said heterodimeric Fc is formed with stability at least comparable to a native homodimeric Fc, and said heterodimeric Fc is formed with purity such that when said multispecific heteromultimer construct is coexpressed from a stable mammalian cell in an expression product, said expression product comprises at least about 75% of said multispecific heteromultimer, and less than 10% monomers or homodimers of said first or second polypeptide constructs.

In some embodiments is the isolated multispecific heteromultimer construct described herein wherein the heterodimer Fc region comprises a variant CH2 domain or hinge comprising amino acid modifications that prevents functionally effective binding to all the Fcgamma receptors.

Provided are isolated multispecific heteromultimer constructs that bind at least one B cell with a valency greater than one, and simultaneously engage said at least one B cell and at least one CD3 expressing cell such that the CD3 expressing cell is activated, thereby inducing killing of the B cell. In certain embodiments, the multispecific heteromultimer comsprises: a first polypeptide construct comprising a first heavy chain polypeptide and a CD3 binding polypeptide construct that binds to a CD3 complex on at least one CD3 expressing cell; and a second polypeptide construct comprising a second heavy chain polypeptide which is different from said first heavy chain polypeptide, and an antigen binding polypeptide construct that binds to a target antigen on at least one B cell; wherein: at least one of said CD3 binding polypeptide construct and said antigen binding polypeptide construct optionally comprises a single chain Fv region; said first and second heavy chain polypeptides form a heterodimeric Fc region comprising a variant immunoglobulin CH3 region comprising at least one amino acid mutation that promotes the formation of said heterodimeric Fc with stability at least comparable to a native homodimeric Fc, and with purity such that when said multispecific heteromultimer construct is expressed from a mammalian cell in an expression product, said expression product comprises greater than 75% of said multispecific heteromultimer, and less than 10% monomers or homodimers of said first or second polypeptide constructs. The said multispecific heteromultimer construct is capable of interacting with the B-cell via the said antigen binding polypeptide construct on the second heavy chain as well as interaction via the said heterodimeric Fc with FcgRIIb receptors on the B-cell to show valency greater than one during B-cell engagement.

In certain embodiments is the isolated multispecific heteromultimer described herein, wherein the heterodimer Fc region comprises a variant CH2 domain comprising amino acid modifications to promote selective binding of a Fcgamma receptor.

In some embodiments is the heteromultimer wherein the variant CH2 domain selectively binds at least one of FcgammaIIIa and FcgammaIIb receptor as compared to wild-type CH2 domain.

In certain embodiments is an isolated multispecific heteromultimer construct described herein wherein the heterodimer Fc is glycosylated.

In some embodiments is an isolated multispecific heteromultimer described herein, wherein the heterodimer Fc is afucosylated.

In certain embodiments is an isolated multispecific heteromultimer construct described herein wherein the heterodimer Fc is aglycosylated.

Fc region modifications promoting heterodimerization:

Provided herein are multispecific heteromultimer constructs that comprise different antigen binding moieties, fused to one or the other of the two subunits of the Fc domain, thus the two subunits of the Fc domain are typically comprised of two non-identical polypeptide chains. To improve the yield and purity of the heteromultimers described herein, the Fc region of the polypeptides is modified to promote the association of the desired polypeptides.

In some embodiments, the first and second heavy chain polypeptides of the heteromultimer constructs described herein form a heterodimeric Fc region comprising a variant immunoglobulin CH3 region comprising at least one amino acid mutation that promotes the formation of said heterodimeric Fc which is formed with stability at least comparable to a native homodimeric Fc, and with purity such that when said multispecific heteromultimer construct is coexpressed from a mammalian cell as an expression product, said expression product comprises at least about 75% of said multispecific heteromultimer, and less than 10% monomers or homodimers of said first or second polypeptide constructs.

In some embodiments, the first and second heavy chain polypeptides of the heteromultimer constructs described herein form a heterodimeric Fc region comprising a variant immunoglobulin CH3 region comprising at least one amino acid mutation that promotes the formation of said heterodimeric Fc which is formed with stability at least comparable to a native homodimeric Fc, and with purity such that when said multispecific heteromultimer construct is co-expressed from a mammalian cell as an expression product, said expression product comprises at least about 90% of said multispecific heteromultimer, and less than 10% monomers or homodimers of said first or second polypeptide constructs.

In some embodiments, the first and second heavy chain polypeptides of the heteromultimer constructs described herein form a heterodimeric Fc region comprising a variant immunoglobulin CH3 region comprising at least one amino acid mutation that promotes the formation of said heterodimeric Fc which is formed with stability at least comparable to a native homodimeric Fc, and with purity such that when said multispecific heteromultimer construct is co-expressed from a mammalian cell as an expression product, said expression product comprises at least about 95% of said multispecific heteromultimer, and less than 10% monomers or homodimers of said first or second polypeptide constructs.

In some embodiments is the isolated multispecific heteromultimer provided herein, wherein the variant CH3 domain has a melting temperature (Tm) of about 73° C. or greater.

In certain embodiments is the isolated multispecific heteromultimer described herein, wherein the heterodimer Fc region is formed with a purity greater than about 78%.

In am embodiment is the isolated multispecific heteromultimer described herein, wherein the heterodimer Fc region is formed with a purity of at least about 78% or greater and the Tm is at least about 75° C.

In some embodiments is the isolated multispecific heteromultimer described herein, wherein the heterodimer Fc region is formed with a purity of at least about 75% and the Tm is about 75° C. or greater.

In certain embodiments are provided isolated multispecific heteromultimer constructs wherein: a) the variant CH3 sequence of the first heavy chain polypeptide comprises the amino acid modifications L351Y, F405A, and Y407V, and the variant CH3 sequence of the second transporter polypeptide comprises the amino acid modifications T366L, K392M, and T394W; b) the variant CH3 sequence of the first heavy chain polypeptide comprises the amino acid modifications L351Y, F405A, and Y407V, and the variant CH3 sequence of the second heavy chain polypeptide comprises the amino acid modifications T366L, K392L, and T394W; c) the variant CH3 sequence of the first heavy chain polypeptide comprises the amino acid modifications T350V, L351Y, F405A, and Y407V, and the variant CH3 sequence of the second heavy chain polypeptide comprises the amino acid modifications T350V, T366L, K392M, and T394W; d) the variant CH3 sequence of the first heavy chain polypeptide comprises the amino acid modifications T350V, L351Y, F405A, and Y407V, and the variant CH3 sequence of the second heavy chain polypeptide comprises the amino acid modifications T350V, T366L, K392L, and T394W; e) the variant CH3 sequence of the first heavy chain polypeptide comprises the amino acid modifications T366L, N390R, K392R, and T394W, and the variant CH3 sequence of the second heavy chain polypeptide comprises the amino acid modifications L351Y, S400E, F405A, and Y407V; or f) the variant CH3 sequence of the first heavy chain polypeptide comprises the amino acid modifications T350V, T366L, N390R, K392R, and T394W, and the variant CH3 sequence of the second heavy chain polypeptide comprises the amino acid modifications T350V, L351Y, S400E, F405A, and Y407V.

Fc region modifications reducing Fc receptor binding and/or effector function:

In certain embodiments the Fc regions of the heteromultimer constructs described herein comprises a variant CH2 domain comprising amino acid modifications to promote selective binding of a Fcgamma receptor. In some embodiments, the isolated multispecific heteromultimer described herein comprise a variant CH2 domain that selectively binds a FcgammaIIb receptor with an affinity greater than that of the wild-type CH2 domain. In some embodiments, the isolated multispecific heteromultimer described herein comprise a variant CH2 domain that selectively binds a FcgammaIIA and/or FcgammaIIlA receptor with an affinity greater than that of the wild-type CH2 domain.

In certain embodiments the Fc regions of the heteromultimer constructs described herein exhibit reduced binding affinity to an Fc receptor and/or reduced effector function, as compared to a native IgG1 Fc region. In one such embodiment the Fc region exhibits less than 50%, alternatively less than 20%, alternatively less than 10% and in some embodiments, less than 5% of the binding affinity to an Fc receptor, as compared to a native IgG1 Fc region, and/or less than 50%, alternatively less than 20%, alternatively less than 10% and in some embodiments less than 5% of the effector function, as compared to a native IgG1 Fc region.

In one embodiment, the Fc region of a heteromutlimer construct described herein does not substantially bind to an Fc receptor or induce appreciable effector function. In a certain embodiment the Fc receptor is an Fcγ receptor. In one embodiment the Fc receptor is a mammalian Fc receptor. In certain embodiments, the mammalian Fc receptor is a human Fc receptor. In one embodiment the Fc receptor is an activating Fc receptor. In a specific embodiment the Fc receptor is an activating human Fcγ receptor, more specifically human FcγRIIIa, FcγRI or FcγRIIa, most specifically human FcγRIIIa. In one embodiment the effector function is one or more function selected from the group consisting of CDC, ADCC, ADCP, and cytokine secretion. In a particular embodiment the effector function is ADCC. In one embodiment the Fc region exhibits binding affinity to neonatal Fc receptor (FcRn). In certain embodiments, the FcRn binding affinity is substantially similar to that of a native IgG1 Fc. In some embodiments, substantially similar binding to FcRn is achieved when the Fc region of a heteromultimer construct described herein exhibits greater than about 70%, or in some embodiments greater than about 80%, and in some particular embodiments greater than about 90% of the binding affinity of a native IgG1 Fc domain to FcRn.

In certain embodiments the Fc region of a heteromultimer construct described herein is engineered to have reduced binding affinity to an Fc receptor and/or reduced effector function, as compared to a non-engineered Fc domain. In some embodiments, the engineered mutations are present in the lower hinge and CH2 domain. In particular embodiments, the Fc region of a heteromultimer described herein comprises one or more amino acid mutation that reduces the binding affinity of the Fc region to an Fc receptor and/or effector function. In some embodiments, the same one or more amino acid mutation is present in each of the two subunits of the Fc region. In some embodiments, different amino acid mutations are introduced in each of the two subunits of the Fc region. In one embodiment the amino acid mutation reduces the binding affinity of the Fc region to an Fc receptor. In one embodiment the amino acid mutation reduces the binding affinity of the Fc region to an Fc receptor by at least 2-fold, or in some embodiments at least 5-fold, or in an embodiment at least 10-fold. In certain embodiments where there is more than one amino acid mutation that reduces the binding affinity of the Fc region to the Fc receptor, the combination of these amino acid mutations reduces the binding affinity of the Fc region to an Fc receptor by at least 10-fold, or in some embodiments at least 20-fold, or in certain embodiments at least 50-fold. In certain embodiment, the binding affinity of the Fc region for the Fc receptor is reduced to an extent where there is no longer any detectable binding for the mutant Fc for the Fc receptor in standard binding assay such as using the SPR instrument. In one embodiment the heteromultimer construct described herein comprising an engineered Fc domain exhibits less than 20%, and in certain embodiments less than 10%, and in select embodiments less than 5% of the binding affinity to an Fc receptor as compared to a corresponding construct comprising an Fc domain which is not engineered to reduce binding to an Fc receptor. In a particular embodiment the Fc receptor is an Fcγ receptor. In some embodiments the Fc receptor is a human Fc receptor. In some embodiments the Fc receptor is an activating Fc receptor. In a specific embodiment the Fc receptor is an activating human Fcγ receptor which in certain embodiments is one of human FcγRIIIa, FcγRI and FcγRIIa. In some embodiments, binding to each of these receptors is reduced. In some embodiments binding affinity to a complement component, for instance, but not restricted to C1q, is also reduced. In one embodiment binding affinity to neonatal Fc receptor (FcRn) is not reduced. In certain embodiments the Fc region of a heteromultimer described herein is engineered to have reduced effector function, as compared to a non-engineered Fc region. In certain embodiments, the reduced effector function can include, but is not limited to, one or more of the following: reduced complement dependent cytotoxicity (CDC), reduced antibody-dependent cell-mediated cytotoxicity (ADCC), reduced antibody-dependent cellular phagocytosis (ADCP), reduced cytokine secretion, reduced immune complex-mediated antigen uptake by antigen-presenting cells, reduced binding to NK cells, reduced binding to macrophages, reduced binding to monocytes, reduced binding to polymorphonuclear cells, reduced direct signaling inducing apoptosis, reduced crosslinking of target-bound antibodies, reduced dendritic cell maturation, or reduced T cell priming. In one embodiment the reduced effector function is one or more selected from the group of reduced CDC, reduced ADCC, reduced ADCP, and reduced cytokine secretion. In certain embodiments the reduced effector function is reduced ADCC. In one embodiment the reduced ADCC is less than 20% of the ADCC induced by a non-engineered Fc domain (or a T cell activating bispecific antigen binding molecule comprising a non-engineered Fc domain). In another embodiment the reduced ADCC is less than 50% of the ADCC induced by a non-engineered Fc domain (or a T cell activating bispecific antigen binding molecule comprising a non-engineered Fc domain). In a further embodiment the reduced ADCC is less than 10% of the ADCC induced by a non-engineered Fc domain (or a T cell activating bispecific antigen binding molecule comprising a non-engineered Fc domain).

In certain embodiments is the isolated multispecific heteromultimer described herein wherein the heterodimer Fc region comprises a variant CH2 domain comprising amino acid modifications to promote selective binding of a Fcgamma receptor as described herein.

In some embodiments is an isolated multispecific heteromultimer described herein, wherein the variant CH2 domain selectively binds at least one of FcgammaIIIa and FcgammaIIb receptor as compared to wild-type CH2 domain.

In certain embodiments the multispecific heteromultimer construct comprises a variant CH2 region that binds at least one B cell such that the heteromultimer construct binds B cells with a valency greater than one.

### Albumin-Based Multispecific Heteromultimer Constructs:

Provided are isolated multispecific heteromultimer constructs comprising: a first polypeptide construct comprising a first transporter polypeptide fused to at least one CD3 binding polypeptide construct that binds to a CD3 complex on at least one CD3 expressing cell; a second polypeptide construct comprising a second transporter polypeptide which is different from said first transporter polypeptide, fused to at least one antigen binding polypeptide construct that binds to a target antigen on at least one B cell; wherein said first and second transporter polypeptides are derived from a protein by segmentation of said protein, each transporter polypeptide comprising an amino acid sequence with at least 90% identity to a segment of said protein, and wherein said transporter polypeptides self-assemble to form a quasi-native structure of said monomeric protein. In some embodiments, each transporter polypeptide comprises an amino acid sequence with at least 85% identity to a segment of the protein. In some embodiments, each transporter polypeptide comprises an amino acid sequence with at least 80% identity to a segment of the protein. In some embodiments, each transporter polypeptide comprises an amino acid sequence with at least 95% identity to a segment of the protein. In some other embodiments, each transporter polypeptide comprises an amino acid sequence with at least 99% identity to a segment of the protein.

In certain embodiments is the isolated multispecific heteromultimer described herein, wherein said transporter polypeptides are not derived from an antibody. In a further embodiment is an isolated multispecific heteromultimer described herein, wherein each transporter polypeptide is an albumin derivative. In some embodiments is an albumin based isolated multispecific heteromultimer, wherein said albumin is human serum albumin. In some embodiments, at least one transporter polypeptide is an allo-albumin derivative. In certain embodiments is an isolated multispecific heteromultimer described herein wherein each transporter polypeptide is derived from a different alloalbumin. In some embodiments, each transporter polypeptide comprises an amino acid sequence with at least 75% identity to a segment of albumin. In some embodiments, each transporter polypeptide comprises an amino acid sequence with at least 80% identity to a segment of albumin. In some embodiments, each transporter polypeptide comprises an amino acid sequence with at least 90% identity to a segment of albumin. In some other embodiments, each transporter polypeptide comprises an amino acid sequence with at least 95% identity to a segment of albumin. In some other embodiments, each transporter polypeptide comprises an amino acid sequence with at least 99% identity to a segment of albumin.

Provided herein is an albumin based isolated multispecific heteromultimer construct comprising: a first monomer comprising a first transporter polypeptide fused to at least one CD3 binding polypeptide construct that binds to a CD3 complex on at least one CD3 expressing cell; a second polypeptide construct comprising a second transporter polypeptide which is different from said first transporter polypeptide, fused to at least one antigen binding polypeptide construct that binds to a target antigen on at least one B cell; wherein said first and second transporter polypeptides are obtained by segmentation of albumin, and each transporter polypeptide comprising an amino acid sequence with at least 90% identity to a segment of albumin such that said transporter polypeptides self-assemble to form quasi-native albumin, and wherein said first cargo polypeptide does not have any binding domain present in said second cargo polypeptide.

Provided herein are albumin based multispecific heteromultimer constructs as described above, wherein said first transporter polypeptide comprising at least one mutation selected from A194C, L198C, W214C, A217C, L331C and A335C. In certain embodiments, the second transporter polypeptide comprises at least one mutation selected from L331C, A335C, V343C, L346C, A350C, V455C, and N458C.

Provided are isolated multispecific heteromultimer constructs described herein wherein said multispecific heteromultimer construct simultaneously engages said at least one B cell and said at least one CD3 expressing cell such that the CD3 expressing cell is activated, thereby inducing killing of the B cell.

In certain embodiments, are provided heteromultimer constructs comprising transporter polypeptides derived from albumin that self-assemble to form a heteromultimer that exhibits native albumin like functional characteristics such as FcRn binding and structural characteristics. In certain embodiments, the albumin based heteromultimer constructs described herein when administered to a person in need, home to tumor cells. In some embodiments, the tumor cells are from a solid tumor. In some embodiments, the heteromultimer constructs described herein home to tumor cells and subsequently bind to said tumor cells. In certain embodiments, the heteromultimer constructs described herein home to at least one tumor cell, bind simultaneously to said at least one tumor cell and and at least one T-cell in a manner that results in the lysis of said tumor cell. In certain embodiments, the heteromultimer constructs described herein home to at least one tumor cell, bind simultaneously to said at least one tumor cell and and at least one T-cell such that the binding to said tumor cell is with a higher valency than the binding to said T-cell, and causes the lysis of said tumor cell.

### CD3 Complex Binding Polypeptide Constructs:

In certain embodiments of the immunoglobulin-based and albumin-based multispecific heteromultimer constructs provided herein, said heteromultimer construct comprises at least one CD3 binding polypeptide construct that binds to a CD3 complex on at least one CD3 expressing cell. In some embodiments, the at least one CD3 binding polypeptide construct comprises at least one CD3 binding domain from a CD3 specific antibody, a nanobody, fibronectin, affibody, anticalin, cysteine knot protein, DARPin, avimer, Kunitz domain or variant or derivative thereof. In some embodiments, the at least one CD3 binding domain comprises at least one amino acid modification that reduces immunogenicity as compared to a corresponding CD3 binding domain not comprising said modification. In an embodiment, the at least one CD3 binding domain comprises at least one amino acid modification that increases its stability as measured by Tm, as compared to a corresponding CD3 binding domain not comprising said modification. In some embodiments, there is about a 3 degree increase in the Tm as compared to the native CD3 binding domain not comprising said at least one modification. In some embodiments, there is about a 5 degree increase in the Tm as compared to the native CD3 binding domain not comprising said at least one modification. In some embodiments, there is about a 8 degree increase in the Tm as compared to the native CD3 binding domain not comprising said at least one modification. In some embodiments, there is about a 10 degree increase in the Tm as compared to the native CD3 binding domain not comprising said at least one modification.

In some embodiments, the at least one CD3 binding polypeptide construct described herein comprises at least one CD3 binding domain from a CD3 specific antibody wherein said CD3 specific antibody is a heavy chain antibody devoid of light chains.

In certain other embodiments, the at least one CD3 binding polypeptide construct described herein comprises at least one CD3 binding domain derived from a non-antibody protein scaffold domain.

In certain embodiments, the CD3 binding polypeptide constructs are CD3 binding Fab constructs (i.e. antigen binding constructs comprising a heavy and a light chain, each comprising a variable and a constant region). In some embodiment said Fab construct is mammalian. In one embodiment said Fab construct is human. In another embodiment said Fab construct is humanized. In yet another embodiment said Fab construct comprises at least one of human heavy and light chain constant regions. In a further embodiment said Fab construct is a single chain Fab (scFab).

In certain embodiments the CD3 binding polypeptide constructs comprise CD3 binding scFab constructs wherein the C-terminus of the Fab light chain is connected to the N-terminus of the Fab heavy chain by a peptide linker. The peptide linker allows arrangement of the Fab heavy and light chain to form a functional CD3 binding moiety. In certain embodiments, the peptide linkers suitable for connecting the Fab heavy and light chain include sequences comprising glycine-serine linkers for instance, but not limited to (GₘS)ₙ-GG (SEQ ID NO: 273), (SGₙ)ₘ (SEQ ID NO: 274), (SEGₙ)ₘ (SEQ ID NO: 275), wherein m and n are between 0-20. In certain embodiments, the scFab construct is a cross-over construct wherein the constant regions of the Fab light chain and the Fab heavy chain are exchanged. In another embodiment of a cross-over Fab, the variable regions of the Fab light chain and the Fab heavy chain are exchanged.

In certain embodiments, the CD3 binding polypeptide constructs comprise CD3 binding Fv constructs (i.e. antigen binding constructs comprising a heavy and a light chain, each comprising a variable region). In some embodiment said Fv construct is mammalian. In one embodiment said Fv construct is human. In another embodiment said Fv construct is humanized. In yet another embodiment said Fv construct comprises at least one of human heavy and light chain variable regions. In a further embodiment said Fv construct is a single chain Fv (scFv).

In some embodiments, the CD3 binding polypeptide construct of a multispecific heteromultimer construct described herein bind to at least one component of the CD3 complex. In a specific embodiment, the CD3 binding polypeptide construct binds to at least one of CD3 epsilon, CD3 gamma, CD3 delta or CD3 zeta of the CD3 complex. In certain embodiments, the CD3 binding polypeptide construct binds the CD3epsilon domain. In certain embodiments, binding polypeptide construct binds a human CD3 complex. In certain embodiments, the CD3 binding polypeptide construct exhibits cross-species binding to a least one member of the CD3 complex.

Provided herein are immunoglobulin-based and albumin-based multispecific heteromultimer constructs comprising at least one CD3 binding polypeptide construct that binds to a CD3 complex on at least one CD3 expressing cell, where in the CD3 expressing cell is a T-cell. In certain embodiments, the CD3 expressing cell is a human cell. In some embodiments, the CD3 expressing cell is a non-human, mammalian cell. In some embodiments, the T cell is a cytotoxic T cell. In some embodiments the T cell is a CD4+ or a CD8+ T cell.

In certain embodiments of the immunoglobulin-based and albumin-based multispecific heteromultimer constructs provided herein, the construct is capable of activating and redirecting cytotoxic activity of a T cell to a target cell such as a B cell. In a particular embodiment, said redirection is independent of MHC-mediated peptide antigen presentation by the target cell and and/or specificity of the T cell.

Provided herein are hetromultimer constructs that are capable of simultaneous binding to a B cell antigen for instance a tumor cell antigen, and an activating T cell antigen. In one embodiment, the heteromultimer construct is capable of crosslinking a T cell and a target B cell by simultaneous binding to a B cell antigen for instance CD19 or CD20 and an activating T cell antigen for instance CD3. In one embodiment, the simultaneous binding results in lysis of a target B cell, for instance a tumor cell. In one embodiment, such simultaneous binding results in activation of the T cell. In other embodiments, such simultaneous binding results in a cellular response of a T lymphocyte, for instance a cytotoxic T lymphocyte, selected from the group of: proliferation, differentiation, cytokine secretion, cytotoxic effector molecule release, cytotoxic activity, and expression of activation markers. In one embodiment, binding of the T cell activating bispecific antigen binding molecule to the activating T cell antigen without simultaneous binding to the target cell antigen does not result in T cell activation.

### B Cell Binding Polypeptide Constructs:

Provided herein are isolated heteromultimer constructs comprising at least one antigen binding polypeptide construct that binds to a target antigen on at least one B cell. In certain embodiments, the antigen binding polypeptide construct binds at least one member of a B cell CD21-CD19-CD81 complex. In some embodiments, the antigen binding polypeptide construct comprises at least one CD19 binding domain or fragment thereof. In an embodiment, the antigen binding polypeptide construct comprises at least one CD20 binding domain.

In some embodiments, the at least one antigen binding domain is a CD19 or CD20 binding domain which is obtained from a CD19 or CD20 specific antibody, a nanobody, fibronectin, affibody, anticalin, cysteine knot protein, DARPin, avimer, Kunitz domain or variant or derivative thereof. In some embodiments, the at least one antigen binding polypeptide construct described herein comprises at least one antigen binding domain which is a CD19 or CD20 binding domain from an antibody which is a heavy chain antibody devoid of light chains.

In some embodiments, the at least one antigen binding domain is a CD19 or CD20 binding domain that comprises at least one amino acid modification that reduces immunogenicity as compared to a corresponding antigen binding domain not comprising said modification. In an embodiment, the at least one antigen binding domain is a CD19 or CD20 binding domain comprising at least one amino acid modification that increases its stability as measured by Tm, as compared to a corresponding domain not comprising said modification.

In certain embodiments, the at least one antigen binding polypeptide construct is a Fab construct that binds at least one of CD19 and CD20 on a B cell. In some embodiment said Fab construct is mammalian. In one embodiment said Fab construct is human. In another embodiment said Fab construct is humanized. In yet another embodiment said Fab construct comprises at least one of human heavy and light chain constant regions. In a further embodiment said Fab construct is a single chain Fab (scFab).

In certain embodiments the CD19 and/or CD20 binding polypeptide construct comprises a scFab construct wherein the C-terminus of the Fab light chain is connected to the N-terminus of the Fab heavy chain by a peptide linker. The peptide linker allows arrangement of the Fab heavy and light chain to form a functional CD19 and/or CD20 binding moiety. In certain embodiments, the peptide linkers suitable for connecting the Fab heavy and light chain include sequences comprising glycine-serine linkers for instance, but not limited to (GₘS)ₙ-GG (SEQ ID NO: 273), (SGₙ)ₘ (SEQ ID NO: 274), (SEGₙ)ₘ (SEQ ID NO: 275), wherein m and n are between 0-20. In certain embodiments, the scFab construct is a cross-over construct wherein the constant regions of the Fab light chain and the Fab heavy chain are exchanged. In another embodiment of a cross-over Fab, the variable regions of the Fab light chain and the Fab heavy chain are exchanged.

In certain embodiments, the at least one antigen binding polypeptide construct is a Fv construct that binds at least one of CD19 and CD20 on a B cell. In some embodiment said Fv construct is mammalian. In one embodiment said Fv construct is human. In another embodiment said Fv construct is humanized. In yet another embodiment said Fv contruct comprises at least one of human heavy and light chain variable regions. In a further embodiment said Fv construct is a single chain Fv (scFv).

In certain embodiments, the antigen binding polypeptide construct exhibits cross-species binding to a least one antigen expressed on the surface of a B cell. In some embodiments, the antigen binding polypeptide construct of a multispecific heteromultimer construct described herein bind to at least one of mammalian CD19 and CD20. In certain embodiments, binding polypeptide construct binds a human CD19 or CD20.

Provided herein are hetromultimer constructs that are capable of simultaneous binding to a B cell antigen for instance a tumor cell antigen, and an activating T cell antigen. In one embodiment, the heteromultimer construct is capable of crosslinking a T cell and a target B cell by simultaneous binding to a B cell antigen for instance CD19 or CD20 and an activating T cell antigen for instance CD3.

In certain embodiments, a heteromultimer described herein comprises at least one antigen binding polypeptide construct that binds to a target antigen such as a CD19 or CD20 on at least one B cell associated with a disease. In some embodiments, the disease is a cancer selected from a carcinoma, a sarcoma, leukaemia, lymphoma and glioma. In an embodiment, the cancer is at least one of squamous cell carcinoma, adenocarcinoma, transition cell carcinoma, osteosarcoma and soft tissue sarcoma. In certain embodiments, the at least one B cell is an autoimmune reactive cell that is a lymphoid or myeloid cell.

### Additional Antigen Binding Constructs:

In certain embodiments, an albumin or immunoglobulin based multispecific heteromultimer construct described herein further comprises at least one binding domain that binds at least one of: EpCAM, EGFR, IGFR, HER-2 neu, HER-3, HER-4, PSMA, CEA, MUC-1 (mucin), MUC2, MUC3, MUC4, MUC5, MUC7, CCR4, CCR5, CD19, CD20, CD33, CD30, ganglioside GD3, 9-O-Acetyl-GD3, GM2, Poly SA, GD2, Carboanhydrase IX (MN/CA IX), CD44v6, Sonic Hedgehog (Shh), Wue-1, Plasma Cell Antigen, (membrane-bound), Melanoma Chondroitin Sulfate Proteoglycan (MCSP), CCR8, TNF-alpha precursor, STEAP, mesothelin, A33 Antigen, Prostate Stem Cell Antigen (PSCA), Ly-6; desmoglein 4, E-cadherin neoepitope, Fetal Acetylcholine Receptor, CD25, CA19-9 marker, CA-125 marker and Muellerian Inhibitory Substance (MIS) Receptor type II, sTn (sialylated Tn antigen; TAG-72), FAP (fibroblast activation antigen), endosialin, LG, SAS, EPHA4 CD63, CD3 BsAb immunocytokines TNF which comprise a CD3 antibody attached to the cytokine, IFNγ, IL-2, and TRAIL.

### Post Translational Modifications:

In certain embodiments are multispecific heteromultimer constructs described herein, which are differentially modified during or after translation. In some embodiments, the modification is at least one of: glycosylation, acetylation, phosphorylation, amidation, derivatization by known protecting/blocking groups, proteolytic cleavage and linkage to an antibody molecule or other cellular ligand. In some embodiments, the heteromultimer construct is chemically modified by known techniques, including but not limited, to specific chemical cleavage by cyanogen bromide, trypsin, chymotrypsin, papain, V8 protease, NaBH₄; acetylation, formylation, oxidation, reduction; and metabolic synthesis in the presence of tunicamycin.

Additional post-translational modifications of heteromultimers described herein include, for example, N-linked or O-linked carbohydrate chains, processing of N-terminal or C-terminal ends), attachment of chemical moieties to the amino acid backbone, chemical modifications of N-linked or O-linked carbohydrate chains, and addition or deletion of an N-terminal methionine residue as a result of procaryotic host cell expression. The heteromultimer constructs described herein are modified with a detectable label, such as an enzymatic, fluorescent, isotopic or affinity label to allow for detection and isolation of the protein. In certain embodiments, examples of suitable enzyme labels include horseradish peroxidase, alkaline phosphatase, beta-galactosidase, or acetylcholinesterase; examples of suitable prosthetic group complexes include streptavidin biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include iodine, carbon, sulfur, tritium, indium, technetium, thallium, gallium, palladium, molybdenum, xenon, fluorine.

In specific embodiments, heteromultimer constructs described herein are attached to macrocyclic chelators that associate with radiometal ions.

In some embodiments, the heteromultimer constructs described herein are modified by either natural processes, such as post-translational processing, or by chemical modification techniques which are well known in the art. In certain embodiments, the same type of modification may be present in the same or varying degrees at several sites in a given polypeptide. In certain embodiments, polypeptides from heteromultimers described herein are branched, for example, as a result of ubiquitination, and in some embodiments are cyclic, with or without branching. Cyclic, branched, and branched cyclic polypeptides are a result from posttranslation natural processes or made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, PROTEINS—STRUCTURE AND MOLECULAR PROPERTIES, 2nd Ed., T. E. Creighton, W. H. Freeman and Company, New York (1993); POST-TRANSLATIONAL COVALENT MODIFICATION OF PROTEINS, B. C. Johnson, Ed., Academic Press, New York, pgs. 1-12 (1983); Seifter et al., Meth. Enzymol. 182:626-646 (1990); Rattan et al., Ann. N.Y. Acad. Sci. 663:48-62 (1992)).

In certain embodiments, heteromultimeric constructs described herein are attached to solid supports, which are particularly useful for immunoassays or purification of polypeptides that are bound by, that bind to, or associate with albumin fusion proteins of the invention. Such solid supports include, but are not limited to, glass, cellulose, polyacrylamide, nylon, polystyrene, polyvinyl chloride or polypropylene.

### Polynucleotides:

Provided herein are polynucleotide constructs encoding multispecific heteromultimer constructs described herein. In certain embodiments the polynucleotide or nucleic acid is DNA. In other embodiments, a polynucleotide described herein is RNA, for example, in the form of messenger RNA (mRNA). RNA of the present invention may be single stranded or double stranded.

In certain embodiments is a set of expression vectors for expressing a multispecific heteromultimer construct described herein which comprises a first and a second polypeptide construct, said set comprising at least a first DNA sequence encoding said first polypeptide construct and at least a second DNA sequence encoding said second polypeptide construct.

In certain embodiments are polynucleotide sequences encoding a heteromultimer construct described herein or a polypeptide construct thereof with sequence as provided herein. In certain embodiments is a polynucleotide comprising a sequence that is at least about 80%, 85%, 90%, 95%, 96%, 97%, 98%, or 99% identical to a nucleotide sequence shown in Figure. In certain embodiments are polynucleotide sequences encoding a heteromultimer construct described herein or a polypeptide construct thereof, wherein said polynucleotide comprises conservative mutations of the sequence as provided herein.

### Methods of Recombinant and Synthetic Production of Multispecific Heteromultimer Constructs:

Provided are methods of producing an expression product containing a multispecific heteromultimer construct as described herein, in stable mammalian cells, the method comprising: transfecting at least one mammalian cell with: at least a first DNA sequence encoding said first polypeptide construct and at least a second DNA sequence encoding said second polypeptide construct, such that said at least one first DNA sequence, said at least one second DNA sequence are transfected in said at least one mammalian cell in a pre-determined ratio to generate stable mammalian cells; culturing said stable mammalian cells to produce said expression product comprising said multispecific heteromultimer. In certain embodiments, said predetermined ratio of the at least one first DNA sequence: at least one second DNA sequence is about 1:1. In certain other embodiments, said predetermined ratio of the at least one first DNA sequence: at least one second DNA sequence is skewed towards a larger amount of the one first DNA sequence such as about 2:1. In yet other embodiments, said predetermined ratio of the at least one first DNA sequence: at least one second DNA sequence is skewed towards a larger amount of the one first DNA sequence such as about 1:2. In select embodiments, the mammalian cell is selected from the group consisting of a VERO, HeLa, HEK, NS0, Chinese Hamster Ovary (CHO), W138, BHK, COS-7, Caco-2 and MDCK cell, and subclasses and variants thereof.

In certain embodiments are heteromultimers produced as recombinant molecules by secretion from yeast, a microorganism such as a bacterium, or a human or animal cell line. In embodiments, the polypeptides are secreted from the host cells.

Embodiments include a cell, such as a yeast cell transformed to express a heteromultimer protein described herein. In addition to the transformed host cells themselves, are provided culture of those cells, preferably a monoclonal (clonally homogeneous) culture, or a culture derived from a monoclonal culture, in a nutrient medium. If the polypeptide is secreted, the medium will contain the polypeptide, with the cells, or without the cells if they have been filtered or centrifuged away. Many expression systems are known and may be used, including bacteria (for example E. coli and Bacillus subtilis), yeasts (for example Saccharomyces cerevisiae, Kluyveromyces lactis and Pichia pastoris, filamentous fungi (for example Aspergillus), plant cells, animal cells and insect cells.

A heteromultimer described herein is produced in conventional ways, for example from a coding sequence inserted in the host chromosome or on a free plasmid. The yeasts are transformed with a coding sequence for the desired protein in any of the usual ways, for example electroporation. Methods for transformation of yeast by electroporation are disclosed in Becker & Guarente (1990) Methods Enzymol. 194, 182.

Successfully transformed cells, i.e., cells that contain a DNA construct of the present invention, can be identified by well known techniques. For example, cells resulting from the introduction of an expression construct can be grown to produce the desired polypeptide. Cells can be harvested and lysed and their DNA content examined for the presence of the DNA using a method such as that described by Southern (1975) J. Mol. Biol. 98, 503 or Berent et al. (1985) Biotech. 3, 208. Alternatively, the presence of the protein in the supernatant can be detected using antibodies.

Useful yeast plasmid vectors include pRS403-406 and pRS413-416 and are generally available from Stratagene Cloning Systems, La Jolla, Calif. 92037, USA. Plasmids pRS403, pRS404, pRS405 and pRS406 are Yeast Integrating plasmids (Ylps) and incorporate the yeast selectable markers HIS3, 7RP1, LEU2 and URA3. Plasmids pRS413-416 are Yeast Centromere plasmids (Ycps).

A variety of methods have been developed to operably link DNA to vectors via complementary cohesive termini. For instance, complementary homopolymer tracts can be added to the DNA segment to be inserted to the vector DNA. The vector and DNA segment are then joined by hydrogen bonding between the complementary honmopolymeric tails to form recombinant DNA molecules.

Synthetic linkers containing one or more restriction sites provide an alternative method of joining the DNA segment to vectors. The DNA segment, generated by endonuclease restriction digestion, is treated with bacteriophage T4 DNA polymerase or E. coli DNA polymerase 1, enzymes that remove protruding, _single-stranded termini with their 3′ 5′-exonucleolytic activities, and fill in recessed 3′-ends with their polymerizing activities.

The combination of these activities therefore generates blunt-ended DNA segments. The blunt-ended segments are then incubated with a large molar excess of linker molecules in the presence of an enzyme that is able to catalyze the ligation of blunt-ended DNA molecules, such as bacteriophage T4 DNA ligase. Thus, the products of the reaction are DNA segments carrying polymeric linker sequences at their ends. These DNA segments are then cleaved with the appropriate restriction enzyme and ligated to an expression vector that has been cleaved with an enzyme that produces termini compatible with those of the DNA segment.

Synthetic linkers containing a variety of restriction endonuclease sites are commercially available from a number of sources including International Biotechnologies Inc, New Haven, Conn., USA.

Exemplary genera of yeast contemplated to be useful in the practice of the present invention as hosts for expressing the albumin, fusion proteins are Pichua (formerly classified as Hansenula), Saccharomyces, Kluyveromyces, Aspergillus, Candida, Torulopsis, Torulaspora, Schizosaccharomyces, Citeromyces, Pachysolen, Zygosaccharomyces, Debaromyces, Trichoderma, Cephalosporium, Humicola, Mucor, Neurospora, Yarrowia, Metschunikowia, Rhodosporidium, Leucosporidium, Botryoascus, Sporidiobolus, Endomycopsis, and the like. Preferred genera are those selected from the group consisting of Saccharomyces, Schizosaccharomyces, Kluyveromyces, Pichia and Torulaspora. Examples of Saccharomyces spp. are S. cerevisiae, S. italicus and S. rouxii.

Examples of Kluyveromyces spp. are K. fragilis, K. lactis and K. marxianus. A suitable Torulaspora species is T. delbrueckii. Examples of Pichia (Hansenula) spp. are P. angusta (formerly H. polymorpha), P. anomala (formerly H. anomala) and P. pastoris. Methods for the transformation of S. cerevisiae are taught generally in EP 251 744, EP 258 067 and WO 90/01063, all of which are incorporated herein by reference.

Exemplary species of Saccharomyces useful for the synthesis of heteromultimer constructs described herein include S. cerevisiae, S. italicus, S. diastaticus, and Zygosaccharomyces rouxii. Preferred exemplary species of Kluyveromyces include K. fragilis and K. lactis. Preferred exemplary species of Hansenula include H. polymorpha (now Pichia angusta), H. anomala (now Pichia anomala), and Pichia capsulata. Additional preferred exemplary species of Pichia include P. pastoris. Preferred exemplary species of Aspergillus include A. niger and A. nidulans. Preferred exemplary species of Yarrowia include Y. lipolytica. Many preferred yeast species are available from the ATCC. For example, the following preferred yeast species are available from the ATCC and are useful in the expression of albumin fusion proteins: Saccharomyces cerevisiae, Hansen, teleomorph strain BY4743 yap3 mutant (ATCC Accession No. 4022731); Saccharomyces cerevisiae Hansen, teleomorph strain BY4743 hsp150 mutant (ATCC Accession No. 4021266); Saccharomyces cerevisiae Hansen, teleomorph strain BY4743 pmt1 mutant (ATCC Accession No. 4023792); Saccharomyces cerevisiae Hansen, teleomorph (ATCC Accession Nos. 20626; 44773; 44774; and 62995); Saccharomyces diastaticus Andrews et Gilliland ex van der Walt, teleomorph (ATCC Accession No. 62987); Kluyveromyces lactis (Dombrowski) van der Walt, teleomorph (ATCC Accession No. 76492); Pichia angusta (Teunisson et al.) Kurtzman, teleomorph deposited as Hansenula polymorpha de Morais et Maia, teleomorph (ATCC Accession No. 26012); Aspergillus niger van Tieghem, anamorph (ATCC Accession No. 9029); Aspergillus niger van Tieghem, anamorph (ATCC Accession No. 16404); Aspergillus nidulans (Eidam) Winter, anamorph (ATCC Accession No. 48756); and Yarrowia lipolytica (Wickerham et al.) van der Walt et von Arx, teleomorph (ATCC Accession No. 201847).

Suitable promoters for S. cerevisiae include those associated with the PGKI gene, GAL1 or GAL10 genes, CYCI, PH05, TRP1, ADH1, ADH2, the genes for glyceraldehyde-3-phosphate dehydrogenase, hexokinase, pyruvate decarboxylase, phosphofructokinase, triose phosphate isomerase, phosphoglucose isomerase, glucokinase, alpha-mating factor pheromone, [a mating factor pheromone], the PRBI promoter, the GUT2 promoter, the GPDI promoter, and hybrid promoters involving hybrids of parts of 5′ regulatory regions with parts of 5′ regulatory regions of other promoters or with upstream activation sites (e.g. the promoter of EP-A-258 067).

Convenient regulatable promoters for use in Schizosaccharomyces pombe are the thiamine-repressible promoter from the nmt gene as described by Maundrell (1990) J. Biol. Chem. 265, 10857-10864 and the glucose repressible jbpl gene promoter as described by Hoffman & Winston (1990) Genetics 124, 807-816.

Methods of transforming Pichia for expression of foreign genes are taught in, for example, Cregg et al. (1993), and various Phillips patents (e.g. U.S. Pat. No. 4,857,467, incorporated herein by reference), and Pichia expression kits are commercially available from Invitrogen BV, Leek, Netherlands, and Invitrogen Corp., San Diego, Calif. Suitable promoters include AOX1 and AOX2. Gleeson et al. (1986) J. Gen. Microbiol. 132, 3459-3465 include information on Hansenula vectors and transformation, suitable promoters being MOX1 and FMD1; whilst EP 361 991, Fleer et al. (1991) and other publications from Rhone-Poulenc Rorer teach how to express foreign proteins in Kluyveromyces spp., a suitable promoter being PGKI.

The transcription termination signal is preferably the 3′ flanking sequence of a eukaryotic gene which contains proper signals for transcription termination and polyadenylation. Suitable 3′ flanking sequences may, for example, be those of the gene naturally linked to the expression control sequence used, i.e. may correspond to the promoter. Alternatively, they may be different in which case the termination signal of the S. cerevisiae ADHI gene is preferred.

In certain embodiments, the desired heteromultimer protein is initially expressed with a secretion leader sequence, which may be any leader effective in the yeast chosen. Leaders useful in S. cerevisiae include that from the mating factor alpha polypeptide (MFa-1) and the hybrid leaders of EP-A-387 319. Such leaders (or signals) are cleaved by the yeast before the mature albumin is released into the surrounding medium. Further such leaders include those of S. cerevisiae invertase (SUC2) disclosed in JP 62-096086 (granted as 911036516), acid phosphatase (PH05), the pre-sequence of MFα-1, 0 glucanase (BGL2) and killer toxin; S. diastaticus glucoarnylase II; S. carlsbergensis α-galactosidase (MEL1); K. lactis killer toxin; and Candida glucoarnylase.

Provided are vectors containing polynucleotides encoding a heteromultimer construct described herein, host cells, and the production of the heteromultimer proteins by synthetic and recombinant techniques. The vector may be, for example, a phage, plasmid, viral, or retroviral vector. Retroviral vectors may be replication competent or replication defective. In the latter case, viral propagation generally will occur only in complementing host cells.

In certain embodiments, the polynucleotides encoding heteromultimer proteins described herein are joined to a vector containing a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells.

In certain embodiments, the polynucleotide insert is operatively linked to an appropriate promoter, such as the phage lambda PL promoter, the E. coli lac, trp, phoA and rac promoters, the SV40 early and late promoters and promoters of retroviral LTRs, to name a few. Other suitable promoters will be known to the skilled artisan. The expression constructs will further contain sites for transcription initiation, termination, and, in the transcribed region, a ribosome binding site for translation. The coding portion of the transcripts expressed by the constructs will preferably include a translation initiating codon at the beginning and a termination codon (UAA, UGA or UAG) appropriately positioned at the end of the polypeptide to be translated.

As indicated, the expression vectors will preferably include at least one selectable marker. Such markers include dihydrofolate reductase, G418, glutamine synthase, or neomycin resistance for eukaryotic cell culture, and tetracycline, kanamycin or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells (e.g., Saccharomyces cerevisiae or Pichia pastoris (ATCC Accession No. 201178)); insect cells such as Drosophila S2 and Spodoptera Sf9 cells; animal cells such as CHO, COS, NS0, 293, and Bowes melanoma cells; and plant cells. Appropriate culture mediums and conditions for the above-described host cells are known in the art.

Among vectors preferred for use in bacteria include pQE70, pQE60 and pQE-9, available from QIAGEN, Inc.; pBluescript vectors, Phagescript vectors, pNH8A, pNH16a, pNH18A; pNH46A, available from Stratagene Cloning Systems, Inc.; and ptrc99a, pKK223-3, pKK233-3, pDR540, pRIT5 available from Pharmacia Biotech, Inc. Among preferred eukaryotic vectors are pWLNEO, pSV2CAT, pOG44, pXT1 and pSG available from Stratagene; and pSVK3, pBPV, pMSG and pSVL available from Pharmacia. Preferred expression vectors for use in yeast systems include, but are not limited to pYES2, pYD1, pTEF1/Zeo, pYES2/GS, pPICZ, pGAPZ, pGAPZalph, pPIC9, pPIC3.5, pHIL-D2, pHIL-S1, pPIC3.5K, pPIC9K, and PAO815 (all available from Invitrogen, Carlbad, CA). Other suitable vectors will be readily apparent to the skilled artisan.

In one embodiment, polynucleotides encoding a multispecific heteromultimer construct described herein are fused to signal sequences that will direct the localization of a protein of the invention to particular compartments of a prokaryotic or eukaryotic cell and/or direct the secretion of a protein of the invention from a prokaryotic or eukaryotic cell. For example, in E. coli, one may wish to direct the expression of the protein to the periplasmic space. Examples of signal sequences or proteins (or fragments thereof) to which the heteromultimeric proteins are fused in order to direct the expression of the polypeptide to the periplasmic space of bacteria include, but are not limited to, the pelB signal sequence, the maltose binding protein (MBP) signal sequence, MBP, the ompA signal sequence, the signal sequence of the periplasmic E. coli heat-labile enterotoxin B-subunit, and the signal sequence of alkaline phosphatase. Several vectors are commercially available for the construction of fusion proteins which will direct the localization of a protein, such as the pMAL series of vectors (particularly the pMAL-. rho. series) available from New England Biolabs. In a specific embodiment, polynucleotides albumin fusion proteins of the invention may be fused to the pelB pectate lyase signal sequence to increase the efficiency of expression and purification of such polypeptides in Gram-negative bacteria. See, U.S. Pat. Nos. 5,576,195 and 5,846,818, the contents of which are herein incorporated by reference in their entireties.

Examples of signal peptides that are fused to a heteromultimeric protein in order to direct its secretion in mammalian cells include, but are not limited to, the MPIF-1 signal sequence (e.g., amino acids 1-21 of GenBank Accession number AAB51134), the stanniocalcin signal sequence (MLQNSAVLLLLVISASA) (SEQ ID NO: 276), and a consensus signal sequence (MPTWAWWLFLVLLLALWAPARG) (SEQ ID NO: 277). A suitable signal sequence that may be used in conjunction with baculoviral expression systems is the gp67 signal sequence (e.g., amino acids 1-19 of GenBank Accession Number AAA72759).

Vectors which use glutamine synthase (GS) or DHFR as the selectable markers can be amplified in the presence of the drugs methionine sulphoximine or methotrexate, respectively. An advantage of glutamine synthase based vectors are the availabilty of cell lines (e.g., the murine myeloma cell line, NS0) which are glutamine synthase negative. Glutamine synthase expression systems can also function in glutamine synthase expressing cells (e.g., Chinese Hamster Ovary (CHO) cells) by providing additional inhibitor to prevent the functioning of the endogenous gene. A glutamine synthase expression system and components thereof are detailed in PCT publications: WO87/04462; WO86/05807; WO89/10036; WO89/10404; and WO91/06657, which are hereby incorporated in their entireties by reference herein. Additionally, glutamine synthase expression vectors can be obtained from Lonza Biologics, Inc. (Portsmouth, N.H.). Expression and production of monoclonal antibodies using a GS expression system in murine myeloma cells is described in Bebbington et al., Bio/technology 10:169 (1992) and in Biblia and Robinson Biotechnol. Prog. 11:1 (1995) which are herein incorporated by reference.

Also provided are host cells containing vector constructs described herein, and additionally host cells containing nucleotide sequences that are operably associated with one or more heterologous control regions (e.g., promoter and/or enhancer) using techniques known of in the art. The host cell can be a higher eukaryotic cell, such as a mammalian cell (e.g., a human derived cell), or a lower eukaryotic cell, such as a yeast cell, or the host cell can be a prokaryotic cell, such as a bacterial cell. A host strain may be chosen which modulates the expression of the inserted gene sequences, or modifies and processes the gene product in the specific fashion desired. Expression from certain promoters can be elevated in the presence of certain inducers; thus expression of the genetically engineered polypeptide may be controlled. Furthermore, different host cells have characteristics and specific mechanisms for the translational and post-translational processing and modification (e.g., phosphorylation, cleavage) of proteins. Appropriate cell lines can be chosen to ensure the desired modifications and processing of the foreign protein expressed.

Introduction of the nucleic acids and nucleic acid constructs of the invention into the host cell can be effected by calcium phosphate transfection, DEAE-dextran mediated transfection, cationic lipid-mediated transfection, electroporation, transduction, infection, or other methods. Such methods are described in many standard laboratory manuals, such as Davis et al., Basic Methods In Molecular Biology (1986). It is specifically contemplated that the polypeptides of the present invention may in fact be expressed by a host cell lacking a recombinant vector.

In addition to encompassing host cells containing the vector constructs discussed herein, the invention also encompasses primary, secondary, and immortalized host cells of vertebrate origin, particularly mammalian origin, that have been engineered to delete or replace endogenous genetic material (e.g., the coding sequence corresponding to a Cargo polypeptide is replaced with a heteromultimer protein corresponding to the Cargo polypeptide), and/or to include genetic material. The genetic material operably associated with the endogenous polynucleotide may activate, alter, and/or amplify endogenous polynucleotides.

In addition, techniques known in the art may be used to operably associate heterologous polynucleotides (e.g., polynucleotides encoding an albumin protein, or a fragment or variant thereof) and/or heterologous control regions (e.g., promoter and/or enhancer) with endogenous polynucleotide sequences encoding a Therapeutic protein via homologous recombination (see, e.g., U.S. Pat. No. 5,641,670, issued Jun. 24, 1997; International Publication Number WO 96/29411; International Publication Number WO 94/12650; Koller et al., Proc. Natl. Acad. Sci. USA 86:8932-8935 (1989); and Zijlstra et al., Nature 342:435-438 (1989), the disclosures of each of which are incorporated by reference in their entireties).

Heteromultimer proteins described herein can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography such as with protein A, hydroxylapatite chromatography, hydrophobic charge interaction chromatography and lectin chromatography. Most preferably, high performance liquid chromatography (“HPLC”) is employed for purification.

In certain embodiments the heteromultimer proteins of the invention are purified using Anion Exchange Chromatography including, but not limited to, chromatography on Q-sepharose, DEAE sepharose, poros HQ, poros DEAF, Toyopearl Q, Toyopearl QAE, Toyopearl DEAE, Resource/Source Q and DEAE, Fractogel Q and DEAE columns.

In specific embodiments the proteins described herein are purified using Cation Exchange Chromatography including, but not limited to, SP-sepharose, CM sepharose, poros HS, poros CM, Toyopearl SP, Toyopearl CM, Resource/Source S and CM, Fractogel S and CM columns and their equivalents and comparables.

In addition, heteromultimer proteins described herein can be chemically synthesized using techniques known in the art (e.g., see Creighton, 1983, Proteins: Structures and Molecular Principles, W. H. Freeman & Co., N.Y and Hunkapiller et al., Nature, 310:105-111 (1984)). For example, a polypeptide corresponding to a fragment of a polypeptide can be synthesized by use of a peptide synthesizer. Furthermore, if desired, nonclassical amino acids or chemical amino acid analogs can be introduced as a substitution or addition into the polypeptide sequence. Non-classical amino acids include, but are not limited to, to the D-isomers of the common amino acids, 2,4diaminobutyric acid, alpha-amino isobutyric acid, 4aminobutyric acid, Abu, 2-amino butyric acid, g-Abu, e-Ahx, 6amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, β-alanine, fluoro-amino acids, designer amino acids such as β-methyl amino acids, Co-methyl amino acids, Nα-methyl amino acids, and amino acid analogs in general. Furthermore, the amino acid can be D (dextrorotary) or L (levorotary).

### Assays:

The heteromultimer constructs described herein can be assayed for functional activity (e.g., biological activity) using or routinely modifying assays known in the art, as well as assays described herein.

For example, in one embodiment where one is assaying for the ability of a heteromultimer construct described herein to bind an antigen or to compete with another polypeptide for binding to an antigen, or bind to an Fc receptor and/or anti-albumin antibody, various immunoassays known in the art can be used, including but not limited to, competitive and non-competitive assay systems using techniques such as radioimmunoassays, ELISA (enzyme linked immunosorbent assay), “sandwich” immunoassays, immunoradiometric assays, gel diffusion precipitation reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention.

In certain embodiments, where a binding partner (e.g., a receptor or a ligand) is identified for an antigen binding domain comprised by a heteromultimer described herein, binding to that binding partner by a heteromultimer described herein is assayed, e.g., by means well-known in the art, such as, for example, reducing and non-reducing gel chromatography, protein affinity chromatography, and affinity blotting. See generally, Phizicky et al., Microbiol. Rev. 59:94-123 (1995). In another embodiment, the ability of physiological correlates of a heteromultimeric protein to bind to a substrate(s) of antigen binding polypeptide constructs of the heteromultimers described herein can be routinely assayed using techniques known in the art.

### Therapeutic Uses:

In an aspect, heteromultimers described herein are directed to antibody-based therapies which involve administering heteromultimers described comprising cargo polypeptide(s) which is an antibody, a fragment or variant of an antibody, to a patient for treating one or more of the disclosed diseases, disorders, or conditions. Therapeutic compounds described herein include, but are not limited to, heteromultimers described herein, nucleic acids encoding heteromultimers described herein.

In certain embodiments is provided a method for the prevention, treatment or amelioration of at least one of: a proliferative disease, a minimal residual cancer, a tumorous disease, an inflammatory disease, an immunological disorder, an autoimmune disease, an infectious disease, viral disease, allergic reactions, parasitic reactions, graft-versus-host diseases or host-versus-graft diseases or cell malignancies, said method comprising administering to a subject in need of such a prevention, treatment or amelioration a pharmaceutical composition comprising a heteromultimer construct described herein.

In certain embodiments is a method of treating cancer in a mammal in need thereof, comprising administering to the mammal a composition comprising an effective amount of the pharmaceutical composition described herein, optionally in combination with other pharmaceutically active molecules. Inc ertain embodiments, the cancer is a solid tumor. In some embodiments, the solid tumor is one or more of sarcoma, carcinoma, and lymphoma. In certain other embodiments, the cancer is a hematological cancer. In some embodiments, the cancer is one or more of B-cell lymphoma, non-Hodgkin's lymphoma, and leukemia.

Provided is a method of treating cancer cells comprising providing to said cell a composition comprising a heteromultimer construct described herein. In some embodiments, the method further comprising providing said heteromultimer in conjugation with another therapeutic agent.

Provided is a method of treating a cancer non-responsive to blinatumomab in a mammal in need thereof, comprising administering to the mammal a composition comprising an effective amount of the pharmaceutical composition comprising a heteromultimer construct described herein.

In some embodiments is a method of treating a cancer cell regressive after treatment with blinatumomab, comprising providing to said cancer cell a composition comprising an effective amount of the pharmaceutical composition comprising a heteromultimer construct described herein.

In some embodiments is a method of treating an individual suffering from a disease characterized by expression of B cells, said method comprising providing to said individual an effective amount of a composition comprising an effective amount of the pharmaceutical composition comprising a heteromultimer construct described herein. In some embodiments the disease is not responsive to treatment with at least one of an anti-CD19 antibody and an anti-CD20 antibody. In certain embodiments the disease is a cancer or autoimmune condition resistant to CD19 or CD20 lytic antibodies

Provided is a method of treating an autoimmune condition in a mammal in need thereof, comprising administering to said mammal a composition comprising an effective amount of the pharmaceutical composition described herein. In certain embodiments, the autoimmune condition is one or more of multiple sclerosis, rheumatoid arthritis, lupus erytematosus, psoriatic arthritis, psoriasis, vasculitis, uveitis, Crohn's disease, and type 1 diabetes.

Provided is a method of treating an inflammatory condition in a mammal in need thereof, comprising administering to said mammal a composition comprising an effective amount of the pharmaceutical composition comprising an heteromultimer described herein.

Armed with the teachings provided herein, one of ordinary skill in the art will know how to use the heteromultimers described herein for diagnostic, monitoring or therapeutic purposes without undue experimentation.

The heteromultimers described herein, comprising at least a fragment or variant of an antibody may be administered alone or in combination with other types of treatments (e.g., radiation therapy, chemotherapy, hormonal therapy, immunotherapy and anti-tumor agents). Generally, administration of products of a species origin or species reactivity (in the case of antibodies) that is the same species as that of the patient is preferred. Thus, in an embodiment, human antibodies, fragments derivatives, analogs, or nucleic acids, are administered to a human patient for therapy or prophylaxis.

### Gene Therapy:

In a specific embodiment, nucleic acids comprising sequences encoding heteromultimer proteins described herein are administered to treat, inhibit or prevent a disease or disorder associated with aberrant expression and/or activity of a protein, by way of gene therapy. Gene therapy refers to therapy performed by the administration to a subject of an expressed or expressible nucleic acid. In this embodiment of the invention, the nucleic acids produce their encoded protein that mediates a therapeutic effect. Any of the methods for gene therapy available in the art can be used.

Though T cell-engaging bispecific single chain antibodies described in the art have great therapeutic potential for the treatment of malignant diseases, most of these bispecific molecules are limited in that they are species specific and recognize only human antigen, and—due to genetic similarity-likely the chimpanzee counterpart. The advantage of the present invention is the provision of a bispecific single chain antibody comprising a binding domain exhibiting cross-species specificity to human and non-chimpanzee primate of the CD3 epsilon chain.

### Demonstration of Therapeutic or Prophylactic Activity:

The heteromultimers or pharmaceutical compositions described herein are tested in vitro, and then in vivo for the desired therapeutic or prophylactic activity, prior to use in humans. For example, in vitro assays to demonstrate the therapeutic or prophylactic utility of a compound or pharmaceutical composition include, the effect of a compound on a cell line or a patient tissue sample. The effect of the compound or composition on the cell line and/or tissue sample can be determined utilizing techniques known to those of skill in the art including, but not limited to, rosette formation assays and cell lysis assays. In accordance with the invention, in vitro assays which can be used to determine whether administration of a specific compound is indicated, include in vitro cell culture assays in which a patient tissue sample is grown in culture, and exposed to or otherwise administered a heteromultimer, and the effect of such heteromultimer upon the tissue sample is observed.

### Therapeutic/Prophylactic Administration and Composition:

Provided are methods of treatment, inhibition and prophylaxis by administration to a subject of an effective amount of a heteromultimer or pharmaceutical composition described herein. In an embodiment, the heteromultimer is substantially purified (e.g., substantially free from substances that limit its effect or produce undesired side-effects). In certain embodiments, the subject is an animal, including but not limited to animals such as cows, pigs, horses, chickens, cats, dogs, etc., and in certain embodiments, a mammal, and most preferably human.

Various delivery systems are known and can be used to administer a heteromultimer formulation described herein, e.g., encapsulation in liposomes, microparticles, microcapsules, recombinant cells capable of expressing the compound, receptor-mediated endocytosis (see, e.g., Wu and Wu, J. Biol. Chem. 262:4429-4432 (1987)), construction of a nucleic acid as part of a retroviral or other vector, etc. Methods of introduction include but are not limited to intradermal, intramuscular, intraperitoneal, intravenous, subcutaneous, intranasal, epidural, and oral routes. The compounds or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, in certain embodiments, it is desirable to introduce the heteromultimer compositions described herein into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it is desirable to administer the heteromultimers, or compositions described herein locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein, including an antibody, of the invention, care must be taken to use materials to which the protein does not absorb.

In another embodiment, the heteromultimers or composition can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.)

In yet another embodiment, the heteromultimers or composition can be delivered in a controlled release system. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J., Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target, e.g., the brain, thus requiring only a fraction of the systemic dose (see, e.g., Goodson, in Medical Applications of Controlled Release, supra, vol. 2, pp. 115-138 (1984)).

Other controlled release systems are discussed in the review by Langer (Science 249:1527-1533 (1990)).

In a specific embodiment comprising a nucleic acid encoding a heteromultimer described herein, the nucleic acid can be administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., Proc. Natl. Acad. Sci. USA 88:1864-1868 (1991)), etc. Alternatively, a nucleic acid can be introduced intracellularly and incorporated within host cell DNA for expression, by homologous recombination.

Also provided herein are pharmaceutical compositions. Such compositions comprise a therapeutically effective amount of a compound, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the patient. The formulation should suit the mode of administration.

In certain embodiments, the composition comprising the heteromultimer is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

In certain embodiments, the compositions described herein are formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxide isopropylamine, triethylamine, 2-ethylamino ethanol, histidine, procaine, etc.

The amount of the composition described herein which will be effective in the treatment, inhibition and prevention of a disease or disorder associated with aberrant expression and/or activity of a Therapeutic protein can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses are extrapolated from dose-response curves derived from in vitro or animal model test systems.

In certain embodiments, a heteromultimer construct described herein is suitably administered to the patient at one time or over a series of treatments. Depending on the type and severity of the disease, about 1 μg/kg to 15 mg/kg (e.g. 0.1 mg/kg-10 mg/kg) of T cell activating bispecific antigen binding molecule can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the heteromultimer described herein would be in the range from about 0.005 mg/kg to about 10 mg/kg. In other non-limiting examples, a dose may also comprise from about 1 microgram/kg body weight, about 5 microgram/kg body weight, about 10 microgram/kg body weight, about 50 microgram/kg body weight, about 100 microgram/kg body weight, about 200 microgram/kg body weight, about 350 microgram/kg body weight, about 500 microgram/kg body weight, about 1 milligram/kg body weight, about 5 milligram/kg body weight, about 10 milligram/kg body weight, about 50 milligram/kg body weight, about 100 milligram/kg body weight, about 200 milligram/kg body weight, about 350 milligram/kg body weight, about 500 milligram/kg body weight, to about 1000 mg/kg body weight or more per administration, and any range derivable therein. In non-limiting examples of a derivable range from the numbers listed herein, a range of about 5 mg/kg body weight to about 100 mg/kg body weight, about 5 microgram kg body weight to about 500 milligram kg body weight, etc., can be administered, based on the numbers described above. Thus, one or more doses of about 0.5 mg/kg, 2.0 mg/kg, 5.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the T cell activating bispecific antigen binding molecule). An initial higher loading dose, followed by one or more lower doses may be administered. However, other dosage regimens may be useful. The progress of this therapy is easily monitored by conventional techniques and assays.

The heteromultimers described herein are generally used in an amount effective to achieve the intended purpose. For use to treat or prevent a disease condition, a heteromultimer described herein, or pharmaceutical compositions thereof, are administered or applied in a therapeutically effective amount. Determination of a therapeutically effective amount is well within the capabilities of those skilled in the art, especially in light of the detailed disclosure provided herein.

For systemic administration, a therapeutically effective dose can be estimated initially from in vitro assays, such as cell culture assays. A dose can then be formulated in animal models to achieve a circulating concentration range that includes the IC₅₀ as determined in cell culture. Such information can be used to more accurately determine useful doses in humans.

Initial dosages can also be estimated from in vivo data, e.g., animal models, using techniques that are well known in the art. One having ordinary skill in the art could readily optimize administration to humans based on animal data.

Dosage amount and interval may be adjusted individually to provide plasma levels of the heteromultimer described herein which are sufficient to maintain therapeutic effect. Usual patient dosages for administration by injection range from about 0.1 to 50 mg/kg/day, typically from about 0.5 to 1 mg/kg/day. Therapeutically effective plasma levels may be achieved by administering multiple doses each day. Levels in plasma may be measured, for example, by HPLC.

In cases of local administration or selective uptake, the effective local concentration of the heteromultimer described herein may not be related to plasma concentration. One having skill in the art will be able to optimize therapeutically effective local dosages without undue experimentation.

A therapeutically effective dose of the heteromultimer constructs described herein will generally provide therapeutic benefit without causing substantial toxicity. Toxicity and therapeutic efficacy of a heteromultimer described herein can be determined by standard pharmaceutical procedures in cell culture or experimental animals. Cell culture assays and animal studies can be used to determine the LD50 (the dose lethal to 50% of a population) and the ED50 (the dose therapeutically effective in 50% of a population). The dose ratio between toxic and therapeutic effects is the therapeutic index, which can be expressed as the ratio LD50/ED50. T cell activating bispecific antigen binding molecules that exhibit large therapeutic indices are preferred. In one embodiment, the heteromultimer construct described herein according to the present invention exhibits a high therapeutic index. The data obtained from cell culture assays and animal studies can be used in formulating a range of dosages suitable for use in humans. The dosage lies preferably within a range of circulating concentrations that include the ED50 with little or no toxicity. The dosage may vary within this range depending upon a variety of factors, e.g., the dosage form employed, the route of administration utilized, the condition of the subject, and the like. The exact formulation, route of administration and dosage can be chosen by the individual physician in view of the patient's condition (see, e.g., Fingl et al, 1975, in: The Pharmacological Basis of Therapeutics, Ch. 1, p. 1, incorporated herein by reference in its entirety).

The attending physician for patients treated with heteromultimer constructs described herein would know how and when to terminate, interrupt, or adjust administration due to toxicity, organ dysfunction, and the like. Conversely, the attending physician would also know to adjust treatment to higher levels if the clinical response were not adequate (precluding toxicity). The magnitude of an administered dose in the management of the disorder of interest will vary with the severity of the condition to be treated, with the route of administration, and the like. The severity of the condition may, for example, be evaluated, in part, by standard prognostic evaluation methods. Further, the dose and perhaps dose frequency will also vary according to the age, body weight, and response of the individual patient.

Also provided is a process for the production of a pharmaceutical composition comprising a eteromutlimer described herein, said process comprising: culturing a host cell under conditions allowing the expression of a heteromultimer; recovering the produced heteromultimer from the culture; and producing the pharmaceutical composition.

### Other Agents and Treatments:

In certain embodiments, the heteromultimer constructs described herein are administered in combination with one or more other agents in therapy. For instance, in one embodiment, a heteromultimer described herein is co-administered with at least one additional therapeutic agent. The term “therapeutic agent” encompasses any agent administered to treat a symptom or disease in an individual in need of such treatment. Such additional therapeutic agent may comprise any active ingredients suitable for the particular indication being treated, preferably those with complementary activities that do not adversely affect each other. In certain embodiments, an additional therapeutic agent is an immunomodulatory agent, a cytostatic agent, an inhibitor of cell adhesion, a cytotoxic agent, an activator of cell apoptosis, or an agent that increases the sensitivity of cells to apoptotic inducers. In a particular embodiment, the additional therapeutic agent is an anti-cancer agent, for example a microtubule disruptor, an antimetabolite, a topoisomerase inhibitor, a DNA intercalator, an alkylating agent, a hormonal therapy, a kinase inhibitor, a receptor antagonist, an activator of tumor cell apoptosis, or an antiangio genie agent.

Such other agents are suitably present in combination in amounts that are effective for the purpose intended. The effective amount of such other agents depends on the amount of T cell activating bispecific antigen binding molecule used, the type of disorder or treatment, and other factors discussed above. The heteromultimers described herein are generally used in the same dosages and with administration routes as described herein, or about from 1 to 99% of the dosages described herein, or in any dosage and by any route that is empirically/clinically determined to be appropriate.

Such combination therapies noted above encompass combined administration (where two or more therapeutic agents are included in the same or separate compositions), and separate administration, in which case, administration of the heteromultimer described herein can occur prior to, simultaneously, and/or following, administration of the additional therapeutic agent and/or adjuvant. Heteromultimer constructs described herein can also be used in combination with radiation therapy.

### Articles of Manufacture:

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is a T cell activating bispecific antigen binding molecule of the invention. The label or package insert indicates that the composition is used for treating the condition of choice. Moreover, the article of manufacture may comprise (a) a first container with a composition contained therein, wherein the composition comprises a heteromultimer described herein; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### EXAMPLES

The following specific and non-limiting examples are to be construed as merely illustrative, and do not limit the present disclosure in any way whatsoever. Without further elaboration, it is believed that one skilled in the art can, based on the description herein, utilize the present disclosure to its fullest extent. All publications cited herein are hereby incorporated by reference in their entirety. Where reference is made to a URL or other such identifier or address, it is understood that such identifiers can change and particular information on the internet can come and go, but equivalent information can be found by searching the internet. Reference thereto evidences the availability and public dissemination of such information.

### Example 1: Bispecific CD3-CD19 scFvs Fused to an Asymmetric IgG1 Fc

Bispecific CD3-CD19 scFvs fused to an asymmetric IgG1 Fc heterodimer that exhibits stability comparable with native Fc homodimer, is a novel composition identified as v873. V873 belongs to a novel family of CD3-based bispecific azymetric IgG1 antibodies that can be expressed and purified with significantly higher yields in mammalian CHO cells compared to Amgen/Micromet bscCD19×CD3 BiTE bispecific. V873 demonstrates unexpected effector: target cell binding, bridging and target cell killing.

V873 and bispecific CD3-based azymetric antibodies have utility in targeted T cell mediated killing of diseased cells and hence may be useful for treating cancers and autoimmune and inflammatory diseases. V873 is a bispecific CD3-CD19 scFvs fused to an azymetric IgG1 Fc. v873 represents a novel bispecific azymetric antibody class comprising one anti-CD3 warhead and a second warhead comprising a cell surface antigen of a target cell, and an antibody Fc heterodimer comprising heterodimer IgG1 Fc. The fusion of the CD3 warhead to chain A or B of the Fc is important for its druggable properties. As additional examples, V874 and V875 are two other Bispecific CD3-CD19 scFvs fused to an asymmetric IgG1 Fc but have different CD3 amino acid compositions.

V873 shows unexpected good mammalian CHO expression and purification yields compared to the Amgen/Micromet blinatumomab CD3-CD19 BiTE tandem scFvs. V873 bridges T and B cells and results in potent killing of cultured human Burkitt lymphoma cells (Raji B-cell lymphoma line) using resting and IL-2 activated human PBMCs.

V873 and its related bispecific CD3-Azymetric antibodies differ from Amgen/Micromet CD3-CD19 BiTE and possess an IgG1 heterodimeric Fc that can bind FcgRs to mediate ADCC, CDC, ADCP effector activities. V873 can bind FcRn and this heterodimeric Fc class shows typical antibody manufacturability characteristics and long-half life of greater than 8 days in cynomolgus monkey. In contrast, blinatumomab has a half-life of less than several hours in non-human monkey and in human patients.

V873 and related bispecific CD3-based constructs address known stability issues with tandem scFvs and generally recognized poor manufacturability of blinatumomab. V873 addresses the short PK of blinatumomab and CNS adverse effects owing to its wildtype FcRn binding affinity and MWT which restricts its distribution to the peripheral compartment. Lastly, the Azymetric heterodimer Fc confers additional tailored FcgR effector ADCC, CDC, and ADCP activities and hence efficacy to drug resistant tumors.

The ability of v873 to mediate PBMC (T cell)-B cell killing is highly unexpected. The properties of blinatumomab bscCD19×CD3 and related BiTEs is reported to rely on the use of flexibly linked single-chain variable fragments (scFv) that are arranged in tandem joined by a short linker will allow for a much closer approximation of opposing cells than is possible with larger bispecific formats such as quadroma antibodies. The flexible linkage is expected to enable free rotation and kinking of the 2 scFv arms, thereby facilitating the simultaneous recognition of 2 epitopes present on 2 opposing cell membranes and the formation of a cytolytic immunologic synapse. Hence, based on what has been reported and generally accepted as unique to BiTE, it is unexpected that a bispecific CD3-CD19 construct as described herein with significantly different structural presentations of CD3 and CD19 scFv warheads on the heterodimer Fc can bind, bridge T and B cells, and mediate PBMC (T cell)-B cell killing. Other applications include depletion of B cells in B cell driven autoimmune and inflammatory diseases such as RA, lupus, MS, IBD. Further, V873 related bispecific CD3-Based azymetric antibodies may be useful for diagnostic purposes.

Additional details regarding the above are found in the following examples.

### Example 2. Design, Expression and Purification of Heteromultimer Constructs with a Heterodimeric Fc

Exemplary bispecific anti-CD3 and anti-CD19 heterodimeric antibodies

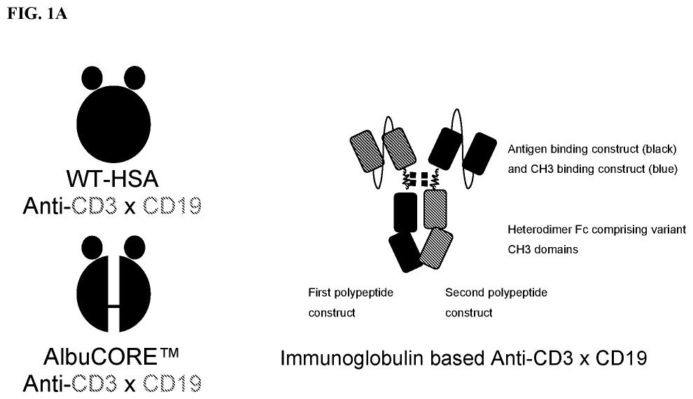

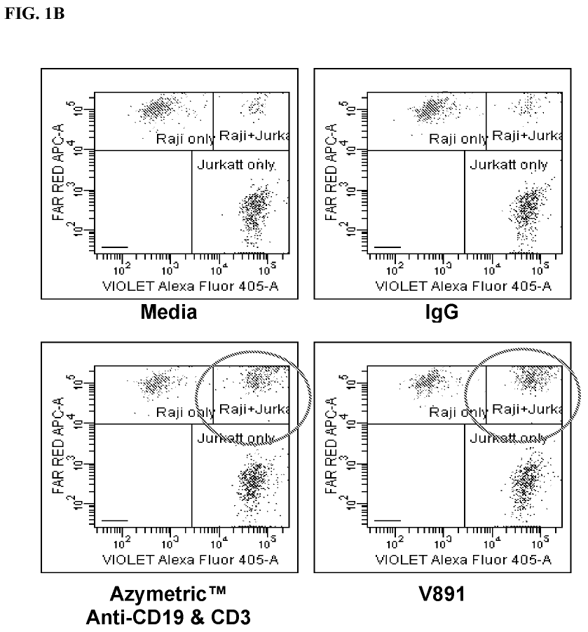

An exemplary schematic representation of an anti-CD3/anti-CD19 antibody is shown in FIG. 1A.

v873, v874, v875 exemplify bispecific anti-CD3/anti-CD19 heterodimeric Fc constructs and were prepared and tested as described below. Where the description includes a reference to BiTE, it refers to the antibody construct having an identical amino acid sequence to either the VH or VL of the anti-CD3 anti-CD19 BiTE molecule with or without modifications to variable heavy and light chain orientation (e.g. VH-VL) as indicated below.

v873 has a anti-CD19 BiTE (VL-VH) scFv on chain A and a CD3 BiTE™ (VH-VL) scFv on chain B of the heterodimer Fc with the following mutations L351Y_F405A_Y407V on chain A and T366L_K392M_T394W on chain B. [Polypeptide sequences correspond to SEQ ID No: 26 and 28]

V874 has a anti-CD19 BiTE™ (VL-VH) scFv on chain A and the CD3 BiTE™ (VLVH) scFv on chain B of the heterodimer Fc with the following mutations L351Y_F405A_Y407V on chain A and T366L_K392M_T394W on chain B. [Polypeptide sequences correspond to SEQ ID No: 30 and 32]

V875 has a anti-CD19 BiTE™ (VL-VH) scFv on chain A and the CD3 OKT3 (VL-VH) scFv on chain B of the heterodimer Fc with the following mutations L351Y_F405A_Y407V on chain A and T366L_K392M_T394W on chain B. [Polypeptide sequences correspond to SEQ ID No: 34 and 36]

v1379 has a anti-CD19 BiTE™ (VL-VH) scFv on chain A and a anti-CD3 (VH-VL) BiTE on chain B of the heterodimer Fc with the following mutations T350V_L351Y_F405A_Y407V on chain A and T350V_T366L_K392L_T394W on chain B. [Polypeptide sequences correspond to SEQ ID No: 87 and 88] Bivalent monospecific anti-CD3 and anti-CD19 antibodies

v865, v866, v867, v868 exemplify monospecific anti-CD3 or CD19 bivalent scFv-Fc constructs and were prepared and tested as described below:

v865 has a anti-CD19 BiTE (VL-VH) scFv on chain A and a anti-CD19 BiTE scFv chain B with WT Fc (105 kDa). [Polypeptide sequence correspond to SEQ ID No: 2]

v866 has a anti-CD3 BiTE™ (VH-VL) on chain A and a anti-CD3 BiTE™ (VH-VL) scFv on chain with a WT Fc (105 kDa). [Polypeptide sequence correspond to SEQ ID No: 4]

v867 has a anti-CD3 BiTE™ (VL-VH) scFv on chain A and a anti-CD3 BiTE™ (VL-VH) scFv on chain B with a WT Fc (105 kDa). [Polypeptide sequence correspond to SEQ ID No: 6]

v868 has a anti-CD3 OKT3 (VL-VH) scFv on chain A and a anti-CD3 OKT3 VL-VH scFv on chain with a WT Fc (105 kDa). [Polypeptide sequence correspond to SEQ ID No: 8].

### Monovalent Monospecific Anti-CD3

v869 has a anti-CD19 BiTE™ scFv (VL-VH) on chain A and a Fc on chain B of the heterodimer Fc with the following mutations L351Y_F405A_Y407V on chain A and T366L_K392M_T394W on chain B (80 kDa). [Polypeptide sequences correspond to SEQ ID No: 10 and 12].

v870 has a anti-CD3 BiTE™ (VH-VL) scFv on chain B and a Fc on chain A of the heterodimer Fc with the following mutations L351Y_F405A_Y407V on chain A and T366L_K392M_T394W on chain B (80 kDa). [Polypeptide sequences correspond to SEQ ID No: 14 and 16].

v871 has a anti-CD3 BiTE™ (VL-VH) scFv on chain B and a Fc on chain A of the heterodimer Fc with the following mutations L351Y_F405A_Y407V on chain A and T366L_K392M_T394W on chain B (80 kDa). [Polypeptide sequences correspond to SEQ ID No: 18 and 20].

v872 has a anti-CD3 OKT3 (VL-VH) scFv on chain B and a Fc on chain A of the heterodimer Fc with the following mutations L351Y_F405A_Y407V on chain A and T366L_K392M_T394W on chain B (80 kDa). [Polypeptide sequences correspond to SEQ ID No: 22 and 24].

### Benchmark Control

v891 has the identical sequence blinatumomab BiTE™ anti-CD3 BiTE scFv and anti-CD19 BiTE scFv (50 kDa) [Polypeptide sequence corresponds to SEQ ID No: 90

Exemplary mono- or bispecific anti-CD3 and anti-CD19 heterodimeric antibodies with a knock-out Fc

v1380 has a anti-CD19 BiTE™ (VL-VH) scFv on chain A and a anti-CD3 VHVL BiTE™ on chain B of the heterodimer Fc with the following mutations L234A_L235A_T350V_L351Y_F405A_Y407V on chain A and L234A_L235A_T350V_T366L_K392L_T394W on chain B (corresponding to polypeptide sequences SEQ ID NOs: 93 and 94).

v1381 has a anti-CD19 BiTE™ (VI-VH) scFv on chain A and a anti-CD3 BiTE™ scFv (VH-VL) on chain B of the heterodimer Fc with the following mutations N297A_T350V_L351Y_F405A_Y407V on chain A and N297A_T350V_T366L_K392L_T394W on chain B. (corresponding to polypeptide sequences SEQ ID NOs: 97 and 98)

Variants with Engineered Anti-CD3 Warheads for Stability Enhancement

The following variants contain mutations to the anti-CD3 scFv that include either changes to the linker length, VH-VL orientations, or point mutations to improve stability and yield.

v1653 has a anti-CD19 BiTE™ (VL-VH) scFv on chain A and a anti-CD3 OKT3 (VL-VH) (with C to S mutation at position 100A of the VH CDR3) on chain B of the heterodimer Fc with the following mutations T350V_L351Y_F405A_Y407V on chain A and T350V_T366L_K392L_T394W on chain B. (corresponding to polypeptide sequences SEQ ID NOs: 101 and 102)

v1654 has a anti-CD19 BiTE™ scFv on chain A and a anti-CD3 OKT3 OKT3 (VH-VL) with 18 amino acid linker chain B of the heterodimer Fc with the following mutations T350V_L351Y_F405A_Y407V on chain A and T350V_T366L_K392L_T394W on chain B. (corresponding to polypeptide sequences SEQ ID NOs: 105 and 106)

v1655 has a anti-CD19 BiTE™ scFv on chain A and a anti-CD3 OKT3 OKT3 (VH-VL) with 10 amino acid linker chain B of the heterodimer Fc with the following mutations T350V_L351Y_F405A_Y407V on chain A and T350V_T366L_K392L_T394W on chain B. (corresponding to polypeptide sequences SEQ ID NOs: 109 and 110)

v1656 has a anti-CD3 BiTE™ VHVL scFv on chain A and a anti-CD19 BiTE™ (VL-VH) on chain B of the heterodimer Fc with the following mutations T350V_L351Y_F405A_Y407V on chain A and T350V_T366L_K392L_T394W on chain B. (corresponding to polypeptide sequences SEQ ID NOs: 113 and 114)

v1657 has a anti-CD3 OKT3 (VL-VH) scFv (with C to S mutation at position 100A of the VH CDR3) on chain A and a anti-CD19 BiTE™ (VL-VH) scFv on chain B of the heterodimer Fc with the following mutations T350V_L351Y_F405A_Y407V on chain A and T350V_T366L_K392L_T394W on chain B. (corresponding to polypeptide sequences SEQ ID NOs: 117 and 118)

v1658 has a anti-CD3 OKT3 (VH-VL) scFv with 18 amino acid linker on chain A and a anti-CD19 BiTE™ (VL-VH) scFv on chain B of the heterodimer Fc with the following mutations T350V_L351Y_F405A_Y407V on chain A and T350V_T366L_K392L_T394W on chain B. (corresponding to polypeptide sequences SEQ ID NOs: 121 and 122)

v1659 has a anti-CD3 OKT3 (VH-VL) scFv with 10 amino acid linker on chain A and a anti-CD19 BiTE™ (VL_VH) scFv on chain B of the heterodimer Fc with the following mutations T350V_L351Y_F405A_Y407V on chain A and T350V_T366L_K392L_T394W on chain B. (corresponding to polypeptide sequences SEQ ID NOs: 125 and 126)

v1660 has a anti-CD3 OKT3 (VH-VL) scFv with 19 amino acid linker on chain A and a anti-CD19 BiTE™ (VL-VH) scFv on chain B of the heterodimer Fc with the following mutations T350V_L351Y_F405A_Y407V on chain A and T350V_T366L_K392L_T394W on chain B. (corresponding to polypeptide sequences SEQ ID NOs: 129 and 130)

Fc Knock-Out Variants with Engineered Anti-CD3 Warheads for Stability Enhancement

The following variants are Fc knoock outs that contain mutations to the anti-CD3 scFv that include either changes to the linker length, VH-VL orientations, or point mutations to improve stability and yield.

v1661 has a anti-CD19 BiTE™ (VL-VH) scFv on chain A and a anti-CD3 OKT3 (VL-VH) scFv on chain B of the heterodimer Fc with the following mutations D265S_L234A_L235A_T350V_L351Y_F405A_Y407V on chain A and D265S_L234A_L235A_T350V_T366L_K392L_T394W on chain B. [Polypeptide sequences correspond to SEQ ID No: 38 and 40]

v1662 has a anti-CD19 BiTE™ (VL-VH) scFv on chain A and a anti-CD3 OKT3 (VL-VH) scFv (with C to S mutation at position 100A of the VH CDR3) on chain B of the heterodimer Fc with the following mutations D265S_L234A_L235A_T350V_L351Y_F405A_Y407V on chain A and D265S_L234A_L235A_T350V_T366L_K392L_T394W on chain B. [Polypeptide sequences correspond to SEQ ID No: 42 and 44]

v1663 has a anti-CD19 BiTE™ (VL-VH) scFv on chain A and a anti-CD3 OKT3 (VL-VH) scFv with 18 amino acid linker on chain B of the heterodimer Fc with the following mutations D265S_L234A_L235A_T350V_L351Y_F405A_Y407V on chain A and D265S_L234A_L235A_T350V_T366L_K392L_T394W on chain B. (corresponding to polypeptide sequences SEQ ID NOs: 133 and 134)

v1664 has a anti-CD19 BiTE™ (VL-VH) scFv on chain A and a anti-CD3 OKT3 (VH-VL) scFv with 10 amino acid linker on chain B of the heterodimer Fc with the following mutations D265S_L234A_L235A_T350V_L351Y_F405A_Y407V on chain A and D265S_L234A_L235A_T350V_T366L_K392L_T394W on chain B. (corresponding to polypeptide sequences SEQ ID NOs: 137 and 138)

v1665 has a anti-CD3 BiTE™ (VH-VL) scFv on chain A and a anti-CD19 BiTE™ (VL-VH) scFv on chain B of the heterodimer Fc with the following mutations D265S_L234A_L235A_T350V_L351Y_F405A_Y407V on chain A and D265S_L234A_L235A_T350V_T366L_K392L_T394W on chain B. (corresponding to polypeptide sequences SEQ ID NOs: 141 and 142)

v1666 has a anti-CD3 OKT3 (VH-VL) scFv with a 19 amino acid linker on chain A and a anti-CD19 BiTE™ (VL-VH) scFv on chain B of the heterodimer Fc with the following mutations D265S_L234A_L235A_T350V_L351Y_F405A_Y407V on chain A and D265S_L234A_L235A_T350V_T366L_K392L_T394W on chain B [Polypeptide sequences correspond to SEQ ID No: 46 and 48].

v1667 has a anti-CD19 BiTE™ (VL-VH) scFv on chain A and a anti-CD3 OKT3 (VL-VH) scFv on chain B of the heterodimer Fc with the following mutations N297A_T350V_L351Y_F405A_Y407V on chain A and N297A_T350V_T366L_K392L_T394W on chain B. (corresponding to polypeptide sequences SEQ ID NOs: 145 and 146)

v1668 has a anti-CD19 BiTE™ (VL-VH) scFv on chain A and a anti-CD3 OKT3 (VL-VH) scFv (with C to S mutation at position 100A of the VH CDR3) on chain B of the heterodimer Fc with the following mutations N297A_T350V_L351Y_F405A_Y407V on chain A and N297A_T350V_T366L_K392L_T394W on chain B. (corresponding to polypeptide sequences SEQ ID NOs: 149 and 150)

v1669 has a anti-CD19 BiTE™ (VL-VH) scFv on chain A and a anti-CD3 OKT3 (VH-VL) scFv with 18 amino acid linker on chain B of the heterodimer Fc with the following mutations N297A_T350V_L351Y_F405A_Y407V on chain A and N297A_T350V_T366L_K392L_T394W on chain B. (corresponding to polypeptide sequences SEQ ID NOs: 153 and 154)

v1670 has a anti-CD19 BiTE™ (VL-VH) scFv on chain A and a anti-CD3 OKT3 (VH-VL) scFv with 10 amino acid linker on chain B of the heterodimer Fc with the following mutations N297A_T350V_L351Y_F405A_Y407V on chain A and N297A T350V_T366L_K392L_T394W on chain B. (corresponding to polypeptide sequences SEQ ID NOs: 157 and 158)

v1671 has a anti-CD3 BiTE™ (VH-VL) scFv on chain A and a anti-CD19 BiTE™ (VL-VH) scFv on chain B of the heterodimer Fc with the following mutations N297A_T350V_L351Y_F405A_Y407V on chain A and N297A_T350V_T366L_K392L_T394W on chain B. (corresponding to polypeptide sequences SEQ ID NOs: 161 and 162)

v1672 has a anti-CD3 OKT3 (VH-VL) scFv with a 19 amino acid linker on chain A and a anti-CD19 BiTE™ (VL-VH) scFv on chain B of the heterodimer Fc with the following mutations N297A_T350V_L351Y_F405A_Y407V on chain A and N297A_T350V_T366L_K392L_T394W on chain B. (corresponding to polypeptide sequences SEQ ID NOs: 165 and 166)

### Monovalent Anti-CD3 Antibodies

The following variants contain mutations to the anti-CD3 scFv that include either changes to the linker length, VH-VL orientations, or point mutations to improve stability and yield.

v1673 has a anti-CD3 OKT3 (VL-VH) scFv (with C to S mutation at position 100A of the VH CDR3) on chain B and a Fc on chain A of the heterodimer Fc with the following mutations T350V_L351Y_F405A_Y407V on chain A and T350V_T366L_K392L_T394W on chain B. (corresponding to polypeptide sequences SEQ ID NOs: 169 and 170)

v1674 has a anti-CD3 OKT3 (VH-VL) scFv with 18 amino acid linker on chain B and a Fc on chain A of the heterodimer Fc with the following mutations T350V_L351Y_F405A_Y407V on chain A and T350V_T366L_K392L_T394W on chain B. (corresponding to polypeptide sequences SEQ ID NOs: 173 and 174)

v1798 has a anti-CD3 OKT3 (VH-VL) scFv with 10 amino acid linker on chain B and a Fc on chain A of the heterodimer Fc with the following mutations T350V_L351Y_F405A_Y407V on chain A and T350V_T366L_K392L_T394W on chain B. (corresponding to polypeptide sequences SEQ ID NOs: 177 and 178)

v1799 has a anti-CD3 OKT3 (VH-VL) scFv with 19 amino acid linker on chain A and a Fc on chain B of the heterodimer Fc with the following mutations T350V_L351Y_F405A_Y407V on chain A and T350V_T366L_K392L_T394W on chain B. (corresponding to polypeptide sequences SEQ ID NOs: 181 and 182)

### Bispecific Disulfide 44-100 Stabilization Variants

The following variants contain point mutations for disulfide stabilization at position 100 in the variable light and position 44 in the variable heavy chain (denoted 44-100SS).

v1800 has a anti-CD19 BiTE™ (VL-VH) scFv on chain A and anti-CD3 OKT3 (VL-VH) 44-100SS on chain B of the heterodimer Fc with the following mutations T350V_L351Y_F405A_Y407V on chain A and T350V_T366L_K392L_T394W on chain B. (corresponding to polypeptide sequences SEQ ID NOs: 185 and 186)

v1801 has a anti-CD19 BiTE™ (VL-VH) scFv on chain A and anti-CD3 OKT3 (VL-VH) (with C to S mutation at position 100A of the VH CDR3) 44-100SS on chain B of the heterodimer Fc with the following mutations T350V_L351Y_F405A_Y407V on chain A and T350V_T366L_K392L_T394W on chain B. (corresponding to polypeptide sequences SEQ ID NOs: 189 and 190)

v1802 has a anti-CD3 BiTE™ (VH-VL) 44-100SS scFv on chain A and anti-CD19 BiTE™ (VL-VH) scFv on chain B of the heterodimer Fc with the following mutations T350V_L351Y_F405A_Y407V on chain A and T350V_T366L_K392L_T394W on chain B. (corresponding to polypeptide sequences SEQ ID NOs: 193 and 194)

v4541 has a anti-CD3 BiTE™ (VH-VL) 44-100SS scFv on chain A and anti-CD19 BiTE™ (VL-VH) scFv on chain B of the heterodimer Fc with the following mutations D265S_L234A_L235A_T350V_L351Y_F405A_Y407V on chain A and D265S_L234A_L235A_T350V_T366L_K392L_T394W on chain B. [Polypeptide sequences correspond to SEQ ID No: 50 and 52]

Cyno/Human Cross-Reactive Anti-CD3 and Anti-CD19 Bispecific Fc Knock-Out Variants with or without Disulfide 44-100 Stabilization

v4542 has a cyno/human cross-reactive anti-CD3 BiTE™ 12C (VH-VL) scFv on chain A and cyno/human cross-reactive anti-CD19 MOR208 (VH-VL) scFv on chain B of the heterodimer Fc with the following mutations D265S_L234A_L235A_T350V_L351Y_F405A_Y407V on chain A and D265S_L234A_L235A_T350V_T366L_K392L_T394W on chain B. [Polypeptide sequences correspond to SEQ ID No: 54 and 56].

v4543 has a cyno/human cross-reactive anti-CD3 BiTE™ 12C (VH-VL) 44-100SS scFv on chain A and cyno/human cross-reactive anti-CD19 MOR208 (VH-VL) 44-100SS scFv on chain B of the heterodimer Fc with the following mutations D265S_L234A_L235A_T350V_L351Y_F405A_Y407V on chain A and D265S_L234A_L235A_T350V_T366L_K392L_T394W on chain B. [Polypeptide sequences correspond to SEQ ID No: 58 and 60]

v4544 has a cyno/human cross-reactive anti-CD3 BiTE™ 12C (VH-VL) scFv on chain A and cyno/human cross-reactive anti-CD19 MOR208 (VL-VH) scFv on chain B of the heterodimer Fc with the following mutations D265S_L234A_L235A_T350V_L351Y_F405A_Y407V on chain A and D265S_L234A_L235A_T350V_T366L_K392L_T394W on chain B. [Polypeptide sequences correspond to SEQ ID No: 62 and 64]

v4545 has a cyno/human cross-reactive anti-CD3 BiTE™ 12C (VH-VL) 44-100SS scFv on chain A and cyno/human cross-reactive anti-CD19 MOR208 (VL-VH) 44-100SS scFv on chain B of the heterodimer Fc with the following mutations D265S_L234A_L235A_T350V_L351Y_F405A_Y407V on chain A and D265S_L234A_L235A_T350V_T366L_K392L_T394W on chain B. [Polypeptide sequences correspond to SEQ ID No: 66 and 68]

v4546 has a cyno/human cross-reactive anti-CD3 BiTE™ 12C (VH-VL) scFv on chain A and cyno/human cross-reactive anti-CD19 MDX-1342 (VH-VL) scFv on chain B of the heterodimer Fc with the following mutations D265S_L234A_L235A_T350V_L351Y_F405A_Y407V on chain A and D265S_L234A_L235A_T350V_T366L_K392L_T394W on chain B. [Polypeptide sequences correspond to SEQ ID No: 70 and 72]

v4547 has a cyno/human cross-reactive anti-CD3 BiTE™ 12C (VH-VL) 44-100SS scFv on chain A and cyno/human cross-reactive anti-CD19 MDX-1342 (VH-VL) 44-100SS scFv on chain B of the heterodimer Fc with the following mutations D265S_L234A_L235A_T350V_L351Y_F405A_Y407V on chain A and D265S_L234A_L235A_T350V_T366L_K392L_T394W on chain B. [Polypeptide sequences correspond to SEQ ID No: 74 and 76].

v4548 has a cyno/human cross-reactive anti-CD3 BiTE™ 12C (VH-VL) scFv on chain A and cyno/human cross-reactive anti-CD19 MDX-1342 (VL-VH) scFv on chain B of the heterodimer Fc with the following mutations D265S_L234A_L235A_T350V_L351Y_F405A_Y407V on chain A and D265S_L234A_L235A_T350V_T366L_K392L_T394W on chain B. [Polypeptide sequences correspond to SEQ ID No: 78 and 80] v4549 has a cyno/human cross-reactive anti-CD3 BiTE™ 12C (VH-VL) 44-100SS scFv on chain A and cyno/human cross-reactive anti-CD19 MDX-1342 (VL-VH) 44-100SS scFv on chain B of the heterodimer Fc with the following mutations D265S_L234A_L235A_T350V_L351Y_F405A_Y407V on chain A and D265S_L234A_L235A_T350V_T366L_K392L_T394W on chain B. [Polypeptide sequences correspond to SEQ ID No: 82 and 84]

The antibodies and antibody controls were cloned and expressed as follows. The genes encoding the antibody heavy and light chains were constructed via gene synthesis using codons optimized for human/mammalian expression. The Fab sequences were generated from a known Her2/neu binding Ab (Carter P. et al. (1992) Humanization of an anti P185 Her2 antibody for human cancer therapy. Proc Natl Acad Sci 89, 4285.) and the Fc was an IgG1 isotype. The scFv-Fc and OAA sequences, were generated from a known anti-CD3 and CD19 scFv Bite antibodies (Kipriyanov et. al., 1998, Int. J Cancer: 77,763-772), anti-CD3 BiTE antibodies (US2011/0275787) anti-CD3 monoclonal antibody OKT3 (Drug Bank reference: DB00075), anti-CD19 antibody MDX-1342 (WO2009054863; WO2007002223).

The final gene products were sub-cloned into the mammalian expression vector pTT5 (NRC-BRI, Canada) and expressed in CHO cells (Durocher, Y., Perret, S. & Kamen, A. High-level and high-throughput recombinant protein production by transient transfection of suspension-growing CHO cells. Nucleic acids research 30, E9 (2002)).

The CHO cells were transfected in exponential growth phase (1.5 to 2 million cells/mL) with aqueous 1 mg/mL 25 kDa polyethylenimine (PEI, Polysciences) at a PEI:DNA ratio of 2.5:1. (Raymond C. et al. A simplified polyethylenimine-mediated transfection process for large-scale and high-throughput applications. Methods. 55 (1): 44-51 (2011)). In order to determine the optimal concentration range for forming heterodimers, the DNA was transfected in optimal DNA ratios of the heavy chain A (HC-A), light chain (LC), and heavy chain B that allow for heterodimer formation (e.g. HC-A/HC-B/ratios=50:50% (OAAs; HC/Fc), 50:50%. Transfected cells were harvested after 5-6 days with the culture medium collected after centrifugation at 4000 rpm and clarified using a 0.45 μm filter.

The clarified culture medium was loaded onto a MabSelect SuRe (GE Healthcare) protein-A column and washed with 10 column volumes of PBS buffer at pH 7.2. The antibody was eluted with 10 column volumes of citrate buffer at pH 3.6 with the pooled fractions containing the antibody neutralized with TRIS at pH 11. The protein was finally desalted using an Econo-Pac 10DG column (Bio-Rad).

In some cases, the protein was futher purified by protein L chromatography by the method as follows. Capto L resin PBS was equilibrated with PBS and protein A purified v875, neutralized with 1 M Tris, was added to resin and incubated at RT for 30 min. Resin washed with PBS and flow through collected, bound protein was eluted with 0.5 ml 0.1 M Glycine, pH 3.

In some cases, the protein was further purified by gel filtration, 3.5 mg of the antibody mixture was concentrated to 1.5 mL and loaded onto a Superdex 200 HiLoad 16/600 200 pg column (GE Healthcare) via an AKTA Express FPLC at a flow-rate of 1 mL/min. PBS buffer at pH 7.4 was used at a flow-rate of 1 mL/min. Fractions corresponding to the purified antibody were collected, concentrated to −1 mg/ml and stored at −80° C.

The transient expression of exemplary v873, 874, 875 and other azymetric antibodies compared to the reference v891 is shown in FIG. 7. The SDS-PAGE in FIG. 7 shows that all exemplary heteromultimer can be expressed transiently in CHO3E7 cells with a cell viability of >80%.

### Example 3: Heteromultimer v873 is Able to Bridge Jurkat CD3 T Cells and Raji CD19 B Cells

The ability of v873 to bridge T cells and B cells was tested by FACS analysis as follows.

### Whole Cell Bridging by FACS

1×10⁶ cells/ml suspended in RPMI were labeled with 0.3 UM of the appropriate CellTrace label and mixed and incubated at 37° C. in a water bath for 25 minutes

Pellets were resuspended in 2 ml of L10+GS1+NaN3 to a final concentration 5××106 cells/ml.

Cell suspensions were analyzed (⅕ dilution) by flow cytometry to verify the appropriate cell labeling and laser settings. Flow-check and flow-set Fluorospheres were used to verify instrument standardization, optical alignment and fluidics.

After flow cytometry verification, and prior to bridging, each cell line was mixed together at the desired ratio, at a final concentration of 1×10⁶ cells/ml.

T:T bridging was assessed with Jurkat-violet+Jurkat-FarRed, B:B was assessed with RAJI-violet+RAJI-FarRed and T:B bridging was assessed with Jurkat-violet+RAJI-FarRed.

Antibodies were diluted to 2× in L10+GS1+NaN3 at room temperature then added to cells followed by gentle mixing and a 30 min incubation.

Following the 30 min incubation 2 μl of propidium iodide was added and slowly mixed and immediately analyze by flow cytometry.

Bridging % was calculated as the percentage of events that are simultaneously labeled violet and Far-red.

FIG. 1B shows the ability of v873 and blinatumomab CD19-CD3 BiTE (v891, MT-103) to bridge Jurkat CD3 T cells (Top left quadrant) with Raji CD19 B cells (bottom right quadrant) by FACS. Bridged T-B cells appear in top right quadrant. This result demonstrates that at 300 nM, heteromultimer v873 is able to specifically bridge Jurkat T cells and Raji B cells to a similar extent (23% of total cells) as BiTE (21% of total cells).

### Example 4: Heteromultimers Bind Selectively to CD3- and CD19-Expressing Cells

The ability of an exemplary heteromultimer, v873, to bind specifically to CD3 and CD19 was assessed by FACS. One-armed antibodies (OAAs) against CD3 and CD19 were also prepared as described in Example 2 and tested as controls in the whole cell FACS binding assay described below.

### Whole Cell Binding by FACS Protocol:

2×10⁶ cells/ml cells (>80% viability) were resuspended in L10+GS1 media, mixed with antibody dilutions, and incubated on ice for 1 h.

Cells were washed by adding 10 ml of cold R-2 buffer, and centrifuging at 233 g for 10 min at 4° C. The cell pellet was resuspended with 100 μl ( 1/100 dilution in L10+GS1 media) of fluorescently labeled anti-mouse or anti-human IgG and incubated for 1 h at RT.

Cell treatments were washed by adding 10 ml of cold R-2 as previously described, and the cell pellet resuspended with 400 μl of cold L-2 and the sample was filtered through Nitex and added to a tube containing 4 μl of propidium iodide.

Samples were Analyzed by Flow Cytometry.

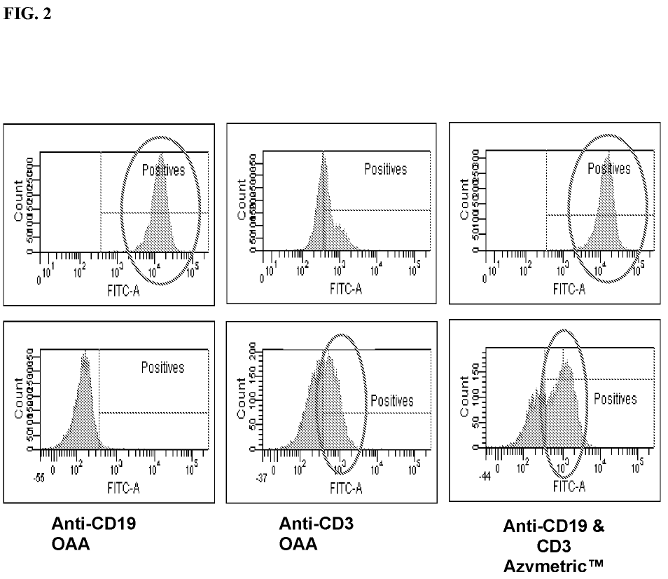

The results are shown in FIG. 2 and demonstrate that v873 is able to selectively bind and bridge to CD3-expressing Jurkat T cells (lower panel) and to CD19-expressing Raji B cells (upper panel). FIG. 2 also demonstrates that the one-armed anti-CD3 antibody specifically binds to Jurkat T cells and does not cross-react to CD19 expressing B cells, and that the one-armed anti-CD19 antibody specifically binds to Raji B cells and does not cross-react to Jurkat T cells.

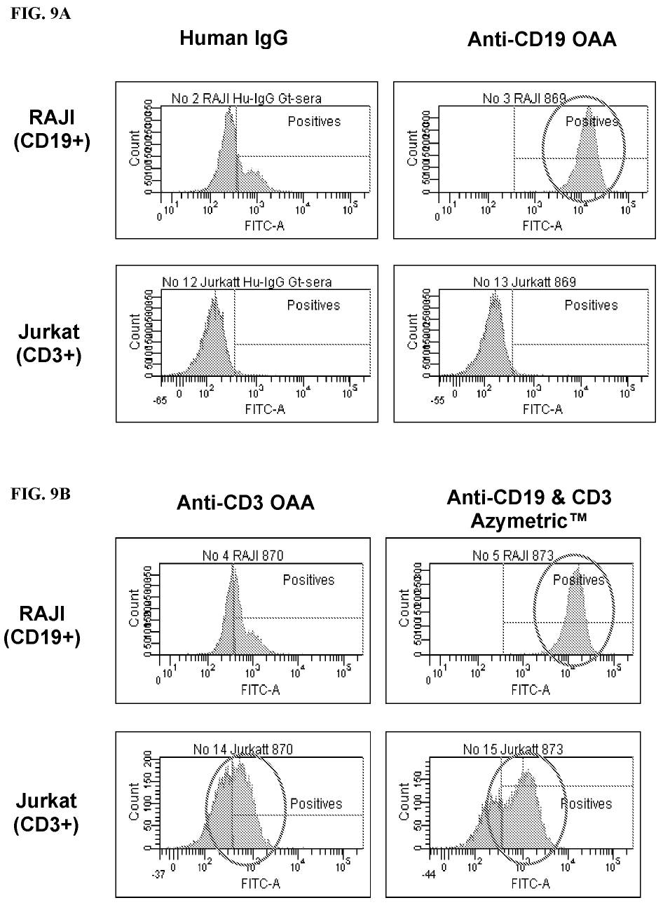

This experiment was repeated and results are shown in FIG. 9. As in the previous experiment, the FACS assay shows that v873 binds selectively to Jurkat T-cells and to Raji B-cells (FIG. 9B). FIG. 9A shows that human IgG (hIgG) does not bind to Jurkat T-cells and has low level binding to Raji B-cells, as expected due to the interactions between the hIgG Fc and CD32B on the Raji B-cells. FIG. 9A also shows that the anti-CD19 OAA binding selectively to the Raji B-cells and does not cross-react to Jurkat T cells.

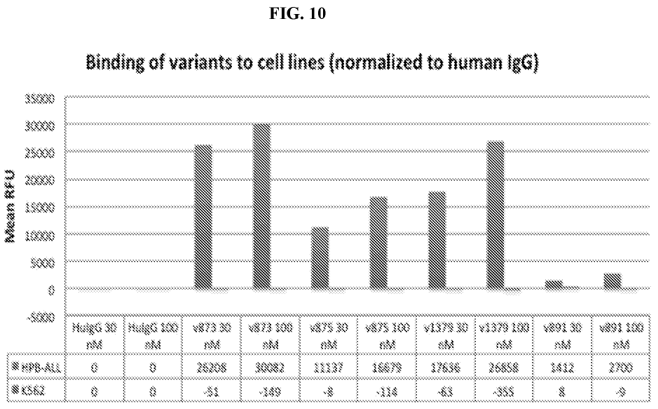

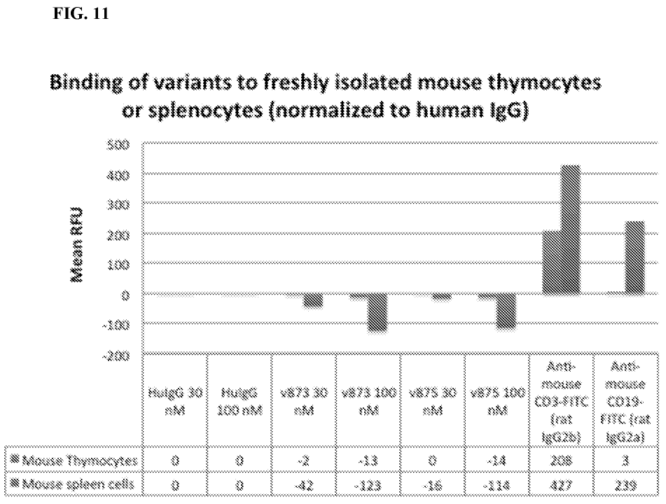

The FACS assay was also carried out to confirm that v873 does not bind to control cell lines that do not express CD3 or CD19. FIG. 10 shows that v873 does not bind to the K562 cell line, which does not express CD19 or CD3. FIG. 11 shows that v873 also does not bind to mouse lymphoid cells which does not express CD19 or CD3.

### Example 5: Heteromultimers Mediate PBMC Killing of Target Raji B Cells

In preliminary experiments, the ability of an exemplary heteromultimer, v873, to mediate T cell cytoxicity against target Raji B-cells was measured using IL-2-stimulated PBMCs as follows.

Human blood (120-140 mL) for individual studies was collected on two subsequent days from selected donors. On both days, PBMC were freshly isolated from donors and where needed a portion of the PMBC were passed through EasySep (STEMCELL Technologies Inc.) columns for CD4+ and CD8+enrichment. On the first day, the PBMCs and the enriched fractions were activated with 1000-3000 units per mL of IL-2 with an overnight incubation. The culture was processed with a negative selection column to isolate activated CD4+ and CD8+ cells. Cells were then labeled and analyzed by cytometry to evaluate the contents of CD69+ cells in the preparations. The PBMCs and the enriched fractions from the second day were used in the assay as is without IL-2 activation or in resting state.

Resting and IL-2 activated PBMCs and purified CD4+ and CD8+ were used as effector cells and Raji human B cells as target cells in the cytotoxicity assay. An effector:target ratio of 30:1 for PBMCs and 15:1 was tested for purified CD4+ and CD8+. The PBMC were also tested in presence of het-FC at 20 μg/mL and cell cytotoxicity was assayed at various concentrations of test items.

After incubating the cells with test articles for 20-26 hours, 50 uL of cell culture supernatant was collected for LDH analysis using a Promega LDH enzyme kit. In some studies, autologous T and B cells and/or allogenic B cells were assessed for their respective proportions in the culture and their 7AAD+ cell contents

CFSE was used and tested as a differential label between Raji and autologous B cells. Raji target cells were pre-labeled with minimal amounts of CFSE before the incubation with effectors, with or without test items. The cell pellets were resuspended in various antibody cocktails for flow cytometry analysis. A Guava 8HT flow cytometer was used for analysis of cell subpopulations. All antibodies were obtained from BD biosciences unless specified.

Each condition tested included appropriate controls; wells with all effector and target cell types separately, for all donors, incubated with all test items at all concentrations used in the potency assay.

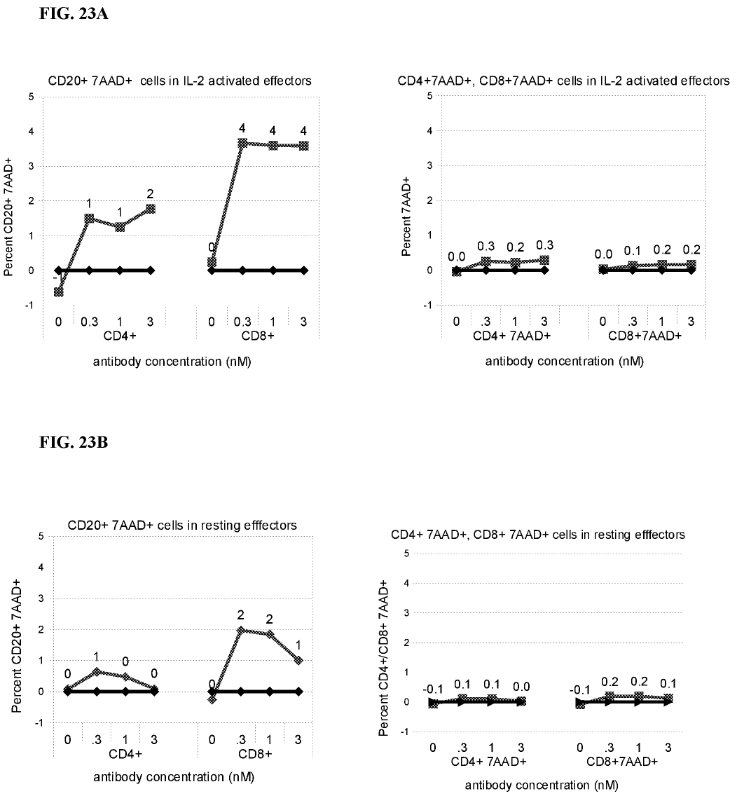

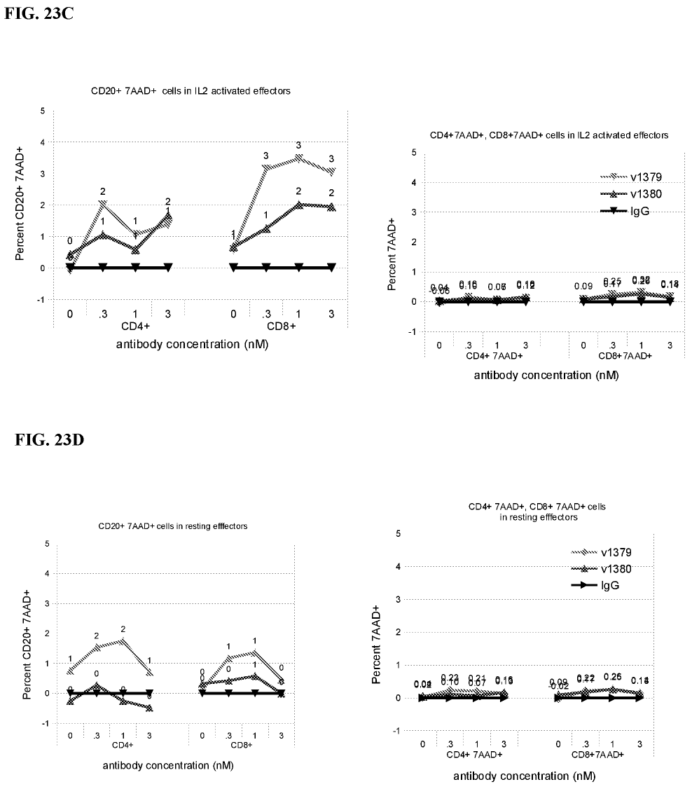

To avoid any hindrance between binding of the test items to their targets (CD3 and CD19), markers used for B and T cell staining were anti-CD20 and anti-CD7, respectively for the study shown in FIG. 23. This assay was performed with resting and IL2-activated PBMCs, purified CD4+ and CD8+ T cells.

### Data Analysis:

For LDH analysis, optical densities (OD) at 490 nm were determined for each well using a Molecular Devices Emax. Data analysis was performed using LibreOffice Calc software. The following analysis scheme is applied to calculate the cytotoxic response:

First, the averaged culture medium background signal (OD values) is subtracted from all wells before evaluating cytotoxic response. For detergent induced maximum release of pure target cells, a volume corrected specific background signal accounting for the presence of detergent is used and subtracted from maximum release OD values.

The spontaneous release of effectors and target combined is obtained from wells without any test items, for each effector population tested: the PBMCs, CD4 and CD8 negatively selected populations. The background LDH activity of the test system is better evaluated in wells containing the experimental mixture of effectors-target populations without any test item present.

Between all plates of all donors, spontaneous and maximum LDH release from Raji cells, media control wells, and LDH positive control wells were used to monitor inter plate variations.

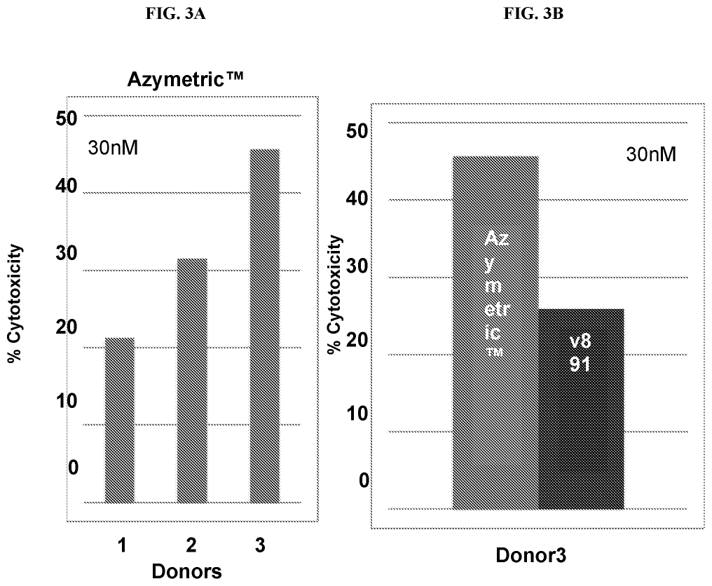

FIG. 3A depicts the ability of v873 to redirect IL-2 activated PBMC to kill target Raji B cells from 3 donors. FIG. 3B demonstrates that v873 is able to mediate higher redirected T-cell cytotoxicity than v891 in one of the donors. These preliminary results indicate that heteromultimers as exemplified by v873 may be able to mediate higher T-cell cytotoxicity than v891.

The ability of v873 to mediate PBMC killing of Raji B cells in 3 donors compared to human IgG1 control was assessed. The methods for this experiment follow that outlined in Example 5, with the following modifications: IL-2 stimulation of PBMC was tested at 1000-3000 units per mL.

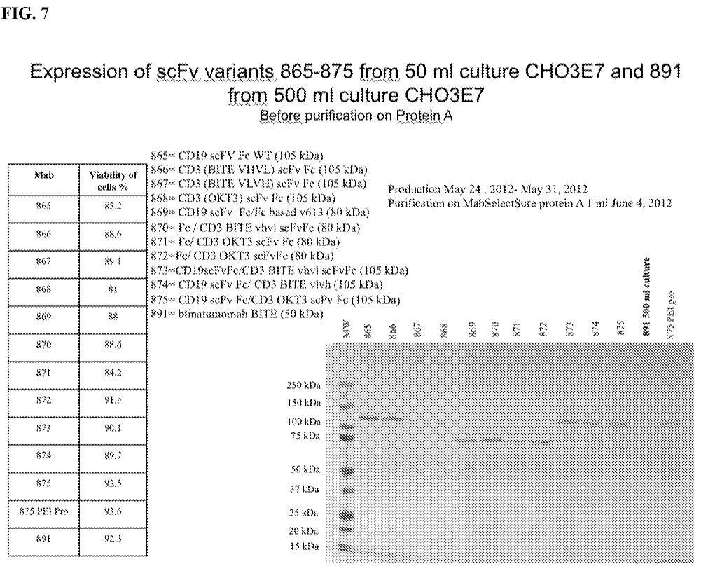

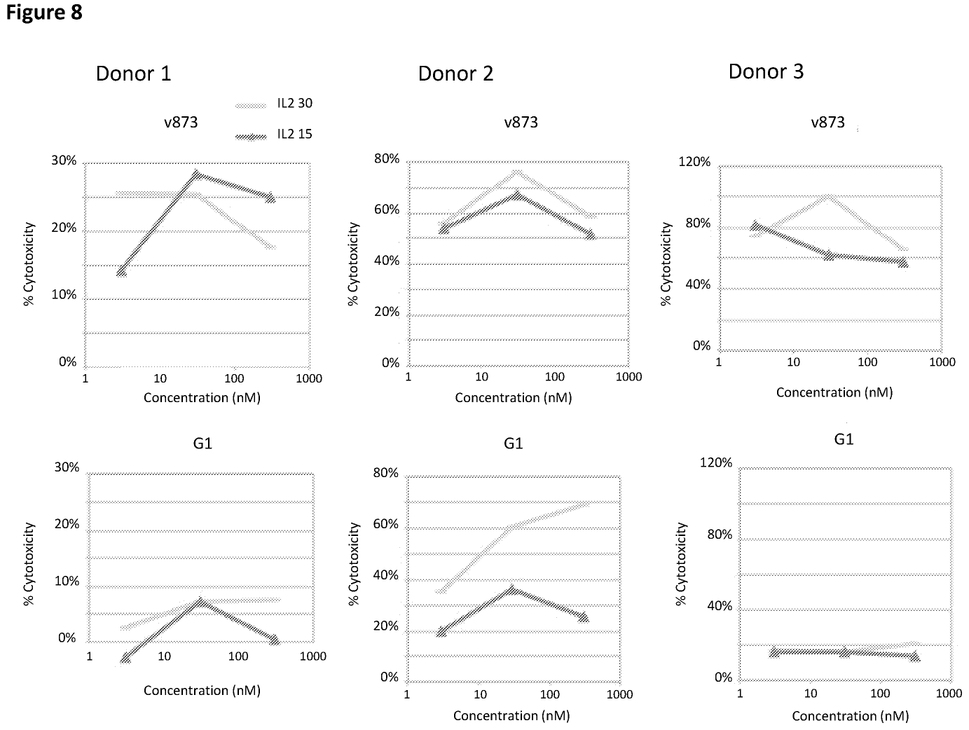

The results shown in FIG. 8 indicated that v873 induces are higher % cytotoxicity to target B cells when compared to negative control human IgG1 (G1) when comparing across individual donors. In some cases, stimulation with a higher concentration of IL-2 at 3000 units per mL, resulted in higher % cytotoxicity to target B cells.

### Example 6: Heteromultimers Mediate Redirected Killing of Target Raji B Cells with Resting and IL-2 Activated CD4+ and CD8+ T Cells

The ability of an exemplary heteromultimers, v875, v1379, v1380 to mediate CD4+ and CD8+ T cell cytoxicity against target Raji B-cells was measured as described in Example 5.

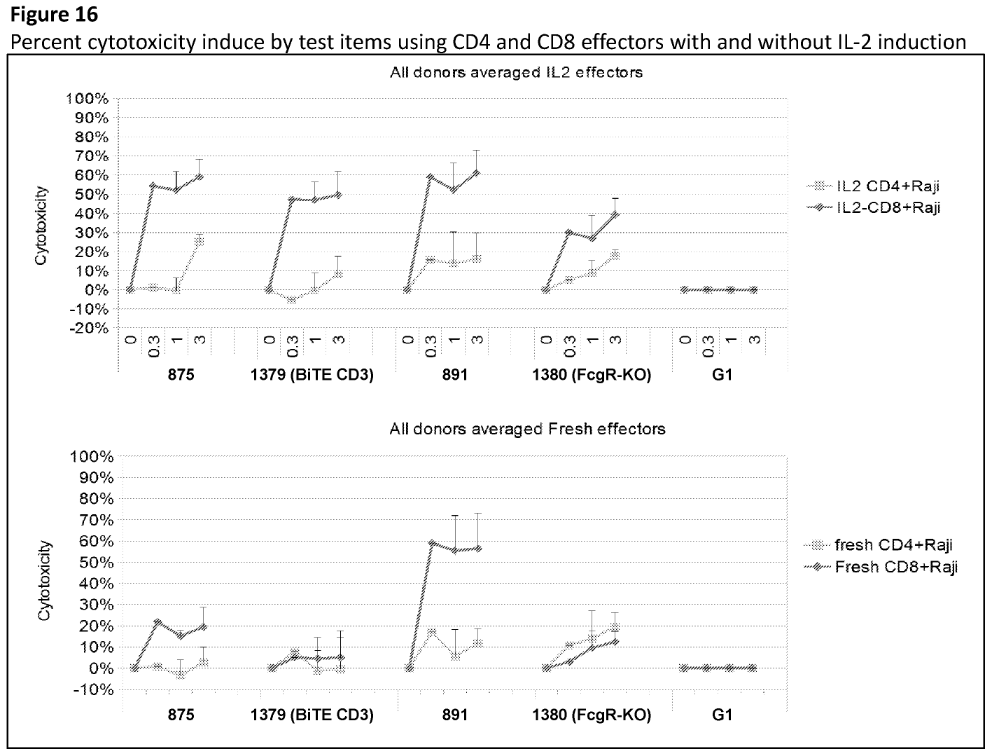

FIG. 16A depicts the ability of v875, v1379 and v1380 to mediate antibody dependent B cell cytoxicity by redirected CD4+ and CD8+ T cell towards Raji B cells. The top panel of FIG. 16 illustrates the cytoxicity of resting CD4+ and CD8+ T cells towards Raji B cells. These results illustrate that v875, v1380 and v891 elicit a concentration dependent cytotoxic response that is more prominent in the CD8+ T cells. The lower panel of FIG. 16 illustrates that the cytoxicity of IL-2 stimulated CD4+ and CD8+ T cells towards target Raji B cells. These results indicate that v875 and v1379 elicit similar Raji B-cells cytoxicity compared to v891 with IL-2 activated CD8+ effector T cells. In all cases, a greater cytotoxic response was elicited with the IL-2 activated CD8+ T cells compared to the IL-2 activated CD4+ T cells. These results also indicate that the FcGr knock-out variant, v1380, is cytotoxic toward target Raji B cells, but is slightly less effective (ca. 40% max cytotoxicity) compared to v875 and v1379 (ca. 60% max) that have WT heterodimers Fc.

FIGS. 16B-E depict representations of the data in FIG. 16A normalized to human IgG, for v875 (FIGS. 16B and C), and v1379 and v1380 (FIGS. 16D and E), and include % cytotoxicity indicated at each test antibody concentration. FIG. 16B shows the % cytotoxicity to target Raji B cells with v875 with IL-2 activated CD4+ and CD8+ effector T cells. FIG. 16C shows the % cytotoxicity to target Raji B cells with v875 with resting CD4+ and CD8+ effector T cells. FIG. 16D and E show direct comparisons of v1379 (WT Fc) and v1380 (L234A_L235A Fc knockout) in IL-2 activated (FIG. 16D), and resting (FIG. 16E) CD4+ and CD8+ T cells. The most significant impact of the L234A_L235A Fc knockout (v1380) to target B cell cytotoxicity is seen in the IL-2 activated CD8+ T cell population (FIG. 16D, left panel), where v1380 is less cytotoxic compared to v1379.

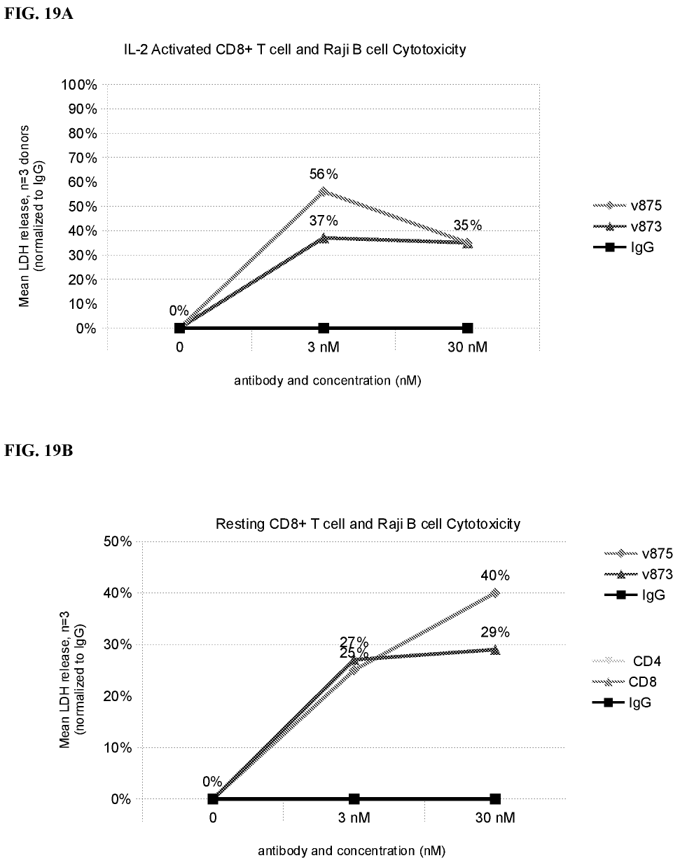

The experiment was repeated as shown in FIG. 19 with v875, v873 and human IgG, using resting (FIG. 19A) and IL-2 activated CD8+ T cells (FIG. 19B) as effectors cells targeting Raji B cells. FIG. 19A shows that v875 and v873 elicit >30% cytotoxicity to target Raji B cells with IL-2 activated CD8+ T cells as effectors, and maximal target cell killing is seen at the 3 nM concentration. FIG. 19B shows that v875 and v873 elicit dose dependent (>20%) cytotoxicity to target Raji B cells with resting CD8+ T cells as effectors. Greater target Raji B cell killing is seen when IL-2 activated CD8+ T cells were used as the effector cells.

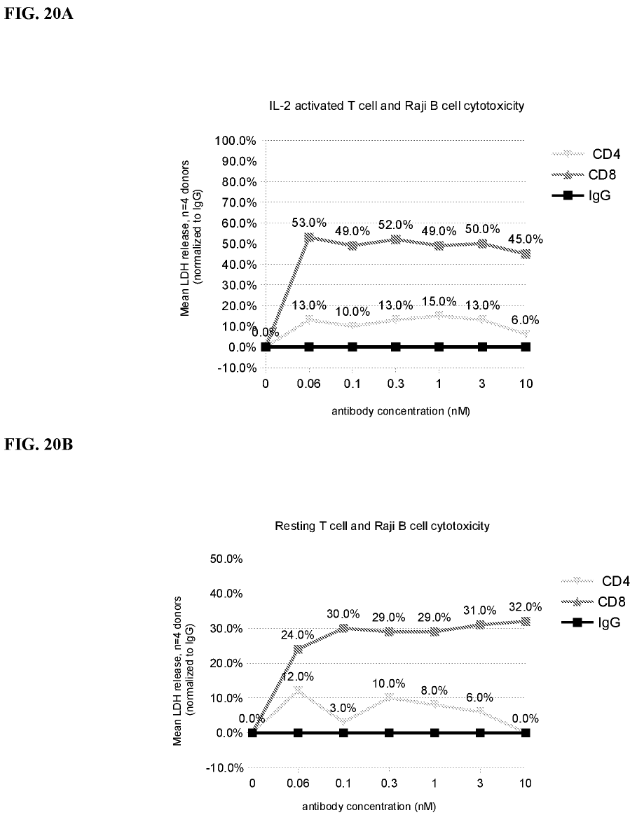

The experiment was repeated as shown in FIG. 20 to compare the relative contributions of CD4+ and CD8+ T cell killing to target Raji B cells at v875 concentrations ranging from 0.06-10.0 nM. FIG. 20A shows the target Raji B cell cytotoxicity of v875 with IL-2 activated CD4+ and CD8+ T cells. The percent target B cell killing elicited with IL-2 activated CD4+ cells and CD8+ T cells, did not increase at v875 concentrations above the 0.06 nM. As expected, target B cell killing is greater with IL-2 activated CD8+ T cells, compared to IL-2 activated CD4+ T cells. FIG. 20B shows the target Raji B cell cytotoxicity of v875 with resting CD4+ and CD8+ T cells. The percent target B cell killing elicited with resting CD8+ cells does not increase greatly at v875 concentrations above 0.1 nM. Dose-dependent B cell killing is seen with v875 and v873 when CD4+ and CD8+ effector T cells are used. As expected, target B cell killing is greater with resting CD8+ T cells, compared to resting CD4+ T cells.

### Example 7: The Heterodimeric Fc Contributes to Target Raji B Cell Cytotoxicity

The ability of exemplary heteromultimers, v875, and v873, to mediate target Raji B-cell cytotoxicity in the presence and absence of Fc was measured as described in Example 5.

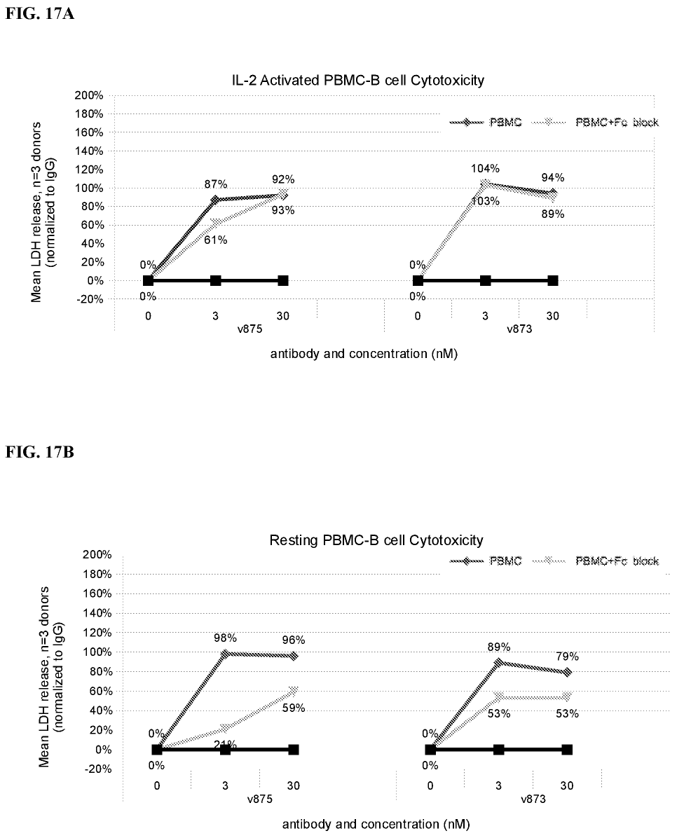

FIG. 17 depicts v875 and v873 Azymetric antibody mediated IL-2 activated PBMC and target Raji B cell cytotoxicity in the presence or absence of Fc blocking as determined by LDH assay. FIG. 17A illustrates that Fc blocking of IL-2 activated PBMC results in a minor (v875) or no (v873) reduction in the % cytotoxicity of target Raji B cells. FIG. 17B illustrates that Fc blocking of resting PBMC results in a reduction in the % cytotoxicity of target Raji B cells for v875 and v873.

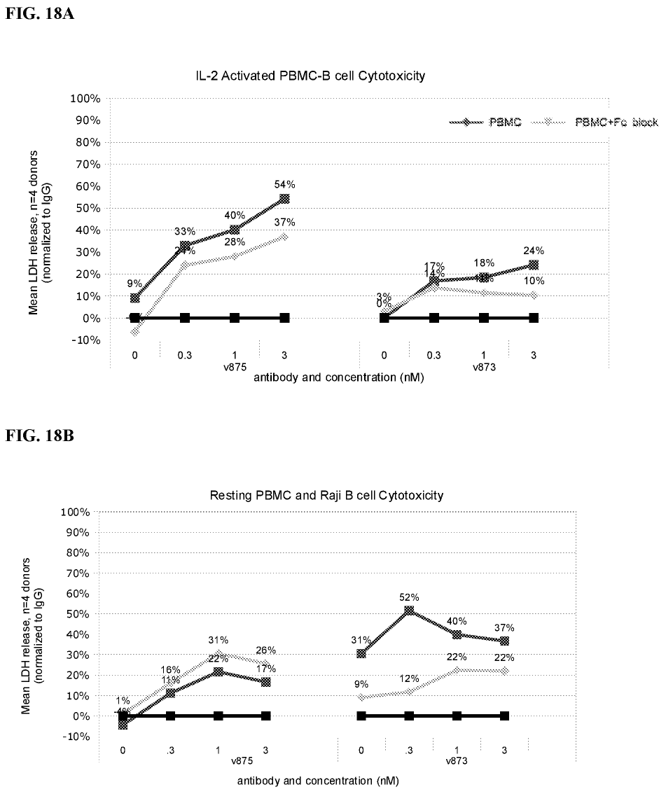

The experiment was repeated and the results shown in FIG. 18. FIG. 18A illustrates that Fc blocking of IL-2 activated PBMC results in a reduction in the % cytotoxicity of target Raji B cells at all antibody concentrations tested for v875 and v873. FIG. 18B illustrates that Fc blocking of resting PBMC results in a reduction in the % cytotoxicity of target Raji B cells at all antibody concentrations tested for v875 and v873. FIGS. 17 and 18 show that the Fc contributes to target Raji B cell cytotoxicity in the heterodimeric v875 and v873 antibodies.

### Example 8: Heteromultimers Mediate Autologous B Cell Cytotoxicity

The ability of exemplary the heteromultimer v875 and v873 to kill autologous B-cells was measured in total and resting IL-2-stimulated PBMCs where the percent of CD19+ 7AAD+ cells following incubation with v875 and v873 (300 nM, n=3 donors) was determined by flow cytometry as described in Example 5.

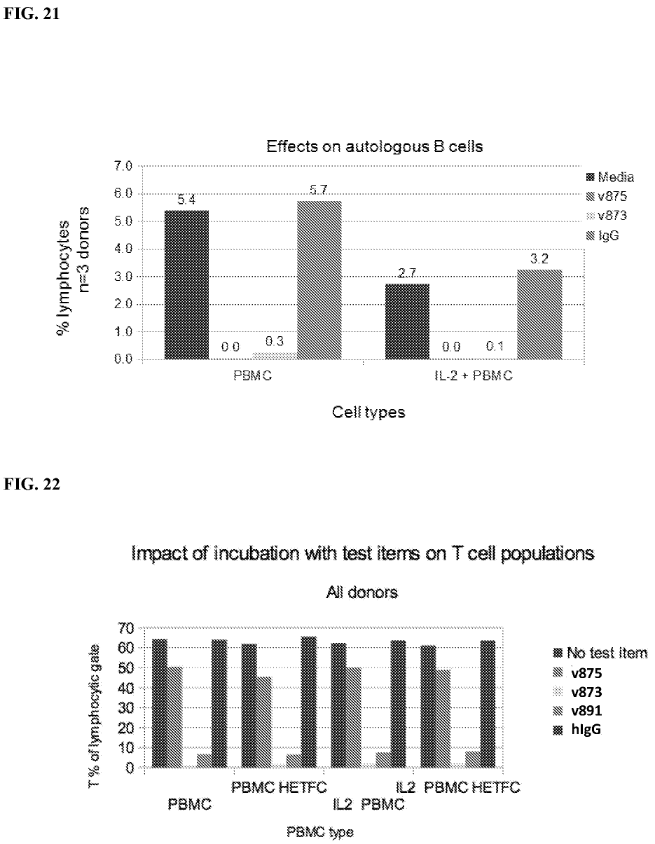

FIG. 21 shows, relative to untreated media and human IgG controls, that v875 and v873 (300 nM) mediate autologous B cell killing in total resting PBMC (left panel) and total IL-2 activated PBMC.

### Example 9: Heteromultimer v875 Spares Autologous T Cell Cytotoxicity Compared to BiTE

The effects of exemplary heteromultimer v875 and v873 treatment on the autologous T cell population was assessed in total and resting IL-2-stimulated PBMCs where the percent of CD3+ 7AAD+ cells following incubation with v875 and v873 (300 nM, n=3 donors) was determined by flow cytometry as described in Example 5.

FIG. 22 shows, relative to untreated media and human IgG controls, v875 has a more selective B cell killing by sparing more autologous T cells compared to v873 and v891.

### Example 10: Design, Expression and Purification of Heteromultimers with an Albumin Scaffold

The following exemplary CD3-CD19 binding heteromultimers based on an albumin scaffold were designed and prepared as follows.

The sequences for the anti-CD19 and anti-CD3 scFvs were chosen from two molecules that are currently in clinical trials and are well documented and tested for stability and production. The anti-CD19 and anti-CD3 scFv were directly adopted from the BiTE molecule blinatumomab. The antiCD3 scFv was chosen in the VH-VL orientation, consistent with what used in BiTE. The benchmark molecule was an scFv molecule based on BiTE (v891). AlbuCORE_1 (ABH2) CD3/CD19 fusions were created by attaching the antiCD3 warhead to the natural N terminus of fragment 1 and the antiCD19 to the C terminus of fragment 2 (v1092, polypeptide sequences corresponding to SEQ ID NO:264 and 266). The linkers used were identical to the ones used for the multivalent HER2 AlbuCORE experiments: GGGS (SEQ ID NO: 278) at the N terminus of fragment 1 and (GGSG) 4GG (SEQ ID NO: 279) at the C terminus of fragment 2. A second molecule was created where the warheads were reversed (i.e. anti-CD19 warhead at the natural N terminus of fragment 1 and the anti-CD3 at the C terminus of fragment 2, v1093. v1094 was designed to accommodate two different fusions at the natural termini of the albumin polypeptide (polypeptide sequences corresponding to SEQ ID NO:268). The scFv fusions were linked to the albumin molecule through a GGS linker at the N terminus and a GGSG (SEQ ID NO: 280) linker at the C terminus. The length of the linkers reflect the ones used in the MM-111 molecule, despite having a different sequence type.

V221 is the albumin-based heteromultimer used to construct v1092, but without the cargo molecules (polypeptide sequences corresponding to SEQ ID NO: 269 and 270).

Expression and purification were performed as previously described for the multivalent HER2.

### Example 11: Heteromultimers with an Albumin Scaffold Bind Specifically to CD3- or CD19-Expressing Cells

The ability of Anti-CD3×CD19 loaded AlbuCORE-1 (v1092) to CD3+ and CD19+ cells was assessed using FACS and compared to WT-HSA loaded with the same anti-CD scFvs (v1094).

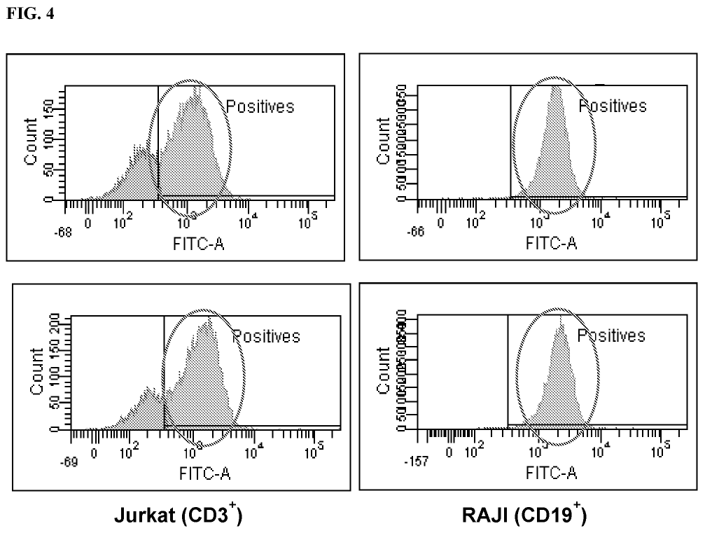

The results are shown in FIG. 4 and demonstrate that both v1092 and v1094 are able to bind to CD3-expressing Jurkat T-cells and to CD19-expressing Raji B-cells.

### Example 12: Heteromultimers with Heterodimeric Fc or Albumin Scaffolds Show Comparable B-Cell Targeting and T-Cell Bridging

The ability of heteromultimers with different scaffolds to direct B-cell targeting and T-cell bridging was compared by FACS analysis, according to the method described in Example 3. The v1092 construct was additionally tested against v873 and the v891 BiTE control for B-cell and T-cell binding according to the method described in Example 4.

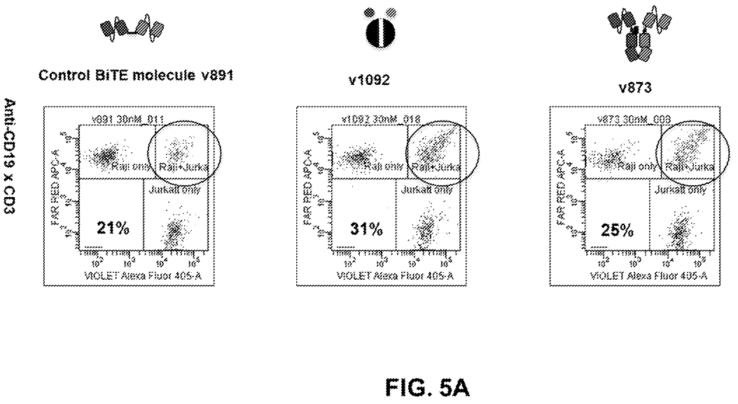

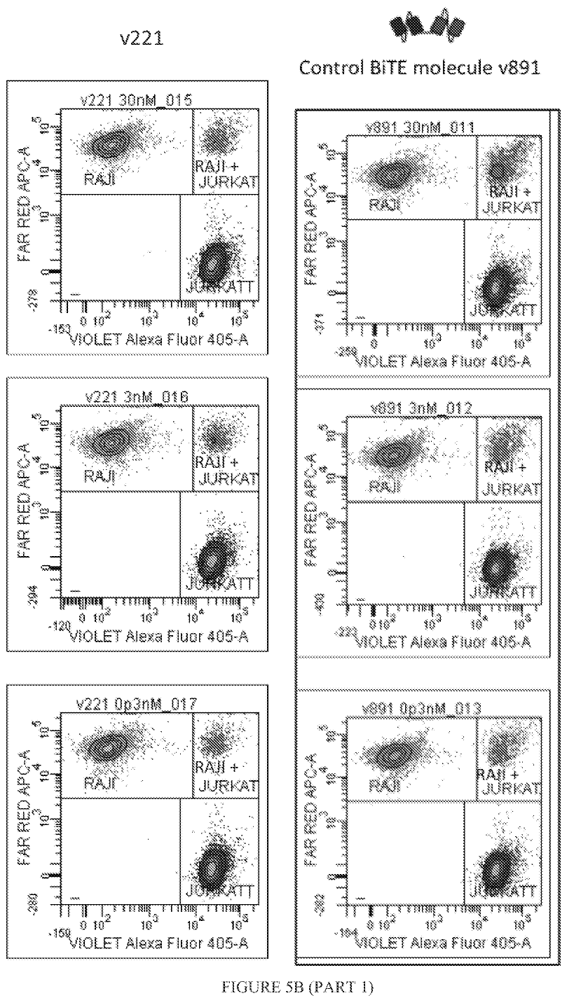

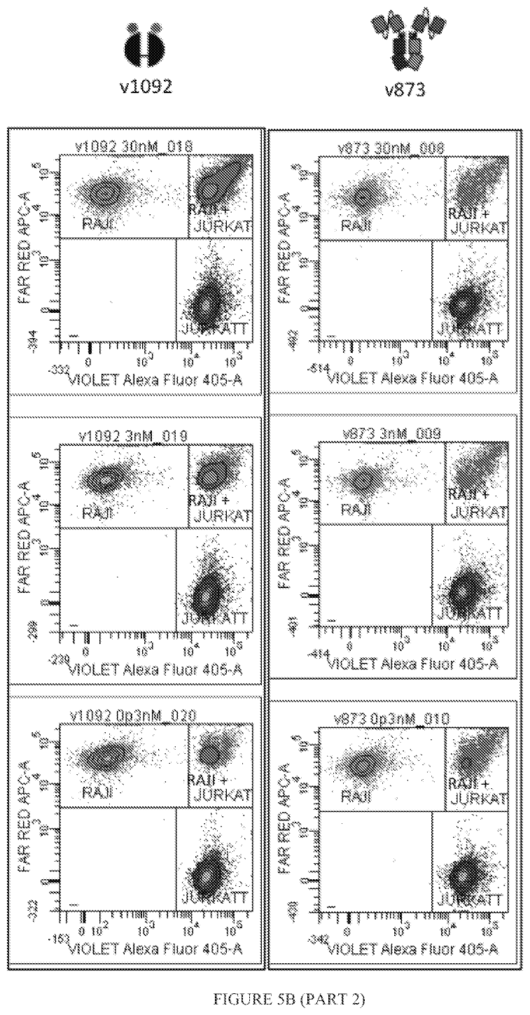

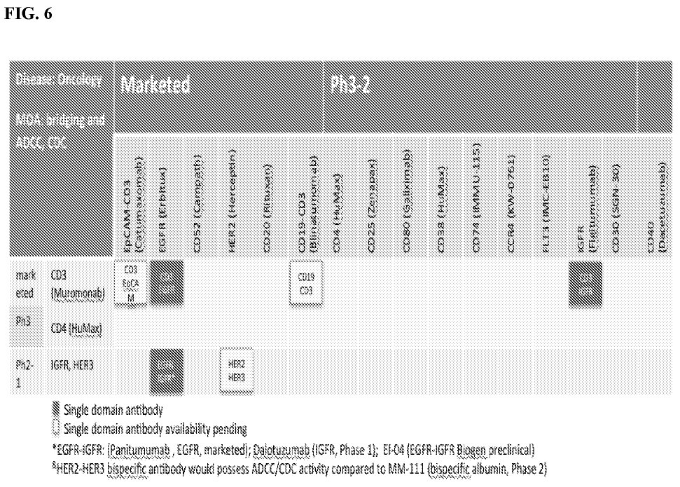

The results are shown in FIG. 5A and indicate that at the concentration tested, v1092 was able to bridge 31% of total cells, and v873 was able to bridge 25% of total cells (lower panels). The upper panels are the results using media as a control. As described in Example 3, the ability of v873 to bridge B-cells and T-cells is comparable to that of the v891 BiTE control. FIG. 5A further indicates that the ability of v1092 to bridge B-cells and T-cells is comparable to that of v873.

In an additional experiment, the ability of v1092 to bridge T-cells and B-cells was directly compared to the v221 control and to v873 and the v891 control. The results are shown in FIG. 5B which demonstrates that v1092 is able to bridge Jurkat T cells and Raji B cells to a greater extent than v221 and similar to v891 and v873.

### Example 13: Exemplary Heteromultimers have Higher Anti-CD3 K_{D} and Higher Bmax in Binding to T and B Cell as Determined by FACS

The K_{D} of exemplary heteromultimers, v873 and v875, was assessed by FACS as described as described in Example 4 with data analysis and curve fitting performed in GraphPad Prism.

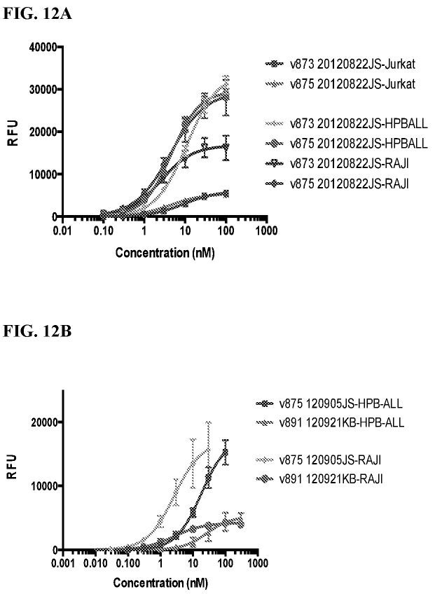

The results of this experiment are shown in FIG. 12 and indicate that both v873 and v875 have a higher anti-CD3 affinity compared to v891. The K_{D} for v873 and v875 binding to CD19 expressing Raji cells is similar across all antibodies and is comparable to v891. FIGS. 12A and B show FACS binding curves of v873, v875 and v891 to CD3 expressing HPB-ALL and Jurkat T cells, and to CD19 expressing Raji B cells. FIG. 12B also illustrates the v875 has a higher Bmax for binding to Raji B cell and Jurkat T cells when compared to v891. Table 1 summarises the K_{D} values for v875, v873, and v891 binding to HPB-ALL, Jurkat and Raji cells.

TABLE 1
KD summary of T and B cell binding determined by FACS

| | Jurkat | HBP-ALL | Raji |
|---|---|---|---|
| | Binding | Binding | Binding |
| | (nM) | (nM) | (nM) |
| v873 | 9.3 ± 0.6 | 11.2 ± 1.0 | 2.7 ± 0.5 |
| v875 | 4.5 ± 0.003 | 4.7 ± 0.3 | 4.5 ± 0.9 |
| v891 | NA | 64.5 ± 70.3 | 2.7 ± 1.2 |

The K_{D} of exemplary heteromultimers v875, v1379, v1380, v1381, and v891 was assessed for binding to CD19 expressing Raji cells by FACS as described as described in Example 4 with data analysis and curve fitting performed in GraphPad Prism.

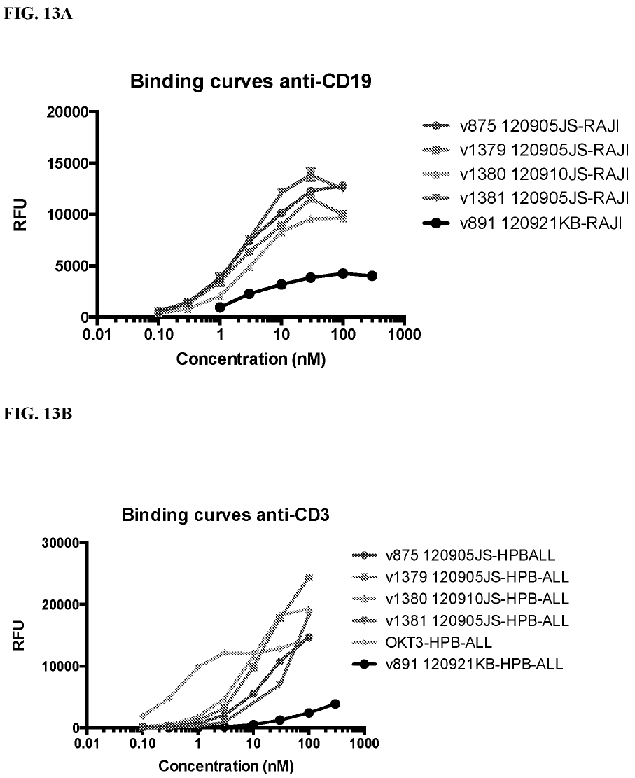

The results of this experiment are shown in FIG. 13A and Table 2 and indicate that heteromultimers v875, v1379, v1380, v1381, and v891 have a similar K_{D} for binding to CD19 expressing Raji cells. FIG. 13A illustrates the FACS binding curve obtained with the antibodies tested in 0.1 to 300 nM range. Table 2 summaries the derived K_{D} in nM, Bmax and Hill slope obtained from the FACS binding experiment. FIG. 13A also shows that all heteromultimers tested have a higher Bmax for binding to CD19 expressing Raji cells compared to BiTE.

TABLE 2
Summary of binding properties of heteromultimers to Raji B cells

| | v875 | v1379 | v1380 | v1381 | v891 |
|---|---|---|---|---|---|
| Bmax | 13140 | 10978 | 9955 | 13892 | 4153 |
| Hill | 0.9789 | 1.067 | 1.230 | 1.206 | 1.072 |
| slope | | | | | |
| KD | 2.491 | 2.124 | 2.946 | 2.360 | 2.841 |

The results of the FACS binding to HBP-ALL T cells are shown in FIG. 13B and Table 3 and indicate that heteromultimers v875, v1379, v1380, have a lower K_{D} compared to v891. FIG. 13B illustrates the FACS binding curve obtained with the antibodies tested in 0.1 to 300 nM range. Table 3 summaries the derived K_{D} in nM, Bmax and Hill slope obtained from the FACS binding experiment. FIG. 13B also shows that all heteromultimers tested have a higher Bmax for binding to HBP-ALL compared to v891.

TABLE 3
Summary of binding properties of heteromultimers to HBP-ALL T cells

| | v875 | v1379 | v1380 | v1381 | OKT3 | v891 |
|---|---|---|---|---|---|---|
| Bmax | 17118 | 27997 | 20420 | 99557 | 13339 | 6295 |
| Hill | 1.099 | 1.104 | 1.296 | 0.9148 | 1.195 | 0.8300 |
| slope | | | | | | |
| KD | 19.05 | 17.89 | 7.547 | 508.4 | 0.4607 | 168.4 |

### Example 14: Heteromultimer v875 is Able to Bridge Jurkat CD3 T Cells and Raji CD19 B Cells

The ability of the heteromultimer, v875, to bridge T cells and B cells was tested by FACS analysis as described in Example 3.

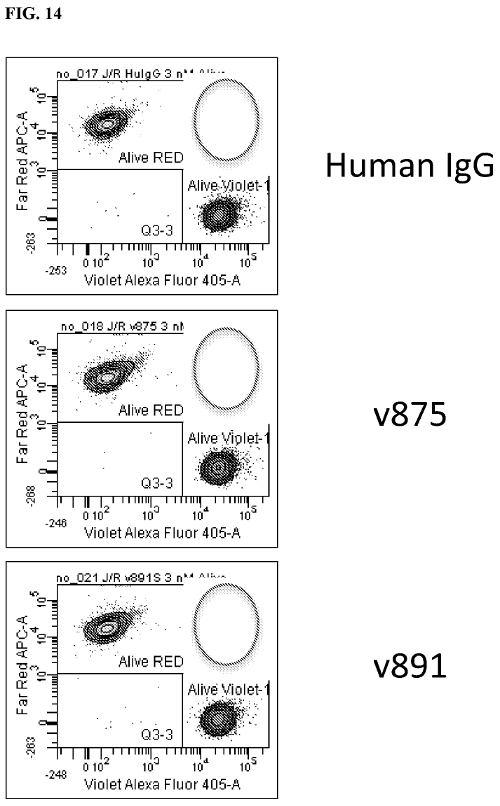

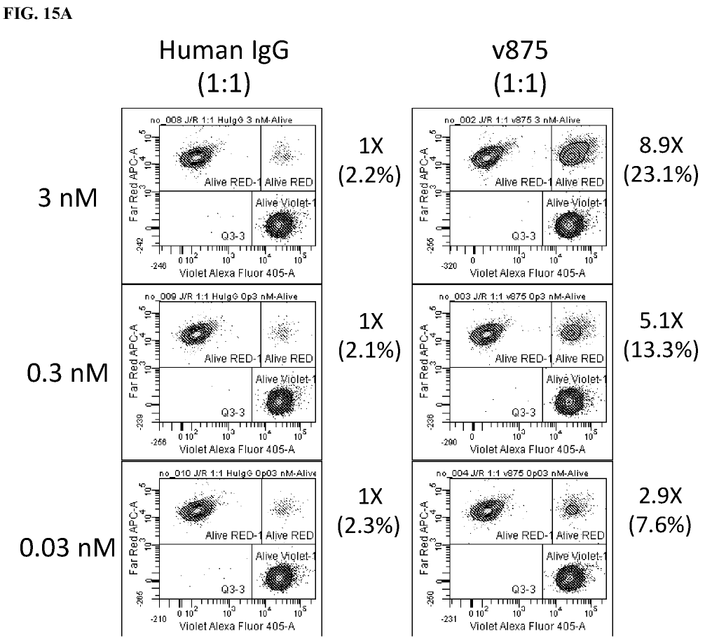

The results of this experiment are shown in FIG. 14 and indicate that both v875 and v891 facilitate comparable bridging between Raji B-cells and Jurkat T-cells. Use of the control human IgG resulted in 2.5% bridging between Raji and Jurkat cells, while v875 facilitated bridging of 22.9% of total cells, and v891 facilitated bridging of 14.5% of total cells. These results were also presented as fold bridging over background, where v875 mediated a 9.2-fold increase in bridging over background, while v891 mediated a 5.8-fold increase in bridging over background.

### Example 15: Bridging of B and T Cells by Heteromultimers is Robust at Varying Antibody Concentrations or Cell Ratios

The ability of v875 to bridge B and T cells at varying concentrations was assessed by FACS as described in Example 3 with modifications including changes to the effector to target (E:T) cell ratios (1:1 or 15:1) at three different concentrations of v875.

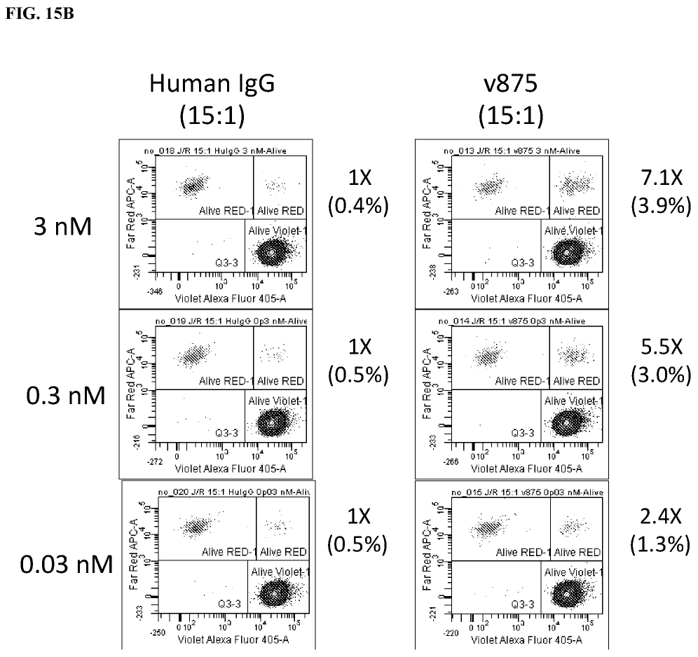

The results are shown in FIGS. 15A and 15B. FIG. 15A shows the amount of bridging using a 1:1 ratio of T-cells to B-cells, with heteromultimer concentrations ranging from 0.3 nM to 3 nM. FIG. 15B shows the amount of bridging using a 15:1 ratio of T-cells to B-cells, with heteromultimer concentrations ranging from 0.3 nM to 3 nM. Both E:T ratios (1:1 and 15:1) tested with v875 resulted in similar total T cell-B cell bridging when expressed as fold over background.

### Example 16: Bridging of B and T Cells is Robust Across Differently Engineered Heteromultimers Constructs

The ability of v875 to bridge Raji B and Jurkat T cells (B:T), as well Raji:Raji B cell bridging (B:B) and Jurkat:Jurkat T cell bridging (T:T) was assessed by FACS as described in Example 3.

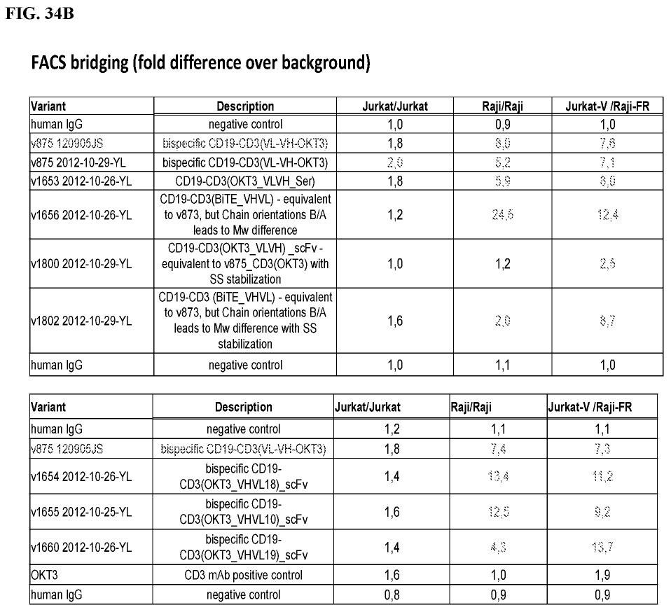

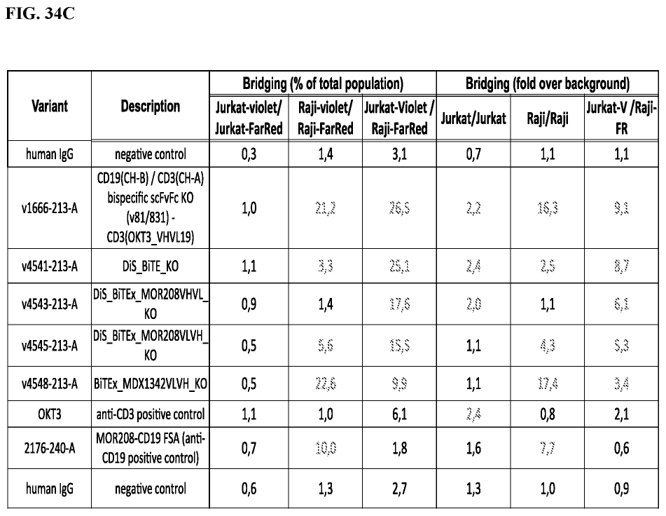

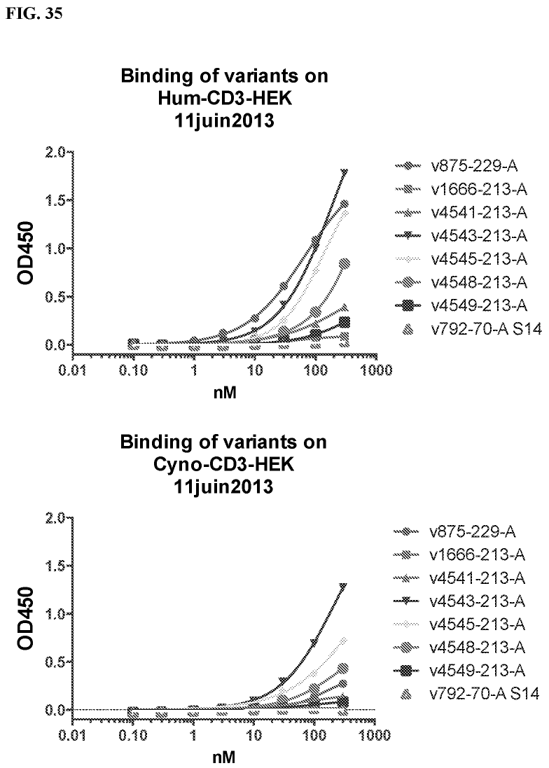

The results are shown in FIGS. 35A, 34B and 34C. FIG. 34A shows the amount of T:B, B:B and T:T bridging of v875, v1379, v1380, v891, v1381, commercial OKT3 and human IgG over three experimental replicates. These results show that all heterodimeric antibodies mediate a high percentage of T:B bridging and that all variants have a % T:B bridging that is higher than v891. These results also show a low percentage of T:T and B:B bridging relative to the % T:B bridging for all variants.

FIG. 34B shows the amount of T:B, B:B and T:T bridging of variants with engineered anti-CD3 warheads for stability enhancement (v1653, v1654, v1655, v1656, v1660, v1800, v1802) and v875, and human IgG. These results show that all heterodimeric antibodies (v875, v1653, v1654, v1655, v1656, v1660, v1800, v1802) mediate a higher percentage of T:B bridging compared to human IgG negative control, and all variants mediate low T:T bridging. These results also show that some variants (v1660, v1654 and v1655) mediate higher B:B bridging relative to T:B bridging.

FIG. 34C shows the amount of T:B, B:B and T:T bridging of Fc knock-out variants that have either engineered anti-CD3 warheads for stability enhancement (v1666), or have human/cynomolgous monkey cross-reactive anti-CD3 and anti-CD19 scFvs (v4541, v4543, v4545, v4548) commercial OKT3 anti-CD3 control, v2176 anti-CD19 control and human IgG negative control, and all variants mediate low T:T bridging. These results show that all heterodimeric antibodies (v1666, v4541, v4543, v4545, v4548) mediate a higher percentage of T:B bridging compared to human IgG negative control. These results also show that some variants (v1666, v4548) mediate higher B:B bridging relative to T:B bridging.

### Example 17: Effects of v875, v1380, v1379 on IL-2 Activated and Resting CD20+, CD4+, CD8+Subsets

The effects of v875, v1379 and v1380 treatment on the viability of CD20+, CD4+, CD8+ subsets in IL-2 activated or resting T and B cell cultures, was examined by 7AAD+staining and FACS and normalized to isotype control (n=4 donors), as described in Example 5.

The results are shown in FIG. 23, and illustrate that the exemplary heteromultimers v875, v1379 and v1380, mediate CD20+ B cell cytotoxicity but not CD4+ or CD8+ T cell cytotoxicity relative to the human IgG control. FIG. 23A shows the effects of v875 on the viability of CD20+, CD4+, CD8+ subsets in IL-2 activated cell cultures. FIG. 23B shows the effects of v875 on the viability of CD20+, CD4+, CD8+ subsets resting cell cultures. FIG. 23C shows the effects of v1379 and v1380 on the viability of CD20+, CD4+, CD8+ subsets in IL-2 activated cell cultures. FIG. 23D shows the effects of v1379 and v1380 on the viability of CD20+, CD4+, CD8+ subsets resting cell cultures. The contribution of the WT Fc to CD20+ B cell cytotoxicity is also show in FIGS. 23C and D, where v1379 mediates a greater to CD20+ B cell cytotoxicity in both IL-2 activated and resting CD4+ and CD8+ T cells.

### Example 18: Exemplary Heteromultimers v875 and v873 Require the Presence of Both Effector T Cell and Target B Cells to Mediate Cytotoxic Effects

The impact of incubating v875 and v873 alone with either effector or target cells was assessed by LDH release as described in Examples 5 with the following modifications. A total of 300,000 resting and IL-2 activated PBMC, 150,000 CD8+ effector cells or 10,000 Raji target cells were incubated overnight with each of the antibodies at 300 nM, along with a control without test item for each condition. The data presented is the averaged results from the 3 donors

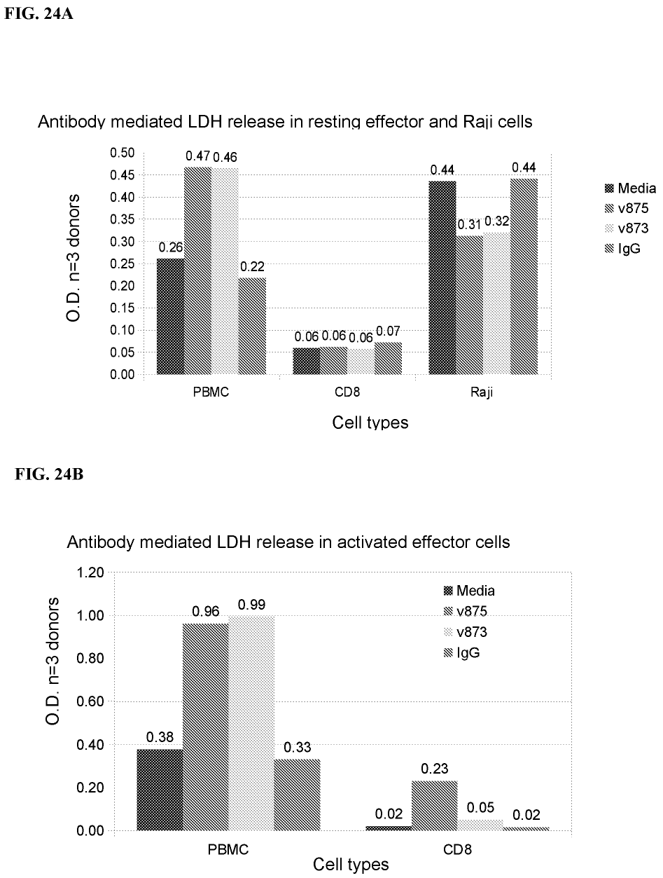

The results of antibody mediated LDH release in resting effector and Raji B cells shown in FIG. 24A and the results of antibody mediated LDH release in activated effector are shown in FIG. 24B. FIG. 24A shows that v875 and v873 are not cytotoxic to CD8+ T cell populations relative to the untreated media and human IgG control. FIG. 24A also shows that v875 and 873 increase the cytoxicity in total PBMC populations, likely due to the redirected killing of effector T cells to target B cells. Additionally, FIG. 24A shows that in the absence of effector T cells, v875 and v873 are not cytotoxic to Raji B cell populations relative to the media and human IgG control.

FIG. 24B shows the results of v875 and v873 mediated LDH release in activated effector PBMC and CD8+ T cells. These results show an increase in cell death when v875 and v873 are incubated with activated PBMC like due to the redirected B cell killing mediated by the heterodimeric antibodies in the presence of effector T and target B cells. FIG. 24B also shows that incubation of v875 with activated CD8+ T cells, results in a higher percentage of cell death relative to v873 and the media and human IgG controls.

### Example 19: Exemplary Heteromultimers can Mediate ADCC or Impaired ADCC to Target Daudi B Cells

Antibody-dependent cell-mediated cytotoxicity assays (ADCC) were performed with v875, v1379 and v1380 using Daudi cells as target B cells and FcRy3a immobilized NK92 cells as the effector cells (GS193761) by the following method.

Dose-response studies were performed with various concentrations of the samples with a pre-optimized Effector/Target (E/T) ratio (5:1). Triton X-100 was added to cell controls without effector cells and antibody in a final concentration of 1% to lyze the target cells and it served as the maximum lysis control; assay buffers were added in to cell controls without effector cells and antibody and it served as the minimum LDH release control. Target cells incubated with effector cells without the presence of antibodies were set as background control of non-specific LDH release when both cells were incubated together.

Test article was incubated with cells at 37° C./5% CO2 for 5-6 hours and cell viability was assessed with a LDH kit. The absorbance was read at OD492 nm and OD650 nm. The percentages of cell lysis (OD492 nm) were calculated according the formula below:

Cell lysis %=100*(1-(ODSample data-ODtumor cells plus effector cells)/(ODMaximum release-ODMinimum release)). Half maximal effective concentration (EC50) values were analyzed withthe Sigmoidal dose-response non-linear regression fit by GraphPad Prism.

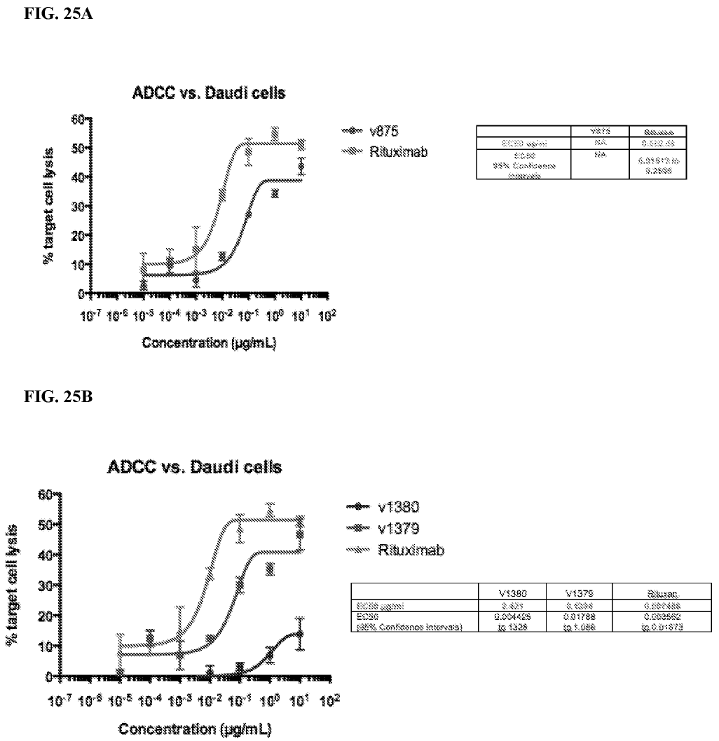

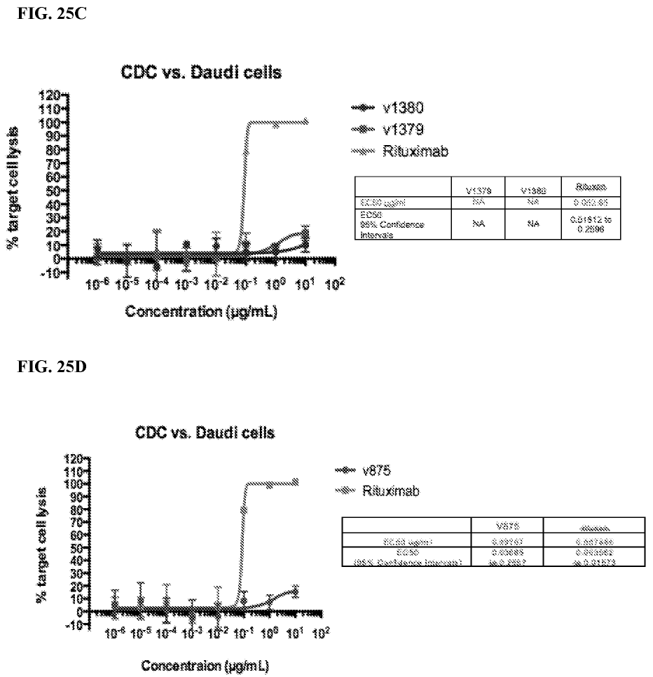

The results are shown in FIGS. 25A and B, and illustrate that heteromultimers with a WT Fc (v875 and v1379) can mediate ADCC (ca. 40% max cell lysis), whereas v1380, with a L234A_L235A knock Fc mutation, is impaired in ADCC to target Daudi B cells. In FIGS. 25A and B, comparison to the internal positive control, Rituximab, are presented.

### Example 20: Exemplary Heteromultimers have Impaired CDC-Mediated Lysis of Daudi B Cells

Cell based complement dependent cytotoxicity assays (CDC) were performed with v875, v1379 and v1380 using Daudi cells as target B cells. Human serum from healthy donors (NHS) was used as the source of complement. 10 μl NHS (10% final concentration in 40 reaction volume) were added to each well to initiate the CDC cascade and incubated for 2 hours. Cell viability was measured with CellTiter-Glo@ Luminescent Cell Viability Assay Kit.

The percentage of cell viability was calculated with the formula:

% Cell viability=100×((RLUsample)/(RLUcell+NHS)), in which NHS stands for normal human serum. Half maximal effective concentration (EC50) values were analyzed with the Sigmoidal dose-response non-linear regression fit by GraphPad Prism.

FIGS. 25C and D show the results of the CDC assay with v1380 and v1379 (FIG. 25C) and v875 (FIG. 25D) of target Daudi B cells with comparisons to positive control Rituximab. All heteromultimers show impaired CDC relative to Rituximab, and mediate a maximum target cell lysis of 15% or less.

### Example 21: Cell Proliferation and Cytokine Release Assessment of Exemplary Heteromultimers

The impact of incubating exemplary heteromultimers on cell proliferation and cytokine release was examined in PBMC and PBMC derived subpopulations. The PBMC derived subpopulations included PBMC, PBMC without B cells (PBMC-B), PBMC without NK cells (PBMC-NK), PBMC without NK and B cells (PBMC-NK-B), CD8+ T cells with B cells, and CD8+ without B cells. Briefly, Four (4) donors were tested for PBMC and PBMC derived subpopulations CD19-, CD56- or CD19/CD56-depleted PBMC populations) and two (2) donors were tested for CD8 and CD8 plus B cells during an incubation period of 4 and 6 days. The proliferation assay was performed with test items at 2 different concentrations (0.3 and 100 nM) each with thymidine incorporation as proliferation readout. The cytokines were measured using the CBA (Cytometric Bead Array) platform on a flow cytometer by the method described below.

A blood sample was collected from 6 normal donors (the CD8 counts of potential donors used in CD8 panel were determined previously), of whom two donors with high CD8 counts were selected for the CD8 experimental panel; one donor was also scheduled as back-up. On Day 1, about 135 mL of blood was collected from each of the 4 donors (for the PBMC panel), whereas on Day 2˜165 ml of blood was collected from each of the 2 donors (for the CD8 panel). On both days, PBMC were freshly isolated and the PMBC were passed through EasySep columns (STEMCELL Technologies Inc.) for CD19 and/or CD56 depletion by positive selection (day 1) and CD8 (±CD19) enrichment by negative selection (day 2).

To verify the composition/purity/viability of the selected subpopulations for both days, an antibody cocktail composed of CD8-FITC/CD56-PE/7AAD/CD19-PECY7/CD20-APC was employed.

The test items were prepared at 2× concentration for a final (after dilution with the cells) of 0.3 and 100 nM; were added in 100 μl of volume. The PBMCs were plated at 250,000 cells/well in 100 μl of suspension; the CD8 fractions were plated at 150,000 cells/well. The mixtures were incubated for 3 and 5 days respectively, after which 50 μl/well of cell supernatant was transferred into low binding plates and freeze for later cytokine analysis. 50 μl of tritiated thymidine was added to the cell containing wells for a final of 0.5 uCi thymidine/well; the plates were incubated for an additional 18 hours, after which the plates were frozen. Total incubation times were 4 and 6 days.

The plates were thawed two days later, filtered and counted (CPMs) using a β-counter.

The cell proliferation was assayed at 2 concentrations of test items (0.3 and 100 nM). From the averages, a Stimulation Index (SI) was calculated as follows and the data was tabulated: average CPM of test item/average CPM of media only

The composition/viability (trypan blue, 7AAD) of PBMCs and CD8-enriched cell populations were assessed for their respective proportions after selection using an antibody cocktail including anti: CD8/CD56/7AAD/CD20/CD56.

Once the proliferation results were analyzed, the supernatant from the replicates were pooled, excluding any identified outlier from the proliferation results. The pooled supernatant was used for cytokine measurements, in duplicates, using the CBA Human Th1/Th2 Cytokine Kit II from BD Biosciences. This kit measures IL-2, IL-4, IL-6, IL-10, TNF and IFg.

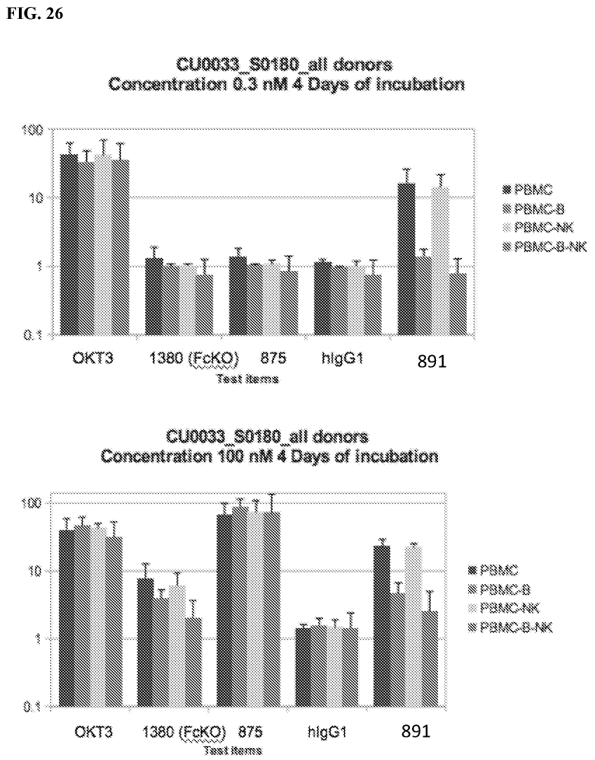

The results from the cell proliferation 4-day incubation assay are shown in FIG. 26 for the 0.3 nM (top panel) and the 100 nM (bottom panel) concentrations. The top panel of FIG. 26 shows that at 0.3 nM, v875 and v1380 do not induce PBMC proliferation compared to human IgG. The lower panel of FIG. 26 shows the results of the 100 nM antibody concentrations, and shows that v875, v1380 and v891 induce higher cell proliferation relative to human IgG. FIG. 26 (lower panel) also shows that at 100 nM, v875 has a similar proliferative index compared to anti-CD3 OKT3 in all four PBMC populations. However, compared to v875, v1380 (L234A_L235A Fc knock out variant) and v891 mediate a reduced cell proliferation and show a trend toward B cell dependence for cell proliferation, where the cell proliferation is lower in the PBMC-B and PBMC-B-NK subpopulations.

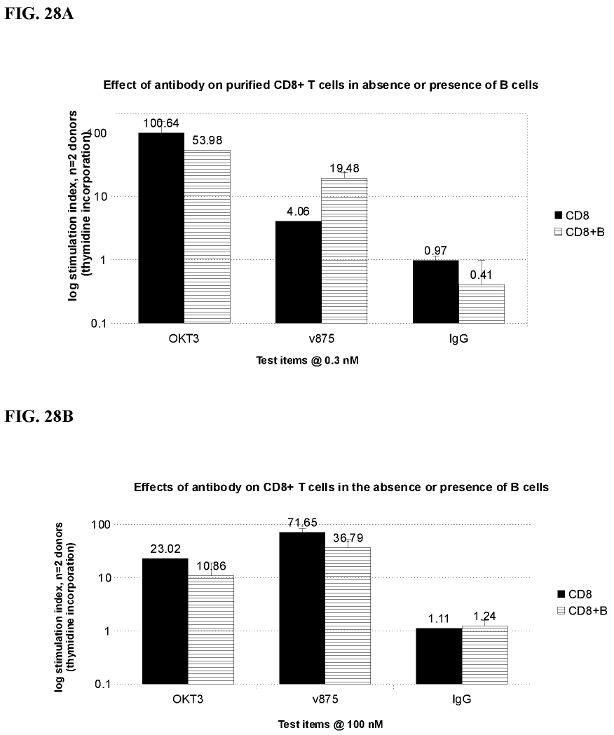

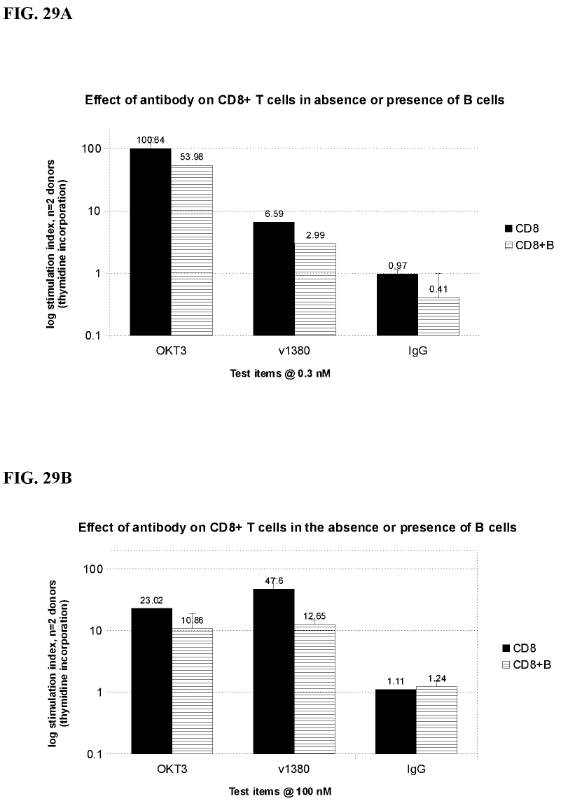

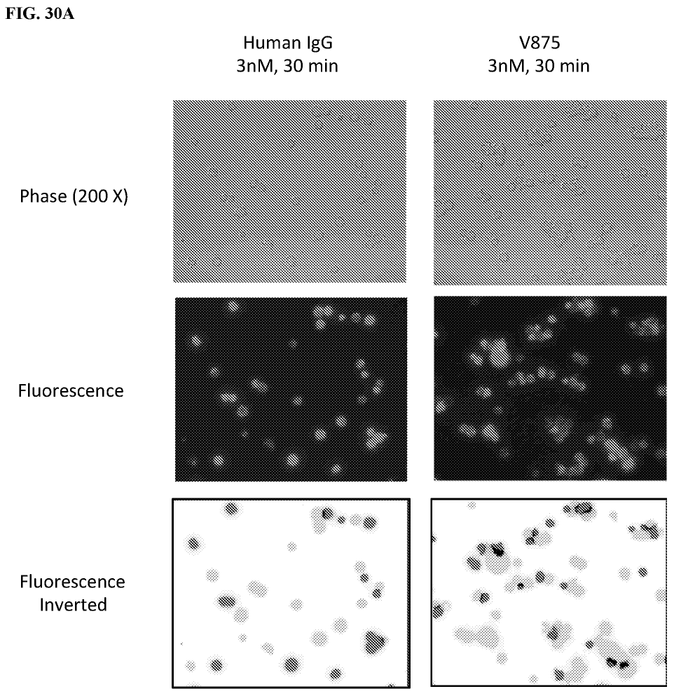

FIG. 28 show the results from the average stimulation index induced by v875 at 0.3 nM (FIG. 28A) and 100 nM (FIG. 28B) concentrations on purified CD8+ T cells in the absence or presence of purified CD19+ B cells at 4 days incubation time-point. FIG. 28A shows that at 0.3 nM, v875 has a higher stimulation index compared to human IgG and lower compared to OKT3. FIG. 28A also shows that v875 has a higher stimulation index of CD8+ T cells in the presence of target B cells (CD8+B). FIG. 28B shows that at 100 nM, v875 has a similar stimulation index compared to OKT3 and higher compared to human IgG with little to no influence of target B cell populations.

FIG. 29 shows the results from the average stimulation index induced by v1380 at 0.3 nM (FIG. 29A) and 100 nM (FIG. 29B) concentrations on purified CD8+ T cells in the absence or presence of purified CD19+ B cells at 4 days incubation time-point. FIG. 29A shows that at 0.3 nM, v1380 has a higher stimulation index compared to human IgG and lower compared to OKT3. FIG. 28A also shows that v1380 has a higher stimulation index of CD8+ T cells in the absence of target B cells (CD8). FIG. 29B shows that at 100 nM, v1380 has a similar stimulation index of CD8+ T cells compared to OKT3 and a higher stimulation index compared to human IgG. Similar to the 0.3 nM data, v1380 has a higher stimulation index of CD8+ T cells in the absence of target B cells (CD8). The L234A_L235A Fc knockout mutation in v1380 seems to reduced the B-cell dependent stimulation of CD8+ T cells that was apparent with the exemplary WT Fc variant v875.

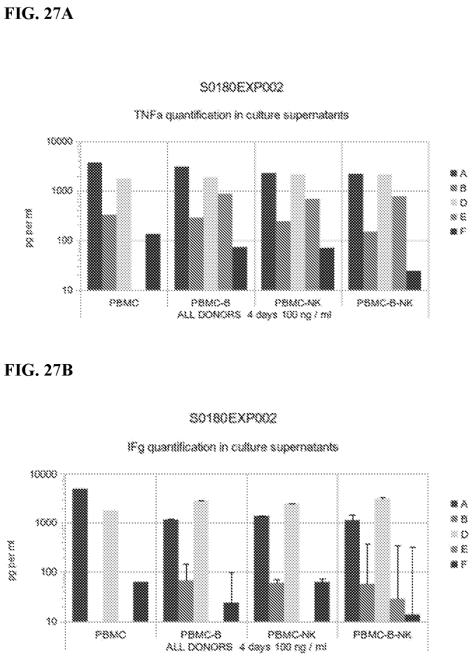

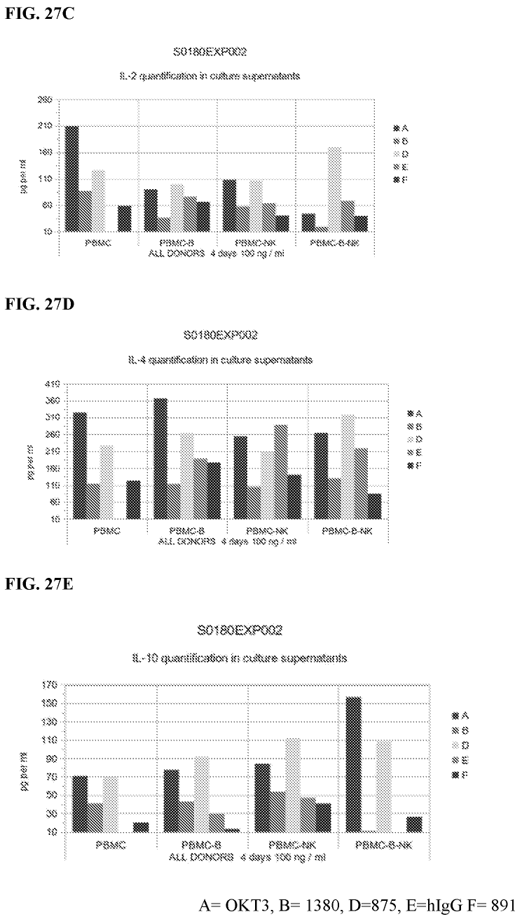

The results from the cytokine release assay are shown in FIG. 27 and include summary plots of PBMC supernatant TNFα (FIG. 27A) INFγ (FIG. 27B), IL-2 (FIG. 27C), IL-4 (FIG. 27D), and IL-10 (FIG. 27E) levels following incubation with test items at 0.3 nM concentrations for 4 days (graph y-axis represents log cytokine levels in pg per mL from 4 donors). FIG. 27 shows that v1380 (L234A_L235A Fc knockout) induces less cytokine release of TNFα, INFγ, IL-2, IL-4, and IL-10 when compared to v875 (WT Fc) and OKT3.

### Example 22: Exemplary Heteromultimers can Bridge Two or More Target B Cells Per Effector T Cell

The ability and ratio of numbers of T cell bridged to B cells was examined with the exemplary heteromultimer v875 by microscopy using the method described in Example 3 with the following modifications.

Labeled Raji B cells (red) and labeled Jurkat T cells (blue) were incubated for 30 min at RT with 3 nM of human IgG or v875. The cell suspension was concentrated by removing 180 μl of supernatant. Cell were resuspended in the remaining volume and imaged at 200× and 400×.

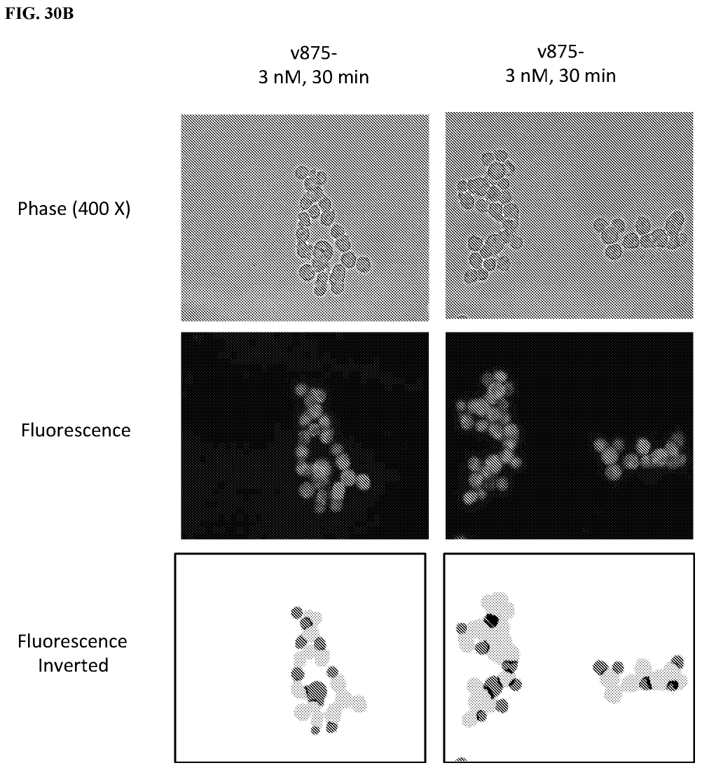

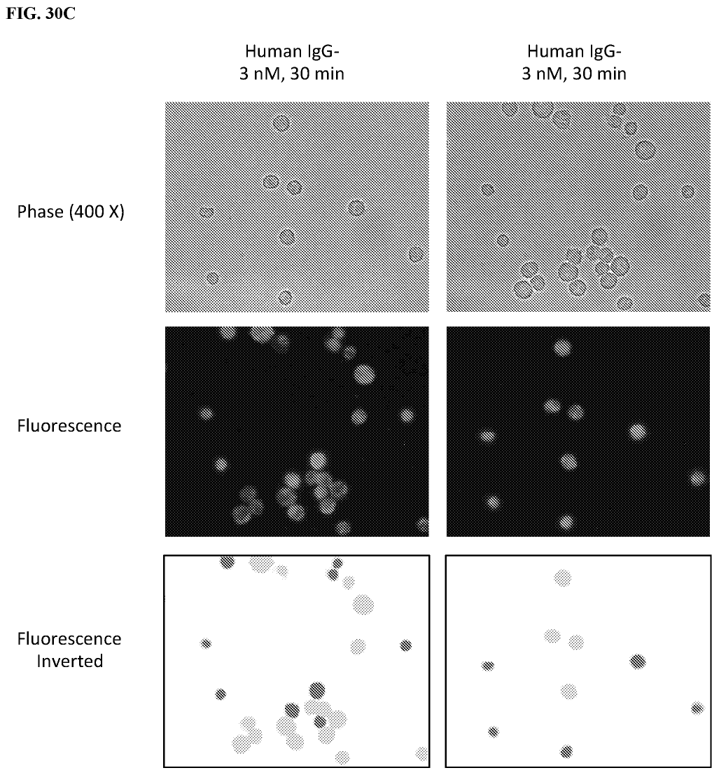

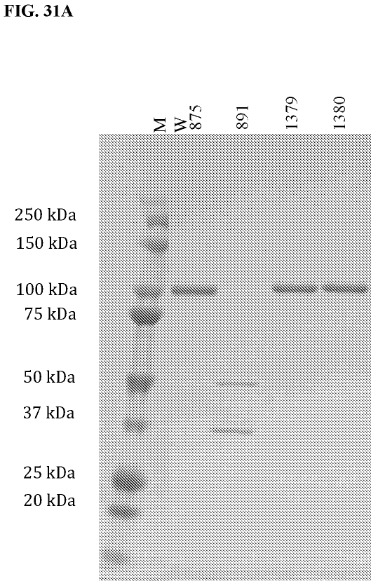

FIG. 30 shows the results from the T:B cell bridging microscopy comparing v875 and human IgG (3 nM) at 200× and 400× magnification; the phase image (top panel), fluorescence image (middle panel) and inverted fluorescence (bottom panel) are presented. FIG. 30A shows a direct comparison of human IgG and v875 at 200× magnification and illustrates a higher amount of bridging visible between Raji B cell and Jurkat T cells compared to human IgG. FIG. 30B and FIG. 30C show two fields of view for v875 (FIG. 30B) and human IgG (FIG. 30C) at 400× magnification. FIG. 30B shows images of v875-mediated immune complex formation between Jurkat T cells (dark grey cells in fluorescence inverted image) and Raji B cells (light grey cells in fluorescence inverted image), and that one Jurkat T cell can bridge 1-3 Raji B cells. FIG. 30C shows images following human IgG incubation with Jurkat T cells and Raji B cells. FIG. 30C shows an absence of the Jurkat-Raji bridging following incubation with human IgG, compared to the v875-mediated Jurkat: Raji bridging that is visible in FIG. 30B.

### Example 23: Exemplary Heteromultimer Binding to Fcγ Receptors as Assessed by Surface Plasmon Resonance

The ability of the exemplary heteromultimeric antibodies to bind to FcγRs CD16a and CD32a/b was examined using Surface Plasmon Resonance (SPR).

Surface Plasmon Resonance Analysis: Affinity of FcγR receptors to antibody Fc was measured by SPR using a ProteOn XPR36 system from BIO-RAD. Purified anti-CD3/anti-CD19-based antibodies were indirectly captured when injected at 25 L/min for 240s (resulting in approx. 500 RUs) following a buffer injection to establish a stable baseline.

FcγR concentrations (10,000, 3333, 1111, 370, 123 nM) were injected at 60 μL/min for 120s with a 180s dissociation phase to obtain a set of binding sensograms. Resultant K_{D} values were determined from binding isotherms were fit globally to a 1:1 Langmuir binding model with reported values as the mean of three independent runs.

The results of the SPR binding studies are shown in Table 4.

TABLE 4
KD for heterodimeric antibody binding to Fc gamma receptors

| | CD16aWT | | CD16aV158 | | CD32aWT | | CD32aR131 | | CD32bWT | | CD32bY163 | |
|---|---|---|---|---|---|---|---|---|---|---|---|---|
| | KD | | KD | | KD | | KD | | KD | | KD | |
| | avg. | SD | avg. | SD | avg. | SD | avg. | SD | avg. | SD | avg. | SD |
| V875 | 1.0E−06 | 1.0E−07 | 3.6E−07 | 4E−08 | 5.9E−07 | 8E−08 | 6.0E−07 | 4E−08 | 4.3E−07 | 6E−08 | 1.3E−06 | 2E−07 |
| V1379 | 1.1E−06 | 4.1E−07 | 4.1E−07 | 4E−08 | 6.0E−07 | 1E−07 | 7.8E−07 | 8E−08 | 4.7E−07 | 6E−08 | 1.1E−06 | 2E−07 |
| V1380 | 5.0E−06 | 3.2E−06 | 3.2E−06 | 8E−07 | low | | NB | | NB | | low | |
| V1381 | NB | | NB | | NB | | NB | | NB | | NB | |
| WT | 2.4E−06 | 1.0E−07 | 8.6E−07 | 5E−08 | 1.2E−06 | 3E−07 | 1.6E−06 | 3E−07 | 9.9E−07 | 3E−08 | 2.9E−06 | 2E−07 |
| herceptin | | | | | | | | | | | | |

Table 4 summarized the K_{D} data for exemplary heteromultimer binding to CD16a, CD16aV158, CS32a, CD32aR131, CD32b and CD32bY163. These results show that WT Fc variants v875, v1379 and WT Herceptin bind to all Fc gamma receptors. These results also show that Fc knock out variants have impaired binding to some (v1380) or all (v1381) Fc gamma receptors.

### Example 24: Exemplary Heteromultimers can Bind to the Human and Cynomolgous CD3 T Cell Receptor

The binding of exemplary heteromultiers for binding to the human CD3 and cynomologous receptors was examined by ELISA by the following method.

Human or cynomologous CD3 receptor antigen was diluted at 20 μg/ml in PBS, in Costar 3690 high binding half area high binding microplate, incubated overnight at 4° C. Wells were washed 3 times with PBS and blocked with BSA 1% in PBS for 30 min (50 μl/well). Primary heteromultimeric antibodies were diluted in BSA 1% at the indicated concentrations and incubated for 2 hours at RT and wells washed 4 times with PBS-0.05% Tween 20. Secondary antibody (Jackson 115-036-062: anti-mouse or 709-036-098 anti-human Fcgamma specific) was diluted 1/5000 in BSA 1% (25 μl/well) and incubated for one hour at RT and washed 4 times with PBS-0.05% Tween 20. TMB substrate was added (25 μl/well) for 25-30 min., and the reactions was stopped with 1M H2SO4 (12.5 μl/well) and OD read at 450 nm.

The ELISA data is shown in FIG. 35 and illustrates binding to human CD3 (top panel) and binding to the cynomologous CD3 receptor (bottom panel) as determined by ELISA. FIG. 35 shows that v4543, v4545, and v4548 show that highest degree of cross-reactivity to the cynomologous CD3 receptor (FIG. 35 bottom panel).

### Example 25: Expression and Purification of Heteromultimers

Description of the methods used in the expression and purification of exemplary heteromultimer are described in Example 2.

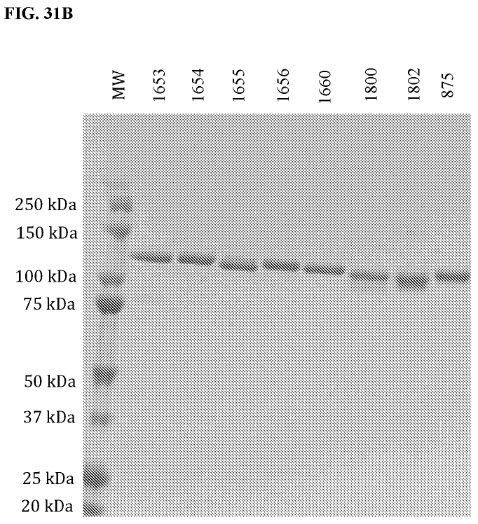

FIG. 31A shows the SDS-PAGE analysis and relative purity of v875, v1380, v1379 and v891 following protein A and SEC purification, and following 47 day storage at 4° C. The two visible protein bands in the v891 sample indicate a breakdown product of this sample due to storage.

FIG. 31B shows the SDS-PAGE analysis and relative purity of additional exemplary hetermultimers including v875, v1653, v1654, v1655, v1656, v1660, v1800, and v1802 following protein A and SEC purification.

### Example 26: Exemplary Heteromultimers can be Purified to >99% Heterodimer Purity and <1% Aggregates

The purity of exemplary heteromultimers was tested by LC-MS. The heteromultimers were first purified by protein A, protein L and SEC purification as described in Example 2. LC-MS analysis for heterodimer purity was performed as described below.

The purified samples were de-glycosylated with PNGase F for 6 hr at 37° C. Prior to MS analysis the samples were injected onto a Poros R2 column and eluted in a gradient with 20-90% ACN, 0.1% FA in 3 minutes, resulting in one single peak.

The peak of the LC column was analyzed with a LTQ-Orbitrap XL mass spectrometer using the following setup: Cone Voltage: 50 V′ Tube lens: 215 V; FT Resolution: 7,500. The mass spectrum was integrated with the software Promass or Max Ent. to generate molecular weight profiles.

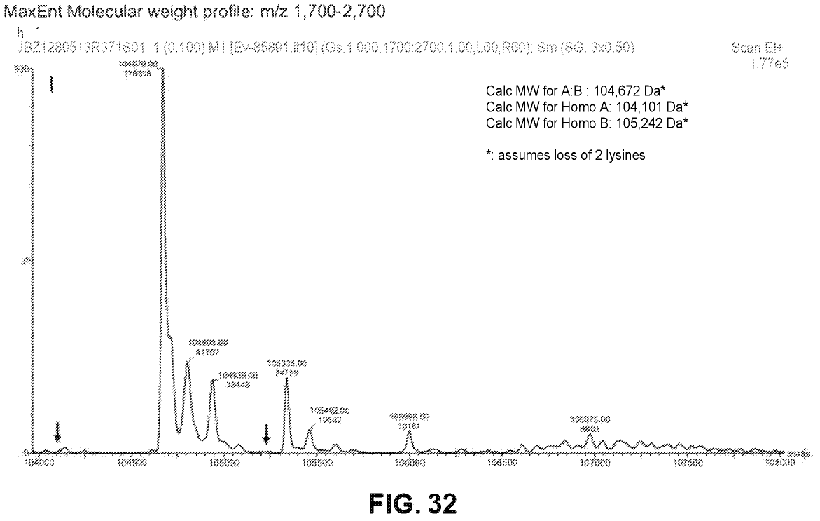

The LC-MS results of the Max Ent. molecular weight profiles for v875 are shown in FIG. 32 and the results summarized in Table 5.

TABLE 5
Heterodimer purity of v875

| | | SEC | | | |
|---|---|---|---|---|---|
| | | fraction | Amount | Amount | Amount |
| Lot | Ratio | Index | H1H1 | H1H2 | H2H2 |
| v875 -229-A | 1:1 | N/A | 0.35 | 99.27 | 0.39 |

Table 5 shows that following protein A and SEC purification, v875 consistend of 99.27% heterodimer purity (H1H2) and less than 1% of the anti-CD3 homodimer (H2H2) and anti-CD19 homodimer (H1H1).

The purity and percent aggregation of exemplary protein A and SEC purified heteromultimers was determined by UPLC-SEC by the method described.

UPLC-SEC analysis was performed using a Waters BEH200 SEC column set to 30° C. (2.5 mL, 4.6×150 mm, stainless steel, 1.7 μm particles) at 0.4 ml/min. Run times consisted of 7 min and a total volume per injection of 2.8 mL with running buffers of 25 mM sodium phosphate, 150 mM sodium acetate, pH 7.1; and, 150 mM sodium phosphate, pH 6.4-7.1. Detection by absorbance was facilitated at190-400 nm and by fluorescence with excitation at 280 nm and emission collected from 300-360 nm. Peak integration was analyzed by Empower 3 software.

The LC-MS and UPLC-SEC results for exemplary heteromultimers is summarized in Table 6. Table 6 shows that all heteromultimers have a heterodimer purity >95% as determined by LC-MS, and all heteromultimers <2% aggregates as determined by UPLC-SEC analysis.

TABLE 6
Summary of LC-MS and UPLC-SEC
analysis of exemplary heterodimers

| | | LCMS | | | | |
|---|---|---|---|---|---|---|
| | | | % | % | % | % |
| | | % | Lighter | Heavier | Lighter | Heavier |
| | | Hetero- | Homo- | Homo- | Mono- | Mono- |
| Variant | UPLC | dimer | dimer | dimer | mer | mer |
| v875 | NA | 99.27 | 0.35 | 0.39 | | |
| 1666 | 98.3 | 96.1 | 0 | 3.9# | 0 | 0 |
| 4541 | 99 | 97.4 | 1.1 | 1.5 | 0 | 0 |
| 4543 | 99.97 | 98.0* | 0 | 2 | 0 | 0 |
| 4545 | 99.94 | 96.6* | 0 | 3.4 | 0 | 0 |
| 4548 | 98.5 | 95.9 | 0 | 4.1# | 0 | 0 |
| v4549 | NA | 97 | 1.5 | 1.5 | 0 | 0 |

#Interference from an adjacent peak making exact quantification impossible.
True relative intensity % of this peak is likely less than presented value.
*by LC-MS after treatment with neuraminidase

### Example 27: Exemplary Heteromultimers have a CH3 Tm that is Greater than 75° C.

CH3 domain stability of exemplary heteromultimers was examined by DSC using the following method. All DSC experiments were carried out using a GE VP-Capillary instrument. The proteins were buffer-exchanged into PBS (pH 7.4) and diluted to 0.3 to 0.7 mg/mL with 0.137 mL loaded into the sample cell and measured with a scan rate of 1° C./min from 20 to 100° C. Data was analyzed using the Origin software (GE Healthcare) with the PBS buffer background subtracted.

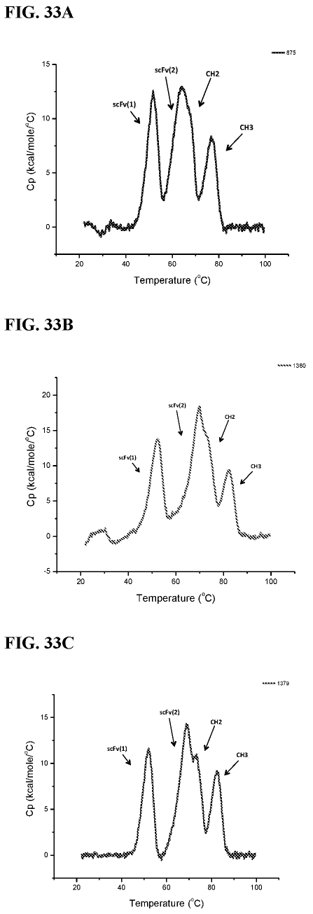

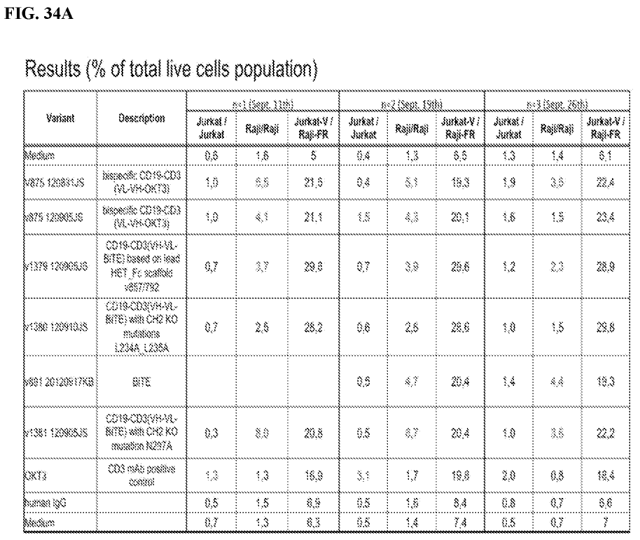

The DSC results shown in FIGS. 33A, B and C show that v875 has an estimated CH3 Tm >76° C. (FIG. 33A), v1380 has an estimated CH3 Tm >82.3° C. (FIG. 33B), and v1379 has an estimated CH3 Tm >82.5° C. (FIG. 33C).

### Example 28: Design, Expression and Purification of CD3/CD20 and Additional CD3/CD19 Heteromultimer Constructs

V5850 (corresponding to polypeptide sequences SEQ ID NOs: 203, 205 and 207), v5851 (corresponding to polypeptide sequences SEQ ID NOs: 209, 211 and 213), v5852 (corresponding to polypeptide sequences SEQ ID NOs: 215, 217 and 219), v6324 (corresponding to polypeptide sequences SEQ ID NOs: 221 and 223), v6325 (corresponding to polypeptide sequences SEQ ID NOs: 225, 227 and 229), v1813 (corresponding to polypeptide sequences SEQ ID NOs: 231, 233 and 235), v1821 (corresponding to polypeptide sequences SEQ ID NOs: 237, 239 and 241), v1823 (corresponding to polypeptide sequences SEQ ID NOs: 243, 245 and 247) exemplify bispecific CD3/CD19 or CD3/CD20 hybrid heterodimeric Fc constructs. Bispecific hybrid variants are composed of a F(ab′) on either chain A or B paired with an scFv-Fc on the alternate polypeptide chain. Chain A of the heterdimer Fc is comprised of the following mutations: T350V_L351Y_F405A_Y407V and Chain B of the heterdimer Fc is comprised of the following mutations: T350V_T366L_K392L_T394W. v6324 exemplifies bispecific CD3/CD20 scFv heterodimeric Fc constructs. V1813, v1821, and v1823 exemplify CD3/CD20 common light chain heterodimeric Fc constructs. Common light chain variants are composed of two different F(ab′) s, each on complimentary heterodimer Fc, which share a single light chain. The specific variant composition is indicated in Table 7.

The anti-CD19 MOR208_scFv-Fc (VHVL) used in v5852 was generated by fusing the published variable heavy chain sequence to the variable light chain sequences indicated in table 7 with a (GGGGS) 3 (SEQ ID NO: 281) linker between the heavy and light chain. The variable domains were fused to Chain B of the heterdimer Fc.

The anti-CD20 Ofatumumab_scFv-Fc (VHVL) used in v6324 and v6325 was generated by fusing the published variable heavy chain sequence to the variable light chain sequences indicated in Table 7 with a (GGGGS) 3 (SEQ ID NO: 281) linker between the heavy and light chain. The variable domains were fused to Chain B of the heterdimer Fc.

Cloning, expression and purification was performed as indicated in Example 2.

Yield and purity of the variants is indicated in Table 8. Heterodimer purity was determined by LCMS analysis according to Example 26. All the variants demonstrated heterodimer purity in excess of 73.8% with an average purity of 89.6% for all variants tested. The samples had low amounts of incorrectly paired homodimers ranging from 0 to 5.3% of the total product. Reported values represent the sum of all observed homodimer species. The presence of half-antibodies was more commonly observed than homodimers and ranged from 0 to 20.7% of the total product. Reported values represent the sum of all observed half-antibody species

TABLE 7
Composition of CD3/CD19 or CD20 hybrid variants

| | v5850 | V5851 | | V5852 | V6324 |
|---|---|---|---|---|---|
| Format | Hybrid | Hybrid | | Hybrid | scFv-Fc |
| Chain A | aCD3- | aCD3- | | aCD3- | aCD3- |
| | BiTEx_I2C_scFvFc | BiTEx_I2C_scFvFc | | Teplizumab- | BiTEx_I2C_scFvFc |
| | (VHVL) | (VHVL) | | hOKT3_Fab | (VHVL) |
| Chain B | aCD20- | aCD19- | | aCD19- | aCD20- |
| | Ofatumumab_Fab | MOR208_Fab | | MOR208_scFvFc_(VHVL) | Ofatumumab_scFvFc |
| | | | | | (VHVL) |
| Light | aCD20- | aCD19- | | aCD3- | N/A |
| Chain | Ofatumumab_Fab | MOR208_Fab | | Teplizumab-hOKT3 | |
| Reference | Chain A- | Chain A- | | Chain A- | Chain A- |
| | US2011/0275787 | US2011/0275787 | | US20070077246 | US2011/0275787 |
| | Chain B- | Chain B- | | Chain B- | Chain B- |
| | WO2004035607 | WO2008022152 | | Light Chain- | Light Chain- |
| | Light Chain- | Light Chain- | | US20070077246 | na |
| | WO2004035607 | WO2008022152 | | | |
| | | V6325 | v1813 | V1821 | V1823 |
| | Format | Hybrid | Common | Common | Common |
| | | | light chain | light chain | light chain |
| | Chain A | aCD3- | aCD3- | aCD3- | aCD3- |
| | | Teplizumab- | foralumab_Fab | 12F6_Fab | 12F6_Fab |
| | | hOKT3_Fab | | | |
| | Chain B | aCD20- | aCD20- | aCD20- | aCD20- |
| | | Ofatumumab_scFvFc | Ofatumumab_Fab | Rituximab_Fab | Tositumumab_Fab |
| | | VHVL) | | | |
| | Light | aCD3- | aCD20- | aCD20- | aCD20- |
| | Chain | Teplizumab- | Ofatumumab_Fab | Rituximab_Fab | Tositumumab_Fab |
| | | hOKT3 | | | |
| | Reference | Chain A- | Chain A- | Chain A- | Chain A- |
| | | US20070077246 | WHO drug | Pubmed ID: | Pubmed ID: |
| | | Chain B- | information | 16313362 | 16313362 |
| | | Light Chain- | Vol. 24, | Chain B- | Chain B- |
| | | US20070077246 | no2, 2010 | Drug bank | Drug bank |
| | | | Chain B- | accession | accession |
| | | | WO2004035607 | number: | number: |
| | | | Light Chain- | DB00073 | DB00081 |
| | | | WO2004035607 | Light Chain- | Light Chain- |
| | | | | Drug bank | Drug bank |
| | | | | accession | accession |
| | | | | number: | number: |
| | | | | DB00073 | DB00081 |

TABLE 8
Variant expression and purity

| | V5850 | V5851 | V5852 | V6324* | V6325 | v1813 | V1821 | V1823 |
|---|---|---|---|---|---|---|---|---|
| Expression | 50 | 50 | 50 | 50 | 50 | 500 | 500 | 500 |
| scale (ml) | | | | | | | | |
| Amount after | 1.25 | 0.72 | 0.57 | 0.34 | 0.42 | 17.4 | 2.16 | 8.8 |
| SEC (mg) | | | | | | | | |
| % | 95.6 | 100 | 95.1 | 85 | 97.5 | 78.4 | 91.4 | 73.8 |
| Heterodimer | | | | | | | | |
| (AB) | | | | | | | | |
| % Homodimer | 0 | 0 | 4.9 | 0 | 0 | 1.36 | 3.7 | 5.3 |
| (AA + BB) | | | | | | | | |
| % half- | 4.4 | 0 | 0 | 5.5 | 2.5 | 20.2 | 4.8 | 20.7 |
| antibody | | | | | | | | |
| (A + B) | | | | | | | | |

*unknown species detected by LCMS resulted in lower purity estimates
Example 29: CD3/CD20 and additional CD3/CD19 heteromultimer variants bind to T cells and B cells.

### Example 29: CD3/CD20 and Additional CD3/CD19 Heteromultimer Variants Bind to T Cells and B Cells

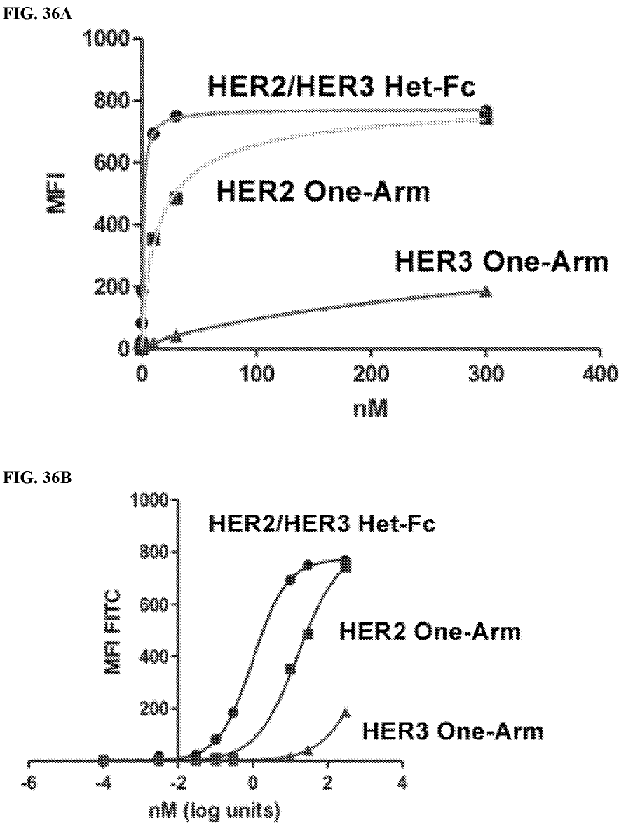

The ability of the exemplary CD3/CD20 heteromultimeters, v5850, v6324, v6325, v1813, v1821, v1823 to bind to CD3 and CD20 cells were assessed via FACS analysis as per procedures described in Example 4. Additionally, the ability of an exemplary CD3/CD19 heteromultimers, v5851 and v5852 to bind to CD3 and CD19 cells were similarly assessed. An additional variant v875, a CD3/CD19 BiTE Fc antibody construct, were also prepared and tested as benchmarks. Representative binding curves for v875, v5850 and v5851 on Raji and Jurkat cells are shown in FIGS. 36A & 38B. The binding results for each variant expressed in kinetic constants Bmax and Kd are listed below in Tables 9 and 10. Table 8 describes the binding to the CD19 and CD20-expressing Raji B cells, while Table 10 describes binding to the CD3-expressing Jurkat T cells. In Raji binding studies (Table 9) all the variants bound with a greater Bmax and a higher Kd compared to 875. In Jurkat binding studies (Table 10) all the variants, except v1823, bound with a higher Bmax than 875 and had a range of KD's.

TABLE 9
(Raji)

| | v875 | v4542 | v5850 | v5851 | v5852 | v6324 | v6325 | v1813 | v1821 | v1823 |
|---|---|---|---|---|---|---|---|---|---|---|
| Bmax | 2.78 | 2.96 | 4.24 | 3.88 | na | 6.91 | 6.44 | 6.40 | 4.71 | 4.14 |
| (OD450) | | | | | | | | | | |
| KD | 0.36 | 0.70 | 3.60 | 1.38 | na | 25.35 | 11.87 | 4.04 | 122.5 | 21.05 |
| (nM) | | | | | | | | | | |

TABLE 10
(Jurkat)

| | v875 | v4542 | v5850 | v5851 | v5852 | v6324 | v6325 | v1813 | v1821 | v1823 |
|---|---|---|---|---|---|---|---|---|---|---|
| Bmax | 1.59 | 2.27 | 2.06 | 2.51 | 2.21 | 2.32 | 2.51 | 2.54 | 2.11 | 0.88 |
| (OD450) | | | | | | | | | | |
| KD | 21.36 | 6.66 | 4.04 | 4.24 | 25.24 | 11.62 | 1.58 | 691.4 | 181.5 | 68.77 |
| (nM) | | | | | | | | | | |

### Example 30: CD3/CD20 and Additional CD3/CD19 Heteromultimer Variants Bridge T Cells and B Cells

The ability of the six exemplary CD3/CD20 heteromultimer variants-namely v5850, v6324, v6325, v1813, v1821 and v1823- and two exemplary CD3/CD19 heteromultimer variants-namely v5851 and v5852-to bridge T cells and B cells were tested via FACS analysis as per procedures described in Example 3. Additional constructs, namely v792 and v875, were also prepared and tested as controls. V792 has identical anti-Her2 F(ab′) based on trastuzumab on chain A and chain B of the heterodimer Fc with the following mutations T350V_L351Y_F405A_Y407V on chain A and T350V_T366L_K392L_T394W on chain B (drug bank accession number-DB00072)

Table 11 and 12 provides the percentage bridging between Jurkat-Jurkat, Raji-Raji, and Jurkat-Raji for each variant, each table represents an individual experiment. All variants were effective at bridging Jurkat and Raji cells. Furthermore, none of the variants bridged two jurkat cells and some Raji-Raji cell bridging was observed to different extents.

TABLE 11

| | % Bridging | | | | | | |
|---|---|---|---|---|---|---|---|
| | v792 | v875 | v5850 | v5851 | v1813 | v1821 | v1823 |
| Jurkat/ | 0.5 | 1.6 | 0.8 | 1.0 | 0.6 | 0.5 | 0.7 |
| Jurkat | | | | | | | |
| Raji/Raji | 2.6 | 10.2 | 2.1 | 1.6 | 7.0 | 2.4 | 2.1 |
| Jurkat/Raji | 2.6 | 17.0 | 11.6 | 23.2 | 16.2 | 7.3 | 8.1 |

TABLE 12

| % Bridging | v792 | v875 | v5852 | V6324 | v6325 |
|---|---|---|---|---|---|
| Jurkat/Jurkat | 0.7 | 0.5 | 0.9 | 0.8 | 1.1 |
| Raji/Raji | 0.7 | 8.6 | 7.2 | 1.5 | 0.7 |
| Jurkat/Raji | 1.9 | 15.7 | 30.4 | 5.7 | 15.7 |

### Example 31: Design, Expression and Purification of HER2/HER3 Heteromultimeric Constructs

To assess various properties of HER2/HER3 bispecific heteromultimeric constructs, two formats and various controls were produced and purified as follows. The first construct was produced by fusing HER2- and HER3-binding scFvs onto a heterodimer Fc region (v878, HER2/HER3 Het-Fc). The second construct was prepared by fusion of Her2 and Her3 scFvs onto an albumin-based platform (v1090, AlbuCORE anti-Her3×Her2). Various controls, including a Her2 One-Armed het-Fc, a Her3 one-armed het-Fc and the control HSA-fusion protein v1087 were also produced as controls.

### Design

The bispecific HER2/HER3 constructs were produced by fusion of Her2 and Her3 scFvs onto the het-Fc or the AlbuCORE platforms.

1. Variant 878: a monovalent one-armed anti-Her2 antibody, where the Her2 binding domain is a scFv on chain A, and the Fc region is a heterodimer having the mutations L351Y_F405A_Y407V in Chain A, and T366L_K392M_T394W in Chain B. The epitope of antigen binding domain is domain 1 of Her2.

2. Variant 879: a monovalent one-armed anti-Her3 antibody, where the Her3 binding domain is a scFv on chain B, and the Fc region is a heterodimer having the mutations L351Y_F405A_Y407V in Chain A, and T366L_K392M_T394W in Chain B.

3. Variant 880: a bispecific anti-Her2, anti-Her3 antibody, where the Her2 binding domain is a scFv on chain A, the Her3 binding domain is a scFv on chain B and the Fc region is a heterodimer having the mutations L351Y_F405A_Y407V in Chain A, and T366L_K392M_T394W in Chain B.

4. Variant 1087: The MM-111 molecule is a single polypeptide fusion protein of two scFvs, anti-Her2 (B1D2) and anti-Her3 (H3), linked to the C and N termini, respectively, of a modified human serum albumin protein, and is produced by Merrimack. The resulting molecule is bispecific and bivalent. As a control, a version of the MM-111 molecule was constructed in which the anti-Her3 (H3) warhead was fused to the N terminus of albumin by a short AAS linker, while anti-Her2 (B1D2) was fused to the C terminus through an AAAL (SEQ ID NO: 282) linker to create the benchmark control variant 1087. This control variant lacks the C34S/N504Q mutation originally introduced by Merrimack in their MM-111 molecule, and has a polypeptide sequence corresponding to SEQ ID NO:258.

5. Variant 1090: An albumin-based heteromultimer was formed by combining fragment 1 fused to antiHER3 at its N terminus through a GGGS (SEQ ID NO: 278) linker [SEQ ID NO:260] and fragment 2 fused to antiHER2 (B1D2) at its C terminus through a GGGS (SEQ ID NO: 278) linker [SEQ ID NO:262] (variant number 1090). This molecule has warheads or cargo polypeptides in cis and is almost identical to v1087. The main differences between the 1087 polypeptide and the 1090 are that 1) the linkers used in the 1087 polypeptide were more hydrophobic, while the linkers used on the 1090 variant were polyGLY) (S) and 2) The 1090 variant lacks the C34S/N504Q mutation originally introduced by Merrimack.

Variants 878, 879, and 880 were expressed and purified as described in Example 2. Variants 1087 and 1090 were expressed and purified as described in Example 10.

### Example 32: Fc-Based HER2/HER3 Heteromultimeric Constructs Bind Bispecifically to Her2 and Her3 Receptors on MALME-3M Cells

The ability of the v878, v879 and v880 HER2 and HER3 constructs to bind to Her2 and Her3 was assessed using FACS analysis:

A dose range of the two one-arm monovalent variants (anti-HER2 one-arm, v878, and anti-HER2 one-arm, v879) and the bispecific anti-HER2/HER3 heterodimer, v880, was inclubated with MALME-3M melanoma cells followed by FACS analysis to determine the binding affinity of each molecule as described in Example 4.

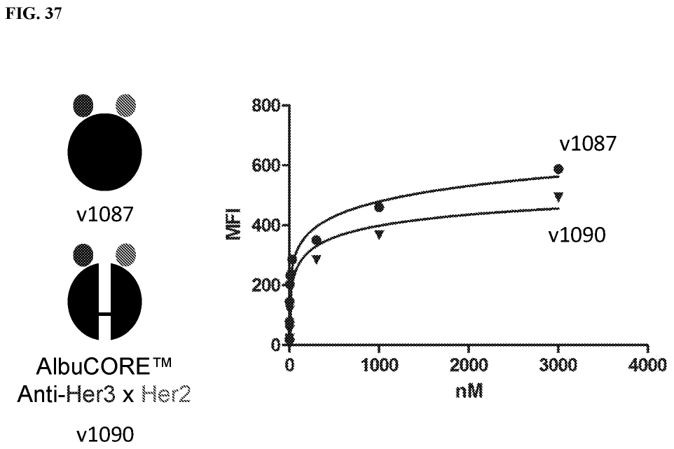

FIGS. 37A and 37B illustrates affinity on linear and log scales respectively. As the figures show, the HER2/HER3 het-Fc construct demonstrates both bi-specificity and avidity.

### Example 33: Albumin-Based HER2/HER3 Based Constructs to Bind Bispecifically to Her2 and Her3 Receptors on MALME-3 Cells

The binding affinity of an albumin-based heteromultimer loaded in a cis configuration with anti-Her2 and anti-Her3 scFvs to MALME-3M cells were assessed and compared to the v1087 control.

The binding affinity of an exemplary albumin-based heteromultimer variant 1090 (anti-Her2×anti-Her3 ABH2) for MALME-3M cells was evaluated using FACS with FITC-labeled anti-HSA antibodies, as described in Example 4.

FIG. 38 depicts the binding of variant 1090 compared to the control 1087 in MALME-3M cells and indicates that v1090 has similar binding as v1087 to target MALME-3M cells.

### Example 34: Monovalent One-Armed Anti-CD3 Antibodies are Able to Bridge Jurkat T Cells and Raji B Cells

The ability of monospecific anti-CD3 antibodies v870, v871, v872 to bridge T cells and B cells was tested by FACS analysis as described in Example 3.

The results are summarized in Table 13 and show that v870, v871 and v872 bridge a higher percentage of Jurkat T and Raji B cells when compared to medium and human IgG controls. Table 13 also shows that v870, v871 and v872 bridge a lower percentage of Jurkat T and Raji B cells when compared to the bispecific anti-CD3 anti-CD19 antibody, v873.

TABLE 13
Monovalent anti-CD3 antibody bridging
of Jurkat T and Raji B cells

| | | Jurkat/Raji |
|---|---|---|
| Variant | Description | (%) |
| Medium | | 7.0 |
| v870 | OAA_anti-CD3 (VH-VL_BiTE) | 9.0 |
| v871 | OAA_anti-CD3 (VL-VH_BiTE) | 15.4 |
| v872 | OAA_anti-CD3 (VL-VH_OKT3) | 14.0 |
| v873 | bispecific CD19-CD3(VH-VL-BiTE) | 27.6 |
| Human IgG | | 6 |
| Medium | | 7.3 |

It is understood that the examples and embodiments described herein are for illustrative purposes only and that various modifications or changes in light thereof will be suggested to persons skilled in the art and are to be included within the spirit and purview of this application and scope of the appended claims.

1. An isolated dimer comprising a first polypeptide and a second polypeptide, each polypeptide comprising a human IgG Fc region comprising a variant CH2 domain, each variant CH2 domain comprising a L234A amino acid substitution, a L235A amino acid substitution, and a D265S amino acid substitution, wherein the amino acid positions are numbered according to the EU index of Kabat.

2. The isolated dimer of claim 1, wherein at least one of the first and second polypeptides further comprises an antigen-binding domain.

3. The isolated dimer of claim 2, wherein the antigen-binding domain comprises an scFv.

4. The isolated dimer of claim 2, wherein the antigen-binding domain comprises a Fab.

5. The isolated dimer of claim 1, wherein one of the first and second polypeptides further comprises an scFv and the other of the polypeptides further comprises a Fab.

6. The isolated dimer of claim 1, wherein one of the first and second polypeptides further comprises amino acid substitutions T350V, L351Y, F405A, and Y407V, and the other of the polypeptides further comprises amino acid substitutions T350V, T366L, K392L, and T394W.

7. The isolated dimer of claim 1, wherein the IgG is an IgG1.

8. An antibody comprising the isolated dimer of claim 1.

9. The antibody of claim 8, wherein the antibody is a monoclonal antibody, a humanized antibody, or a human antibody.

10. The antibody of claim 8, wherein the antibody is multispecific.

11. The antibody of claim 8, wherein the antibody is bispecific.

12. The isolated dimer of claim 1, wherein the dimer is a heterodimer.

13. A pharmaceutical composition comprising the isolated dimer of claim 1 and a pharmaceutically acceptable carrier.

14. A pharmaceutical composition comprising the antibody of claim 8 and a pharmaceutically acceptable carrier.

15. An isolated nucleotide sequence encoding the isolated dimer of claim 1.

16. A vector comprising the nucleotide sequence of claim 15.

17. A host cell comprising the vector of claim 16.

18. A method of producing a polypeptide, comprising culturing the host cell of claim 17, and producing the polypeptide.

19. The isolated dimer of claim 12, wherein each of the first and second polypeptides further comprises an antigen-binding domain.

20. The isolated dimer of claim 19, wherein each antigen-binding domain binds a different antigen.

21. The isolated dimer of claim 20, wherein one of the antigen-binding domains binds to a CD3 complex.


SEQUENCE LISTING (282 sequences)

SEQ ID NO: 1 - DNA, 1548 bp, synthetic construct
  source 1..1548: mol_type = other DNA
gaattcgccaccatggccgtgatggcacctcgaacactggtcctgctgctgagcggggcactggcactgacacagacttgggctggagacattcagctgacacagagtcctgcttcactggcagtgagcctgggacagcgagcaactatctcctgcaaagctagtcagtcagtggactatgatggcgactcctatctgaactggtaccagcagatcccagggcagccccctaagctgctgatctacgacgcctcaaatctggtgagcggcatcccaccacgattcagcggcagcggctctgggactgattttaccctgaacattcacccagtcgagaaggtggacgccgctacctaccattgccagcagtctaccgaggacccctggacattcggcgggggaactaaactggaaatcaagggaggaggaggcagtggcggaggagggtcaggaggaggaggaagccaggtgcagctgcagcagagcggagcagagctggtcagaccaggaagctccgtgaaaatttcctgtaaggcatctggctatgccttttctagttactggatgaattgggtgaagcagaggccaggacagggcctggaatggatcgggcagatttggcccggggatggagacactaactataatggaaagttcaaaggcaaggctacactgactgcagacgagtcaagctccaccgcatatatgcagctgtctagtctggccagcgaggattccgctgtctacttttgcgcacggagagaaaccacaactgtgggcaggtactattacgccatggactactggggccaggggaccacagtcaccgtgtcaagcgcagccgaacccaaatcctctgataagacccacacatgccctccatgtccagctcctgagctgctgggaggaccaagcgtgttcctgtttccacctaaacctaaggacaccctgatgatctctcggacacccgaagtcacttgtgtggtcgtggatgtgagccacgaggaccctgaagtcaaattcaactggtacgtggatggcgtcgaggtgcataatgccaaaacaaagcctagggaggaacagtataactccacttaccgcgtcgtgtctgtcctgaccgtgctgcatcaggactggctgaacggaaaggagtacaaatgcaaggtgagcaacaaggccctgccagctcccatcgagaagaccatttccaaagctaagggccagcctcgagaaccacaggtgtatacactgccacccagccgggacgagctgaccaaaaaccaggtctccctgacatgtctggtgaagggattctacccttctgatattgcagtggagtgggaaagtaatggccagccagaaaacaattataagactacccctccagtcctggattctgacgggagtttctttctgtacagtaaactgacagtggataagtcacggtggcagcagggaaacgtctttagttgttcagtgatgcacgaggccctgcacaatcattacactcagaaaagcctgtccctgtctcccggcaagtgaggatcc

SEQ ID NO: 2 - protein, 513 aa, synthetic construct
EFATMAVMAPRTLVLLLSGALALTQTWAGDIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - DNA, 1527 bp, synthetic construct
  source 1..1527: mol_type = other DNA
gaattcgccaccatggctgtgatggctcctagaaccctggtcctgctgctgtccggggcactggcactgactcagacttgggccggggatatcaaactgcagcagagcggagctgagctggcacgaccaggagccagtgtgaaaatgtcatgcaagacaagcggctacaccttcacacggtatactatgcactgggtgaaacagagacccggccaggggctggaatggatcggatatattaacccttcccgaggctacaccaactataatcagaagtttaaagacaaggccaccctgaccacagataagagctcctctacagcttacatgcagctgagttcactgactagtgaggactcagctgtgtactattgcgcaaggtactatgacgatcattactgtctggattattggggacagggcactaccctgacagtcagctccgtggaaggagggagcggaggctccggaggatctggcgggagtggaggcgtggacgatatccagctgactcagtccccagcaattatgtccgcctctcccggcgagaaagtcactatgacctgccgcgcttctagttcagtgagctacatgaactggtatcagcagaaatcaggcaccagccccaagagatggatctacgacacatccaaggtcgcatctggggtgccttataggttcagtgggtcaggaagcggcacttcctactctctgaccattagctccatggaggcagaagatgccgctacctactattgtcagcagtggtctagtaatccactgacatttggggccggaactaaactggagctgaaggcagccgaacccaaatcaagcgacaagacacacacttgccccccttgtccagcacccgaactgctggggggacctagcgtgttcctgtttccacccaaaccaaaggatacactgatgatcagccggacccctgaggtcacatgcgtggtcgtggacgtgagccacgaggaccccgaagtcaagttcaactggtacgtggacggcgtcgaagtgcataatgctaaaaccaagcctagggaggaacagtacaatagtacttatcgcgtcgtgtcagtcctgaccgtgctgcatcaggattggctgaacgggaaggagtacaaatgcaaggtgtccaacaaggccctgcctgctccaatcgagaagactatttctaaagccaagggccagccccgagaacctcaggtgtataccctgcctccatcccgggacgagctgaccaaaaaccaggtctctctgacatgtctggtgaaggggttctacccatctgatattgctgtggagtgggaaagtaatggacagcccgagaacaattataagacaactccccctgtcctggactccgatggatctttctttctgtacagcaaactgacagtggacaagtccagatggcagcagggcaacgtctttagttgttcagtgatgcacgaggccctgcacaatcattacacccagaaaagcctgtccctgtctcccggcaagtgaggatcc

SEQ ID NO: 4 - protein, 506 aa, synthetic construct
EFATMAVMAPRTLVLLLSGALALTQTWAGDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - DNA, 1518 bp, synthetic construct
  source 1..1518: mol_type = other DNA
gaattcgccaccatggctgtgatggctccacgcaccctggtcctgctgctgtccggggcactggcactgactcagacttgggctggggatattcagctgacccagtctccagcaatcatgtccgcctctcccggcgagaaagtcaccatgacatgccgggctagctcctctgtgtcctacatgaactggtatcagcagaaaagtggaacatcacccaagagatggatctacgacacttcaaaggtcgcaagcggcgtgccttataggttcagtggatcaggcagcgggacttcctactctctgaccattagttcaatggaggcagaagatgccgctacctactattgccagcagtggagctccaatccactgacatttggagccggcactaaactggaactgaagggcggaggaggctcaggaggaggagggagcggcggaggaggcagcgacatcaagctgcagcagagcggagctgagctggcacgaccaggagcctccgtgaaaatgtcttgtaagaccagtggctacactttcacccggtatacaatgcactgggtgaagcagagacccgggcagggactggaatggatcgggtatattaaccctagccgaggatacacaaactacaaccagaagtttaaagacaaggccactctgaccaccgataagtctagttcaaccgcttacatgcagctgagctccctgacaagcgaggactccgctgtgtactattgcgcaaggtactatgacgatcactactgtctggattattggggccaggggactaccctgaccgtgtctagtgcagccgaacctaaatcaagcgacaagacacatacttgccccccttgtccagcaccagagctgctgggaggaccttccgtgttcctgtttccacccaaaccaaaggatacactgatgatctctcgcacccctgaagtcacatgcgtggtcgtggacgtgagccacgaggaccccgaagtcaagttcaactggtacgtggacggcgtcgaggtgcataatgctaaaaccaagcccagggaggaacagtacaattctacttatcgcgtcgtgagtgtcctgaccgtgctgcaccaggattggctgaacggcaaggagtacaaatgcaaggtgagcaacaaggccctgcctgctccaatcgagaagactatttccaaagccaaggggcagccccgagaacctcaggtgtataccctgcctccaagtcgggacgagctgaccaaaaaccaggtctccctgacatgtctggtgaagggcttctacccatccgatattgctgtggagtgggaatctaatgggcagcccgaaaacaattataagacaactccccctgtcctggactctgatgggagtttctttctgtactcaaaactgacagtggataagagcagatggcagcagggaaacgtctttagttgttcagtgatgcatgaggccctgcacaatcattacactcagaaaagcctgtccctgtctcccggcaagtgaggatcc

SEQ ID NO: 6 - protein, 502 aa, synthetic construct
EFATMAVMAPRTLVLLLSGALALTQTWAGDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGGGGSGGGGSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQNVFSCSVMHEALHNHYTQKSLSLSPGK

SEQ ID NO: 7 - DNA, 1518 bp, synthetic construct
  source 1..1518: mol_type = other DNA
gaattcgccaccatggctgtgatggctccaagaacactggtcctgctgctgtccggggcactggcactgactcagacatgggccgggcagattgtcctgacacagagcccagcaatcatgtcagccagccccggcgagaaagtcaccatgacatgctcagcaagctcctctgtgagctacatgaactggtatcagcagaaaagcggaacatcccccaagagatggatctacgacacttccaagctggcttctggagtgcctgcacacttcaggggcagcggctctgggactagttattcactgaccatttccggcatggaggctgaagatgccgctacctactattgccagcagtggagttcaaacccattcacatttggatctggcactaagctggaaattaatggcggaggaggctccggaggaggagggtctggaggaggaggaagtcaggtccagctgcagcagtccggagctgagctggcacgaccaggagcaagtgtgaaaatgtcctgtaaggccagcggctacactttcacccggtatactatgcattgggtgaaacagagacccgggcagggactggaatggatcgggtacattaatcctagccgaggatacaccaactacaaccagaagtttaaagacaaggctaccctgaccacagataagagctcctctacagcatatatgcagctgagttcactgacttctgaggacagtgccgtgtactattgcgctaggtactatgacgatcactactgtctggattattggggccaggggactaccctgaccgtgagctccgcagccgaacctaaatctagtgacaagacacatacttgcccaccttgtccagcaccagagctgctgggaggaccttccgtgttcctgtttccacccaaaccaaaggatacactgatgatctcccggacccctgaagtcacatgcgtggtcgtggacgtgtctcacgaggaccccgaagtcaagtttaactggtacgtggacggcgtcgaggtgcataatgccaaaaccaagcccagggaggaacagtacaactccacttatcgcgtcgtgtctgtcctgaccgtgctgcaccaggattggctgaacggcaaggagtacaaatgcaaggtgagcaacaaggccctgcctgctccaatcgagaagaccattagcaaagccaaggggcagccccgagaacctcaggtgtacacactgcctccatctcgggacgagctgaccaaaaaccaggtcagtctgacatgtctggtgaagggcttctatccaagcgatattgctgtggagtgggaatccaatgggcagcccgaaaacaattacaagacaactccccctgtcctggactcagatgggagcttctttctgtatagtaaactgacagtggacaagtcacggtggcagcagggaaacgtctttagctgttccgtgatgcatgaggccctgcacaatcattacactcagaaatctctgagtctgtcacccggcaagtgaggatcc

SEQ ID NO: 8 - protein, 503 aa, synthetic construct
EFATMAVMAPRTLVLLLSGALALTQTWAGQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINGGGGSGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - DNA, 1548 bp, synthetic construct
  source 1..1548: mol_type = other DNA
gaattcgccaccatggccgtgatggcacctagaacactggtcctgctgctgagcggagccctggcactgacacagacttgggccggagacattcagctgacacagagtcctgcttcactggcagtgagcctgggacagcgagcaactatctcctgcaaagctagtcagtcagtggactatgatggcgactcctatctgaactggtaccagcagatcccagggcagccccctaagctgctgatctacgacgcctcaaatctggtgagcggcatcccaccacgattcagcggcagcggctctgggactgattttaccctgaacattcacccagtcgagaaggtggacgccgctacctaccattgccagcagtctaccgaggacccctggacattcggcgggggaactaaactggaaatcaagggaggaggaggcagtggcggaggagggtcaggaggaggaggaagccaggtgcagctgcagcagagcggagcagagctggtcagaccaggaagctccgtgaaaatttcctgtaaggcatctggctatgccttttctagttactggatgaattgggtgaagcagaggccaggacagggcctggaatggatcgggcagatttggcccggggatggagacactaactataatggaaagttcaaaggcaaggctacactgactgcagacgagtcaagctccaccgcttatatgcagctgtctagtctggccagcgaggattccgctgtctacttttgcgcacggagagaaaccacaactgtgggcaggtactattacgcaatggactactggggccaggggaccacagtcaccgtgtcaagcgcagccgaacccaaatcctctgataagacccacacatgccctccatgtccagcacctgagctgctgggaggaccaagcgtgttcctgtttccacctaaacctaaggacaccctgatgatctctcggacacccgaagtcacttgtgtggtcgtggatgtgagccacgaggaccctgaagtcaaattcaactggtacgtggatggcgtcgaggtgcataatgccaaaacaaagcctagggaggaacagtataactccacttaccgcgtcgtgtctgtcctgaccgtgctgcatcaggactggctgaacggaaaggagtacaaatgcaaggtgagcaacaaggccctgccagctcccatcgagaagaccatttccaaagctaagggccagcctcgagaaccacaggtgtatacatacccacccagccgggacgagctgaccaaaaaccaggtctccctgacatgtctggtgaagggattttatccttctgatattgccgtggagtgggaaagtaatggccagccagaaaacaattacaagactacccctccagtgctggattctgacgggagtttcgcactggtcagtaaactgacagtggataagtcacggtggcagcagggaaacgtctttagttgttcagtgatgcacgaggccctgcacaatcattacactcagaaaagcctgtccctgtctcccggcaagtgaggatcc

SEQ ID NO: 10 - protein, 513 aa, synthetic construct
EFATMAVMAPRTLVLLLSGALALTQTWAGDIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 11 - DNA, 792 bp, synthetic construct
  source 1..792: mol_type = other DNA
gaattcgctacaatggccgtgatggcaccccgaacactggtcctgctgctgagcggcgcactggcactgacacagacttgggctggagaacctaagagctccgacaaaacccacacatgccccccttgtccagctccagaactgctgggaggaccatccgtgttcctgtttccacccaagcccaaagatacactgatgatctctcgaactcccgaggtcacctgcgtggtcgtggacgtcagtcacgaggaccccgaagtcaagttcaactggtacgtggacggcgtcgaagtgcataatgcaaagactaaaccacgggaggaacagtacaactcaacatatagagtcgtgagcgtcctgactgtgctgcatcaggattggctgaacggcaaggagtataagtgcaaagtgagcaataaggccctgcctgctccaatcgagaaaaccattagcaaggcaaaagggcagcccagggaacctcaggtgtacaccctgcctccaagccgcgacgagctgacaaagaaccaggtctccctgctgtgtctggtgaaaggattctatcctagtgatattgccgtggagtgggaatcaaatggccagccagagaacaattacatgacttggccccctgtgctggactctgatgggagtttctttctgtattccaagctgaccgtggacaaatctagatggcagcagggaaacgtcttttcttgtagtgtgatgcacgaagccctgcacaatcattacacacagaagtcactgagcctgtcccctggcaaatgaggatcc

SEQ ID NO: 12 - protein, 261 aa, synthetic construct
EFATMAVMAPRTLVLLLSGALALTQTWAGEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYMTWPPVLDSDGSFFLYSKLTVDKSRWQQGNVFSCSVMHEALHNHYTQKSLSLSPGK

SEQ ID NO: 13 - DNA, 792 bp, synthetic construct
  source 1..792: mol_type = other DNA
gaattcgccaccatggccgtgatggctcctagaaccctggtcctgctgctgagcggggcactggcactgacacagacttgggctggggaacctaaatctagcgacaagactcacacctgcccaccttgtccagcaccagaactgctgggaggaccaagcgtgttcctgtttccacccaagcccaaagatactctgatgatcagccgaacacccgaggtcacttgcgtggtcgtggacgtgtcccacgaggaccccgaagtcaagttcaactggtacgtggacggcgtcgaagtgcataatgctaagacaaaaccacgggaggaacagtacaactctacttatagagtcgtgagtgtcctgaccgtgctgcatcaggattggctgaacggcaaagagtataagtgcaaagtgtctaataaggccctgcctgctccaatcgagaaaacaattagtaaggctaaagggcagcccagggaacctcaggtgtacacttatcctccaagtcgcgacgagctgaccaagaaccaggtctcactgacatgtctggtgaaaggattttacccttccgatattgcagtggagtgggaatctaatggccagccagagaacaattataagaccacaccccctgtgctggacagcgatgggtccttcgcactggtctcaaagctgaccgtggacaaaagcagatggcagcagggaaacgtctttagctgttccgtgatgcacgaagccctgcacaatcattacacacagaagtctctgagtctgtcacctggcaaatgaggatcc

SEQ ID NO: 14 - protein, 261 aa, synthetic construct
EFATMAVMAPRTLVLLLSGALALTQTWAGEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 15 - DNA, 1527 bp, synthetic construct
  source 1..1527: mol_type = other DNA
gaattcgccaccatggctgtgatggcacctagaaccctggtcctgctgctgagcggggcactggcactgacacagacatgggccggagatattaagctgcagcagagcggagctgagctggcacgaccaggagccagtgtgaaaatgtcatgcaagaccagcggctacacattcactcggtatacaatgcactgggtgaagcagagaccaggacagggactggaatggatcggatatattaacccttcccgaggctacaccaactataatcagaagtttaaagacaaggccactctgaccacagataagagctcctctaccgcttacatgcagctgagttcactgacaagtgaggactcagctgtgtactattgcgcaaggtactatgacgatcattactgtctggattattggggacagggcactaccctgactgtcagctccgtggaaggagggagcggaggctccggaggatctggcgggagtggaggcgtggacgatatccagctgacccagtccccagcaattatgtccgcctctcccggcgagaaagtcaccatgacatgccgcgcttctagttcagtgagctacatgaactggtatcagcagaaatcaggcactagccccaagagatggatctacgacacctccaaggtcgcatctggggtgccttataggttcagtgggtcaggaagcggcacctcctactctctgacaattagctccatggaggcagaagatgccgctacctactattgtcagcagtggtctagtaatccactgacttttggggccggaaccaaactggagctgaaggcagccgaacccaaatcaagcgacaagactcacacctgccccccttgtccagcacccgaactgctggggggacctagcgtgttcctgtttccacccaaaccaaaggatacactgatgatcagccggacacctgaggtcacttgcgtggtcgtggacgtgagccacgaggaccccgaagtcaagttcaactggtacgtggacggcgtcgaagtgcataatgctaaaactaagcctagggaggaacagtacaatagtacatatagagtcgtgtcagtgctgaccgtcctgcatcaggattggctgaacgggaaggagtacaaatgcaaggtgtccaacaaggccctgcctgctccaatcgagaagacaatttctaaagccaagggccagccccgagaacctcaggtgtatacactgcctccatcccgggacgagctgactaaaaaccaggtctctctgctgtgtctggtgaaggggttctacccatctgatattgctgtggagtgggaaagtaatggacagcccgagaacaattatatgacctggccccctgtcctggactccgatggatctttctttctgtacagcaaactgacagtggacaagtccagatggcagcagggcaacgtctttagttgttcagtgatgcacgaggccctgcacaatcattacacccagaaaagcctgtccctgtctcccggcaagtgaggatcc

SEQ ID NO: 16 - protein, 506 aa, synthetic construct
EFATMAVMAPRTLVLLLSGALALTQTWAGDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYMTWPPVLDSDGSFFLYSKLTVDKSRWQQGNVFSCSVMHEALHNHYTQKSLSLSPGK

SEQ ID NO: 17 - DNA, 792 bp, synthetic construct; identical to SEQ ID NO: 13
  source 1..792: mol_type = other DNA

SEQ ID NO: 18 - protein, 260 aa, synthetic construct
EFATMAVMAPRTLVLLLSGALALTQTWAGEPKSSDKTHTCPPCPAPELLGGPSVFLFPPKPKDTLMISRTPEVCVVVDVSHEDPEVKFNWYVDGVEVHNAKTKPREEQYNSTYRVVSVLTVLHQDWLNGKEYKCKVSNKALPAPIEKTISKAKGQPREPQVYTYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 19 - DNA, 1518 bp, synthetic construct
  source 1..1518: mol_type = other DNA
gaattcgccaccatggccgtgatggcacctagaaccctggtcctgctgctgagcggggcactggcactgacacagacttgggctggagacattcagctgacacagtctccagcaatcatgtccgcctctcccggcgagaaagtcacaatgacttgccgggctagctcctctgtgtcctacatgaactggtatcagcagaaaagtggaacctcacccaagagatggatctacgacacatcaaaggtcgcaagcggcgtgccttataggttcagtggatcaggcagcgggacctcctactctctgacaattagttcaatggaggcagaagatgccgctacctactattgccagcagtggagctccaatccactgacttttggagccggcaccaaactggaactgaagggcggaggaggctcaggaggaggagggagcggcggaggaggcagcgacatcaagctgcagcagagcggagctgagctggcacgaccaggagcctccgtgaaaatgtcttgtaagactagtggctacaccttcacacggtataccatgcactgggtgaagcagagacccgggcagggactggaatggatcgggtatattaaccctagccgaggatacacaaactacaaccagaagtttaaagacaaggccacactgaccaccgataagtctagttcaactgcttacatgcagctgagctccctgaccagcgaggactccgctgtgtactattgcgcaaggtactatgacgatcactactgtctggattattggggccaggggactaccctgactgtgtctagtgcagccgaacctaaatcaagcgacaagactcatacctgcccaccttgtccagcaccagagctgctgggaggaccttccgtgttcctgtttccacccaaaccaaaggatactctgatgatctctcgcacacctgaagtcacttgcgtggtcgtggacgtgagccacgaggaccccgaagtcaagttcaactggtacgtggacggcgtcgaggtgcataatgctaaaaccaagcccagggaggaacagtacaattctacatatcgcgtcgtgagtgtcctgactgtgctgcaccaggattggctgaacggcaaggagtacaaatgcaaggtgagcaacaaggccctgcctgctccaatcgagaagaccatttccaaagccaaggggcagccccgagaacctcaggtgtatacactgcctccaagtcgggacgagctgactaaaaaccaggtctccctgctgtgtctggtgaagggcttctacccatccgatattgctgtggagtgggaatctaatgggcagcccgaaaacaattatatgacctggccccctgtcctggactctgatgggagtttctttctgtactcaaaactgacagtggataagagcagatggcagcagggaaacgtctttagttgttcagtgatgcatgaggccctgcacaatcattacacccagaaaagcctgtccctgtctcccggcaagtgaggatcc

SEQ ID NO: 20 - protein, 503 aa, synthetic construct
EFATMAVMAPRTLVLLLSGALALTQTWAGDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGGGGSGGGGSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYMTWPPVLDSDGSFFLYSKLTVDKSRWQQGNVFSCSVMHEALHNHYTQKSLSLSPGK

SEQ ID NO: 21 - DNA, 792 bp, synthetic construct; identical to SEQ ID NO: 13
  source 1..792: mol_type = other DNA

SEQ ID NO: 22 - protein, 261 aa, synthetic construct; identical to SEQ ID NO: 14

SEQ ID NO: 23 - DNA, 1518 bp, synthetic construct
  source 1..1518: mol_type = other DNA
gaattcgccaccatggccgtgatggctccaagaacactggtcctgctgctgagcggggcactggcactgacacagacatgggctgggcagatcgtcctgacacagagcccagcaatcatgtcagccagccccggcgagaaagtcacaatgacttgctcagcaagctcctctgtgagctacatgaactggtatcagcagaaaagcggaacctcccccaagagatggatctacgacacatccaagctggcttctggagtgcctgcacacttcaggggcagcggctctgggaccagttattcactgacaatttccggcatggaggctgaagatgccgctacctactattgccagcagtggagttcaaacccattcacttttggatctggcaccaagctggaaattaatggcggaggaggctccggaggaggagggtctggaggaggaggaagtcaggtccagctgcagcagtccggagctgagctggcacgaccaggagcaagtgtgaaaatgtcctgtaaggccagcggctacaccttcacacggtataccatgcattgggtgaaacagagacccgggcagggactggaatggatcgggtacattaatcctagccgaggatacacaaactacaaccagaagtttaaagacaaggctactctgaccacagataagagctcctctaccgcatatatgcagctgagttcactgacatctgaggacagtgccgtgtactattgcgctaggtactatgacgatcactactgtctggattattggggccaggggactaccctgaccgtgagctccgcagccgaacctaaatctagtgacaagactcatacctgccccccttgtccagcaccagagctgctgggaggaccttccgtgttcctgtttccacccaaaccaaaggatactctgatgatctcccggacacctgaagtcacttgcgtggtcgtggacgtgtctcacgaggaccccgaagtcaagtttaactggtacgtggacggcgtcgaggtgcataatgccaaaaccaagcccagggaggaacagtacaactccacatatcgcgtcgtgtctgtcctgactgtgctgcaccaggattggctgaacggcaaggagtacaaatgcaaggtgagcaacaaggccctgcctgctccaatcgagaagacaattagcaaagccaaggggcagccccgagaacctcaggtgtacactctgcctccatctcgggacgagctgaccaaaaaccaggtcagtctgctgtgtctggtgaagggcttctatccaagcgatattgctgtggagtgggaatccaatgggcagcccgaaaacaattacatgacatggccccctgtcctggactcagatgggagcttctttctgtatagtaaactgactgtggacaagtcacggtggcagcagggaaacgtctttagctgttccgtgatgcatgaggccctgcacaatcattacacccagaaatctctgagtctgtcacccggcaagtgaggatcc

SEQ ID NO: 24 - protein, 503 aa, synthetic construct
EFATMAVMAPRTLVLLLSGALALTQTWAGQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINGGGGSGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYMTWPPVLDSDGSFFLYSKLTVDKSRWQQGNVFSCSVMHEALHNHYTQKSLSLSPGK

SEQ ID NO: 25 - DNA, 1548 bp, synthetic construct; identical to SEQ ID NO: 9
  source 1..1548: mol_type = other DNA

SEQ ID NO: 26 - protein, 513 aa, synthetic construct; identical to SEQ ID NO: 10

SEQ ID NO: 27 - DNA, 1527 bp, synthetic construct; identical to SEQ ID NO: 15
  source 1..1527: mol_type = other DNA

SEQ ID NO: 28 - protein, 506 aa, synthetic construct; identical to SEQ ID NO: 16

SEQ ID NO: 29 - DNA, 1548 bp, synthetic construct; identical to SEQ ID NO: 9
  source 1..1548: mol_type = other DNA

SEQ ID NO: 30 - protein, 513 aa, synthetic construct; identical to SEQ ID NO: 10

SEQ ID NO: 31 - DNA, 1518 bp, synthetic construct; identical to SEQ ID NO: 19
  source 1..1518: mol_type = other DNA

SEQ ID NO: 32 - protein, 503 aa, synthetic construct; identical to SEQ ID NO: 20

SEQ ID NO: 33 - DNA, 1548 bp, synthetic construct; identical to SEQ ID NO: 9
  source 1..1548: mol_type = other DNA

SEQ ID NO: 34 - protein, 513 aa, synthetic construct; identical to SEQ ID NO: 10

SEQ ID NO: 35 - DNA, 1518 bp, synthetic construct; identical to SEQ ID NO: 23
  source 1..1518: mol_type = other DNA

SEQ ID NO: 36 - protein, 503 aa, synthetic construct; identical to SEQ ID NO: 24

SEQ ID NO: 37 - DNA, 1548 bp, synthetic construct
  source 1..1548: mol_type = other DNA
gaattcgccaccatggccgtgatggcaccccgcaccctggtcctgctgctgtccggggcactggcactgactcagacttgggctggcgatattcagctgacacagagtcctgcatcactggctgtgagcctgggacagcgagcaactatctcctgcaaagccagtcagtcagtggactatgatggcgactcctatctgaactggtaccagcagatcccagggcagccccctaagctgctgatctacgacgcctcaaatctggtgagcggcatcccaccacgattcagcggcagcggctctgggactgattttaccctgaacattcacccagtcgagaaggtggacgccgctacctaccattgccagcagtctaccgaggacccctggacattcggcgggggaactaaactggaaatcaagggaggaggaggcagtggcggaggagggtcaggaggaggaggaagccaggtgcagctgcagcagagcggagcagagctggtcagaccaggaagctccgtgaaaatttcctgtaaggcttctggctatgcattttctagttactggatgaattgggtgaagcagaggccaggacagggcctggaatggatcgggcagatttggcccggggatggagacaccaactataatggaaagttcaaaggcaaggccacactgactgctgacgagtcaagctccacagcctatatgcagctgtctagtctggcaagcgaggattccgccgtgtacttttgcgctcggagagaaaccacaactgtgggcaggtactattacgctatggactactggggccaggggaccacagtcaccgtgtcaagcgcagccgaacccaaatcctctgataagacccacacatgccctccatgtccagctcctgaggctgcaggaggaccaagcgtgttcctgtttccccctaaacctaaggacacactgatgatctctcggacacccgaagtcacttgtgtggtcgtgagcgtgagccacgaggaccctgaagtcaaattcaactggtacgtggatggcgtcgaggtgcataatgccaaaactaagcctagggaggaacagtataactccacttaccgcgtcgtgtctgtcctgaccgtgctgcatcaggactggctgaacggaaaggagtacaaatgcaaggtgagcaacaaggcactgccagcccccatcgagaagacaatttccaaagcaaagggccagcctcgagaaccacaggtctatgtgtacccacccagccgggacgagctgaccaaaaaccaggtctccctgacatgtctggtgaagggattttatccttctgatattgccgtggagtgggaaagtaatggccagccagaaaacaattacaagactacccctccagtgctggattctgacgggagtttcgctctggtcagtaaactgactgtggataagtcacggtggcagcagggaaacgtctttagttgttcagtgatgcacgaggcactgcacaatcattacacccagaaaagcctgtccctgtctcccggcaagtgaggatcc

SEQ ID NO: 38 - protein, 513 aa, synthetic construct
EFATMAVMAPRTLVLLLSGALALTQTWAGDIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 39 - DNA, 1518 bp, synthetic construct
  source 1..1518: mol_type = other DNA
gaattcgccaccatggccgtgatggcacctcgcaccctggtcctgctgctgagcggggcactggcactgacacagacttgggccggacagatcgtcctgacacagagcccagctatcatgtcagcaagccccggcgagaaagtcacaatgacttgctcagccagctcctctgtgagctacatgaactggtatcagcagaaaagcggaacctcccccaagagatggatctacgacacatccaagctggcctctggagtgcctgctcacttcaggggcagcggctctgggaccagttattcactgacaatttccggcatggaggccgaagatgccgctacctactattgccagcagtggagttcaaacccattcacttttggatctggcaccaagctggaaattaatggcggaggaggctccggaggaggagggtctggaggaggaggaagtcaggtgcagctgcagcagtccggagcagagctggctcgaccaggagctagtgtgaaaatgtcctgtaaggcaagcggctacaccttcacacggtataccatgcattgggtgaaacagagacccgggcagggactggaatggatcgggtacattaatcctagccgaggatacacaaactacaaccagaagtttaaagacaaggccactctgaccacagataagagctcctctaccgcttatatgcagctgagttcactgacatctgaggacagtgcagtgtactattgcgccaggtactatgacgatcactactgtctggattattggggccaggggactaccctgacagtgagctccgcagccgaacctaaatctagtgacaagactcatacctgccccccttgtccagcaccagaggctgcaggaggaccttccgtgttcctgtttccacccaaaccaaaggatactctgatgatctcccggacacctgaagtcacttgcgtggtcgtgagcgtgtctcacgaggaccccgaagtcaagtttaactggtacgtggacggcgtcgaggtgcataatgccaaaaccaagcccagggaggaacagtacaactccacatatcgcgtcgtgtctgtcctgactgtgctgcaccaggattggctgaacggcaaggagtacaaatgcaaggtgagcaacaaggcactgcctgccccaatcgagaagacaattagcaaagcaaaggggcagccccgagaacctcaggtctacgtgctgcctccatctcgggacgagctgactaaaaaccaggtcagtctgctgtgtctggtgaagggcttctatccaagcgatattgctgtggagtgggaatccaatgggcagcccgaaaacaattacctgacttggccccctgtcctggactcagatgggagcttctttctgtatagtaaactgaccgtggacaagtcacggtggcagcagggaaacgtctttagctgttccgtgatgcatgaggccctgcacaatcattacacccagaaatctctgagtctgtcacccggcaagtgaggatcc

SEQ ID NO: 40 - protein, 503 aa, synthetic construct
EFATMAVMAPRTLVLLLSGALALTQTWAGQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINGGGGSGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 41 - DNA, 1548 bp, synthetic construct; identical to SEQ ID NO: 37
  source 1..1548: mol_type = other DNA

SEQ ID NO: 42 - protein, 513 aa, synthetic construct; identical to SEQ ID NO: 38

SEQ ID NO: 43 - DNA, 1518 bp, synthetic construct
  source 1..1518: mol_type = other DNA
gaattcgccaccatggccgtgatggcacctcgcaccctggtcctgctgctgagcggggcactggcactgacacagacttgggctgggcagatcgtcctgacacagagcccagctatcatgtcagcaagccccggcgagaaagtcacaatgacttgctcagccagctcctctgtgagctacatgaactggtatcagcagaaaagcggaacctcccccaagagatggatctacgacacatccaagctggcctctggagtgcctgctcacttcaggggcagcggctctgggaccagttattcactgacaatttccggcatggaggccgaagatgccgctacctactattgccagcagtggagttcaaacccattcacttttggatctggcaccaagctggaaattaatggcggaggaggctccggaggaggagggtctggaggaggaggaagtcaggtgcagctgcagcagagcggagcagagctggctcgaccaggagctagtgtgaaaatgtcctgtaaggcaagcggctacaccttcacacggtataccatgcattgggtgaaacagagacccgggcagggactggaatggatcgggtacattaatccttcccgaggatacacaaactacaaccagaagtttaaagacaaggccactctgaccacagataagagctcctctaccgcttatatgcagctgagttcactgacatctgaggacagtgcagtgtactattgcgccaggtactatgacgatcactactccctggattattggggccaggggactaccctgacagtgagctccgcagccgaacctaaatctagtgacaagactcatacctgccccccttgtccagcaccagaggctgcaggaggacctagcgtgttcctgtttccacccaaaccaaaggatactctgatgatctcccggacacctgaagtcacttgtgtggtcgtgagcgtgtctcacgaggaccccgaagtcaagtttaactggtacgtggacggcgtcgaggtgcataatgccaaaaccaagcccagggaggaacagtacaactccacatatcgcgtcgtgtctgtcctgactgtgctgcaccaggattggctgaacggcaaggagtacaaatgcaaggtgagcaacaaggcactgcctgccccaatcgagaagacaattagcaaagcaaaggggcagccccgagaacctcaggtctacgtgctgcctccatctcgggacgagctgactaaaaaccaggtcagtctgctgtgtctggtgaagggcttctatccaagcgatattgctgtggagtgggaatccaatgggcagcccgaaaacaattacctgacttggccccctgtcctggactcagatgggagcttctttctgtatagtaaactgaccgtggacaagtcacggtggcagcagggaaacgtctttagctgttccgtgatgcatgaggccctgcacaatcattacacccagaaatctctgagtctgtcacccggcaagtgaggatcc

SEQ ID NO: 44 - protein, 503 aa, synthetic construct
EFATMAVMAPRTLVLLLSGALALTQTWAGQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINGGGGSGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 45 - DNA, 1536 bp, synthetic construct
  source 1..1536: mol_type = other DNA
gaattcgccaccatggccgtgatggctcctcgaacactggtcctgctgctgagcggggcactggcactgacacagacttgggctgggcaggtccagctgcagcagagcggagcagagctggctcgaccaggagctagtgtgaaaatgtcatgcaaggcaagcggctacaccttcacacggtatactatgcactgggtgaaacagagacccggacagggcctggaatggatcgggtacattaaccctagccgaggatacaccaactacaaccagaagtttaaagacaaggccaccctgaccacagataagagctcctctacagcttatatgcagctgagttcactgacttctgaggacagtgccgtgtactattgtgctcggtactatgacgatcattactccctggattattgggggcagggaactaccctgaccgtgagctcctctagtacaggaggaggaggcagtggaggaggagggtcaggcggaggaggaagcgacatccagattgtgctgacacagtctccagctatcatgtccgcatctcccggcgagaaagtcactatgacctgctccgcctcaagctccgtgtcttacatgaattggtatcagcagaaatcaggaaccagccccaagagatggatctacgacacatccaagctggcatctggagtgcctgcacacttcaggggcagtgggtcaggaactagctattccctgaccattagcggcatggaggccgaagatgccgctacctactattgtcagcagtggtctagtaacccattcacatttggcagcgggactaagctggagatcaatagggcagccgaacccaaatcaagcgacaagacacatacttgccccccttgtccagctccagaagctgcaggaggaccttccgtgttcctgtttccacccaaaccaaaggatacactgatgattagccgcacccctgaggtcacatgcgtggtcgtgagcgtgagccacgaggaccccgaagtcaagttcaactggtacgtggacggcgtcgaagtgcataatgccaaaaccaagcctagggaggaacagtacaacagtacatatagagtcgtgtcagtgctgaccgtcctgcaccaggattggctgaacggcaaggagtacaaatgcaaggtgtccaacaaggcactgcctgccccaatcgagaagaccatttctaaagctaaggggcagccccgagaacctcaggtctacgtgtatcctccatcccgggacgagctgactaaaaaccaggtctctctgacctgtctggtgaagggcttttacccatctgatattgcagtcgagtgggaaagtaatgggcagcccgagaacaattataagacaactccccctgtgctggactccgatgggtctttcgcactggtcagcaaactgacagtggataagtccagatggcagcagggaaacgtcttttcttgtagtgtgatgcatgaagccctgcacaatcattacactcagaaatcactgagcctgtcccccggcaagtgaggatcc

SEQ ID NO: 46 - protein, 509 aa, synthetic construct
EFATMAVMAPRTLVLLLSGALALTQTWAGQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSSSTGGGGSGGGGSGGGGSDIQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 47 - DNA, 1548 bp, synthetic construct
  source 1..1548: mol_type = other DNA
gaattcgccaccatggctgtgatggcacctagaaccctggtcctgctgctgtccggggcactggcactgactcagacttgggctggggatattcagctgacccagagtcctgcatcactggctgtgagcctgggacagcgagcaacaatctcctgcaaagccagtcagtcagtggactatgatggcgactcctatctgaactggtaccagcagatcccagggcagccccctaagctgctgatctacgacgcttcaaatctggtgagcggcatcccaccacgattcagcggcagcggctctggaaccgattttacactgaacattcacccagtcgagaaggtggacgccgctacctaccattgccagcagtctacagaggacccctggactttcggcgggggaaccaaactggaaatcaagggaggaggaggcagtggcggaggagggtcaggaggaggaggaagccaggtgcagctgcagcagagcggagcagagctggtcagaccaggaagctccgtgaaaatttcctgtaaggcttctggctatgcattttctagttactggatgaattgggtgaagcagaggccaggacagggcctggaatggatcgggcagatttggcccggggatggagacacaaactataatggaaagttcaaaggcaaggccactctgaccgctgacgagtcaagctccactgcttatatgcagctgtctagtctggcaagcgaggattccgccgtctacttttgcgctcggagagaaaccacaactgtgggcaggtactattacgcaatggactactggggccaggggaccacagtcaccgtgtcaagcgcagccgaacccaaatcctctgataagacacacacttgccctccatgtccagcacctgaggctgcaggaggaccaagcgtgttcctgtttccccctaaacctaaggacactctgatgatctctcggactcccgaagtcacctgtgtggtcgtgagcgtgagccacgaggaccctgaagtcaaattcaactggtacgtggatggcgtcgaggtgcataatgccaaaacaaagcctagggaggaacagtataactccacataccgcgtcgtgtctgtcctgactgtgctgcatcaggactggctgaacggaaaggagtacaaatgcaaggtgagcaacaaggcactgccagcccccatcgagaagaccatttccaaagccaagggccagcctcgagaaccacaggtctatgtgctgccacccagccgggacgagctgacaaaaaaccaggtctccctgctgtgtctggtgaagggattctacccttctgatattgctgtggagtgggaaagtaatggccagccagaaaacaattatctgacttggcctccagtgctggattctgacgggagtttctttctgtacagtaaactgaccgtggataagtcacggtggcagcagggaaacgtctttagttgttcagtgatgcacgaggccctgcacaatcattacacccagaaaagcctgtccctgtctcccggcaagtgaggatcc

SEQ ID NO: 48 - protein, 513 aa, synthetic construct
EFATMAVMAPRTLVLLLSGALALTQTWAGDIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 49 - DNA, 1527 bp, synthetic construct
  source 1..1527: mol_type = other DNA
gaattcgcgaccatggctgtgatggctccacgaaccctggtcctgctgctgtccggggctctggcactgactcagacttgggctggggatattaagctgcagcagagcggagcagagctggctcgacctggagcctctgtgaaaatgagttgcaagacatcaggctacaccttcacacggtatactatgcactgggtgaaacagagacccggccagtgtctggaatggatcgggtatattaaccctagccgaggatacaccaactacaaccagaagtttaaagacaaggcaaccctgaccacagataagagctcctctacagcctacatgcagctgagttcactgacttctgaggacagtgccgtgtactattgcgctaggtactatgacgatcattactgtctggattattggggccaggggactaccctgactgtcagctccgtggaaggagggtcaggaggcagcggaggcagcggagggtctggaggcgtggacgatatccagctgacccagtctccagctattatgtccgcatctcccggcgagaaagtgactatgacctgccgcgcctctagttcagtgtcctacatgaactggtatcagcagaaaagtggaacctcacccaagagatggatctacgacacaagcaaggtcgcttccggcgtgccttataggttcagtggatcaggcagcgggacttcctactctctgaccattagctccatggaggcagaagatgccgctacctactattgccagcagtggtctagtaatccactgacatttggatgtggcactaaactggagctgaaggcagccgaacccaaatcaagcgacaagacacacacttgcccaccttgtccagcaccagaagctgcaggaggacctagcgtgttcctgtttccacccaaaccaaaggatacactgatgatctcccggacccctgaggtcacatgcgtggtggtgagcgtgagccacgaggacccagaagtcaagttcaactggtacgtggatggcgtcgaagtgcataatgcaaaaactaagcccagggaggaacagtacaactctacttatcgcgtcgtgagtgtcctgaccgtgctgcatcaggactggctgaacggcaaggagtacaaatgcaaggtgtctaacaaggccctgcctgccccaatcgagaagacaattagcaaagctaaggggcagccccgagaacctcaggtctacgtgtatcctccaagtcgggacgagctgaccaaaaaccaggtctccctgacatgtctggtgaagggcttttatccatccgatattgccgtggagtgggaatctaatgggcagcccgagaacaattacaagacaactccccctgtgctggacagcgatgggtccttcgctctggtctcaaaactgaccgtggataagagcagatggcagcagggaaatgtctttagctgctccgtgatgcacgaggcactgcacaaccattacacccagaagtcactgtcactgtccccaggcaagtgaggatcc

SEQ ID NO: 50 - protein, 506 aa, synthetic construct
EFATMAVMAPRTLVLLLSGALALTQTWAGDIKLQQSGAELARPGASVKMSCKTSGYTFTRYTMHWVKQRPGQCLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGCGTKLELKAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 51 - DNA, 1548 bp, synthetic construct; identical to SEQ ID NO: 47
  source 1..1548: mol_type = other DNA

SEQ ID NO: 52 - protein, 513 aa, synthetic construct; identical to SEQ ID NO: 48

SEQ ID NO: 53 - DNA, 1545 bp, synthetic construct
  source 1..1545: mol_type = other DNA
gaattcgccaccatggccgtgatggcacctagaaccctggtcctgctgctgagcggggcactggcactgacacagacatgggccggagaagtccagctggtcgagtccggaggaggactggtgcagccaggagggtcactgaaactgagctgcgccgcttccggcttcacttttaacaagtatgcaatgaattgggtgcggcaggcaccagggaagggactggaatgggtggcccggatcagatctaagtacaacaactacgctacctactatgcagacagtgtgaaggataggttcacaatttctcgcgacgatagtaaaaacactgcttacctgcagatgaacaatctgaagacagaggacactgcagtctactattgcgtgagacacggaaactttggcaatagctacatctcctattgggcatactggggacagggaaccctggtcacagtgagctccggaggaggaggcagcggaggaggaggctctgggggaggcgggagtcagactgtggtcacccaggagccctcactgacagtcagccctggaggcactgtgaccctgacatgtgggtctagtaccggagccgtgacatctggcaactatcccaattgggtgcagcagaaacctggacaggctccacgaggactgattggaggaacaaagttcctggcccccggaactcctgctcgattttccggctctctgctgggagggaaagcagcactgaccctgagcggagtgcagcctgaggatgaagccgagtactattgcgtgctgtggtacagcaacagatgggtgttcggaggcgggacaaagctgactgtgctggctgcagagccaaagtcaagcgacaaaactcacacctgcccaccttgtccagctccagaagcagctggaggaccatccgtgttcctgtttccacccaagcccaaagatacactgatgatctctcgcactcccgaggtcacctgtgtggtcgtgagtgtgtcacacgaagaccctgaggtcaagtttaactggtacgtggatggcgtcgaagtgcataatgccaagaccaaacctcgagaggaacagtataattcaacttaccgggtcgtgagcgtcctgaccgtgctgcatcaggactggctgaacggaaaggagtacaagtgcaaagtgagcaataaggcactgcctgccccaatcgaaaaaaccattagcaaggctaaagggcagccaagagagccccaggtctacgtgtatcctccaagcagggacgaactgaccaagaaccaggtctccctgacatgtctggtgaaagggttctatcctagtgatattgcagtggaatgggagtcaaatggacagccagagaacaattacaagaccacaccccctgtgctggactctgatggcagtttcgcactggtctccaagctgaccgtggataaatctaggtggcagcaggggaacgtctttagctgttccgtgatgcatgaagccctgcacaatcattacacacagaagtctctgagtctgtcacccggcaaatgaggatcc

SEQ ID NO: 54 - protein, 512 aa, synthetic construct
EFATMAVMAPRTLVLLLSGALALTQTWAGEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 55 - DNA, 1542 bp, synthetic construct
  source 1..1542: mol_type = other DNA
gaattcgccaccatggccgtgatggcacctagaacactggtcctgctgctgagcggagccctggcactgacacagacatgggccggggaagtccagctggtcgagtctggaggaggactggtgaagccaggagggagtctgaaactgtcatgcgccgctagcgggtataccttcacaagctacgtcatgcactgggtgaggcaggcaccagggaagggactggaatggatcggctatattaatccctacaacgacgggactaagtataatgagaaatttcagggcagggtgaccatcagctccgataagtctattagtacagcctacatggagctgtctagtctgcgcagcgaagacacagcaatgtactattgcgccagggggacatactattacggaactcgcgtgttcgattactggggccaggggaccctggtcacagtgtcaagcggaggcgggggaagtggaggaggaggctcaggaggaggagggagcgacatcgtgatgacccagtcccctgctacactgtcactgagcccaggcgagcgggcaactctgtcctgtagatcctctaagtctctgcagaacgtgaatggaaacacctatctgtactggtttcagcagaaaccaggccagagcccccagctgctgatctatagaatgtccaatctgaactctggcgtgcctgataggttctccggatctggcagtgggaccgagttcaccctgaccattagttcactggagccagaagacttcgccgtgtattactgcatgcagcacctggagtaccccatcacttttggagctggcaccaagctggagatcaaggcagccgaaccaaagagctccgataaaacacatacttgcccaccttgtccagcaccagaagctgcaggaggaccaagcgtgttcctgtttccacccaagcctaaagacaccctgatgatctcccggactcccgaggtcacctgtgtggtcgtgtcagtgagccacgaggaccctgaagtcaagttcaattggtacgtggatggcgtcgaagtgcataacgctaagacaaaaccccgagaggaacagtataacagtacataccgggtcgtgtcagtgctgaccgtcctgcaccaggattggctgaatggaaaggagtacaagtgcaaagtgtctaacaaggccctgcctgctccaatcgagaaaaccattagcaaggctaaaggccagccccgcgaacctcaggtctatgtgctgcctccaagccgagatgagctgacaaagaatcaggtctccctgctgtgtctggtgaaagggttctacccttctgacattgcagtggagtgggaaagtaacggacagccagagaacaattatctgacatggccccctgtcctggactccgatggctctttctttctgtacagcaagctgactgtggacaaatccagatggcagcaggggaatgtcttttcctgttctgtgatgcatgaagccctgcacaaccattacacccagaagagtctgtcactgagccctggcaaatgaggatcc

SEQ ID NO: 56 - protein, 511 aa, synthetic construct
EFATMAVMAPRTLVLLLSGALALTQTWAGEVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSSGGGGSGGGGSGGGGSDIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 57 - DNA, 1545 bp, synthetic construct
  source 1..1545: mol_type = other DNA
gaattcgccaccatggccgtgatggcacctagaacactggtcctgctgctgagcggggcactggcactgacacagacatgggccggagaagtccagctggtcgagtccggaggaggactggtgcagccaggagggtcactgaaactgagctgcgccgcttccggcttcacttttaacaagtatgcaatgaattgggtgcggcaggcacctgggaagtgtctggaatgggtggcccggatcagatctaagtacaacaactacgctacctactatgcagacagtgtgaaggataggttcacaatttctcgcgacgatagtaaaaacactgcttacctgcagatgaacaatctgaagacagaggacactgcagtctactattgcgtgagacacggaaactttggcaatagctacatctcctattgggcctactgggggcagggaaccctggtcacagtgagctccggaggaggaggcagcggaggaggaggctctgggggaggcgggagtcagactgtggtcacccaggagccctcactgacagtcagccctggaggcactgtgaccctgacatgtgggtctagtaccggagccgtgacatctggcaactatcccaattgggtgcagcagaaacctggacaggctccacgaggactgattggaggaacaaagttcctggcccccggaactcctgctcgattttccggctctctgctgggagggaaagcagcactgaccctgagcggagtgcagcctgaggatgaagccgagtactattgcgtgctgtggtacagcaacagatgggtgttcggctgtgggacaaagctgactgtgctggctgcagagccaaagtcaagcgacaaaactcacacctgcccaccttgtccagctccagaagcagctggaggaccatccgtgttcctgtttccacccaagcccaaagatacactgatgatctctcgcactcccgaggtcacctgcgtggtcgtgagtgtgtcacacgaagaccctgaggtcaagtttaactggtacgtggatggcgtcgaagtgcataatgccaagaccaaacctcgagaggaacagtataattcaacttaccgggtcgtgagcgtcctgaccgtgctgcatcaggactggctgaacgggaaggagtacaagtgcaaagtgagcaataaggcactgcctgccccaatcgaaaaaaccattagcaaggctaaaggccagccaagagagccccaggtctacgtgtatcctccaagcagggacgaactgaccaagaaccaggtctccctgacatgtctggtgaaagggttctatcctagtgatattgcagtggaatgggagtcaaatggacagccagagaacaattacaagaccacaccccctgtgctggactctgatggaagtttcgcactggtctccaagctgaccgtggataaatctaggtggcagcagggcaacgtctttagctgttccgtgatgcatgaagccctgcacaatcattacacacagaagtctctgagtctgtcacccggcaaatgaggatcc

SEQ ID NO: 58 - protein, 512 aa, synthetic construct
EFATMAVMAPRTLVLLLSGALALTQTWAGEVQLVESGGGLVQPGGSLKLSCAASGFTFNKYAMNWVRQAPGKCLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGCGTKLTVLAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 59 - DNA, 1542 bp, synthetic construct
  source 1..1542: mol_type = other DNA
gaattcgccaccatggctgtgatggcacctagaacactggtcctgctgctgagcggggcactggcactgacacagacatgggccggggaagtccagctggtcgagtctggaggaggactggtgaagccaggagggagtctgaaactgtcatgcgccgctagcggctataccttcacaagctacgtcatgcactgggtgaggcaggcccctgggaagtgtctggaatggatcggatatattaatccctacaacgacggcactaagtataatgagaaatttcaggggagggtgaccatcagctccgataagtctattagtacagcttacatggagctgtctagtctgcgcagcgaagacacagcaatgtactattgcgccaggggaacatactattacggcactcgcgtgttcgattactgggggcagggaaccctggtcacagtgtcaagcggaggcgggggaagtggaggaggaggctcaggaggaggagggagcgacatcgtgatgacccagtcccctgctacactgtcactgagcccaggggagcgggcaactctgtcctgtagatcctctaagtctctgcagaacgtgaatggaaacacctatctgtactggtttcagcagaaaccaggccagagcccccagctgctgatctatagaatgtccaatctgaactctggcgtgcctgataggttctccggctctgggagtggaaccgagttcaccctgaccattagttcactggagccagaagacttcgctgtgtattactgcatgcagcacctggagtaccccatcacttttggctgtgggaccaagctggagatcaaggcagccgaaccaaagagctccgataaaacacatacttgcccaccttgtccagcaccagaagctgcaggaggaccaagcgtgttcctgtttccacccaagcctaaagacaccctgatgatctcccgaactccagaggtcacctgcgtggtcgtgtcagtgagccacgaggaccctgaagtcaagttcaattggtacgtggatggcgtcgaagtgcataacgccaagacaaaaccccgagaggaacagtataacagtacataccgggtcgtgtcagtgctgaccgtcctgcaccaggattggctgaatggaaaggagtacaagtgcaaagtgtctaacaaggccctgcctgctccaatcgagaaaaccattagcaaggcaaaaggccagccccgcgaacctcaggtctatgtgctgcctccaagccgagatgagctgacaaagaatcaggtctccctgctgtgtctggtgaaagggttctacccttctgacattgccgtggagtgggaaagtaacggacagccagagaacaattatctgacatggccccctgtcctggactccgatggctctttctttctgtacagcaagctgactgtggacaaatccagatggcagcaggggaatgtcttttcctgttctgtgatgcatgaagccctgcacaaccattacacccagaagagtctgtcactgagccctggcaaatgaggatcc

SEQ ID NO: 60 - protein, 511 aa, synthetic construct
EFATMAVMAPRTLVLLLSGALALTQTWAGEVQLVESGGGLVKPGGSLKLSCAASGYTFTSYVMHWVRQAPGKCLEWIGYINPYNDGTKYNEKFQGRVTISSDKSISTAYMELSSLRSEDTAMYYCARGTYYYGTRVFDYWGQGTLVTVSSGGGGSGGGGSGGGGSDIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGCGTKLEIKAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 61 - DNA, 1535 bp, synthetic construct
  source 1..1535: mol_type = other DNA
ccatggccgtgatggcacctagaaccctggtcctgctgctgagcggggcactggcactgacacagacatgggccggagaagtccagctggtcgagtccggaggaggactggtgcagccaggagggtcactgaaactgagctgcgccgcttccggcttcacttttaacaagtatgcaatgaattgggtgcggcaggcaccagggaagggactggaatgggtggcccggatcagatctaagtacaacaactacgctacctactatgcagacagtgtgaaggataggttcacaatttctcgcgacgatagtaaaaacactgcttacctgcagatgaacaatctgaagacagaggacactgcagtctactattgcgtgagacacggaaactttggcaatagctacatctcctattgggcatactggggacagggaaccctggtcacagtgagctccggaggaggaggcagcggaggaggaggctctgggggaggcgggagtcagactgtggtcacccaggagccctcactgacagtcagccctggaggcactgtgaccctgacatgtgggtctagtaccggagccgtgacatctggcaactatcccaattgggtgcagcagaaacctggacaggctccacgaggactgattggaggaacaaagttcctggcccccggaactcctgctcgattttccggctctctgctgggagggaaagcagcactgaccctgagcggagtgcagcctgaggatgaagccgagtactattgcgtgctgtggtacagcaacagatgggtgttcggaggcgggacaaagctgactgtgctggctgcagagccaaagtcaagcgacaaaactcacacctgcccaccttgtccagctccagaagcagctggaggaccatccgtgttcctgtttccacccaagcccaaagatacactgatgatctctcgcactcccgaggtcacctgtgtggtcgtgagtgtgtcacacgaagaccctgaggtcaagtttaactggtacgtggatggcgtcgaagtgcataatgccaagaccaaacctcgagaggaacagtataattcaacttaccgggtcgtgagcgtcctgaccgtgctgcatcaggactggctgaacggaaaggagtacaagtgcaaagtgagcaataaggcactgcctgccccaatcgaaaaaaccattagcaaggctaaagggcagccaagagagccccaggtctacgtgtatcctccaagcagggacgaactgaccaagaaccaggtctccctgacatgtctggtgaaagggttctatcctagtgatattgcagtggaatgggagtcaaatggacagccagagaacaattacaagaccacaccccctgtgctggactctgatggcagtttcgcactggtctccaagctgaccgtggataaatctaggtggcagcaggggaacgtctttagctgttccgtgatgcatgaagccctgcacaatcattacacacagaagtctctgagtctgtcacccggcaaatgaggatcc

SEQ ID NO: 62 - protein, 512 aa, synthetic construct; identical to SEQ ID NO: 54

SEQ ID NO: 63 - DNA, 1542 bp, synthetic construct
  source 1..1542: mol_type = other DNA
gaattcgccaccatggccgtgatggcacctagaaccctggtcctgctgctgagcggggcactggcactgacacagacttgggctggggatatcgtgatgacacagtcccctgctactctgtctctgagtccaggcgagcgggcaactctgtcctgcagaagctccaagtctctgcagaacgtgaatggaaacacctatctgtactggttccagcagaaaccaggccagagcccccagctgctgatctatagaatgagtaatctgaactcaggcgtgcccgataggttctcagggagcggcagcggcaccgagtttaccctgacaatttctagtctggagccagaagacttcgccgtgtactattgcatgcagcacctggagtaccccatcacctttggggctggaacaaagctggagatcaagggaggaggaggcagtggcggaggagggtcaggaggaggaggaagcgaggtccagctggtggaatctggcggaggactggtgaagcctggagggagcctgaaactgtcctgtgccgcttctgggtatactttcaccagctacgtcatgcattgggtgcggcaggcaccaggcaagggactggaatggatcggctatattaatccctacaacgacgggactaagtataatgagaaatttcagggcagggtgaccatctcaagcgataagtctattagtacagcctacatggagctgtcctctctgcgcagcgaagacacagcaatgtactattgcgccagggggacttactattacggaacccgcgtgttcgattactggggacagggcaccctggtcacagtgagttcagcagccgaacctaagagctccgacaaaacacacacttgcccaccttgtccagcaccagaggctgcaggaggaccaagcgtgttcctgtttccacccaagcctaaagataccctgatgatctccagaactcccgaagtcacctgtgtggtcgtgtcagtgagccacgaggaccctgaagtcaagtttaattggtacgtggatggcgtcgaggtgcataacgctaagacaaaaccacgagaggaacagtataacagtacataccgggtcgtgtcagtgctgaccgtcctgcatcaggactggctgaatggaaaggagtacaagtgcaaagtgtctaacaaggccctgcctgctccaatcgagaaaaccattagcaaggctaaaggccagccccgcgaacctcaggtctatgtgctgcctccaagccgagatgagctgacaaagaatcaggtctccctgctgtgtctggtgaaagggttctacccttctgacattgcagtggagtgggaaagtaacggacagccagaaaacaattatctgacatggccccctgtcctggactccgatggctctttctttctgtacagcaagctgactgtggataaatccaggtggcagcaggggaatgtcttttcctgttctgtgatgcacgaggccctgcacaaccattacacccagaagagtctgtcactgagccccggcaaatgaggatcc

SEQ ID NO: 64 - protein, 511 aa, synthetic construct
EFATMAVMAPRTLVLLLSGALALTQTWAGDIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKGGGGSGGGGSGGGGSEVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSSAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 65 - DNA, 1545 bp, synthetic construct; identical to SEQ ID NO: 57
  source 1..1545: mol_type = other DNA

SEQ ID NO: 66 - protein, 512 aa, synthetic construct; identical to SEQ ID NO: 58

SEQ ID NO: 67 - DNA, 1542 bp, synthetic construct
  source 1..1542: mol_type = other DNA
gaattcgccaccatggctgtgatggcacctagaacactggtcctgctgctgtccggggcactggcactgactcagacttgggctggggatattgtgatgacacagtcccctgctactctgtctctgagtccaggggagcgggcaactctgtcctgcagaagctccaagtctctgcagaacgtgaatggaaacacctatctgtactggttccagcagaaaccaggccagagcccccagctgctgatctatagaatgagtaatctgaactcaggcgtgcccgataggttctcagggagcggcagcggcaccgagtttaccctgacaatttctagtctggagccagaagacttcgccgtgtactattgcatgcagcacctggagtaccccatcacctttgggtgtggaacaaagctggagatcaagggaggaggaggcagtggcggaggagggtcaggaggaggaggaagcgaggtccagctggtggaatctggcggaggactggtgaagcctggagggagcctgaaactgtcctgcgccgcttctggctatactttcaccagctacgtcatgcattgggtgcggcaggcacctgggaagtgtctggaatggatcggatatattaatccctacaacgacggcactaagtataatgagaaatttcaggggagggtgaccatctcaagcgataagtctattagtacagcttacatggagctgtcctctctgcgcagcgaagacacagcaatgtactattgcgccaggggaacttactattacggcacccgcgtgttcgattactggggccaggggaccctggtcacagtgagttcagcagccgaacctaagagctccgacaaaacacacacttgcccaccttgtccagctccagaggctgcaggaggaccaagcgtgttcctgtttccacccaagcctaaagataccctgatgatctccagaactcccgaagtcacctgtgtggtcgtgtcagtgagccacgaggaccctgaagtcaagtttaattggtacgtggatggcgtcgaggtgcataacgccaagacaaaaccacgagaggaacagtataacagtacataccgggtcgtgtcagtgctgaccgtcctgcatcaggactggctgaatggaaaggagtacaagtgcaaagtgtctaacaaggccctgcctgctccaatcgagaaaaccattagcaaggcaaaaggccagccccgcgaacctcaggtctatgtgctgcctccaagccgagatgagctgacaaagaatcaggtctccctgctgtgtctggtgaaagggttctacccttctgacattgccgtggagtgggaaagtaacggacagccagaaaacaattatctgacatggccccctgtcctggactccgatggctctttctttctgtacagcaagctgactgtggataaatccaggtggcagcaggggaatgtcttttcctgttctgtgatgcacgaggccctgcacaaccattacacccagaagagtctgtcactgagccccggcaaatgaggatcc

SEQ ID NO: 68 - protein, 511 aa, synthetic construct
EFATMAVMAPRTLVLLLSGALALTQTWAGDIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGCGTKLEIKGGGGSGGGGSGGGGSEVQLVESGGGLVKPGGSLKLSCAASGYTFTSYVMHWVRQAPGKCLEWIGYINPYNDGTKYNEKFQGRVTISSDKSISTAYMELSSLRSEDTAMYYCARGTYYYGTRVFDYWGQGTLVTVSSAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 69 - DNA, 1545 bp, synthetic construct; identical to SEQ ID NO: 53
  source 1..1545: mol_type = other DNA

SEQ ID NO: 70 - protein, 512 aa, synthetic construct; identical to SEQ ID NO: 54

SEQ ID NO: 71 - DNA, 1527 bp, synthetic construct
  source 1..1527: mol_type = other DNA
gaattcgccaccatggctgtgatggcacctcgcaccctggtcctgctgctgagcggggcactggcactgacacagacatgggctggggaagtccagctggtccagagcggggctgaggtgaagaaaccaggagaaagcctgaaaatctcctgcaagggcagcggctactctttcagctcctcttggattggatgggtgcgccagatgccagggaagggactggagtggatgggcatcatctaccctgacgattctgacaccagatattctccaagttttcagggccaagtgactatctctgctgataaaagtattaggaccgcatatctgcagtggagttcactgaaggcaagcgacacagccatgtactattgcgcccgacacgtcactatgatctggggcgtgatcattgatttctggggccagggaaccctggtcacagtgagctccggcggaggaggctccggaggaggagggtctggaggcgggggaagtgcaattcagctgacacagagcccttctagtctgtcagcaagcgtcggcgaccgagtgaccatcacatgtagagcttcccaggggatttcaagcgctctggcatggtaccagcagaagccaggcaaagcccccaagctgctgatctatgatgcttcctctctggagtcaggggtgccaagcaggttctccggatctggcagtgggaccgactttactctgaccattagttcactgcagcccgaagatttcgctacatactattgccagcagttcaactcctacccttatacatttggacagggcactaaactggagatcaaggccgctgaacccaaaagctccgacaagacacacacttgccccccttgtccagcaccagaagcagcaggaggaccatctgtgttcctgtttccacccaagcctaaagataccctgatgattagccggactcctgaggtgacctgtgtggtcgtgtcagtgagccacgaggacccagaagtcaagtttaactggtacgtggatggcgtcgaagtgcataatgccaagacaaaacctagggaggaacagtacaattcaacctatcgcgtcgtgagcgtcctgacagtgctgcatcaggactggctgaacgggaaggagtataagtgcaaagtgagcaacaaggccctgcctgctccaatcgagaagacaatttccaaggcaaaaggccagccccgagaacctcaggtctacgtgctgcctccatctcgggacgagctgactaaaaaccaggtcagcctgctgtgtctggtgaaggggttctatccaagtgatatcgccgtggagtgggaatccaatggacagcccgagaacaattacctgacttggccccctgtgctggacagtgatggatcattctttctgtattccaaactgaccgtggataagtctcggtggcagcagggcaacgtcttttcctgttctgtgatgcatgaagccctgcacaatcattacacccagaaaagtctgtcactgagccccggcaagtgaggatcc

SEQ ID NO: 72 - protein, 506 aa, synthetic construct
EFATMAVMAPRTLVLLLSGALALTQTWAGEVQLVQSGAEVKKPGESLKISCKGSGYSFSSSWIGWVRQMPGKGLEWMGIIYPDDSDTRYSPSFQGQVTISADKSIRTAYLQWSSLKASDTAMYYCARHVTMIWGVIIDFWGQGTLVTVSSGGGGSGGGGSGGGGSAIQLTQSPSSLSASVGDRVTITCRASQGISSALAWYQQKPGKAPKLLIYDASSLESGVPSRFSGSGSGTDFTLTISSLQPEDFATYYCQQFNSYPYTFGQGTKLEIKAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 73 - DNA, 1545 bp, synthetic construct; identical to SEQ ID NO: 57
  source 1..1545: mol_type = other DNA

SEQ ID NO: 74 - protein, 512 aa, synthetic construct; identical to SEQ ID NO: 58

SEQ ID NO: 75 - DNA, 1527 bp, synthetic construct
  source 1..1527: mol_type = other DNA
gaattcgccaccatggccgtgatggctccaagaaccctggtcctgctgctgagtggggctctggctctgacacagacctgggctggggaagtgcagctggtccagagcggagctgaggtgaagaaaccaggcgaaagcctgaaaatctcctgcaagggcagcggctactctttcagctcctcttggattgggtgggtgcgccagatgcccggaaagtgtctggagtggatgggcatcatctaccctgacgattctgacaccagatattctccaagttttcagggccaagtgactatctctgctgataaaagtattaggaccgcatatctgcagtggagttcactgaaggcaagcgacacagccatgtactattgcgcccgacacgtcactatgatctggggcgtgatcattgatttctgggggcagggaaccctggtcacagtgagctccggcggaggaggctccggaggaggagggtctggaggcgggggaagtgcaattcagctgacacagagcccttctagtctgtcagcaagcgtcggggaccgagtgaccatcacatgtagagcttcccagggaatttcaagcgctctggcatggtaccagcagaagccaggaaaagcccccaagctgctgatctatgatgcttcctctctggagtcaggcgtgccaagcaggttctccggctctgggagtggaaccgactttactctgaccattagttcactgcagcccgaagatttcgctacatactattgccagcagttcaactcctacccttatacatttggctgtgggactaaactggagatcaaggccgctgaacccaaaagctccgacaagacacacacttgccccccttgtccagcaccagaagcagcaggaggaccatctgtgttcctgtttccacccaagcctaaagataccctgatgattagccggactcctgaggtgacctgcgtggtcgtgtcagtgagccacgaggacccagaagtcaagtttaactggtacgtggatggcgtcgaagtgcataatgccaagacaaaacctagggaggaacagtacaattcaacctatcgcgtcgtgagcgtcctgacagtgctgcatcaggactggctgaacggcaaggagtataagtgcaaagtgagcaacaaggccctgcctgctccaatcgagaagacaatttccaaggcaaaagggcagccccgagaacctcaggtctacgtgctgcctccatctcgggacgagctgactaaaaaccaggtcagcctgctgtgtctggtgaagggcttctatccaagtgatatcgccgtggagtgggaatccaatgggcagcccgagaacaattacctgacttggccccctgtgctggacagtgatgggtcattctttctgtattccaaactgaccgtggataagtctcggtggcagcagggaaacgtcttttcctgttctgtgatgcatgaagccctgcacaatcattacacccagaaaagtctgtcactgagccccggcaagtgaggatcc

SEQ ID NO: 76 - protein, 506 aa, synthetic construct
EFATMAVMAPRTLVLLLSGALALTQTWAGEVQLVQSGAEVKKPGESLKISCKGSGYSFSSSWIGWVRQMPGKCLEWMGIIYPDDSDTRYSPSFQGQVTISADKSIRTAYLQWSSLKASDTAMYYCARHVTMIWGVIIDFWGQGTLVTVSSGGGGSGGGGSGGGGSAIQLTQSPSSLSASVGDRVTITCRASQGISSALAWYQQKPGKAPKLLIYDASSLESGVPSRFSGSGSGTDFTLTISSLQPEDFATYYCQQFNSYPYTFGCGTKLEIKAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 77 - DNA, 1545 bp, synthetic construct; identical to SEQ ID NO: 53
  source 1..1545: mol_type = other DNA

SEQ ID NO: 78 - protein, 512 aa, synthetic construct; identical to SEQ ID NO: 54

SEQ ID NO: 79 - DNA, 1527 bp, synthetic construct
  source 1..1527: mol_type = other DNA
gaattcgccaccatggccgtcatggcacctagaacactggtcctgctgctgtccggggcactggcactgactcagacttgggctggggctattcagctgacccagagccctagctccctgtctgcaagtgtcggcgaccgggtgaccatcacatgcagagcctcccaggggatttctagtgctctggcatggtaccagcagaagccaggcaaagcccccaagctgctgatctatgatgcttcaagcctggaaagcggggtgccatcccggttctcaggaagcggctccggaaccgactttactctgaccatttcctctctgcagcccgaggatttcgcaacatactattgccagcagttcaacagctacccttatacatttggacagggcactaaactggaaatcaagggcggaggaggctccggaggaggagggtctggaggaggaggaagtgaggtccagctggtgcagtccggggctgaggtgaagaaaccaggagaatctctgaaaatcagttgtaaggggtccggatactctttcagttcaagctggattggatgggtgcgccagatgccaggcaagggactggagtggatgggcatcatctaccctgacgattctgacacaagatattctccaagttttcagggccaagtgactatctcagctgataaaagcattaggaccgcatatctgcagtggtcctctctgaaggcaagcgacacagccatgtactattgcgcccgacacgtcactatgatctggggcgtgatcattgatttctggggacagggcaccctggtcacagtgagttcagccgctgaacccaaaagctccgacaagacacatacttgccccccttgtccagctccagaggcagcaggaggaccatctgtgttcctgtttccacccaagcctaaagataccctgatgattagcaggactcctgaagtcacctgtgtggtcgtgtcagtgagccacgaggacccagaagtcaagtttaactggtacgtggatggcgtcgaggtgcataatgccaagacaaaacctagggaggaacagtacaattcaacctatcgcgtcgtgagcgtcctgacagtgctgcaccaggactggctgaacgggaaggagtataagtgcaaagtgagcaacaaggccctgcctgctccaatcgagaagacaatttccaaggctaaaggccagccccgagaacctcaggtctacgtgctgcctccatctcgggacgagctgactaaaaaccaggtcagcctgctgtgtctggtgaaggggttctatccaagtgatatcgcagtggagtgggaatccaatggacagcccgaaaacaattacctgacttggccccctgtgctggacagtgatggatcattctttctgtattccaaactgaccgtggataagtctcggtggcagcagggcaacgtcttttcctgttctgtgatgcatgaggccctgcacaatcattacacccagaaaagtctgtcactgagccccggcaagtgaggatcc

SEQ ID NO: 80 - protein, 506 aa, synthetic construct
EFATMAVMAPRTLVLLLSGALALTQTWAGAIQLTQSPSSLSASVGDRVTITCRASQGISSALAWYQQKPGKAPKLLIYDASSLESGVPSRFSGSGSGTDFTLTISSLQPEDFATYYCQQFNSYPYTFGQGTKLEIKGGGGSGGGGSGGGGSEVQLVQSGAEVKKPGESLKISCKGSGYSFSSSWIGWVRQMPGKGLEWMGIIYPDDSDTRYSPSFQGQVTISADKSIRTAYLQWSSLKASDTAMYYCARHVTMIWGVIIDFWGQGTLVTVSSAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 81 - DNA, 1545 bp, synthetic construct; identical to SEQ ID NO: 57
  source 1..1545: mol_type = other DNA

SEQ ID NO: 82 - protein, 512 aa, synthetic construct; identical to SEQ ID NO: 58

SEQ ID NO: 83 - DNA, 1527 bp, synthetic construct
  source 1..1527: mol_type = other DNA
gaattcgccaccatggccgtgatggctcctcgcaccctggtcctgctgctgagcggggcactggcactgacacagacttgggctggggctattcagctgacccagagccctagctccctgtctgcaagtgtcggggaccgggtgaccatcacatgcagagcctcccagggaatttctagtgctctggcatggtaccagcagaagccaggaaaagcccccaagctgctgatctatgatgcttcaagcctggaaagcggcgtgccatcccggttctcaggaagcggctccggaaccgactttactctgaccatttcctctctgcagcccgaggatttcgcaacatactattgccagcagttcaacagctacccttatacatttggatgtggcactaaactggaaatcaagggcggaggaggctccggaggaggagggtctggaggaggaggaagtgaggtccagctggtgcagtccggagctgaggtgaagaaaccaggcgaatctctgaaaatcagttgcaaggggtccggatactctttcagttcaagctggattgggtgggtgcgccagatgcccggaaagtgtctggagtggatgggcatcatctaccctgacgattctgacacaagatattctccaagttttcagggccaagtgactatctcagctgataaaagcattaggaccgcatatctgcagtggtcctctctgaaggcaagcgacacagccatgtactattgcgcccgacacgtcactatgatctggggcgtgatcattgatttctggggccaggggaccctggtcacagtgagttcagccgctgaacccaaaagctccgacaagacacatacttgccccccttgtccagctccagaggcagcaggaggaccatctgtgttcctgtttccacccaagcctaaagataccctgatgattagcaggactcctgaagtcacctgtgtggtcgtgtcagtgagccacgaggacccagaagtcaagtttaactggtacgtggatggcgtcgaggtgcataatgccaagacaaaacctagggaggaacagtacaattcaacctatcgcgtcgtgagcgtcctgacagtgctgcaccaggactggctgaacggcaaggagtataagtgcaaagtgagcaacaaggccctgcctgctccaatcgagaagacaatttccaaggctaaagggcagccccgagaacctcaggtctacgtgctgcctccatctcgggacgagctgactaaaaaccaggtcagcctgctgtgtctggtgaagggcttctatccaagtgatatcgcagtggagtgggaatccaatgggcagcccgaaaacaattacctgacttggccccctgtgctggacagtgatgggtcattctttctgtattccaaactgaccgtggataagtctcggtggcagcagggaaacgtcttttcctgttctgtgatgcatgaggccctgcacaatcattacacccagaaaagtctgtcactgagccccggcaagtgaggatcc

SEQ ID NO: 84 - protein, 506 aa, synthetic construct
EFATMAVMAPRTLVLLLSGALALTQTWAGAIQLTQSPSSLSASVGDRVTITCRASQGISSALAWYQQKPGKAPKLLIYDASSLESGVPSRFSGSGSGTDFTLTISSLQPEDFATYYCQQFNSYPYTFGCGTKLEIKGGGGSGGGGSGGGGSEVQLVQSGAEVKKPGESLKISCKGSGYSFSSSWIGWVRQMPGKCLEWMGIIYPDDSDTRYSPSFQGQVTISADKSIRTAYLQWSSLKASDTAMYYCARHVTMIWGVIIDFWGQGTLVTVSSAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 85 - DNA, 1548 bp, synthetic construct
  source 1..1548: mol_type = other DNA
gaattcgccaccatggctgtgatggctcctagaaccctggtcctgctgctgtccggggcactggcactgactcagacttgggctggggatattcagctgacacagagtcctgcttcactggcagtgagcctgggacagcgagcaactatctcctgcaaagctagtcagtcagtggactatgatggcgactcctatctgaactggtaccagcagatcccagggcagccccctaagctgctgatctacgacgcctcaaatctggtgagcggcatcccaccacgattcagcggcagcggctctgggactgattttaccctgaacattcacccagtcgagaaggtggacgccgctacctaccattgccagcagtctaccgaggacccctggacattcggcgggggaactaaactggaaatcaagggaggaggaggcagtggcggaggagggtcaggaggaggaggaagccaggtgcagctgcagcagagcggagcagagctggtcagaccaggaagctccgtgaaaatttcctgtaaggcatctggctatgccttttctagttactggatgaattgggtgaagcagaggccaggacagggcctggaatggatcgggcagatttggcccggggatggagacaccaactataatggaaagttcaaaggcaaggctacactgactgcagacgagtcaagctccacagcttatatgcagctgtctagtctggccagcgaggattccgctgtgtacttttgcgcacggagagaaaccacaactgtgggcaggtactattacgcaatggactactggggccaggggaccacagtcaccgtgtcaagcgcagccgaacccaaatcctctgataagacccacacatgccctccatgtccagcacctgagctgctgggaggaccaagcgtgttcctgtttccacctaaacctaaggacacactgatgatctctcggacacccgaagtcacttgtgtggtcgtggatgtgagccacgaggaccctgaagtcaaattcaactggtacgtggatggcgtcgaggtgcataatgccaaaactaagcctagggaggaacagtataactccacttaccgcgtcgtgtctgtcctgaccgtgctgcatcaggactggctgaacggaaaggagtacaaatgcaaggtgagcaacaaggccctgccagctcccatcgagaagacaatttccaaagctaagggccagcctcgagaaccacaggtctatgtgtacccacccagccgggacgagctgaccaaaaaccaggtctccctgacatgtctggtgaagggattttatccttctgatattgccgtggagtgggaaagtaatggccagccagaaaacaattacaagactacccctccagtgctggattctgacgggagtttcgcactggtcagtaaactgactgtggataagtcacggtggcagcagggaaacgtctttagttgttcagtgatgcacgaggccctgcacaatcattacacccagaaaagcctgtccctgtctcccggcaagtgaggatcc

SEQ ID NO: 86 - DNA, 1527 bp, synthetic construct
  source 1..1527: mol_type = other DNA
gaattcgccaccatggctgtgatggctcctagaacactggtcctgctgctgtccggggcactggcactgactcagacttgggctggggatattaagctgcagcagagcggagctgagctggcacgaccaggagccagtgtgaaaatgtcatgcaagaccagcggctacacattcactcggtatacaatgcactgggtgaagcagagaccaggacagggactggaatggatcggatatattaacccttcccgaggctacacaaactacaaccagaagtttaaagacaaggccactctgaccacagataagagctcctctaccgcttacatgcagctgagttcactgacaagtgaggactcagctgtgtactattgcgcaaggtactatgacgatcattactgtctggattattggggacagggcactaccctgactgtcagctccgtggaaggagggagcggaggctccggaggatctggcgggagtggaggcgtggacgatatccagctgacccagtccccagcaattatgtccgcctctcccggcgagaaagtcaccatgacatgccgcgcttctagttcagtgagctacatgaactggtatcagcagaaatcaggcactagccccaagagatggatctacgacacctccaaggtcgcatctggggtgccttataggttcagtgggtcaggaagcggcacctcctactctctgacaattagctccatggaggcagaagatgccgctacctactattgtcagcagtggtctagtaatccactgacttttggggccggaaccaaactggagctgaaggcagccgaacccaaatcaagcgacaagactcacacctgccccccttgtccagcacccgaactgctggggggacctagcgtgttcctgtttccacccaaaccaaaggatacactgatgatcagccggacacctgaggtcacttgcgtggtcgtggacgtgagccacgaggaccccgaagtcaagttcaactggtacgtggacggcgtcgaagtgcataatgctaaaaccaagcctagggaggaacagtacaatagtacatatagagtcgtgtcagtgctgaccgtcctgcatcaggattggctgaacgggaaggagtacaaatgcaaggtgtccaacaaggccctgcctgctccaatcgagaagaccatttctaaagccaagggccagccccgagaacctcaggtctatgtgctgcctccatcccgggacgagctgacaaaaaaccaggtctctctgctgtgtctggtgaaggggttctacccatctgatattgctgtggagtgggaaagtaatggacagcccgagaacaattatctgacatggccccctgtgctggactccgatggatctttctttctgtacagcaaactgactgtggacaagtccagatggcagcagggcaacgtctttagttgttcagtgatgcacgaggccctgcacaatcattacacccagaaaagcctgtccctgtctcccggcaagtgaggatcc

SEQ ID NO: 87 - protein, 513 aa, synthetic construct
EFATMAVMAPRTLVLLLSGALALTQTWAGDIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 88 - protein, 506 aa, synthetic construct
EFATMAVMAPRTLVLLLSGALALTQTWAGDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 89 - DNA, 1608 bp, synthetic construct
  source 1..1608: mol_type = other DNA
gaattcgcgaccatggccgtgatggcacctagaacactggtcctgctgctgagcggggcactggcactgacacagacttgggctggagatattcagctgacacagtctccagctagtctggcagtgagcctgggccagcgggctactatcagctgcaaggcaagccagtccgtcgactacgatggggacagctatctgaactggtaccagcagatccccggacagccccctaaactgctgatctacgacgcctcaaatctggtgagcggcatcccacccagattctctggaagtggctcagggaccgattttacactgaacattcaccccgtggagaaggtcgacgccgctacctaccattgccagcagtccactgaggacccctggaccttcggaggaggaacaaagctggaaatcaaaggcggaggaggcagtggaggaggagggagcggaggaggaggaagccaggtgcagctgcagcagagcggagcagaactggtgagacctggaagctccgtcaagatttcctgtaaagcatctggctatgccttttctagttactggatgaattgggtgaagcagaggccaggacagggactggagtggatcggacagatttggcctggggatggagacaccaactacaatggaaagttcaaaggcaaggctaccctgacagcagacgaatcaagctccacagcttacatgcagctgtctagtctggcatcagaggatagcgccgtgtatttttgcgctcggagagaaaccacaactgtcggccgctactattacgccatggactactggggccaggggaccacagtgacagtctcaagcggcgggggaggctccgatatcaagctgcagcagtctggagcagagctggctcgaccaggagccagtgtgaagatgtcatgtaaaaccagcggctatactttcaccaggtacacaatgcactgggtgaaacagcgcccaggacagggcctggaatggatcggatacattaacccctccaggggctataccaactacaatcagaagttcaaggataaagccactctgactaccgacaagtcctctagtaccgcttatatgcagctgtcaagcctgacatccgaggactctgcagtgtattactgcgcccgctattacgacgatcattattgtctggattactgggggcagggaacaactctgactgtgtcctctgtcgaagggggaagtggagggtcaggaggcagcggaggcagcggaggggtggacgatatccagctgacccagtcccctgccattatgagcgcttccccaggcgagaaggtgacaatgacttgcagggctagttcaagcgtctcttatatgaattggtatcagcagaagtctggcactagtcctaaacgatggatctatgacacctccaaagtggcatctggggtcccataccggttctctggcagtgggtcaggaactagctattccctgaccatttcctctatggaggcagaagatgcagccacctattactgtcagcagtggagttcaaatcccctgacatttggcgccgggactaagctggagctgaaacaccatcaccatcaccattgaggatcc

SEQ ID NO: 90 - protein, 533 aa, synthetic construct
EFATMAVMAPRTLVLLLSGALALTQTWAGDIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 91 - DNA, 1548 bp, synthetic construct
  source 1..1548: mol_type = other DNA
gaattcgccaccatggccgtgatggcaccccgcaccctggtcctgctgctgtccggggcactggcactgactcagacttgggctggcgatattcagctgacacagagtcctgcatcactggctgtgagcctgggacagcgagcaactatctcctgcaaagccagtcagtcagtggactatgatggcgactcctatctgaactggtaccagcagatcccagggcagccccctaagctgctgatctacgacgcctcaaatctggtgagcggcatcccaccacgattcagcggcagcggctctgggactgattttaccctgaacattcacccagtcgagaaggtggacgccgctacctaccattgccagcagtctaccgaggacccctggacattcggcgggggaactaaactggaaatcaagggaggaggaggcagtggcggaggagggtcaggaggaggaggaagccaggtgcagctgcagcagagcggagcagagctggtcagaccaggaagctccgtgaaaatttcctgtaaggcttctggctatgcattttctagttactggatgaattgggtgaagcagaggccaggacagggcctggaatggatcgggcagatttggcccggggatggagacaccaactataatggaaagttcaaaggcaaggccacactgactgctgacgagtcaagctccacagcctatatgcagctgtctagtctggcaagcgaggattccgccgtgtacttttgcgctcggagagaaaccacaactgtgggcaggtactattacgctatggactactggggccaggggaccacagtcaccgtgtcaagcgcagccgaacccaaatcctctgataagacccacacatgccctccatgtccagctcctgaggctgcaggaggaccaagcgtgttcctgtttccccctaaacctaaggacacactgatgatctctcggacacccgaagtcacttgtgtggtcgtggatgtgagccacgaggaccctgaagtcaaattcaactggtacgtggatggcgtcgaggtgcataatgccaaaactaagcctagggaggaacagtataactccacttaccgcgtcgtgtctgtcctgaccgtgctgcatcaggactggctgaacggaaaggagtacaaatgcaaggtgagcaacaaggcactgccagcccccatcgagaagacaatttccaaagcaaagggccagcctcgagaaccacaggtctatgtgtacccacccagccgggacgagctgaccaaaaaccaggtctccctgacatgtctggtgaagggattttatccttctgatattgccgtggagtgggaaagtaatggccagccagaaaacaattacaagactacccctccagtgctggattctgacgggagtttcgctctggtcagtaaactgactgtggataagtcacggtggcagcagggaaacgtctttagttgttcagtgatgcacgaggcactgcacaatcattacacccagaaaagcctgtccctgtctcccggcaagtgaggatcc

SEQ ID NO: 92 - DNA, 1527 bp, synthetic construct
  source 1..1527: mol_type = other DNA
gaattcgccaccatggccgtcatggcacctagaaccctggtcctgctgctgagcggggcactggcactgacacagacttgggccggagacatcaaactgcagcagagcggagcagagctggctcgaccaggagccagtgtgaaaatgtcatgcaagaccagcggctacacattcactcggtatacaatgcactgggtgaagcagagaccaggacagggactggaatggatcggatatattaacccttcccgaggctacacaaactacaaccagaagtttaaagacaaggcaactctgaccacagataagagctcctctaccgcctacatgcagctgagttcactgacaagtgaggactcagccgtgtactattgcgctaggtactatgacgatcattactgtctggattattggggacagggcactaccctgactgtcagctccgtggaaggagggagcggaggctccggaggatctggcgggagtggaggcgtggacgatatccagctgacccagtccccagctattatgtccgcatctcccggcgagaaagtcaccatgacatgccgcgcctctagttcagtgagctacatgaactggtatcagcagaaatcaggcactagccccaagagatggatctacgacacctccaaggtcgcttctggggtgccttataggttcagtgggtcaggaagcggcacctcctactctctgacaattagctccatggaggctgaagatgccgctacctactattgtcagcagtggtctagtaatccactgacttttggggcaggaaccaaactggagctgaaggcagccgaacccaaatcaagcgacaagactcacacctgcccaccttgtccagcaccagaagctgcaggaggacctagcgtgttcctgtttccacccaaaccaaaggatacactgatgatcagccggacacctgaggtcacttgcgtggtcgtggacgtgagccacgaggaccccgaagtcaagttcaactggtacgtggacggcgtcgaagtgcataatgccaaaaccaagcctagggaggaacagtacaatagtacatatagagtcgtgtcagtgctgaccgtcctgcatcaggattggctgaacgggaaggagtacaaatgcaaggtgtccaacaaggcactgcctgccccaatcgagaagaccatttctaaagcaaagggccagccccgagaacctcaggtctatgtgctgcctccatcccgggacgagctgacaaaaaaccaggtctctctgctgtgtctggtgaaggggttctacccatctgatattgctgtggagtgggaaagtaatggacagcccgagaacaattatctgacatggccccctgtgctggactccgatggatctttctttctgtacagcaaactgactgtggacaagtccagatggcagcagggcaacgtctttagttgttcagtgatgcacgaggccctgcacaatcattacacccagaaaagcctgtccctgtctcccggcaagtgaggatcc

SEQ ID NO: 93 - protein, 513 aa, synthetic construct
EFATMAVMAPRTLVLLLSGALALTQTWAGDIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSAAEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 94 - protein, 506 aa, synthetic construct
EFATMAVMAPRTLVLLLSGALALTQTWAGDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKAAEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 95 - DNA, 1548 bp, synthetic construct
  source 1..1548: mol_type = other DNA
gaattcgccaccatggctgtgatggcacctagaaccctggtcctgctgctgagcggggcactggcactgacccagacttgggccggagacattcagctgacacagagtcctgcttcactggcagtgagcctgggacagcgagcaactatctcctgcaaagctagtcagtcagtggactatgatggcgactcctatctgaactggtaccagcagatcccagggcagccccctaagctgctgatctacgacgcttcaaatctggtgagcggcatcccaccacgattcagcggcagcggctctgggactgattttaccctgaacattcacccagtcgagaaggtggacgccgctacctaccattgccagcagtctaccgaggacccctggacattcggcgggggaactaaactggaaatcaagggaggaggaggcagtggcggaggagggtcaggaggaggaggaagccaggtgcagctgcagcagagcggagcagagctggtcagaccaggaagctccgtgaaaatttcctgtaaggcatctggctatgccttttctagttactggatgaattgggtgaagcagaggccaggacagggcctggaatggatcgggcagatttggcccggggatggagacaccaactataatggaaagttcaaaggcaaggctacactgactgcagacgagtcaagctccacagcctatatgcagctgtctagtctggccagcgaggattccgctgtgtacttttgcgcacggagagaaaccacaactgtgggcaggtactattacgctatggactactggggccaggggaccacagtcaccgtgtcaagcgcagccgaacccaaatcctctgataagacccacacatgccctccatgtccagcacctgagctgctgggaggaccaagcgtgttcctgtttccacctaaacctaaggacacactgatgatctctcggacacccgaagtcacttgtgtggtcgtggatgtgagccacgaggaccctgaagtcaaattcaactggtacgtggatggcgtcgaggtgcataatgcaaaaactaagcctagggaggaacagtatgcctccacttaccgcgtcgtgtctgtcctgaccgtgctgcatcaggactggctgaacggaaaggagtacaaatgcaaggtgagcaacaaggccctgccagctcccatcgagaagacaatttccaaagcaaagggccagcctcgagaaccacaggtctatgtgtacccacccagccgggacgagctgaccaaaaaccaggtctccctgacatgtctggtgaagggattttatccttctgatattgccgtggagtgggaaagtaatggccagccagaaaacaattacaagactacccctccagtgctggattctgacgggagtttcgctctggtcagtaaactgactgtggataagtcacggtggcagcagggaaacgtctttagttgttcagtgatgcacgaggcactgcacaatcattacacccagaaaagcctgtccctgtctcccggcaagtgaggatcc

SEQ ID NO: 96 - DNA, 1527 bp, synthetic construct
  source 1..1527: mol_type = other DNA
gaattcgccaccatggccgtcatggcacctagaaccctggtcctgctgctgagcggggcactggcactgacacagacttgggctggagacatcaaactgcagcagagcggagctgagctggcacgaccaggagccagtgtgaaaatgtcatgcaagaccagcggctacacattcactcggtatacaatgcactgggtgaagcagagaccaggacagggactggaatggatcggatatattaacccttcccgaggctacacaaactacaaccagaagtttaaagacaaggcaactctgaccacagataagagctcctctaccgcctacatgcagctgagttcactgacaagtgaggactcagctgtgtactattgcgcaaggtactatgacgatcattactgtctggattattggggacagggcactaccctgactgtcagctccgtggaaggagggagcggaggctccggaggatctggcgggagtggaggcgtggacgatatccagctgacccagtccccagcaattatgtccgcctctcccggcgagaaagtcaccatgacatgccgcgcctctagttcagtgagctacatgaactggtatcagcagaaatcaggcactagccccaagagatggatctacgacacctccaaggtcgcttctggggtgccttataggttcagtgggtcaggaagcggcacctcctactctctgacaattagctccatggaggctgaagatgccgctacctactattgtcagcagtggtctagtaatccactgacttttggggcaggaaccaaactggagctgaaggcagccgaacccaaatcaagcgacaagactcacacctgcccaccttgtccagcaccagaactgctgggaggacctagcgtgttcctgtttccacccaaaccaaaggatacactgatgatcagccggacacctgaggtcacttgcgtggtcgtggacgtgagccacgaggaccccgaagtcaagttcaactggtacgtggacggcgtcgaagtgcataatgccaaaaccaagcctagggaggaacagtacgctagtacatatagagtcgtgtcagtgctgaccgtcctgcatcaggattggctgaacgggaaggagtacaaatgcaaggtgtccaacaaggccctgcctgctccaatcgagaagaccatttctaaagcaaagggccagccccgagaacctcaggtctatgtgctgcctccatcccgggacgagctgacaaaaaaccaggtctctctgctgtgtctggtgaaggggttctacccatctgatattgctgtggagtgggaaagtaatggacagcccgagaacaattatctgacatggccccctgtgctggactccgatggatctttctttctgtacagcaaactgactgtggacaagtccagatggcagcagggcaacgtctttagttgttcagtgatgcacgaggccctgcacaatcattacacccagaaaagcctgtccctgtctcccggcaagtgaggatcc

SEQ ID NO: 97 - protein, 513 aa, synthetic construct
EFATMAVMAPRTLVLLLSGALALTQTWAGDIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSAAEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 98 - protein, 506 aa, synthetic construct
EFATMAVMAPRTLVLLLSGALALTQTWAGDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKAAEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 99 - DNA, 1548 bp, synthetic construct; identical to SEQ ID NO: 85
  source 1..1548: mol_type = other DNA

SEQ ID NO: 100 - DNA, 1518 bp, synthetic construct
  source 1..1518: mol_type = other DNA
gaattcgccaccatggccgtgatggcaccccgaacactggtcctgctgctgagcggggcactggcactgacacagacttgggctggacagatcgtcctgacacagagcccagcaatcatgtcagccagccccggcgagaaagtcacaatgacttgctcagcaagctcctctgtgagctacatgaactggtatcagcagaaaagcggaacctcccccaagagatggatctacgacacatccaagctggcttctggagtgcctgcacacttcaggggcagcggctctgggaccagttattcactgacaatttccggcatggaggctgaagatgccgctacctactattgccagcagtggagttcaaacccattcacttttggatctggcaccaagctggaaattaatggcggaggaggctccggaggaggagggtctggaggaggaggaagtcaggtgcagctgcagcagagcggagctgagctggcacgaccaggagcaagtgtgaaaatgtcctgtaaggccagcggctacaccttcacacggtataccatgcattgggtgaaacagagacccgggcagggactggaatggatcgggtacattaatccttcccgaggatacacaaactacaaccagaagtttaaagacaaggctactctgaccacagataagagctcctctaccgcatatatgcagctgagttcactgacatctgaggacagtgccgtgtactattgcgctaggtactatgacgatcactactccctggattattggggccaggggactaccctgacagtgagctccgcagccgaacctaaatctagtgacaagactcatacctgccccccttgtccagcaccagagctgctgggaggacctagcgtgttcctgtttccacccaaaccaaaggatactctgatgatctcccggacacctgaagtcacttgtgtggtcgtggacgtgtctcacgaggaccccgaagtcaagtttaactggtacgtggacggcgtcgaggtgcataatgccaaaaccaagcccagggaggaacagtacaactccacatatcgcgtcgtgtctgtcctgactgtgctgcaccaggattggctgaacggcaaggagtacaaatgcaaggtgagcaacaaggccctgcctgctccaatcgagaagacaattagcaaagccaaggggcagccccgagaacctcaggtctacgtgctgcctccatctcgggacgagctgactaaaaaccaggtcagtctgctgtgtctggtgaagggcttctatccaagcgatattgctgtggagtgggaatccaatgggcagcccgaaaacaattacctgacttggccccctgtcctggactcagatgggagcttctttctgtatagtaaactgaccgtggacaagtcacggtggcagcagggaaacgtctttagctgttccgtgatgcatgaggccctgcacaatcattacacccagaaatctctgagtctgtcacccggcaagtgaggatcc

SEQ ID NO: 101 - protein, 513 aa, synthetic construct; identical to SEQ ID NO: 87

SEQ ID NO: 102 - protein, 503 aa, synthetic construct
EFATMAVMAPRTLVLLLSGALALTQTWAGQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINGGGGSGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 103 - DNA, 1548 bp, synthetic construct; identical to SEQ ID NO: 85
  source 1..1548: mol_type = other DNA

SEQ ID NO: 104 - DNA, 1527 bp, synthetic construct
  source 1..1527: mol_type = other DNA
gaattcgccaccatggccgtgatggctcctcggaccctggtgctgctgctgagtggggctctggctctgacacagacttgggccggacaggtgcagctgcagcagtctggagctgagctggcaaggcctggcgcatccgtgaaaatgtcttgcaaggccagtggctacaccttcacacggtatactatgcactgggtgaaacagagacccggacagggcctggaatggatcgggtacattaacccttcccgcggatacaccaactacaaccagaagtttaaagacaaggctactctgaccacagataagagctcctctaccgcatatatgcagctgagttcactgacaagcgaggactccgctgtgtactattgcgcacgctactatgacgatcattacagcctggattattgggggcagggaactaccctgacagtgagctccgccaaaacaactccaaagctggaggaaggcgagttctccgaagctcgagtcgacatcgtgctgactcagtctccagcaattatgtcagccagccccggggagaaagtcactatgacctgttccgcctctagttcagtgtcttacatgaattggtatcagcagaaatctggaactagtcccaagagatggatctacgacacctcaaagctggccagcggcgtgcctgctcactttaggggctccgggtctggaactagttattcactgaccattagcgggatggaggccgaagatgccgctacctactattgccagcagtggagctccaacccattcacatttggctccgggactaagctggagatcaatcgggcagccgaacccaaatctagtgacaagacacatacttgccccccttgtccagcaccagaactgctgggaggacctagcgtgttcctgtttccacccaaaccaaaggatacactgatgattagtagaacccctgaggtcacatgtgtggtcgtggacgtgtcccacgaggaccccgaagtcaagttcaactggtacgtggacggcgtcgaagtgcataatgccaaaaccaagcccagggaggaacagtacaacagtacctatcgcgtcgtgtcagtcctgacagtgctgcaccaggattggctgaacggaaaggagtacaaatgcaaggtgtctaacaaggccctgcctgctccaatcgagaagaccattagcaaagcaaagggccagccccgagaacctcaggtctacgtgctgcctccatcacgggacgagctgacaaaaaaccaggtcagcctgctgtgtctggtgaagggcttctatccatctgatatcgctgtggagtgggaaagtaatgggcagcccgagaacaattacctgacatggccccctgtcctggactcagatgggagcttctttctgtatagcaaactgactgtggataagtccaggtggcagcagggaaacgtctttagctgttccgtgatgcatgaagccctgcacaatcattacacccagaaatctctgagtctgtcacctggcaagtgaggatcc

SEQ ID NO: 105 - protein, 513 aa, synthetic construct; identical to SEQ ID NO: 87

SEQ ID NO: 106 - protein, 506 aa, synthetic construct
EFATMAVMAPRTLVLLLSGALALTQTWAGQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSAKTTPKLEEGEFSEARVDIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 107 - DNA, 1548 bp, synthetic construct; identical to SEQ ID NO: 85
  source 1..1548: mol_type = other DNA

SEQ ID NO: 108 - DNA, 1503 bp, synthetic construct
  source 1..1503: mol_type = other DNA
gaattcgccaccatggccgtgatggctcctagaacactggtcctgctgctgagcggggcactggcactgacacagacttgggctgggcaggtccagctgcagcagagcggagctgagctggcacgacctggagcatccgtgaaaatgtcttgcaaggccagtggctacaccttcacacggtatacaatgcactgggtgaaacagagacccgggcagggactggaatggatcggctacattaacccttcccgagggtacactaactacaaccagaagtttaaagacaaggctactctgaccacagataagagctcctctaccgcatatatgcagctgagttcactgacaagcgaggactccgctgtgtactattgcgcacggtactatgacgatcattactccctggattattggggccaggggactaccctgacagtcagctccgccaaaacaactccaaagctgggcggggacatcgtgctgactcagtctccagcaattatgtcagccagccccggggagaaagtcactatgacctgttccgcttctagttcagtgtcttacatgaattggtatcagcagaaatctgggactagtcccaagagatggatctacgacacctcaaagctggccagcggagtgcctgctcacttcaggggcagcggctctgggactagttattcactgaccatttctggcatggaggccgaagatgccgctacctactattgccagcagtggagctccaacccattcacatttggaagcggcactaagctggagatcaatagggcagccgaacccaaatctagtgacaagacacatacttgcccaccttgtccagcaccagaactgctgggaggacctagcgtgttcctgtttccacccaaaccaaaggatacactgatgatttctcgcacccctgaggtcacatgtgtggtcgtggacgtcagtcacgaggaccccgaagtcaagtttaactggtacgtggacggcgtcgaagtgcataatgccaaaaccaagcccagggaggaacagtacaacagtacctatcgcgtcgtgtcagtcctgacagtgctgcaccaggattggctgaacggaaaggagtacaaatgcaaggtgagcaacaaggccctgcctgctccaatcgagaagactatttccaaagcaaagggccagccccgagaacctcaggtctacgtgctgcctccatcacgggacgagctgaccaaaaaccaggtctccctgctgtgtctggtgaagggattctatccatctgatatcgctgtggagtgggaaagtaatggccagcccgagaacaattacctgacctggccccctgtgctggactcagatggcagcttctttctgtatagcaaactgacagtggataagtccagatggcagcaggggaacgtctttagctgttccgtgatgcatgaagccctgcacaatcattacacccagaaatctctgagtctgtcacctggcaagtgaggatcc

SEQ ID NO: 109 - protein, 513 aa, synthetic construct; identical to SEQ ID NO: 87

SEQ ID NO: 110 - protein, 498 aa, synthetic construct
EFATMAVMAPRTLVLLLSGALALTQTWAGQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSAKTTPKLGGDIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 111 - DNA, 1527 bp, synthetic construct
  source 1..1527: mol_type = other DNA
gaattcgccaccatggccgtcatggcacctagaaccctggtcctgctgctgagcggggcactggcactgacacagacttgggctggagacatcaaactgcagcagagcggagctgagctggcacgaccaggagccagtgtgaaaatgtcatgcaagacaagcggctacaccttcacacggtatactatgcactgggtgaaacagagacccggccaggggctggaatggatcggatatattaacccttcccgaggctacaccaactataatcagaagtttaaagacaaggccaccctgaccacagataagagctcctctacagcttacatgcagctgagttcactgactagtgaggactcagctgtgtactattgcgcaaggtactatgacgatcattactgtctggattattggggacagggcactaccctgactgtcagctccgtggaaggagggagcggaggctccggaggatctggcgggagtggaggcgtggacgatatccagctgacccagtccccagcaattatgtccgcctctcccggcgagaaagtgactatgacctgccgcgcctctagttcagtgagctacatgaactggtatcagcagaaatcaggcaccagccccaagagatggatctacgacacatccaaggtcgcttctggggtgccttataggttcagtgggtcaggaagcggcacttcctactctctgaccattagctccatggaggcagaagatgccgctacatactattgtcagcagtggtctagtaatccactgacatttggggccggaactaaactggagctgaaggcagccgaacccaaatcaagcgacaagacacacacttgcccaccttgtccagcaccagaactgctgggaggacctagcgtgttcctgtttccacccaaaccaaaggatacactgatgatcagccggacccctgaggtcacatgcgtggtcgtggacgtgagccacgaggaccccgaagtcaagttcaactggtacgtggacggcgtcgaagtgcataatgccaaaaccaagcctagggaggaacagtacaatagtacttatcgcgtcgtgtcagtcctgaccgtgctgcatcaggattggctgaacgggaaggagtacaaatgcaaggtgtccaacaaggccctgcctgctccaatcgagaagaccatttctaaagcaaagggccagccccgagaacctcaggtctacgtgtatcctccatcccgggacgagctgaccaaaaaccaggtctctctgacatgtctggtgaaggggttttatccatctgatattgctgtggagtgggaaagtaatggacagcccgagaacaattacaagacaactccccctgtgctggactccgatggatctttcgctctggtcagcaaactgacagtggacaagtccagatggcagcagggcaacgtctttagttgttcagtgatgcacgaggcactgcacaatcattacactcagaaaagcctgtccctgtctcccggcaagtgaggatcc

SEQ ID NO: 112 - DNA, 1548 bp, synthetic construct
  source 1..1548: mol_type = other DNA
gaattcgccaccatggctgtgatggcacctcgaacactggtcctgctgctgagcggggcactggcactgacacagacttgggccggagacattcagctgacccagagtcctgcttcactggcagtgagcctgggacagcgagcaacaatctcctgcaaagctagtcagtcagtggactatgatggcgactcctatctgaactggtaccagcagatcccagggcagccccctaagctgctgatctacgacgcctcaaatctggtgagcggcatcccaccacgattcagcggcagcggctctggaaccgattttacactgaacattcacccagtcgagaaggtggacgccgctacctaccattgccagcagtctacagaggacccctggactttcggcgggggaaccaaactggaaatcaagggaggaggaggcagtggcggaggagggtcaggaggaggaggaagccaggtgcagctgcagcagagcggagcagagctggtcagaccaggaagctccgtgaaaatttcctgtaaggcatctggctatgccttttctagttactggatgaattgggtgaagcagaggccaggacagggcctggaatggatcgggcagatttggcccggggatggagacacaaactataatggaaagttcaaaggcaaggctactctgaccgcagacgagtcaagctccactgcatatatgcagctgtctagtctggccagcgaggattccgctgtctacttttgcgcacggagagaaaccacaactgtgggcaggtactattacgccatggactactggggccaggggaccacagtcaccgtgtcaagcgcagccgaacccaaatcctctgataagacacacacttgccctccatgtccagctcctgagctgctgggaggaccaagcgtgttcctgtttccacctaaacctaaggacactctgatgatctctcggactcccgaagtcacctgtgtggtcgtggatgtgagccacgaggaccctgaagtcaaattcaactggtacgtggatggcgtcgaggtgcataatgccaaaacaaagcctagggaggaacagtataactccacataccgcgtcgtgtctgtcctgactgtgctgcatcaggactggctgaacggaaaggagtacaaatgcaaggtgagcaacaaggccctgccagctcccatcgagaagaccatttccaaagctaagggccagcctcgagaaccacaggtctatgtgctgccacccagccgggacgagctgacaaaaaaccaggtctccctgctgtgtctggtgaagggattctacccttctgatattgcagtggagtgggaaagtaatggccagccagaaaacaattatctgacttggcctccagtgctggattctgacgggagtttctttctgtacagtaaactgaccgtggataagtcacggtggcagcagggaaacgtctttagttgttcagtgatgcacgaggccctgcacaatcattacacccagaaaagcctgtccctgtctcccggcaagtgaggatcc

SEQ ID NO: 113 - protein, 506 aa, synthetic construct
EFATMAVMAPRTLVLLLSGALALTQTWAGDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 114 - protein, 513 aa, synthetic construct
EFATMAVMAPRTLVLLLSGALALTQTWAGDIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 115 - DNA, 1518 bp, synthetic construct
  source 1..1518: mol_type = other DNA
gaattcgccaccatggccgtgatggcaccccgcacactggtcctgctgctgagcggagcactggcactgacacagacctgggctgggcagatcgtcctgacacagagcccagcaatcatgtcagccagccccggcgagaaagtcaccatgacatgctcagccagctcctctgtgagctacatgaactggtatcagcagaaaagcggaacatcccccaagagatggatctacgacacttccaagctggcttctggagtgcctgcacacttcaggggcagcggctctgggactagttattcactgaccatttccggcatggaggccgaagatgccgctacctactattgccagcagtggagttcaaacccattcacatttggatctggcactaagctggaaattaatggcggaggaggctccggaggaggagggtctggaggaggaggaagtcaggtccagctgcagcagagcggagctgagctggcacgaccaggagcaagtgtgaaaatgtcctgtaaggccagcggctacactttcacccggtataccatgcattgggtgaaacagagacccgggcagggactggaatggatcgggtacattaatccttcccgaggatacacaaactacaaccagaagtttaaagacaaggctaccctgaccacagataagagctcctctacagcatatatgcagctgagttcactgacttctgaggacagtgccgtgtactattgcgctaggtactatgacgatcactactccctggattattggggccaggggactaccctgaccgtgagctccgcagccgaacctaaatctagtgacaagacacatacttgcccaccttgtccagcaccagagctgctgggaggacctagcgtgttcctgtttccacccaaaccaaaggatacactgatgatctcccggacccctgaagtcacatgtgtggtcgtggacgtgtctcacgaggaccccgaagtcaagttcaactggtacgtggacggcgtcgaggtgcataatgccaaaactaagcccagggaggaacagtacaactccacttatcgcgtcgtgtctgtcctgaccgtgctgcaccaggattggctgaacggcaaggagtacaaatgcaaggtgagcaacaaggccctgcctgctccaatcgagaagaccattagcaaagcaaaggggcagccccgagaacctcaggtctacgtgtatcctccatctcgggacgagctgaccaaaaaccaggtcagtctgacatgtctggtgaagggcttttacccaagcgatattgctgtggagtgggaatccaatgggcagcccgaaaacaattataagacaactccccctgtgctggactcagatgggagcttcgccctggtcagtaaactgactgtggacaagtcacggtggcagcagggaaacgtctttagctgttccgtgatgcatgaggctctgcacaatcattacacccagaaatctctgagtctgtcacccggcaagtgaggatcc

SEQ ID NO: 116 - DNA, 1548 bp, synthetic construct; identical to SEQ ID NO: 112
  source 1..1548: mol_type = other DNA

SEQ ID NO: 117 - protein, 503 aa, synthetic construct
EFATMAVMAPRTLVLLLSGALALTQTWAGQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINGGGGSGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 118 - protein, 513 aa, synthetic construct; identical to SEQ ID NO: 114

SEQ ID NO: 119 - DNA, 1527 bp, synthetic construct
  source 1..1527: mol_type = other DNA
gaattcgccaccatggctgtgatggctccacgaacactggtcctgctgctgtccggggcactggcactgactcagacttgggctgggcaggtccagctgcagcagtctggagctgagctggcaaggcctggcgcatccgtgaaaatgtcttgcaaggccagtggctacactttcacccggtatactatgcactgggtgaaacagagacccggacagggcctggaatggatcgggtacattaacccttcccgcggatacaccaactacaaccagaagtttaaagacaaggctaccctgaccacagataagagctcctctacagcatatatgcagctgagttcactgactagcgaggactccgctgtgtactattgcgcacgctactatgacgatcattacagcctggattattgggggcagggaactaccctgacagtgagctccgccaaaacaactccaaagctggaggaaggcgagttctccgaagctcgagtcgacatcgtgctgactcagtctccagcaattatgtcagccagccccggggagaaagtcacaatgacttgttccgcctctagttcagtgtcttacatgaattggtatcagcagaaatctggaaccagtcccaagagatggatctacgacacatcaaagctggccagcggcgtgcctgctcactttaggggctccgggtctggaacaagttattcactgactattagcgggatggaggccgaagatgccgctacctactattgccagcagtggagctccaacccattcacctttggctccgggacaaagctggagatcaatcgggcagccgaacccaaatctagtgacaagacccatacatgccccccttgtccagctccagaactgctgggaggacctagcgtgttcctgtttccacccaaaccaaaggatacactgatgattagtagaactcctgaggtcacctgtgtggtcgtggacgtgtcccacgaggaccccgaagtcaagttcaactggtacgtggacggcgtcgaagtgcataatgccaaaaccaagcccagggaggaacagtacaacagtacatatagagtcgtgtcagtgctgaccgtcctgcaccaggattggctgaacggaaaggagtacaaatgcaaggtgtctaacaaggccctgcctgctccaatcgagaagaccattagcaaagctaagggccagccccgagaacctcaggtctacgtgtatcctccatcacgggacgagctgactaaaaaccaggtcagcctgacctgtctggtgaagggcttttacccatctgatatcgcagtggagtgggaaagtaatgggcagcccgagaacaattataagaccacaccccctgtgctggactcagatgggagcttcgcactggtcagcaaactgacagtggataagtccaggtggcagcagggaaacgtctttagctgttccgtgatgcatgaagccctgcacaatcattacacccagaaatctctgagtctgtcacctggcaagtgaggatcc

SEQ ID NO: 120 - DNA, 1548 bp, synthetic construct; identical to SEQ ID NO: 112
  source 1..1548: mol_type = other DNA

SEQ ID NO: 121 - protein, 506 aa, synthetic construct
EFATMAVMAPRTLVLLLSGALALTQTWAGQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSAKTTPKLEEGEFSEARVDIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 122 - protein, 513 aa, synthetic construct; identical to SEQ ID NO: 114

SEQ ID NO: 123 - DNA, 1503 bp, synthetic construct
  source 1..1503: mol_type = other DNA
gaattcgccaccatggccgtgatggctccaagaacactggtcctgctgctgagcggggcactggcactgacacagacatgggccggacaggtccagctgcagcagagcggagctgagctggcacgacctggagcatccgtgaaaatgtcttgcaaggccagtggctacactttcacccggtatacaatgcactgggtgaaacagagacccgggcagggactggaatggatcggctacattaacccttcccgagggtacactaactacaaccagaagtttaaagacaaggctaccctgaccacagataagagctcctctacagcatatatgcagctgagttcactgactagcgaggactccgctgtgtactattgcgcacggtactatgacgatcattactccctggattattggggccaggggactaccctgactgtcagctccgcaaaaacaactccaaagctgggcggggacatcgtgctgacccagtctccagcaattatgtcagccagccccggggagaaagtcacaatgacttgttccgcttctagttcagtgtcttacatgaattggtatcagcagaaatctgggaccagtcccaagagatggatctacgacacatcaaagctggccagcggagtgcctgctcacttcaggggcagcggctctgggacaagttattcactgactatttctggcatggaggccgaagatgccgctacctactattgccagcagtggagctccaacccattcacctttggaagcggcacaaagctggagatcaatagggcagccgaacccaaatctagtgacaagacccatacatgcccaccttgtccagcaccagaactgctgggaggacctagcgtgttcctgtttccacccaaaccaaaggatacactgatgatttctcgcactcctgaggtcacctgtgtggtcgtggacgtcagtcacgaggaccccgaagtcaagttcaactggtacgtggacggcgtcgaagtgcataatgccaaaaccaagcccagggaggaacagtacaacagtacatatagagtcgtgtcagtgctgaccgtcctgcaccaggattggctgaacggaaaggagtacaaatgcaaggtgagcaacaaggccctgcctgctccaatcgagaagaccatttccaaagccaagggccagccccgagaacctcaggtctacgtgtatcctccatcacgggacgagctgactaaaaaccaggtctccctgacctgtctggtgaagggattttacccatctgatatcgccgtggagtgggaaagtaatggccagcccgagaacaattataagaccacaccccctgtgctggactcagatggcagcttcgccctggtcagcaaactgacagtggataagtccagatggcagcaggggaacgtctttagctgttccgtgatgcatgaagctctgcacaatcattacacccagaaatctctgagtctgtcacctggcaagtgaggatcc

SEQ ID NO: 124 - DNA, 1548 bp, synthetic construct; identical to SEQ ID NO: 112
  source 1..1548: mol_type = other DNA

SEQ ID NO: 125 - protein, 498 aa, synthetic construct
EFATMAVMAPRTLVLLLSGALALTQTWAGQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSAKTTPKLGGDIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 126 - protein, 513 aa, synthetic construct; identical to SEQ ID NO: 114

SEQ ID NO: 127 - DNA, 1536 bp, synthetic construct
  source 1..1536: mol_type = other DNA
gaattcgccaccatggccgtgatggcaccaagaacactggtcctgctgctgagcggggcactggcactgacacagacatgggccggacaggtccagctgcagcagagcggagctgagctggcacgaccaggagcaagtgtgaaaatgtcatgcaaggccagcggctacaccttcacacggtatactatgcactgggtgaaacagagacccggacagggcctggaatggatcgggtacattaaccctagccgaggatacaccaactacaaccagaagtttaaagacaaggctaccctgaccacagataagagctcctctacagcatatatgcagctgagttcactgacttctgaggacagtgctgtgtactattgtgcacggtactatgacgatcattactccctggattattgggggcagggaactaccctgaccgtgagctcctctagtacaggaggaggaggcagtggaggaggagggtcaggcggaggaggaagcgacatccagattgtgctgacacagtctccagcaatcatgtccgcctctcccggcgagaaagtcactatgacctgctccgcctcaagctccgtgtcttacatgaattggtatcagcagaaatcaggaaccagccccaagagatggatctacgacacatccaagctggcctctggcgtgcctgctcacttcaggggcagtgggtcaggaactagctattccctgaccattagcggcatggaggccgaagatgccgctacctactattgtcagcagtggtctagtaacccattcacatttggcagcgggactaagctggagatcaatagggcagccgaacccaaatcaagcgacaagacacatacttgccccccttgtccagcaccagaactgctgggaggaccttccgtgttcctgtttccacccaaaccaaaggatacactgatgattagccgcacccctgaggtcacatgcgtggtcgtggacgtgagccacgaggaccccgaagtcaagttcaactggtacgtggacggcgtcgaagtgcataatgccaaaaccaagcctagggaggaacagtacaacagtacatatagagtcgtgtcagtgctgaccgtcctgcaccaggattggctgaacggcaaggagtacaaatgcaaggtgtccaacaaggccctgcctgctccaatcgagaagaccatttctaaagcaaaggggcagccccgagaacctcaggtctacgtgtatcctccatcccgggacgagctgactaaaaaccaggtctctctgacctgtctggtgaagggcttttacccatctgatattgctgtcgagtgggaaagtaatgggcagcccgagaacaattataagacaactccccctgtgctggactccgatgggtctttcgccctggtcagcaaactgacagtggataagtccagatggcagcagggaaacgtcttttcttgtagtgtgatgcatgaagctctgcacaatcattacactcagaaatcactgagcctgtcccccggcaagtgaggatcc

SEQ ID NO: 128 - DNA, 1548 bp, synthetic construct; identical to SEQ ID NO: 112
  source 1..1548: mol_type = other DNA

SEQ ID NO: 129 - protein, 509 aa, synthetic construct
EFATMAVMAPRTLVLLLSGALALTQTWAGQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSSSTGGGGSGGGGSGGGGSDIQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 130 - protein, 513 aa, synthetic construct; identical to SEQ ID NO: 114

SEQ ID NO: 131 - DNA, 1548 bp, synthetic construct; identical to SEQ ID NO: 37
  source 1..1548: mol_type = other DNA

SEQ ID NO: 132 - DNA, 1527 bp, synthetic construct
  source 1..1527: mol_type = other DNA
gaattcgccaccatggctgtgatggctcctagaacactggtcctgctgctgtccggggcactggcactgactcagacttgggccgggcaggtccagctgcagcagtctggagcagagctggctaggcctggagcttccgtgaaaatgtcttgcaaagcaagtggctacaccttcacacggtatactatgcactgggtgaaacagagacccggacagggcctggaatggatcgggtacattaacccttcccgcggatacaccaactacaaccagaagtttaaagacaaggccactctgaccacagataagagctcctctaccgcttatatgcagctgagttcactgacaagcgaggactccgccgtgtactattgcgctcgctactatgacgatcattacagcctggattattgggggcagggaactaccctgacagtgagctccgcaaaaacaactccaaagctggaggaaggcgagttctccgaagcccgagtcgacatcgtgctgactcagtctccagctattatgtcagcaagccccggggagaaagtcactatgacctgttccgcctctagttcagtgtcttacatgaattggtatcagcagaaatctggaactagtcccaagagatggatctacgacacctcaaagctggcaagcggagtgcctgcacactttaggggctccgggtctggaactagttattcactgaccattagcgggatggaggccgaagatgccgctacctactattgccagcagtggagctccaacccattcacatttggctccgggactaagctggagatcaatcgggcagccgaacccaaatctagtgacaagacacatacttgccccccttgtccagctccagaagctgcaggaggacctagcgtgttcctgtttccacccaaaccaaaggatacactgatgattagtagaacccctgaggtcacatgtgtggtcgtgagcgtgtcccacgaggaccccgaagtcaagttcaactggtacgtggacggcgtcgaagtgcataatgccaaaaccaagcccagggaggaacagtacaacagtacctatcgcgtcgtgtcagtcctgacagtgctgcaccaggattggctgaacggaaaggagtacaaatgcaaggtgtctaacaaggcactgcctgccccaatcgagaagaccattagcaaagctaagggccagccccgagaacctcaggtctacgtgctgcctccatcacgggacgagctgacaaaaaaccaggtcagcctgctgtgtctggtgaagggcttctatccatctgatatcgcagtggagtgggaaagtaatgggcagcccgagaacaattacctgacatggccccctgtcctggactcagatgggagcttctttctgtatagcaaactgactgtggataagtccaggtggcagcagggaaacgtctttagctgttccgtgatgcatgaagccctgcacaatcattacacccagaaatctctgagtctgtcacctggcaagtgaggatcc

SEQ ID NO: 133 - protein, 513 aa, synthetic construct; identical to SEQ ID NO: 38

SEQ ID NO: 134 - protein, 506 aa, synthetic construct
EFATMAVMAPRTLVLLLSGALALTQTWAGQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSAKTTPKLEEGEFSEARVDIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 135 - DNA, 1548 bp, synthetic construct; identical to SEQ ID NO: 37
  source 1..1548: mol_type = other DNA

SEQ ID NO: 136 - DNA, 1503 bp, synthetic construct
  source 1..1503: mol_type = other DNA
gaattcgccaccatggccgtgatggctcctagaacactggtcctgctgctgtccggggctctggcactgactcagacttgggctgggcaggtccagctgcagcagagcggagcagagctggctcgacctggagcttccgtgaaaatgtcttgcaaagcaagtggctacaccttcacacggtatacaatgcactgggtgaaacagagacccgggcagggactggaatggatcggctacattaacccttcccgagggtacactaactacaaccagaagtttaaagacaaggccactctgaccacagataagagctcctctaccgcttatatgcagctgagttcactgacaagcgaggactccgccgtgtactattgcgctcggtactatgacgatcattactccctggattattggggccaggggactaccctgacagtcagctccgcaaaaacaactccaaagctgggcggggacatcgtgctgactcagtctccagctattatgtcagcaagccccggggagaaagtcactatgacctgttccgcctctagttcagtgtcttacatgaattggtatcagcagaaatctgggactagtcccaagagatggatctacgacacctcaaagctggcaagcggagtgcctgcacacttcaggggcagcggctctgggactagttattcactgaccatttctggcatggaggccgaagatgccgctacctactattgccagcagtggagctccaacccattcacatttggaagcggcactaagctggagatcaatagggcagccgaacccaaatctagtgacaagacacatacttgccccccttgtccagctcccgaagctgcaggaggccctagcgtgttcctgtttccacccaaaccaaaggatacactgatgatttctcgcacccctgaggtcacatgtgtggtcgtgagcgtcagtcacgaggaccccgaagtcaagtttaactggtacgtggacggcgtcgaagtgcataatgccaaaaccaagcccagggaggaacagtacaacagtacctatcgcgtcgtgtcagtcctgacagtgctgcaccaggattggctgaacggaaaggagtacaaatgcaaggtgagcaacaaggcactgcctgccccaatcgagaagactatttccaaagctaagggccagccccgagaacctcaggtctacgtgctgcctccatcacgggacgagctgaccaaaaaccaggtctccctgctgtgtctggtgaagggattctatccatctgatatcgcagtggagtgggaaagtaatggccagcccgagaacaattacctgacctggccccctgtgctggactcagatggcagcttctttctgtatagcaaactgacagtggataagtccagatggcagcaggggaacgtctttagctgttccgtgatgcatgaagccctgcacaatcattacacccagaaatctctgagtctgtcacctggcaagtgaggatcc

SEQ ID NO: 137 - protein, 513 aa, synthetic construct; identical to SEQ ID NO: 38

SEQ ID NO: 138 - protein, 498 aa, synthetic construct
EFATMAVMAPRTLVLLLSGALALTQTWAGQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSAKTTPKLGGDIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 139 - DNA, 1527 bp, synthetic construct
  source 1..1527: mol_type = other DNA
gaattcgccaccatggctgtgatggcacctagaaccctggtcctgctgctgtccggggcactggcactgactcagacttgggctggggatattaagctgcagcagagcggagcagagctggctcgaccaggagccagtgtgaaaatgtcatgcaagacaagcggctacaccttcacacggtatactatgcactgggtgaaacagagacccggccaggggctggaatggatcggatatattaacccttcccgaggctacaccaactataatcagaagtttaaagacaaggcaaccctgaccacagataagagctcctctacagcctacatgcagctgagttcactgactagtgaggactcagccgtgtactattgcgctaggtactatgacgatcattactgtctggattattggggacagggcactaccctgactgtcagctccgtggaaggagggagcggaggctccggaggatctggcgggagtggaggcgtggacgatatccagctgacccagtccccagctattatgtccgcatctcccggcgagaaagtgactatgacctgccgcgcatctagttcagtgagctacatgaactggtatcagcagaaatcaggcaccagccccaagagatggatctacgacacatccaaggtcgcttctggggtgccttataggttcagtgggtcaggaagcggcacttcctactctctgaccattagctccatggaggctgaagatgccgctacatactattgtcagcagtggtctagtaatccactgacatttggggcaggaactaaactggagctgaaggcagccgaacccaaatcaagcgacaagacacacacttgcccaccttgtccagcaccagaagctgcaggaggacctagcgtgttcctgtttccacccaaaccaaaggatacactgatgatcagccggacccctgaggtcacatgcgtggtcgtgagcgtgagccacgaggaccccgaagtcaagttcaactggtacgtggacggcgtcgaagtgcataatgccaaaaccaagcctagggaggaacagtacaatagtacttatcgcgtcgtgtcagtcctgaccgtgctgcatcaggattggctgaacgggaaggagtacaaatgcaaggtgtccaacaaggcactgcctgccccaatcgagaagaccatttctaaagccaagggccagccccgagaacctcaggtctacgtgtatcctccatcccgggacgagctgaccaaaaaccaggtctctctgacatgtctggtgaaggggttttatccatctgatattgccgtggagtgggaaagtaatggacagcccgagaacaattacaagacaactccccctgtgctggactccgatggatctttcgccctggtcagcaaactgacagtggacaagtccagatggcagcagggcaacgtctttagttgttcagtgatgcacgaggctctgcacaatcattacactcagaaaagcctgtccctgtctcccggcaagtgaggatcc

SEQ ID NO: 140 - DNA, 1548 bp, synthetic construct; identical to SEQ ID NO: 47
  source 1..1548: mol_type = other DNA

SEQ ID NO: 141 - protein, 506 aa, synthetic construct
EFATMAVMAPRTLVLLLSGALALTQTWAGDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 142 - protein, 513 aa, synthetic construct; identical to SEQ ID NO: 48

SEQ ID NO: 143 - DNA, 1548 bp, synthetic construct; identical to SEQ ID NO: 95
  source 1..1548: mol_type = other DNA

SEQ ID NO: 144 - DNA, 1518 bp, synthetic construct
  source 1..1518: mol_type = other DNA
gaattcgccaccatggccgtgatggcacctcgcaccctggtcctgctgctgagcggggcactggcactgacacagacttgggccggacagatcgtcctgacacagagcccagctatcatgtcagcaagccccggcgagaaagtcacaatgacttgctcagccagctcctctgtgagctacatgaactggtatcagcagaaaagcggaacctcccccaagagatggatctacgacacatccaagctggcctctggagtgcctgctcacttcaggggcagcggctctgggaccagttattcactgacaatttccggcatggaggccgaagatgccgctacctactattgccagcagtggagttcaaacccattcacttttggatctggcaccaagctggaaattaatggcggaggaggctccggaggaggagggtctggaggaggaggaagtcaggtgcagctgcagcagtccggagcagagctggctcgaccaggagctagtgtgaaaatgtcctgtaaggcaagcggctacaccttcacacggtataccatgcattgggtgaaacagagacccgggcagggactggaatggatcgggtacattaatcctagccgaggatacacaaactacaaccagaagtttaaagacaaggccactctgaccacagataagagctcctctaccgcttatatgcagctgagttcactgacatctgaggacagtgcagtgtactattgcgccaggtactatgacgatcactactgtctggattattggggccaggggactaccctgacagtgagctccgcagccgaacctaaatctagtgacaagactcatacctgccccccttgtccagcaccagagctgctgggaggaccttccgtgttcctgtttccacccaaaccaaaggatactctgatgatctcccggacacctgaagtcacttgcgtggtcgtggacgtgtctcacgaggaccccgaagtcaagtttaactggtacgtggacggcgtcgaggtgcataatgccaaaaccaagcccagggaggaacagtacgcctccacatatcgcgtcgtgtctgtcctgactgtgctgcaccaggattggctgaacggcaaggagtacaaatgcaaggtgagcaacaaggcactgcctgccccaatcgagaagacaattagcaaagcaaaggggcagccccgagaacctcaggtctacgtgctgcctccatctcgggacgagctgactaaaaaccaggtcagtctgctgtgtctggtgaagggcttctatccaagcgatattgctgtggagtgggaatccaatgggcagcccgaaaacaattacctgacttggccccctgtcctggactcagatgggagcttctttctgtatagtaaactgaccgtggacaagtcacggtggcagcagggaaacgtctttagctgttccgtgatgcatgaggccctgcacaatcattacacccagaaatctctgagtctgtcacccggcaagtgaggatcc

SEQ ID NO: 145 - protein, 513 aa, synthetic construct; identical to SEQ ID NO: 97

SEQ ID NO: 146 - protein, 503 aa, synthetic construct
EFATMAVMAPRTLVLLLSGALALTQTWAGQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINGGGGSGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSAAEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 147 - DNA, 1548 bp, synthetic construct; identical to SEQ ID NO: 95
  source 1..1548: mol_type = other DNA

SEQ ID NO: 148 - DNA, 1518 bp, synthetic construct
  source 1..1518: mol_type = other DNA
gaattcgccaccatggccgtgatggcaccccgaacactggtcctgctgctgagcggggcactggcactgacacagacttgggctggacagatcgtcctgacacagagcccagcaatcatgtcagccagccccggcgagaaagtcacaatgacttgctcagcaagctcctctgtgagctacatgaactggtatcagcagaaaagcggaacctcccccaagagatggatctacgacacatccaagctggcttctggagtgcctgcacacttcaggggcagcggctctgggaccagttattcactgacaatttccggcatggaggctgaagatgccgctacctactattgccagcagtggagttcaaacccattcacttttggatctggcaccaagctggaaattaatggcggaggaggctccggaggaggagggtctggaggaggaggaagtcaggtgcagctgcagcagagcggagctgagctggcacgaccaggagcaagtgtgaaaatgtcctgtaaggccagcggctacaccttcacacggtataccatgcattgggtgaaacagagacccgggcagggactggaatggatcgggtacattaatccttcccgaggatacacaaactacaaccagaagtttaaagacaaggctactctgaccacagataagagctcctctaccgcatatatgcagctgagttcactgacatctgaggacagtgccgtgtactattgcgctaggtactatgacgatcactactccctggattattggggccaggggactaccctgacagtgagctccgcagccgaacctaaatctagtgacaagactcatacctgccccccttgtccagcaccagagctgctgggaggacctagcgtgttcctgtttccacccaaaccaaaggatactctgatgatctcccggacacctgaagtcacttgtgtggtcgtggacgtgtctcacgaggaccccgaagtcaagtttaactggtacgtggacggcgtcgaggtgcataatgccaaaaccaagcccagggaggaacagtacgcctccacatatcgcgtcgtgtctgtcctgactgtgctgcaccaggattggctgaacggcaaggagtacaaatgcaaggtgagcaacaaggccctgcctgctccaatcgagaagacaattagcaaagccaaggggcagccccgagaacctcaggtctacgtgctgcctccatctcgggacgagctgactaaaaaccaggtcagtctgctgtgtctggtgaagggcttctatccaagcgatattgctgtggagtgggaatccaatgggcagcccgaaaacaattacctgacttggccccctgtcctggactcagatgggagcttctttctgtatagtaaactgaccgtggacaagtcacggtggcagcagggaaacgtctttagctgttccgtgatgcatgaggccctgcacaatcattacacccagaaatctctgagtctgtcacccggcaagtgaggatcc

SEQ ID NO: 149 - protein, 513 aa, synthetic construct; identical to SEQ ID NO: 97

SEQ ID NO: 150 - protein, 503 aa, synthetic construct
EFATMAVMAPRTLVLLLSGALALTQTWAGQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINGGGGSGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSAAEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 151 - DNA, 1548 bp, synthetic construct; identical to SEQ ID NO: 95
  source 1..1548: mol_type = other DNA

SEQ ID NO: 152 - DNA, 1527 bp, synthetic construct
  source 1..1527: mol_type = other DNA
gaattcgccaccatggccgtgatggctcctcggaccctggtgctgctgctgagtggggctctggctctgacacagacttgggccggacaggtgcagctgcagcagtctggagctgagctggcaaggcctggcgcatccgtgaaaatgtcttgcaaggccagtggctacaccttcacacggtatactatgcactgggtgaaacagagacccggacagggcctggaatggatcgggtacattaacccttcccgcggatacaccaactacaaccagaagtttaaagacaaggctactctgaccacagataagagctcctctaccgcatatatgcagctgagttcactgacaagcgaggactccgctgtgtactattgcgcacgctactatgacgatcattacagcctggattattgggggcagggaactaccctgacagtgagctccgccaaaacaactccaaagctggaggaaggcgagttctccgaagctcgagtcgacatcgtgctgactcagtctccagcaattatgtcagccagccccggggagaaagtcactatgacctgttccgcctctagttcagtgtcttacatgaattggtatcagcagaaatctggaactagtcccaagagatggatctacgacacctcaaagctggccagcggcgtgcctgctcactttaggggctccgggtctggaactagttattcactgaccattagcgggatggaggccgaagatgccgctacctactattgccagcagtggagctccaacccattcacatttggctccgggactaagctggagatcaatcgggcagccgaacccaaatctagtgacaagacacatacttgccccccttgtccagcaccagaactgctgggaggacctagcgtgttcctgtttccacccaaaccaaaggatacactgatgattagtagaacccctgaggtcacatgtgtggtcgtggacgtgtcccacgaggaccccgaagtcaagttcaactggtacgtggacggcgtcgaagtgcataatgccaaaaccaagcccagggaggaacagtacgccagtacctatcgcgtcgtgtcagtcctgacagtgctgcaccaggattggctgaacggaaaggagtacaaatgcaaggtgtctaacaaggccctgcctgctccaatcgagaagaccattagcaaagcaaagggccagccccgagaacctcaggtctacgtgctgcctccatcacgggacgagctgacaaaaaaccaggtcagcctgctgtgtctggtgaagggcttctatccatctgatatcgctgtggagtgggaaagtaatgggcagcccgagaacaattacctgacatggccccctgtcctggactcagatgggagcttctttctgtatagcaaactgactgtggataagtccaggtggcagcagggaaacgtctttagctgttccgtgatgcatgaagccctgcacaatcattacacccagaaatctctgagtctgtcacctggcaagtgaggatcc

SEQ ID NO: 153 - protein, 513 aa, synthetic construct; identical to SEQ ID NO: 97

SEQ ID NO: 154 - protein, 506 aa, synthetic construct
EFATMAVMAPRTLVLLLSGALALTQTWAGQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSAKTTPKLEEGEFSEARVDIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRAAEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 155 - DNA, 1548 bp, synthetic construct; identical to SEQ ID NO: 95
  source 1..1548: mol_type = other DNA

SEQ ID NO: 156 - DNA, 1503 bp, synthetic construct
  source 1..1503: mol_type = other DNA
gaattcgccaccatggccgtgatggctcctagaacactggtcctgctgctgagcggggcactggcactgacacagacttgggctgggcaggtccagctgcagcagagcggagctgagctggcacgacctggagcatccgtgaaaatgtcttgcaaggccagtggctacaccttcacacggtatacaatgcactgggtgaaacagagacccgggcagggactggaatggatcggctacattaacccttcccgagggtacactaactacaaccagaagtttaaagacaaggctactctgaccacagataagagctcctctaccgcatatatgcagctgagttcactgacaagcgaggactccgctgtgtactattgcgcacggtactatgacgatcattactccctggattattggggccaggggactaccctgacagtcagctccgccaaaacaactccaaagctgggcggggacatcgtgctgactcagtctccagcaattatgtcagccagccccggggagaaagtcactatgacctgttccgcttctagttcagtgtcttacatgaattggtatcagcagaaatctgggactagtcccaagagatggatctacgacacctcaaagctggccagcggagtgcctgctcacttcaggggcagcggctctgggactagttattcactgaccatttctggcatggaggccgaagatgccgctacctactattgccagcagtggagctccaacccattcacatttggaagcggcactaagctggagatcaatagggcagccgaacccaaatctagtgacaagacacatacttgcccaccttgtccagcaccagaactgctgggaggacctagcgtgttcctgtttccacccaaaccaaaggatacactgatgatttctcgcacccctgaggtcacatgtgtggtcgtggacgtcagtcacgaggaccccgaagtcaagtttaactggtacgtggacggcgtcgaagtgcataatgccaaaaccaagcccagggaggaacagtacgccagtacctatcgcgtcgtgtcagtcctgacagtgctgcaccaggattggctgaacggaaaggagtacaaatgcaaggtgagcaacaaggccctgcctgctccaatcgagaagactatttccaaagcaaagggccagccccgagaacctcaggtctacgtgctgcctccatcacgggacgagctgaccaaaaaccaggtctccctgctgtgtctggtgaagggattctatccatctgatatcgctgtggagtgggaaagtaatggccagcccgagaacaattacctgacctggccccctgtgctggactcagatggcagcttctttctgtatagcaaactgacagtggataagtccagatggcagcaggggaacgtctttagctgttccgtgatgcatgaagccctgcacaatcattacacccagaaatctctgagtctgtcacctggcaagtgaggatcc

SEQ ID NO: 157 - protein, 513 aa, synthetic construct; identical to SEQ ID NO: 97

SEQ ID NO: 158 - protein, 498 aa, synthetic construct
EFATMAVMAPRTLVLLLSGALALTQTWAGQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSAKTTPKLGGDIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRAAEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 159 - DNA, 1527 bp, synthetic construct
  source 1..1527: mol_type = other DNA
gaattcgccaccatggccgtcatggcacctagaaccctggtcctgctgctgagcggggcactggcactgacacagacttgggctggagacatcaaactgcagcagagcggagctgagctggcacgaccaggagccagtgtgaaaatgtcatgcaagacaagcggctacaccttcacacggtatactatgcactgggtgaaacagagacccggccaggggctggaatggatcggatatattaacccttcccgaggctacaccaactataatcagaagtttaaagacaaggccaccctgaccacagataagagctcctctacagcttacatgcagctgagttcactgactagtgaggactcagctgtgtactattgcgcaaggtactatgacgatcattactgtctggattattggggacagggcactaccctgactgtcagctccgtggaaggagggagcggaggctccggaggatctggcgggagtggaggcgtggacgatatccagctgacccagtccccagcaattatgtccgcctctcccggcgagaaagtgactatgacctgccgcgcctctagttcagtgagctacatgaactggtatcagcagaaatcaggcaccagccccaagagatggatctacgacacatccaaggtcgcttctggggtgccttataggttcagtgggtcaggaagcggcacttcctactctctgaccattagctccatggaggcagaagatgccgctacatactattgtcagcagtggtctagtaatccactgacatttggggccggaactaaactggagctgaaggcagccgaacccaaatcaagcgacaagacacacacttgcccaccttgtccagcaccagaactgctgggaggacctagcgtgttcctgtttccacccaaaccaaaggatacactgatgatcagccggacccctgaggtcacatgcgtggtcgtggacgtgagccacgaggaccccgaagtcaagttcaactggtacgtggacggcgtcgaagtgcataatgccaaaaccaagcctagggaggaacagtacgccagtacttatcgcgtcgtgtcagtcctgaccgtgctgcatcaggattggctgaacgggaaggagtacaaatgcaaggtgtccaacaaggccctgcctgctccaatcgagaagaccatttctaaagcaaagggccagccccgagaacctcaggtctacgtgtatcctccatcccgggacgagctgaccaaaaaccaggtctctctgacatgtctggtgaaggggttttatccatctgatattgctgtggagtgggaaagtaatggacagcccgagaacaattacaagacaactccccctgtgctggactccgatggatctttcgctctggtcagcaaactgacagtggacaagtccagatggcagcagggcaacgtctttagttgttcagtgatgcacgaggcactgcacaatcattacactcagaaaagcctgtccctgtctcccggcaagtgaggatcc

SEQ ID NO: 160 - DNA, 1548 bp, synthetic construct
  source 1..1548: mol_type = other DNA
gaattcgccaccatggctgtgatggcacctcgaacactggtcctgctgctgagcggggcactggcactgacacagacttgggccggagacattcagctgacccagagtcctgcttcactggcagtgagcctgggacagcgagcaacaatctcctgcaaagctagtcagtcagtggactatgatggcgactcctatctgaactggtaccagcagatcccagggcagccccctaagctgctgatctacgacgcctcaaatctggtgagcggcatcccaccacgattcagcggcagcggctctggaaccgattttacactgaacattcacccagtcgagaaggtggacgccgctacctaccattgccagcagtctacagaggacccctggactttcggcgggggaaccaaactggaaatcaagggaggaggaggcagtggcggaggagggtcaggaggaggaggaagccaggtgcagctgcagcagagcggagcagagctggtcagaccaggaagctccgtgaaaatttcctgtaaggcatctggctatgccttttctagttactggatgaattgggtgaagcagaggccaggacagggcctggaatggatcgggcagatttggcccggggatggagacacaaactataatggaaagttcaaaggcaaggctactctgaccgcagacgagtcaagctccactgcatatatgcagctgtctagtctggccagcgaggattccgctgtctacttttgcgcacggagagaaaccacaactgtgggcaggtactattacgccatggactactggggccaggggaccacagtcaccgtgtcaagcgcagccgaacccaaatcctctgataagacacacacttgccctccatgtccagctcctgagctgctgggaggaccaagcgtgttcctgtttccacctaaacctaaggacactctgatgatctctcggactcccgaagtcacctgtgtggtcgtggatgtgagccacgaggaccctgaagtcaaattcaactggtacgtggatggcgtcgaggtgcataatgccaaaacaaagcctagggaggaacagtatgcctccacataccgcgtcgtgtctgtcctgactgtgctgcatcaggactggctgaacggaaaggagtacaaatgcaaggtgagcaacaaggccctgccagctcccatcgagaagaccatttccaaagctaagggccagcctcgagaaccacaggtctatgtgctgccacccagccgggacgagctgacaaaaaaccaggtctccctgctgtgtctggtgaagggattctacccttctgatattgcagtggagtgggaaagtaatggccagccagaaaacaattatctgacttggcctccagtgctggattctgacgggagtttctttctgtacagtaaactgaccgtggataagtcacggtggcagcagggaaacgtctttagttgttcagtgatgcacgaggccctgcacaatcattacacccagaaaagcctgtccctgtctcccggcaagtgaggatcc

SEQ ID NO: 161 - protein, 506 aa, synthetic construct
EFATMAVMAPRTLVLLLSGALALTQTWAGDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKAAEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 162 - protein, 513 aa, synthetic construct
EFATMAVMAPRTLVLLLSGALALTQTWAGDIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSAAEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 163 - DNA, 1536 bp, synthetic construct
  source 1..1536: mol_type = other DNA
gaattcgccaccatggccgtgatggcaccaagaacactggtcctgctgctgagcggggcactggcactgacacagacatgggccggacaggtccagctgcagcagagcggagctgagctggcacgaccaggagcaagtgtgaaaatgtcatgcaaggccagcggctacaccttcacacggtatactatgcactgggtgaaacagagacccggacagggcctggaatggatcgggtacattaaccctagccgaggatacaccaactacaaccagaagtttaaagacaaggctaccctgaccacagataagagctcctctacagcatatatgcagctgagttcactgacttctgaggacagtgctgtgtactattgtgcacggtactatgacgatcattactccctggattattgggggcagggaactaccctgaccgtgagctcctctagtacaggaggaggaggcagtggaggaggagggtcaggcggaggaggaagcgacatccagattgtgctgacacagtctccagcaatcatgtccgcctctcccggcgagaaagtcactatgacctgctccgcctcaagctccgtgtcttacatgaattggtatcagcagaaatcaggaaccagccccaagagatggatctacgacacatccaagctggcctctggcgtgcctgctcacttcaggggcagtgggtcaggaactagctattccctgaccattagcggcatggaggccgaagatgccgctacctactattgtcagcagtggtctagtaacccattcacatttggcagcgggactaagctggagatcaatagggcagccgaacccaaatcaagcgacaagacacatacttgccccccttgtccagcaccagaactgctgggaggaccttccgtgttcctgtttccacccaaaccaaaggatacactgatgattagccgcacccctgaggtcacatgcgtggtcgtggacgtgagccacgaggaccccgaagtcaagttcaactggtacgtggacggcgtcgaagtgcataatgccaaaaccaagcctagggaggaacagtacgccagtacatatagagtcgtgtcagtgctgaccgtcctgcaccaggattggctgaacggcaaggagtacaaatgcaaggtgtccaacaaggccctgcctgctccaatcgagaagaccatttctaaagcaaaggggcagccccgagaacctcaggtctacgtgtatcctccatcccgggacgagctgactaaaaaccaggtctctctgacctgtctggtgaagggcttttacccatctgatattgctgtcgagtgggaaagtaatgggcagcccgagaacaattataagacaactccccctgtgctggactccgatgggtctttcgccctggtcagcaaactgacagtggataagtccagatggcagcagggaaacgtcttttcttgtagtgtgatgcatgaagctctgcacaatcattacactcagaaatcactgagcctgtcccccggcaagtgaggatcc

SEQ ID NO: 164 - DNA, 1548 bp, synthetic construct; identical to SEQ ID NO: 160
  source 1..1548: mol_type = other DNA

SEQ ID NO: 165 - protein, 509 aa, synthetic construct
EFATMAVMAPRTLVLLLSGALALTQTWAGQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSSSTGGGGSGGGGSGGGGSDIQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRAAEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 166 - protein, 513 aa, synthetic construct; identical to SEQ ID NO: 162

SEQ ID NO: 167 - DNA, 792 bp, synthetic construct
  source 1..792: mol_type = other DNA
gaattcgccaccatggccgtgatggcacctagaaccctggtcctgctgctgagcggggcactggcactgacacagacttgggctggggaacctaagagcagcgacaagactcacacctgcccaccttgtccagcaccagaactgctgggaggaccaagcgtgttcctgtttccacccaagcccaaagataccctgatgatcagccgaacacccgaagtgacttgcgtggtcgtggacgtgtcccacgaggaccccgaagtcaagttcaactggtacgtggacggcgtcgaagtgcataatgctaagacaaaaccacgggaggaacagtacaactctacttatagagtcgtgagtgtcctgaccgtgctgcatcaggattggctgaacggcaaagagtataagtgcaaagtgtctaataaggccctgcctgctccaatcgagaaaaccattagtaaggctaaagggcagcccagggaacctcaggtctacgtgtatcctccaagtcgcgacgagctgaccaagaaccaggtctcactgacatgtctggtgaaaggattttacccttccgatattgcagtggagtgggaatctaatggccagccagagaacaattataagaccacaccccctgtgctggacagcgatgggtccttcgcactggtctcaaagctgacagtggacaaaagcagatggcagcagggaaacgtctttagctgttccgtgatgcacgaagccctgcacaatcattacactcagaagtctctgagtctgtcacctggcaaatgaggatcc

SEQ ID NO: 168 - DNA, 1518 bp, synthetic construct; identical to SEQ ID NO: 100
  source 1..1518: mol_type = other DNA

SEQ ID NO: 169 - protein, 261 aa, synthetic construct
EFATMAVMAPRTLVLLLSGALALTQTWAGEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 170 - protein, 503 aa, synthetic construct; identical to SEQ ID NO: 102

SEQ ID NO: 171 - DNA, 792 bp, synthetic construct; identical to SEQ ID NO: 167
  source 1..792: mol_type = other DNA

SEQ ID NO: 172 - DNA, 1527 bp, synthetic construct; identical to SEQ ID NO: 104
  source 1..1527: mol_type = other DNA

SEQ ID NO: 173 - protein, 261 aa, synthetic construct; identical to SEQ ID NO: 169

SEQ ID NO: 174 - protein, 506 aa, synthetic construct; identical to SEQ ID NO: 106

SEQ ID NO: 175 - DNA, 792 bp, synthetic construct; identical to SEQ ID NO: 167
  source 1..792: mol_type = other DNA

SEQ ID NO: 176 - DNA, 1503 bp, synthetic construct; identical to SEQ ID NO: 108
  source 1..1503: mol_type = other DNA

SEQ ID NO: 177 - protein, 261 aa, synthetic construct; identical to SEQ ID NO: 169

SEQ ID NO: 178 - protein, 498 aa, synthetic construct; identical to SEQ ID NO: 110

SEQ ID NO: 179 - DNA, 1536 bp, synthetic construct; identical to SEQ ID NO: 127
  source 1..1536: mol_type = other DNA

SEQ ID NO: 180 - DNA, 792 bp, synthetic construct
  source 1..792: mol_type = other DNA
gaattcgccaccatggctgtgatggctccacgcaccctggtcctgctgctgtccggggcactggcactgactcagacttgggctggggaacctaaaagcagcgacaagacccacacatgccccccttgtccagctccagaactgctgggaggaccaagcgtgttcctgtttccacccaagcccaaagatacactgatgatcagccgaactcccgaggtcacctgcgtggtcgtggacgtgtcccacgaggaccccgaagtcaagttcaactggtacgtggacggcgtcgaagtgcataatgcaaagactaaaccacgggaggaacagtacaactctacatatagagtcgtgagtgtcctgactgtgctgcatcaggattggctgaacggcaaagagtataagtgcaaagtgtctaataaggccctgcctgctccaatcgagaaaactattagtaaggcaaaagggcagcccagggaacctcaggtctacgtgctgcctccaagtcgcgacgagctgaccaagaaccaggtctcactgctgtgtctggtgaaaggattctatccttccgatattgccgtggagtgggaatctaatggccagccagagaacaattacctgacctggccccctgtgctggacagcgatgggtccttctttctgtattcaaagctgacagtggacaaaagcagatggcagcagggaaacgtctttagctgttccgtgatgcacgaagccctgcacaatcattacacccagaagtctctgagtctgtcacctggcaaatgaggatcc

SEQ ID NO: 181 - protein, 509 aa, synthetic construct; identical to SEQ ID NO: 129

SEQ ID NO: 182 - protein, 261 aa, synthetic construct
EFATMAVMAPRTLVLLLSGALALTQTWAGEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 183 - DNA, 1548 bp, synthetic construct; identical to SEQ ID NO: 85
  source 1..1548: mol_type = other DNA

SEQ ID NO: 184 - DNA, 1518 bp, synthetic construct
  source 1..1518: mol_type = other DNA
gaattcgccaccatggccgtgatggcaccccgaaccctggtcctgctgctgagcggagccctggcactgacacagacttgggctggacagatcgtcctgacacagtccccagcaatcatgtcagccagccccggggagaaagtcacaatgacttgctcagcaagctcctctgtgagctacatgaactggtatcagcagaaaagcgggacctcccccaagagatggatctacgacacatccaagctggcttctggagtgcctgcacacttcaggggcagcggctctgggaccagttattcactgacaattagcggcatggaggctgaagatgccgctacctactattgccagcagtggagttcaaacccattcacttttggatgtggcaccaagctggaaattaatggcggaggaggctccggaggaggagggtctggaggaggaggaagtcaggtgcagctgcagcagtccggagctgagctggcacgaccaggagcaagtgtgaaaatgtcatgcaaggccagcggctacaccttcacacggtataccatgcattgggtgaaacagagacccggacagtgtctggaatggatcggctacattaatccttctcgagggtacacaaactacaaccagaagtttaaagacaaggctactctgaccacagataagagctcctctaccgcatatatgcagctgagttcactgacatctgaggacagtgccgtgtactattgcgctaggtactatgacgatcactactgtctggattattgggggcagggaactaccctgacagtgagctccgcagccgaacctaaatctagtgacaagactcatacctgccccccttgtccagcaccagagctgctgggaggacctagcgtgttcctgtttccacccaaaccaaaggatactctgatgatctcccggacacctgaagtcacttgcgtggtcgtggacgtgtctcacgaggaccccgaagtcaagtttaactggtacgtggacggcgtcgaggtgcataatgccaaaaccaagcccagggaggaacagtacaactccacatatcgcgtcgtgtctgtcctgactgtgctgcaccaggattggctgaacggaaaggagtacaaatgcaaggtgagcaacaaggccctgcctgctccaatcgagaagacaattagcaaagccaagggccagccccgagaacctcaggtctacgtgctgcctccatctcgggacgagctgactaaaaaccaggtcagtctgctgtgtctggtgaagggattctatccaagcgatattgctgtggagtgggaatccaatggccagcccgaaaacaattacctgacttggccccctgtcctggactcagatggcagcttctttctgtatagtaaactgaccgtggacaagtcacggtggcagcaggggaacgtctttagctgttccgtgatgcatgaggccctgcacaatcattacacccagaaatctctgagtctgtcacccggcaagtgaggatcc

SEQ ID NO: 185 - protein, 513 aa, synthetic construct; identical to SEQ ID NO: 87

SEQ ID NO: 186 - protein, 503 aa, synthetic construct
EFATMAVMAPRTLVLLLSGALALTQTWAGQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGCGTKLEINGGGGSGGGGSGGGGSQVQLQQSGAELARPGASVKMSCKASGYTFTRYTMHWVKQRPGQCLEWIGYINPSRGYTNYNQKFKDKATLTTDKSSSTAYMQLSSLTSEDSAVYYCARYYDDHYCLDYWGQGTTLTVSSAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 187 - DNA, 1548 bp, synthetic construct; identical to SEQ ID NO: 85
  source 1..1548: mol_type = other DNA

SEQ ID NO: 188 - DNA, 1518 bp, synthetic construct
  source 1..1518: mol_type = other DNA
gaattcgccaccatggctgtgatggcacctagaacactggtcctgctgctgagcggggcactggcactgacacagacttgggctgggcagatcgtcctgacacagagcccagcaatcatgtcagccagccccggggagaaagtcacaatgacttgctcagcaagctcctctgtgagctacatgaactggtatcagcagaaaagcgggacctcccccaagagatggatctacgacacatccaagctggcttctggagtgcctgcacacttcaggggcagcggctctgggaccagttattcactgacaatttccggcatggaggctgaagatgccgctacctactattgccagcagtggagttcaaacccattcacttttggatgtggcaccaagctggaaattaatggcggaggaggctccggaggaggagggtctggaggaggaggaagtcaggtgcagctgcagcagtccggagctgagctggcacgaccaggagcaagtgtgaaaatgtcatgcaaggccagcggctacaccttcacacggtataccatgcattgggtgaaacagagacccggacagtgtctggaatggatcggctacattaatcctagccgagggtacacaaactacaaccagaagtttaaagacaaggctactctgaccacagataagagctcctctaccgcatatatgcagctgagttcactgacatctgaggacagtgccgtgtactattgcgctaggtactatgacgatcactactccctggattattgggggcagggaactaccctgacagtgagctccgcagccgaacctaaatctagtgacaagactcatacctgcccaccttgtccagcaccagagctgctgggcgggccttctgtgttcctgtttccacccaaaccaaaggatactctgatgatctcccggacacctgaagtcacttgtgtggtcgtggacgtgtctcacgaggaccccgaagtcaagtttaactggtacgtggacggcgtcgaggtgcataatgccaaaaccaagcccagggaggaacagtacaactccacatatcgcgtcgtgtctgtcctgactgtgctgcaccaggattggctgaacggaaaggagtacaaatgcaaggtgagcaacaaggccctgcctgctccaatcgagaagacaattagcaaagccaagggccagccccgagaacctcaggtctacgtgctgcctccatctcgggacgagctgactaaaaaccaggtcagtctgctgtgtctggtgaagggattctatccaagcgatattgctgtggagtgggaatccaatggccagcccgaaaacaattacctgacttggccccctgtcctggactcagatggcagcttctttctgtatagtaaactgaccgtggacaagtcacggtggcagcaggggaacgtctttagctgttccgtgatgcatgaggccctgcacaatcattacacccagaaatctctgagtctgtcacccggcaagtgaggatcc

SEQ ID NO: 189 - protein, 513 aa, synthetic construct; identical to SEQ ID NO: 87

SEQ ID NO: 190 - protein, 503 aa, synthetic construct
EFATMAVMAPRTLVLLLSGALALTQTWAGQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGCGTKLEINGGGGSGGGGSGGGGSQVQLQQSGAELARPGASVKMSCKASGYTFTRYTMHWVKQRPGQCLEWIGYINPSRGYTNYNQKFKDKATLTTDKSSSTAYMQLSSLTSEDSAVYYCARYYDDHYSLDYWGQGTTLTVSSAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 191 - DNA, 1527 bp, synthetic construct
  source 1..1527: mol_type = other DNA
gaattcgccaccatggctgtgatggcacctagaacactggtcctgctgctgtccggggcactggcactgactcagacttgggctggggatattaagctgcagcagagcggagctgagctggcacgaccaggagccagtgtgaaaatgtcatgcaagacaagcggctacaccttcacacggtatactatgcactgggtgaaacagagacccggccagtgtctggaatggatcgggtatattaacccttcccgaggatacaccaactacaaccagaagtttaaagacaaggccaccctgaccacagataagagctcctctacagcttacatgcagctgagttcactgactagtgaggactcagctgtgtactattgcgcaaggtactatgacgatcattactgtctggattattggggccaggggactaccctgactgtcagctccgtggaaggagggagcggaggctccggaggatctggcgggagtggaggcgtggacgatatccagctgacccagtccccagcaattatgtccgcctctcccggcgagaaagtgactatgacctgccgcgcctctagttcagtgagctacatgaactggtatcagcagaaatcaggaaccagccccaagagatggatctacgacacatccaaggtcgcatctggcgtgccttataggttcagtggatcaggcagcgggacttcctactctctgaccattagctccatggaggccgaagatgccgctacatactattgccagcagtggtctagtaatccactgacatttggatgtggcactaaactggagctgaaggcagccgaacccaaatcaagcgacaagacacacacttgccccccttgtccagcacccgaactgctggggggacctagcgtgttcctgtttccacccaaaccaaaggatacactgatgatcagccggacccctgaggtcacatgtgtggtcgtggacgtgagccacgaggaccccgaagtcaagttcaactggtacgtggacggcgtcgaagtgcataatgctaaaaccaagcctagggaggaacagtacaatagtacttatcgcgtcgtgtcagtcctgaccgtgctgcatcaggattggctgaacggcaaggagtacaaatgcaaggtgtccaacaaggccctgcctgctccaatcgagaagaccatttctaaagccaaggggcagccccgagaacctcaggtctacgtgtatcctccatcccgggacgagctgaccaaaaaccaggtctctctgacatgtctggtgaagggcttttatccatctgatattgctgtggagtgggaaagtaatgggcagcccgagaacaattacaagacaactccccctgtgctggactccgatgggtctttcgctctggtcagcaaactgacagtggacaagtccagatggcagcagggaaacgtctttagttgctcagtgatgcacgaggcactgcacaatcattacactcagaaaagcctgtccctgtctcccggcaagtgaggatcc

SEQ ID NO: 192 - DNA, 1548 bp, synthetic construct; identical to SEQ ID NO: 112
  source 1..1548: mol_type = other DNA

SEQ ID NO: 193 - protein, 506 aa, synthetic construct
EFATMAVMAPRTLVLLLSGALALTQTWAGDIKLQQSGAELARPGASVKMSCKTSGYTFTRYTMHWVKQRPGQCLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGCGTKLELKAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 194 - protein, 513 aa, synthetic construct; identical to SEQ ID NO: 114

SEQ ID NO: 195 - DNA, 792 bp, synthetic construct; identical to SEQ ID NO: 13
  source 1..792: mol_type = other DNA

SEQ ID NO: 196 - DNA, 1542 bp, synthetic construct
  source 1..1542: mol_type = other DNA
gaattcgccaccatggctgtgatggctccaagaacactggtcctgctgctgagcggggcactggcactgacacagacatgggccgggcaggtccagctgcaggaatctggaggaggactggtgaagccaggagggtctctgagactgagttgcgccgcttcaggcttcacctttagctcctactggatgagctgggtgcggcaggcacctggcaaaggactggagtgggtggccaacatcaatagagacgggagtgcttcatactatgtcgatagcgtgaagggaaggttcaccattagtcgcgacgatgcaaaaaactcactgtacctgcagatgaatagcctgagggccgaagacacagctgtctactattgcgcccgggacagaggagtgggctattttgatctgtgggggcgcggaacactggtcactgtgtctagtgcttccacaggaggcgggggatcaggcggaggaggcagcggaggaggagggagccagtccgctctgactcagcctgcatctgtgagtggctcaccagggcagagcatcaccatttcctgtaccggcacatcaagcgatgtcggaggctacaacttcgtgagctggtatcagcagcacccagggaaggcacccaaactgatgatctacgacgtctccgatcgaccaagtggagtgtcagaccggtttagcggcagcaagtctggcaatactgcctctctgatcattagtggactgcaggcagacgatgaggccgattactattgctcctcttatggcagttcaagcacccacgtgattttcgggggaggcaccaaggtcacagtgctgggcgcagccgaacccaagtcctctgacaaaactcatacctgcccaccttgtccagctccagagctgctgggaggaccaagcgtgttcctgtttccacccaagcctaaagatacactgatgatcagccgaactcctgaagtcacctgtgtggtcgtggacgtgtcccacgaggaccccgaagtcaagtttaactggtacgtggacggcgtcgaggtgcataatgccaagactaaacctagagaggaacagtacaactccacatatagggtcgtgtctgtcctgactgtgctgcaccaggattggctgaacggaaaggagtataagtgcaaagtgtctaataaggctctgcctgcaccaatcgagaaaaccattagcaaggctaaagggcagccccgcgaacctcaggtgtacaccctgcctccatctcgagacgagctgacaaagaaccaggtcagcctgctgtgtctggtgaaaggcttctatccaagtgatattgcagtggagtgggaatccaatgggcagcccgaaaacaattacatgacatggccccctgtgctggacagcgatggctccttctttctgtattccaagctgactgtggacaaatctcgctggcagcaggggaacgtctttagttgttcagtgatgcatgaggccctgcacaatcattacacccagaagagcctgtccctgtctcccggcaaatgaggatcc

SEQ ID NO: 197 - protein, 261 aa, synthetic construct; identical to SEQ ID NO: 14

SEQ ID NO: 198 - protein, 511 aa, synthetic construct
EFATMAVMAPRTLVLLLSGALALTQTWAGQVQLQESGGGLVKPGGSLRLSCAASGFTFSSYWMSWVRQAPGKGLEWVANINRDGSASYYVDSVKGRFTISRDDAKNSLYLQMNSLRAEDTAVYYCARDRGVGYFDLWGRGTLVTVSSASTGGGGSGGGGSGGGGSQSALTQPASVSGSPGQSITISCTGTSSDVGGYNFVSWYQQHPGKAPKLMIYDVSDRPSGVSDRFSGSKSGNTASLIISGLQADDEADYYCSSYGSSSTHVIFGGGTKVTVLGAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYMTWPPVLDSDGSFFLYSKLTVDKSRWQQGNVFSCSVMHEALHNHYTQKSLSLSPGK

SEQ ID NO: 199 - DNA, 1563 bp, synthetic construct
  source 1..1563: mol_type = other DNA
gaattcgccaccatggccgtgatggcacctagaacactggtcctgctgctgagcggagccctggcactgacacagacttgggccggacaggtccagctggtgcagagcggggcagaggtcaagaaacccggagaaagtctgaagatctcatgcaaagggagtggatactcattcaccagctattggattgcctgggtgaggcagatgcctggcaaggggctggaatacatgggcctgatctatccaggggacagcgatacaaaatactccccctctttccagggccaggtcacaatttccgtggacaagagtgtctcaactgcctatctgcagtggagctccctgaaacctagcgattccgcagtgtacttttgtgccaggcacgacgtcgggtattgcacagatcgcacttgtgctaagtggccagagtggctgggagtgtggggacagggaaccctggtcacagtgtctagtggaggaggaggctcaagcggaggaggctctggaggaggagggtctcagagtgtgctgactcagccaccttcagtcagcgcagctcctggacagaaggtgaccatctcctgctctggcagctctagtaacattggcaacaattacgtgagctggtatcagcagctgcctggcaccgccccaaagctgctgatctacgaccacacaaatcggcccgctggggtgcctgatagattcagtgggtcaaaaagcggaacctccgcttctctggcaattagcggctttcgctccgaggacgaagctgattactattgtgcatcttgggactacacactgagtggctgggtgttcggaggcgggactaagctgaccgtgctgggggcagccgaaccaaagtcaagcgataaaactcatacctgcccaccatgtcctgcaccagagctgctgggaggaccttccgtgttcctgtttcctccaaagccaaaagacaccctgatgatcagccgaacaccagaagtgacttgcgtggtcgtggacgtctcccacgaggaccccgaagtgaagtttaactggtacgtggatggcgtcgaggtgcataatgccaagaccaaaccccgagaggaacagtacaactcaacttatcgggtcgtgagcgtcctgaccgtgctgcaccaggactggctgaacgggaaagagtataagtgcaaagtgtctaataaggccctgcccgctcctatcgagaaaacaattagcaaggcaaaaggccagccaagagaaccccaggtgtacacttatcccccttctagggacgagctgaccaagaaccaggtgagcctgacatgtctggtcaaaggattttaccccagtgatattgctgtggagtgggaatccaatggccagcctgaaaacaattataagaccacaccacccgtgctggactccgatggatctttcgctctggtgtccaagctgactgtcgataaatctcggtggcagcagggcaacgtgtttagttgttcagtcatgcatgaggcactgcacaatcattacacacagaagagcctgtccctgtctcccggcaaatgaggatcc

SEQ ID NO: 200 - DNA, 1542 bp, synthetic construct; identical to SEQ ID NO: 196
  source 1..1542: mol_type = other DNA

SEQ ID NO: 201 - protein, 518 aa, synthetic construct [sequence omitted]

SEQ ID NO: 202 - protein, 511 aa, synthetic construct; identical to SEQ ID NO: 198

SEQ ID NO: 203 - protein, 483 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 204 - DNA, 1455 bp, synthetic construct
  source 1..1455: mol_type = other DNA
ggagaagtccagctggtcgagtccggaggaggactggtgcagccaggagggtcactgaaactgagctgcgccgcttccggcttcacttttaacaagtatgcaatgaattgggtgcggcaggcaccagggaagggactggaatgggtggcccggatcagatctaagtacaacaactacgctacctactatgcagacagtgtgaaggataggttcacaatttctcgcgacgatagtaaaaacactgcttacctgcagatgaacaatctgaagacagaggacactgcagtctactattgcgtgagacacggaaactttggcaatagctacatctcctattgggcatactggggacagggaaccctggtcacagtgagctccggaggaggaggcagcggaggaggaggctctgggggaggcgggagtcagactgtggtcacccaggagccctcactgacagtcagccctggaggcactgtgaccctgacatgtgggtctagtaccggagccgtgacatctggcaactatcccaattgggtgcagcagaaacctggacaggctccacgaggactgattggaggaacaaagttcctggcccccggaactcctgctcgattttccggctctctgctgggagggaaagcagcactgaccctgagcggagtgcagcctgaggatgaagccgagtactattgcgtgctgtggtacagcaacagatgggtgttcggaggcgggacaaagctgactgtgctggctgcagagccaaagtcaagcgacaaaactcacacctgcccaccttgtccagctccagaagcagctggaggaccatccgtgttcctgtttccacccaagcccaaagatacactgatgatctctcgcactcccgaggtcacctgtgtggtcgtgagtgtgtcacacgaagaccctgaggtcaagtttaactggtacgtggatggcgtcgaagtgcataatgccaagaccaaacctcgagaggaacagtataattcaacttaccgggtcgtgagcgtcctgaccgtgctgcatcaggactggctgaacggaaaggagtacaagtgcaaagtgagcaataaggcactgcctgccccaatcgaaaaaaccattagcaaggctaaagggcagccaagagagccccaggtctacgtgtatcctccaagcagggacgaactgaccaagaaccaggtctccctgacatgtctggtgaaagggttctatcctagtgatattgcagtggaatgggagtcaaatggacagccagagaacaattacaagaccacaccccctgtgctggactctgatggcagtttcgcactggtctccaagctgaccgtggataaatctaggtggcagcaggggaacgtctttagctgttccgtgatgcatgaagccctgcacaatcattacacacagaagtctctgagtctgtcacccggcaaatga

SEQ ID NO: 205 - protein, 452 aa, synthetic construct
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 206 - DNA, 1362 bp, synthetic construct
  source 1..1362: mol_type = other DNA
ggggaagtccagctggtcgaatctggaggaggactggtgcagcctggacgatccctgagactgtcttgcgccgctagtggcttcacttttaacgactatgcaatgcactgggtgcgccaggcaccagggaagggactggagtgggtgagcaccatctcctggaacagcggatctattggctatgcagacagcgtgaaaggcaggttcacaatcagtcgcgataacgccaagaaatcactgtacctgcagatgaatagcctgcgagccgaagacacagctctgtactattgcgccaaggatattcagtatgggaactactattacggaatggacgtgtggggccaggggaccacagtcaccgtgagctccgcctcaacaaaggggcccagcgtgtttccactggctccctctagtaaaagtacctcaggcgggacagcagccctgggatgtctggtgaaggattacttcccagagcccgtcaccgtgtcttggaacagtggcgctctgacaagcggggtccatacttttccagcagtgctgcagtcaagcggcctgtattccctgtcctctgtggtcactgtgcccagttcaagcctggggactcagacctacatctgcaacgtgaatcacaagccatctaataccaaagtcgacaagaaagtggaacccaagagttgtgataaaacacatacttgcccaccttgtcctgcaccagagctgctgggaggaccatccgtgttcctgtttccacccaagcctaaagacaccctgatgattagcaggactcccgaagtcacctgcgtggtcgtggacgtgtcccacgaggaccccgaagtcaagttcaactggtacgtggatggcgtcgaggtgcataatgctaagacaaaaccccgagaggaacagtataattccacttaccgggtcgtgtctgtcctgaccgtgctgcaccaggactggctgaacggcaaggagtacaagtgcaaagtgtctaataaggctctgcccgcacctatcgagaaaacaattagcaaggctaaagggcagcctagagaaccacaggtctatgtgctgcctccaagcagggacgagctgactaagaaccaggtctccctgctgtgtctggtgaaagggttctaccctagtgatatcgcagtggagtgggaatcaaatggacagccagaaaacaattatctgacatggccccctgtgctggactcagatggaagcttctttctgtactccaagctgactgtggataaatctcggtggcagcagggcaacgtctttagctgttccgtgatgcatgaggccctgcacaatcattacacccagaagtctctgagtctgtcacctggcaaatga

SEQ ID NO: 207 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 208 - DNA, 648 bp, synthetic construct
  source 1..648: mol_type = other DNA
ggggaaatcgtcctgacacagtcccctgccactctgagtctgtcaccaggcgagagggctaccctgtcttgccgcgcaagccagtccgtgagctcctacctggcctggtatcagcagaagccagggcaggctcccagactgctgatctacgacgcatccaaccgagcaaccggcatccccgcacggttctctggcagtgggtcaggaacagactttaccctgacaatctctagtctggagcccgaagatttcgctgtgtactattgccagcagaggtctaattggcctatcacctttggccaggggacacggctggagattaagagaactgtggccgctccaagtgtcttcatttttccccctagcgacgaacagctgaaatccggcacagcctctgtggtctgtctgctgaacaatttctacccccgcgaggcaaaggtgcagtggaaagtcgataacgccctgcagagcggcaacagccaggagtctgtgactgaacaggacagtaaggattcaacctatagcctgtcaagcactctgaccctgagcaaagctgattacgagaagcacaaagtgtatgcatgcgaagtcacacatcagggactgtcctctcccgtcactaaaagctttaaccgaggcgaatgttga

SEQ ID NO: 209 - protein, 483 aa, synthetic construct; identical to SEQ ID NO: 203

SEQ ID NO: 210 - DNA, 1455 bp, synthetic construct; identical to SEQ ID NO: 204
  source 1..1455: mol_type = other DNA

SEQ ID NO: 211 - protein, 451 aa, synthetic construct
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 212 - DNA, 1359 bp, synthetic construct
  source 1..1359: mol_type = other DNA
ggcgaggtccagctggtcgaatccggaggaggactggtgaagccaggagggagtctgaaactgtcatgcgccgctagcggctataccttcacatcttacgtcatgcactgggtgaggcaggcacctggcaagggactggaatggatcggatatattaacccatacaatgacggcactaagtataacgagaaatttcagggcagagtgaccatcagctccgataagagcatttccacagcttacatggagctgtctagtctgaggagcgaagacaccgccatgtactattgcgctcgggggacctactattacggaacaagagtgttcgattattggggacagggcaccctggtcacagtgtcaagcgcttccacaaaggggccttctgtgtttccactggcaccctcctctaaatctactagtggaggcaccgcagccctgggatgtctggtgaaggactacttcccagagcccgtcacagtgtcatggaacagcggcgcactgactagcggggtccatacctttcctgccgtgctgcagagttcaggcctgtatagcctgagctccgtggtcacagtgccatctagttcactggggactcagacctacatctgcaacgtgaatcacaagccatccaatactaaagtcgacaagaaagtggaacccaagtcttgtgataaaacacatacttgcccaccttgtcctgcaccagagctgctgggaggaccatccgtgttcctgtttccacccaagcctaaagatactctgatgattagtcgcacaccagaagtgacttgcgtggtcgtggacgtgagccacgaggaccccgaagtcaagttcaactggtacgtggacggcgtcgaggtgcataatgccaagaccaaacccagggaggaacagtataatagtacatacagagtcgtgtcagtgctgaccgtcctgcaccaggattggctgaacggcaaggagtacaagtgcaaagtgtccaataaggctctgcccgcacctatcgagaaaaccatttctaaggcaaaagggcagcctcgagaaccacaggtctatgtgctgcctccatcacgggatgagctgacaaagaaccaggtcagcctgctgtgtctggtgaaagggttctacccctctgacatcgctgtggagtgggaaagtaatggacagcctgaaaacaattatctgacttggccccctgtgctggactccgatggatctttctttctgtacagcaagctgaccgtggacaaatcccgatggcagcagggcaacgtcttttcatgtagcgtgatgcatgaggccctgcacaatcattacacccagaagtccctgtctctgagtcccggcaaatga

SEQ ID NO: 213 - protein, 219 aa, synthetic construct
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 214 - DNA, 663 bp, synthetic construct
  source 1..663: mol_type = other DNA
ggagacattgtgatgacacagtcccctgccactctgagtctgtcaccaggcgagcgggctaccctgagttgcagaagctccaagagcctgcagaacgtgaatggaaacacatacctgtattggttccagcagaaaccaggccagtctccccagctgctgatctacaggatgtcaaatctgaacagcggagtgcctgaccgcttcagcggctccgggtctggaaccgagttcaccctgacaatttctagtctggagcccgaagatttcgcagtctactattgcatgcagcacctggagtatcctatcacctttggcgctgggacaaagctggagatcaagcgaactgtggccgctccatccgtcttcatctttcccccttctgacgagcagctgaagtccggcacagcctctgtggtctgtctgctgaacaatttctaccccagagaagcaaaggtgcagtggaaagtcgataatgccctgcagagtgggaactcacaggagagcgtgactgaacaggactccaaggattctacctatagtctgtcaagcactctgaccctgagcaaagctgactacgagaagcacaaagtgtatgcatgcgaagtcacacatcaggggctgtcctctcccgtgactaaaagctttaatcggggagagtgttga

SEQ ID NO: 215 - protein, 449 aa, synthetic construct
QVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 216 - DNA, 1353 bp, synthetic construct
  source 1..1353: mol_type = other DNA
ggacaggtccagctggtgcagagcggaggaggagtggtccagccaggacggtctctgagactgagttgcaaggcatcagggtacactttcacccgatataccatgcactgggtgcggcaggcaccagggaaaggactggaatggatcgggtacattaacccttccaggggatacacaaactataatcagaaggtgaaagacaggttcactatcagccgcgataactccaagaataccgcttttctgcagatggactctctgcgccccgaggatacaggcgtgtatttctgcgcacgatactatgacgatcactactgtctggactattggggccaggggactccagtcaccgtgagctccgcctctactaagggacccagtgtgtttccactggctccctctagtaaatccacatctggaggaactgcagctctgggatgcctggtgaaggattacttcccagagcccgtcaccgtgagttggaactcaggagctctgactagcggcgtccatacctttcccgcagtgctgcagtcaagcgggctgtacagcctgtcctctgtggtcacagtgcctagttcaagcctgggaacacagacttatatctgcaacgtgaatcacaagccttctaatactaaagtcgacaagaaagtggaaccaaagagttgtgataaaacccatacatgcccaccttgtcctgcaccagagctgctgggaggaccaagcgtgttcctgtttccacccaagcctaaagacaccctgatgattagccggacccctgaagtcacatgtgtggtcgtggacgtgagccacgaggaccccgaagtcaagttcaactggtacgtggatggcgtcgaggtgcataatgccaagacaaaacctagagaggaacagtacaattcaacctatagggtcgtgagcgtcctgacagtgctgcaccaggactggctgaacgggaaggagtataagtgcaaagtgtccaataaggcactgcccgcccctatcgagaaaaccatttctaaggcaaaaggccagcctagggaaccacaggtctacgtgtatcctccaagccgcgacgagctgacaaagaaccaggtctccctgacttgtctggtgaaaggattttacccaagtgatattgctgtggagtgggaatcaaatggccagcccgaaaacaattataagaccacaccccctgtgctggacagcgatggctccttcgccctggtctccaagctgactgtggataaatctagatggcagcaggggaacgtctttagttgttcagtgatgcatgaggctctgcacaatcattacacccagaagagcctgtccctgtctcccggcaaatga

SEQ ID NO: 217 - protein, 482 aa, synthetic construct
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSSGGGGSGGGGSGGGGSDIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 218 - DNA, 1452 bp, synthetic construct
  source 1..1452: mol_type = other DNA
ggggaagtccagctggtcgagtctggaggaggactggtgaagccaggagggagtctgaaactgtcatgcgccgctagcgggtataccttcacaagctacgtcatgcactgggtgaggcaggcaccagggaagggactggaatggatcggctatattaatccctacaacgacgggactaagtataatgagaaatttcagggcagggtgaccatcagctccgataagtctattagtacagcctacatggagctgtctagtctgcgcagcgaagacacagcaatgtactattgcgccagggggacatactattacggaactcgcgtgttcgattactggggccaggggaccctggtcacagtgtcaagcggaggcgggggaagtggaggaggaggctcaggaggaggagggagcgacatcgtgatgacccagtcccctgctacactgtcactgagcccaggcgagcgggcaactctgtcctgtagatcctctaagtctctgcagaacgtgaatggaaacacctatctgtactggtttcagcagaaaccaggccagagcccccagctgctgatctatagaatgtccaatctgaactctggcgtgcctgataggttctccggatctggcagtgggaccgagttcaccctgaccattagttcactggagccagaagacttcgccgtgtattactgcatgcagcacctggagtaccccatcacttttggagctggcaccaagctggagatcaaggcagccgaaccaaagagctccgataaaacacatacttgcccaccttgtccagcaccagaagctgcaggaggaccaagcgtgttcctgtttccacccaagcctaaagacaccctgatgatctcccggactcccgaggtcacctgtgtggtcgtgtcagtgagccacgaggaccctgaagtcaagttcaattggtacgtggatggcgtcgaagtgcataacgctaagacaaaaccccgagaggaacagtataacagtacataccgggtcgtgtcagtgctgaccgtcctgcaccaggattggctgaatggaaaggagtacaagtgcaaagtgtctaacaaggccctgcctgctccaatcgagaaaaccattagcaaggctaaaggccagccccgcgaacctcaggtctatgtgctgcctccaagccgagatgagctgacaaagaatcaggtctccctgctgtgtctggtgaaagggttctacccttctgacattgcagtggagtgggaaagtaacggacagccagagaacaattatctgacatggccccctgtcctggactccgatggctctttctttctgtacagcaagctgactgtggacaaatccagatggcagcaggggaatgtcttttcctgttctgtgatgcatgaagccctgcacaaccattacacccagaagagtctgtcactgagccctggcaaatga

SEQ ID NO: 219 - protein, 213 aa, synthetic construct
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 220 - DNA, 645 bp, synthetic construct
  source 1..645: mol_type = other DNA
ggggatattcagatgacccagagcccaagctccctgagtgcctcagtgggcgaccgagtcaccatcacatgctccgcttctagttcagtgtcttacatgaactggtatcagcagactccagggaaggcacccaaacggtggatctacgatacctcaaagctggccagcggagtgccctccagattcagcggctccgggtctggaacagactatacttttaccatcagctccctgcagcctgaggatattgctacttactattgccagcagtggtctagtaatccattcacttttggccaggggaccaagctgcagatcacaaggactgtggccgctcccagcgtcttcatttttccccctagcgacgagcagctgaaatctggcacagccagtgtggtctgtctgctgaacaatttctaccctcgcgaagcaaaggtgcagtggaaagtcgataacgccctgcagagtggcaacagccaggagagcgtgacagaacaggactccaaggattctacttatagtctgtcaagcaccctgacactgtccaaagctgactacgagaagcacaaagtgtatgcatgcgaagtcacccatcagggactgtcctctcctgtgacaaaatcttttaacagaggcgaatgttga

SEQ ID NO: 221 - protein, 483 aa, synthetic construct; identical to SEQ ID NO: 203

SEQ ID NO: 222 - DNA, 1455 bp, synthetic construct; identical to SEQ ID NO: 204
  source 1..1455: mol_type = other DNA

SEQ ID NO: 223 - protein, 478 aa, synthetic construct
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 224 - DNA, 1440 bp, synthetic construct
  source 1..1440: mol_type = other DNA
ggggaagtccagctggtcgagagcggaggaggactggtgcagccaggacggtccctgagactgtcttgcgccgctagtgggttcacctttaacgactatgccatgcactgggtccgacaggctccaggaaagggactggaatgggtgtctaccatcagttggaatagtggatcaattggctatgctgactccgtgaaaggcaggttcacaatctcacgcgataacgcaaagaaaagcctgtacctgcagatgaacagcctgcgcgccgaggacacagctctgtactattgcgccaaggatattcagtacgggaactactattacggaatggacgtgtgggggcagggaaccacagtcactgtgagctccggcgggggaggctcaggaggaggagggagcggaggaggaggcagcgaaatcgtgctgactcagagccctgcaaccctgagcctgtccccaggagagcgagctacactgagctgtcgggcatctcagagtgtgtctagttatctggcatggtaccagcagaagccagggcaggcccccagactgctgatctacgatgcatccaacagagccactggcatccccgcaaggttctcaggcagcgggtccggaaccgactttactctgaccatctcaagcctggagcccgaagatttcgctgtgtattactgccagcagaggtctaattggcctatcacatttggccaggggactcgcctggagattaaggcagccgaaccaaagtcctctgacaaaacacacacttgccccccttgtccagcaccagaactgctgggaggaccaagcgtgttcctgtttccacccaagcctaaagataccctgatgattagtaggacccctgaggtcacatgtgtggtcgtggacgtgagccacgaggaccccgaagtcaagtttaactggtacgtggacggcgtcgaagtgcataatgccaagacaaaaccccgcgaggaacagtataattctacctaccgagtcgtgagtgtcctgacagtgctgcatcaggattggctgaacggaaaagagtacaagtgcaaagtgtccaataaggctctgcctgcaccaatcgagaaaactatttctaaggcaaaagggcagccccgggaacctcaggtctatgtgctgcctccatccagagacgagctgaccaagaaccaggtctctctgctgtgtctggtgaaaggattctacccatcagatatcgctgtggagtgggaaagcaatggccagcccgagaacaattatctgacatggccccctgtgctggactcagatggcagcttctttctgtactctaagctgactgtggataaaagtcggtggcagcaggggaacgtcttttcttgtagtgtgatgcatgaggccctgcacaatcattacacccagaagtcactgagcctgtcccctggcaaatga

SEQ ID NO: 225 - protein, 449 aa, synthetic construct; identical to SEQ ID NO: 215

SEQ ID NO: 226 - DNA, 1353 bp, synthetic construct; identical to SEQ ID NO: 216
  source 1..1353: mol_type = other DNA

SEQ ID NO: 227 - protein, 478 aa, synthetic construct; identical to SEQ ID NO: 223

SEQ ID NO: 228 - DNA, 1440 bp, synthetic construct; identical to SEQ ID NO: 224
  source 1..1440: mol_type = other DNA

SEQ ID NO: 229 - protein, 213 aa, synthetic construct; identical to SEQ ID NO: 219

SEQ ID NO: 230 - DNA, 645 bp, synthetic construct; identical to SEQ ID NO: 220
  source 1..645: mol_type = other DNA

SEQ ID NO: 231 - protein, 448 aa, synthetic construct
QVQLVESGGGVVQPGRSLRLSCAASGFKFSGYGMHWVRQAPGKGLEWVAVIWYDGSKKYYVDSVKGRFTISRDNSKNTLYLQMNSLRAEDTAVYYCARQMGYWHFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 232 - DNA, 1350 bp, synthetic construct
  source 1..1350: mol_type = other DNA
gggcaggtccagctggtggaatccggaggaggagtggtccagcctggacgatctctgagactgagttgcgccgcttcagggttcaagtttagcgggtacggaatgcactgggtgaggcaggcaccaggcaaagggctggagtgggtcgccgtgatctggtatgacggcagcaagaagtactatgtcgattctgtgaagggcaggttcaccattagccgcgacaactccaaaaatacactgtacctgcagatgaactccctgagagccgaagacaccgctgtgtactattgcgccaggcagatgggctattggcacttcgatctgtggggacgaggaaccctggtcacagtgagctccgcatctacaaaggggcccagtgtgtttccactggctccctctagtaaatccacttctggaggaaccgcagcactgggatgtctggtgaaggattacttcccagagcccgtcaccgtgagttggaactcaggggctctgacctccggagtccatacatttccagcagtgctgcagtcaagcggcctgtacagcctgtcctctgtggtcactgtgcccagttcaagcctggggactcagacctatatctgcaacgtgaatcacaagccatcaaataccaaagtcgacaagaaagtggaacccaagagctgtgataaaacacatacttgcccaccttgtcctgcaccagagctgctgggaggaccaagcgtgttcctgtttccacccaagcctaaagacactctgatgatttcccggacacccgaagtgacttgcgtggtcgtggacgtgtctcacgaggaccccgaagtcaagttcaactggtacgtggatggcgtcgaggtgcataatgctaagacaaaaccccgagaggaacagtacaattcaacatatcgggtcgtgagcgtcctgactgtgctgcaccaggactggctgaacggcaaggagtataagtgcaaagtgagtaataaggctctgcccgcacctatcgagaaaaccatttctaaggctaaagggcagcctcgcgaaccacaggtctacgtgtatcctccatctcgagacgagctgactaagaaccaggtcagtctgacctgtctggtgaaagggttttaccctagcgatatcgcagtggagtgggaatccaatggacagccagaaaacaattataagaccacaccccctgtgctggacagcgatggcagcttcgcactggtcagtaagctgacagtggataaatcaagatggcagcagggcaacgtctttagttgttcagtgatgcatgaggccctgcacaatcattacactcagaagagcctgtccctgtctcctggcaaatga

SEQ ID NO: 233 - protein, 452 aa, synthetic construct; identical to SEQ ID NO: 205

SEQ ID NO: 234 - DNA, 1362 bp, synthetic construct; identical to SEQ ID NO: 206
  source 1..1362: mol_type = other DNA

SEQ ID NO: 235 - protein, 214 aa, synthetic construct; identical to SEQ ID NO: 207

SEQ ID NO: 236 - DNA, 648 bp, synthetic construct; identical to SEQ ID NO: 208
  source 1..648: mol_type = other DNA

SEQ ID NO: 237 - protein, 449 aa, synthetic construct
QVQLVQSGGGVVQPGRSLRLSCKASGYTFTSYTMHWVRQAPGKGLEWIGYINPSSGYTKYNQKFKDRFTISADKSKSTAFLQMDSLRPEDTGVYFCARWQDYDVYFDYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 238 - DNA, 1353 bp, synthetic construct
  source 1..1353: mol_type = other DNA
gggcaggtccagctggtgcagtccggaggaggagtggtccagccaggacggtccctgagactgtcttgcaaggctagtgggtatactttcacctcttacaccatgcactgggtgcgccaggcaccagggaagggactggaatggatcgggtatattaaccctagctccggatacacaaagtacaaccagaagttcaaagaccggttcaccatctccgctgataagagtaaatcaaccgcattcctgcagatggactctctgcgacccgaggatacaggcgtgtacttctgcgcccggtggcaggactacgatgtgtattttgactactggggccaggggactccagtcaccgtgtctagtgcatcaactaagggacccagcgtgtttccactggccccctcaagcaaaagcacatccggaggaactgcagctctgggatgtctggtgaaggattatttcccagagcccgtcaccgtgtcttggaacagtggagccctgactagcggcgtccatacctttcccgctgtgctgcagtcctctgggctgtatagcctgagttcagtggtcacagtgcctagctcctctctgggaacacagacttacatctgcaacgtgaatcacaagccttcaaatactaaagtcgacaagaaagtggaaccaaagagctgtgataaaacccatacatgcccaccttgtcctgcaccagagctgctgggaggaccaagcgtgttcctgtttccacccaagcctaaagacaccctgatgatttccaggacccctgaagtcacatgcgtggtcgtggacgtgtctcacgaggaccccgaagtcaagttcaactggtacgtggatggcgtcgaggtgcataatgccaagacaaaacctagggaggaacagtataactccacctaccgcgtcgtgtctgtcctgacagtgctgcaccaggactggctgaacgggaaggagtacaagtgcaaagtgagtaataaggcactgcccgcccctatcgagaaaaccattagcaaggcaaaaggccagcctagagaaccacaggtctacgtgtatcctccatctagggacgagctgacaaagaaccaggtcagtctgacttgtctggtgaaaggattttatccaagcgatattgctgtggagtgggaatccaatggccagcccgaaaacaattacaagaccacaccccctgtgctggactcagatggcagcttcgccctggtcagtaagctgactgtggataaatcacggtggcagcaggggaacgtcttttcttgtagtgtgatgcatgaggctctgcacaatcattacacccagaagtcactgagcctgtcccccggcaaatga

SEQ ID NO: 239 - protein, 451 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 240 - DNA, 1359 bp, synthetic construct
  source 1..1359: mol_type = other DNA
ggacaggtccagctgcagcagcccggagctgaactggtcaaacctggcgcatccgtgaaaatgtcttgcaaggctagtggctacacattcacttcctataacatgcactgggtgaagcagacaccaggacgaggactggagtggatcggagcaatctaccctggaaacggcgacacttcttataatcagaagtttaaaggcaaggccaccctgacagctgataagagctcctctaccgcctacatgcagctgagttcactgacaagtgaagactcagcagtgtactattgcgccagaagcacctactatggcggggattggtacttcaacgtgtggggggcaggaaccacagtcaccgtgagcgccgcttccacaaaaggaccaagcgtgtttccactggcaccaagctccaagtcaaccagcggaggaacagcagccctgggatgtctggtgaaggactacttcccagagcccgtcaccgtgtcttggaacagtggcgccctgacaagcggggtccatacttttcccgctgtgctgcagtctagtggcctgtacagcctgtcaagcgtggtcaccgtcccttcctctagtctggggactcagacctatatctgcaacgtgaatcacaaaccttctaatacaaaggtcgacaagaaagtggaaccaaaaagttgtgataagacacatacttgcccaccttgtcctgcaccagagctgctgggaggaccatccgtgttcctgtttccacccaaacccaaggacactctgatgattagccggactcctgaagtcacctgcgtggtcgtggacgtgagccacgaggaccccgaagtcaaattcaactggtacgtggatggcgtcgaggtgcataatgccaaaacaaagccccgggaggaacagtacaactcaacatatagagtcgtgagcgtcctgactgtgctgcaccaggactggctgaacggcaaggagtataaatgcaaggtgtccaacaaggccctgcccgcacctatcgagaagactatttctaaagccaagggccagcctagggaaccacaggtgtacgtgctgcctccaagccgcgacgagctgactaaaaaccaggtctccctgctgtgtctggtgaaggggttctatccaagtgatatcgctgtggagtgggaatcaaatggacagcccgagaacaattacctgacttggccccctgtgctggactcagatgggagcttctttctgtattccaaactgaccgtggataagtctcggtggcagcagggaaatgtcttttcctgttctgtgatgcacgaagcactgcacaatcactacacccagaagtccctgagcctgtcacccggcaaatga

SEQ ID NO: 241 - protein, 213 aa, synthetic construct
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 242 - DNA, 645 bp, synthetic construct
  source 1..645: mol_type = other DNA
gggcagattgtcctgtctcagagtcccgctatcctgtcagcaagccctggggagaaggtgaccatgacatgccgagccagctcctctgtcagctacatccactggttccagcagaagccaggcagttcacctaaaccatggatctacgccacatctaacctggctagtggagtgcccgtccggttttccggctctgggagtggaacatcatacagcctgactatttccagagtggaggccgaagacgccgctacctactattgccagcagtggacctctaatccccctacattcggcgggggaactaagctggagatcaaaaggactgtggcagccccttctgtcttcatttttccacccagtgacgaacagctgaaatcaggaaccgcttccgtggtctgtctgctgaacaacttctacccccgcgaggcaaaggtgcagtggaaagtcgataacgccctgcagtccggcaattctcaggagagtgtgaccgaacaggactcaaaggatagcacatattccctgagctccactctgaccctgtccaaagctgattacgaaaagcataaagtgtatgcatgtgaggtcacccaccaggggctgagtagtcccgtcacaaagagtttcaatagaggagagtgttga

SEQ ID NO: 243 - protein, 449 aa, synthetic construct; identical to SEQ ID NO: 237

SEQ ID NO: 244 - DNA, 1353 bp, synthetic construct; identical to SEQ ID NO: 238
  source 1..1353: mol_type = other DNA

SEQ ID NO: 245 - protein, 452 aa, synthetic construct
QAYLQQSGAELVRPGASVKMSCKASGYTFTSYNMHWVKQTPRQGLEWIGAIYPGNGDTSYNQKFKGKATLTVDKSSSTAYMQLSSLTSEDSAVYFCARVVYYSNSYWYFDVWGTGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 246 - DNA, 1362 bp, synthetic construct
  source 1..1362: mol_type = other DNA
ggacaggcttacctgcagcagtccggagcagaactggtccgaccaggagcttccgtgaaaatgtcttgcaaagcaagtggctacactttcaccagctataacatgcactgggtgaaacagacacctcgacagggactggagtggatcggagcaatctacccagggaacggagacactagctataatcagaagtttaaagggaaggctacactgactgtggataagagctcctctactgcatacatgcagctgagttcactgaccagcgaagactccgctgtgtatttctgcgcaagggtggtctactactccaattcttactggtacttcgatgtgtggggcactgggaccacagtcaccgtgagctccgcctcaaccaaaggacctagcgtgttcccactggctccctctagtaagagtacatcaggaggaactgcagctctgggatgtctggtgaaggactacttcccagagcccgtcacagtgtcttggaacagtggggcactgacatctggagtccatacttttcctgccgtgctgcagtcaagcgggctgtacagcctgtcctctgtggtcactgtgccaagttcaagcctgggaacccagacatatatctgcaacgtgaatcacaaaccaagcaataccaaggtcgacaagaaagtggaacccaaatcctgtgataagactcatacctgcccaccttgtcctgcaccagagctgctgggaggaccatccgtgttcctgtttccacccaaacctaaggacaccctgatgatttctagaaccccagaagtcacatgcgtggtcgtggacgtgagccacgaggaccccgaagtcaagtttaactggtacgtggatggcgtcgaggtgcataatgctaaaacaaagccccgggaggaacagtacaactccacctatagagtcgtgtctgtcctgacagtgctgcaccaggactggctgaacgggaaggagtataaatgcaaggtgagcaacaaggcactgcccgcccctatcgagaagacaatttccaaagctaagggacagcctagggaaccacaggtctacgtgctgcctccatctcgcgacgagctgactaaaaaccaggtcagtctgctgtgtctggtgaagggattctatcccagcgatatcgcagtggagtgggaatccaatggccagcctgaaaacaattacctgacctggccccctgtgctggactcagatggcagcttctttctgtatagtaaactgacagtggataagtcacgctggcagcaggggaacgtctttagctgttccgtgatgcatgaggccctgcacaatcattacacccagaaatctctgagtctgtcacccggcaagtga

SEQ ID NO: 247 - protein, 213 aa, synthetic construct
QIVLSQSPAILSASPGEKVTMTCRASSSVSYMHWYQQKPGSSPKPWIYAPSNLASGVPARFSGSGSGTSYSLTISRVEAEDAATYYCQQWSFNPPTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 248 - DNA, 645 bp, synthetic construct
  source 1..645: mol_type = other DNA
ggacagatcgtcctgtcacagagccccgctatcctgtccgcatctcctggcgagaaggtgaccatgacatgccgagctagctcctctgtctcctacatgcactggtatcagcagaagcccgggagttcacctaaaccatggatctacgccccatcaaacctggctagcggagtgccagcacggttcagtggctcagggagcggaacatcctattctctgactatttctagagtggaggctgaagacgccgctacctactattgccagcagtggtccttcaatccccctacctttggcgcagggacaaagctggagctgaaaaggaccgtggcagcccctagtgtcttcatttttccaccctccgacgaacagctgaagtccggcacagcctctgtggtctgtctgctgaacaatttctacccacgcgaggccaaggtgcagtggaaagtcgataacgctctgcagagtggcaacagccaggagagcgtgactgaacaggactccaaggattctacctatagtctgagctccactctgaccctgagcaaagcagattacgagaagcacaaagtgtatgcctgcgaagtcacacatcagggactgtctagtcctgtgactaaaagctttaacagaggcgaatgttga

SEQ ID NO: 249 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 250 - DNA, 1353 bp, synthetic construct
  source 1..1353: mol_type = other DNA
gaggtgcagctggtggaaagcggaggaggactggtgcagccaggaggatctctgcgactgagttgcgccgcttcaggattcaacatcaaggacacctacattcactgggtgcgacaggctccaggaaaaggactggagtgggtggctcgaatctatcccactaatggatacacccggtatgccgactccgtgaaggggaggtttactattagcgccgatacatccaaaaacactgcttacctgcagatgaacagcctgcgagccgaagataccgctgtgtactattgcagtcgatggggaggagacggattctacgctatggattattggggacaggggaccctggtgacagtgagctccgcctctaccaagggccccagtgtgtttcccctggctccttctagtaaatccacctctggagggacagccgctctgggatgtctggtgaaggactatttccccgagcctgtgaccgtgagttggaactcaggcgccctgacaagcggagtgcacacttttcctgctgtgctgcagtcaagcgggctgtactccctgtcctctgtggtgacagtgccaagttcaagcctgggcacacagacttatatctgcaacgtgaatcataagccctcaaatacaaaagtggacaagaaagtggagcccaagagctgtgataagacccacacctgccctccctgtccagctccagaactgctgggaggacctagcgtgttcctgtttccccctaagccaaaagacactctgatgatttccaggactcccgaggtgacctgcgtggtggtggacgtgtctcacgaggaccccgaagtgaagttcaactggtacgtggatggcgtggaagtgcataatgctaagacaaaaccaagagaggaacagtacaactccacttatcgcgtcgtgagcgtgctgaccgtgctgcaccaggactggctgaacgggaaggagtataagtgcaaagtcagtaataaggccctgcctgctccaatcgaaaaaaccatctctaaggccaaaggccagccaagggagccccaggtgtacgtgtacccacccagcagagacgaactgaccaagaaccaggtgtccctgacatgtctggtgaaaggcttctatcctagtgatattgctgtggagtgggaatcaaatggacagccagagaacaattacaagaccacacctccagtgctggacagcgatggcagcttcgccctggtgtccaagctgacagtggataaatctcgatggcagcaggggaacgtgtttagttgttcagtgatgcatgaagccctgcacaatcattacactcagaagagcctgtccctgtctcccggcaaatga

SEQ ID NO: 251 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 252 - DNA, 1353 bp, synthetic construct
  source 1..1353: mol_type = other DNA
gaggtgcagctggtggaaagcggaggaggactggtgcagccaggaggatctctgcgactgagttgcgccgcttcaggattcaacatcaaggacacctacattcactgggtgcgacaggctccaggaaaaggactggagtgggtggctcgaatctatcccactaatggatacacccggtatgccgactccgtgaaggggaggtttactattagcgccgatacatccaaaaacactgcttacctgcagatgaacagcctgcgagccgaagataccgctgtgtactattgcagtcgatggggaggagacggattctacgctatggattattggggacaggggaccctggtgacagtgagctccgcctctaccaagggccccagtgtgtttcccctggctccttctagtaaatccacctctggagggacagccgctctgggatgtctggtgaaggactatttccccgagcctgtgaccgtgagttggaactcaggcgccctgacaagcggagtgcacacttttcctgctgtgctgcagtcaagcgggctgtactccctgtcctctgtggtgacagtgccaagttcaagcctgggcacacagacttatatctgcaacgtgaatcataagccctcaaatacaaaagtggacaagaaagtggagcccaagagctgtgataagacccacacctgccctccctgtccagctccagaactgctgggaggacctagcgtgttcctgtttccccctaagccaaaagacactctgatgatttccaggactcccgaggtgacctgcgtggtggtggacgtgtctcacgaggaccccgaagtgaagttcaactggtacgtggatggcgtggaagtgcataatgctaagacaaaaccaagagaggaacagtacaactccacttatcgcgtcgtgagcgtgctgaccgtgctgcaccaggactggctgaacgggaaggagtataagtgcaaagtcagtaataaggccctgcctgctccaatcgaaaaaaccatctctaaggccaaaggccagccaagggagccccaggtgtacgtgctgccacccagcagagacgaactgaccaagaaccaggtgtccctgctgtgtctggtgaaaggcttctatcctagtgatattgctgtggagtgggaatcaaatggacagccagagaacaattacctgacctggcctccagtgctggacagcgatggcagcttcttcctgtattccaagctgacagtggataaatctcgatggcagcaggggaacgtgtttagttgttcagtgatgcatgaagccctgcacaatcattacactcagaagagcctgtccctgtctcccggcaaatga

SEQ ID NO: 253 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 254 - DNA, 645 bp, synthetic construct
  source 1..645: mol_type = other DNA
gacatccagatgacccagtctccatcctccctgtctgcatctgtaggagacagagtcaccatcacttgccgggcaagtcaggacgttaacaccgctgtagcttggtatcagcagaaaccagggaaagcccctaagctcctgatctattctgcatcctttttgtacagtggggtcccatcaaggttcagtggcagtcgatctgggacagatttcactctcaccatcagcagtctgcaacctgaagattttgcaacttactactgtcaacagcattacactaccccacccactttcggccaagggaccaaagtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaagagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 255 - DNA, 1563 bp, synthetic construct; identical to SEQ ID NO: 199
  source 1..1563: mol_type = other DNA

SEQ ID NO: 256 - protein, 518 aa, synthetic construct [sequence omitted]

SEQ ID NO: 257 - DNA, 3381 bp, synthetic construct
  source 1..3381: mol_type = other DNA
gaattcgcgaccatggccgtgatggctcctcggacactggtgctgctgctgagtggggctctggctctgacacagacttgggctggacaggtgcagctgcaggagtccggaggaggactggtgaagccaggagggtccctgagactgtcttgcgccgctagtggcttcacctttagctcctactggatgtcttgggtgaggcaggcacctggcaagggactggagtgggtggcaaacatcaatcgcgacggatcagccagctactatgtggatagcgtcaagggcaggtttacaattagtcgcgacgatgctaaaaactcactgtacctgcagatgaatagcctgcgggcagaggatactgccgtgtactattgcgccagagacaggggagtcggctatttcgatctgtgggggagaggaactctggtgaccgtctctagtgctagcaccggaggcgggggatctggcggaggaggcagtggaggaggagggtcccagtctgctctgacacagccagcaagtgtgtcaggcagccccgggcagtcaatcactattagctgtactggcacctcaagcgacgtgggaggctacaactttgtcagctggtatcagcagcaccctggaaaagccccaaagctgatgatctacgacgtgagcgatcgaccttccggcgtctctgatcggttctccgggtctaagagtggaaatactgcctccctgatcatttctgggctgcaggctgacgatgaggcagactactattgctcctcttatggaagttcaagcacccatgtgatcttcgggggaggcacaaaagtgactgtcctgggcgcagcctcagatgctcacaaaagcgaggtggcacatcggttcaaggacctgggggaggaaaactttaaagccctggtgctgattgcattcgcccagtacctgcagcagagcccatttgaggaccacgtgaagctggtcaacgaggtgaccgagttcgccaaaacatgcgtggccgacgagtccgctgaaaattgtgataagtctctgcatactctgtttggagataaactgtgcaccgtggccacactgcgagagacctacggcgaaatggcagactgctgtgccaagcaggagccagaaagaaacgagtgcttcctgcagcacaaagacgataacccaaatctgccacgactggtgcgaccagaagtggacgtcatgtgtactgctttccacgataatgaggaaacctttctgaagaaatacctgtatgagatcgcccggagacatccctacttttatgcacctgaactgctgttctttgccaaaagatacaaggctgcattcaccgagtgctgtcaggccgctgataaagcagcctgcctgctgccaaagctggacgagctgcgagatgaaggcaaggcctcctctgctaaacagagactgaagtgtgccagcctgcagaaattcggcgagagggcttttaaggcttgggcagtggcacgactgtcccagagattccctaaggcagagtttgccgaagtctctaaactggtgactgacctgaccaaggtgcacaccgagtgctgtcatggcgacctgctggaatgcgccgacgatcgcgctgatctggcaaagtacatctgtgagaaccaggacagcattagttcaaagctgaaagagtgctgtgaaaaacccctgctggagaagagccactgcatcgcagaggtggaaaacgacgaaatgcccgccgatctgcctagtctggctgcagacttcgtggagtcaaaagatgtctgtaagaattacgctgaagcaaaggatgtgttcctgggcatgtttctgtacgagtatgctaggcgccacccagactactccgtggtcctgctgctgaggctggccaagacctatgagaccacactggaaaaatgctgtgccgctgcagatccccatgagtgctatgccaaagtgttcgacgagttcaagccactggtcgaggaaccccagaacctgattaagcagaattgtgagctgtttgaacagctgggcgagtacaaattccagaacgccctgctggtgcgctatacaaagaaagtccctcaggtgagcacaccaactctggtggaagtctccaggaatctgggaaaggtcggctctaaatgctgtaagcaccccgaggccaaacgcatgccttgcgctgaagattacctgtccgtggtcctgaaccagctgtgtgtgctgcatgagaagaccccagtctctgaccgggtgacaaaatgctgtactgaaagtctggtgaatcgacggccctgctttagcgccctggaggtggatgaaacatatgtccctaaggagttccaggctgaaaccttcacatttcacgcagacatctgtactctgtccgagaaagaaagacagattaagaaacagaccgccctggtcgagctggtgaagcataaacccaaggccacaaaagaacagctgaaggctgtgatggacgatttcgccgcttttgtcgagaaatgctgtaaggctgacgataaggaaacttgctttgcagaggaagggaagaaactggtggcagcatcccaggctgcactgggactggcagctgcactgcaggtccagctggtgcagtctggcgccgaggtgaagaaacctggggaaagtctgaaaatctcctgtaagggcagtgggtactcattcaccagctattggattgcctgggtgaggcagatgccaggaaagggcctggagtacatgggactgatctatcctggcgacagcgatacaaaatactcaccaagctttcagggccaggtcacaattagcgtggataagtccgtctctactgcctatctgcagtggagctccctgaaacctagtgactcagccgtgtacttctgcgctcgccacgacgtcggctattgcacagatcgaacttgtgccaagtggccagagtggctgggagtgtggggacagggaaccctggtgacagtctctagtgggggaggcgggtcaagcggaggagggtccggaggaggaggaagccagtccgtgctgacccagcccccttctgtcagtgccgctcctggccagaaggtgacaatctcatgcagcgggtcctctagtaacattggaaacaattacgtgagctggtatcagcagctgccagggaccgctcccaagctgctgatctacgatcatacaaatagacctgcaggagtgccagacaggttttccggctctaaaagtgggacctcagccagcctggctattagcggcttccggtccgaggacgaagcagattactattgtgcctcctgggactatacactgtctggctgggtgttcggcgggggaactaagctgaccgtcctggggtgaggatcc

SEQ ID NO: 258 - protein, 1124 aa, synthetic construct [sequence omitted]

SEQ ID NO: 259 - DNA, 1860 bp, synthetic construct
  source 1..1860: mol_type = other DNA
gaattcgcgaccatggccgtgatggctcctagaacactggtgctgctgctgtccggggctctggctctgactcagacttgggctgggcaggtgcagctgcaggagagcggaggaggactggtgaaaccaggagggtctctgcgactgagttgcgcagcttcaggcttcacctttagctcctactggatgtcctgggtccgacaggcacctgggaagggactggaatgggtggccaacatcaatcgggacggaagcgcttcctactatgtggatagcgtcaaaggccgctttaccattagtcgagacgatgctaagaactcactgtacctgcagatgaatagcctgcgcgctgaggatacagcagtctactattgcgcaagggaccgaggagtgggatatttcgatctgtggggacgaggcactctggtgaccgtctctagtgccagcacaggcggaggaggatcaggaggaggaggcagcggaggaggcgggtctcagagtgccctgactcagccagcttcagtgagcgggtcccccggacagagcatcaccatttcctgtaccggcacatcaagcgacgtcggaggctacaactttgtgtcctggtatcagcagcacccaggaaaggcccccaaactgatgatctacgacgtctctgatcggcctagcggcgtgtccgatagattctctggcagtaagtcagggaatactgcctctctgatcattagtggcctgcaggccgacgatgaggctgactactattgctcctcttatgggagttcaagcacccatgtgatctttgggggaggcacaaaagtgactgtcctggggggaggctccgatgcacacaagtctgaggtcgcccataggttcaaagacctgggcgaggaaaactttaaggccctggtgctgattgctttcgcacagtacctgcagcagtgcccatttgaagatcacgtgaaactggtcaacgaggtgacagagttcgccaagacttgcgtggcagacgagtccgccgaaaattgtgataaatctctgcatacactgtttggggataagctgtgtaccgtggccacactgagagagacttatggagaaatggctgactgctgtgcaaaacaggagcctgaaaggaacgagtgcttcctgcagcacaaggacgataaccccaatctgcctcgactggtgcggccagaagtggacgtcatgtgtaccgctttccacgataatgaggaaacatttctgaagaaatacctgtatgagatcgcccggagacatccctacttttatgctcctgaactgctgttctttgcaaagcgctacaaagcagccttcacagagtgctgtcaggctgcagataaggccgcttgcctgctgccaaaactggacgagctgagggatgaagggaaagcttcctctgcaaagcagcgcctgaaatgtgcatccctgcagaagttcggcgagcgggcctttaaagcctgggctgtggcaagactgtcccagaggttccccaaggccgagtttgctgaagtctctaagctggtgactgacctgaccaaagtgcacactgagtgctgtcatggcgacctgctggaatgcgccgacgatagagcagatctggccaagtacatctgtgagaatcaggacagcattagttcaaagctgaaagagtgctgtgaaaagcccctgctggagaaaagccactgcattgctgaggtggaaaacgacgaaatgcctgcagatctgccaagtctggcagccgacttcgtcgagagcaaggatgtgtgtaaaaattatgccgaagctaaggacgtgttcctgggcatgtttctgtacgagtatgcaagagcctgaggatcc

SEQ ID NO: 260 - protein, 617 aa, synthetic construct
EFATMAVMAPRTLVLLLSGALALTQTWAGQVQLQESGGGLVKPGGSLRLSCAASGFTFSSYWMSWVRQAPGKGLEWVANINRDGSASYYVDSVKGRFTISRDDAKNSLYLQMNSLRAEDTAVYYCARDRGVGYFDLWGRGTLVTVSSASTGGGGSGGGGSGGGGSQSALTQPASVSGSPGQSITISCTGTSSDVGGYNFVSWYQQHPGKAPKLMIYDVSDRPSGVSDRFSGSKSGNTASLIISGLQADDEADYYCSSYGSSSTHVIFGGGTKVTVLGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARA

SEQ ID NO: 261 - DNA, 1605 bp, synthetic construct
  source 1..1605: mol_type = other DNA
gaattcgcgaccatggctgtgatggctcctcgaaccctggtgctgctgctgtccggcgctctggctctgactcagacttgggctggctccgtggtgctgctgctgaggctggccaaaacctacgagaccacactggaaaagtgctgtgccgctgcagaccctcacgagtgctatgctaaagtcttcgatgagttcaagcccctggtggaggaacctcagaacctgatcaaacagaattgtgagctgttcgaacagctgggcgagtacaagtttcagaacgccctgctggtgcgctataccaagaaagtgccacaggtcagcactcccaccctggtggaggtctccagaaatctggggaaagtgggatctaaatgctgtaagcaccctgaggcaaagaggatgccatgcgccgaagactacctgagcgtggtcctgaaccagctgtgtgtgctgcatgagaaaaccccagtgtccgatcgcgtcaccaagtgctgtacagaaagcctggtgaaccggagaccatgcttctccgccctggaggtcgacgaaacatacgtgcccaaggagtttaatgctgaaacattcactttccacgcagatatctgtaccctgagcgagaaggaacgacagattaagaaacagacagccctggtggagctggtcaagcataaacccaaggccaccaaagaacagctgaaggctgtcatggacgatttcgccgcttttgtggagaaatgctgtaaggcagacgataaggaaacatgcttcgccgaggaaggcaagaaactggtggcagcatcccaggctgcactgggactgggagggagtggacaggtgcagctggtccagagcggagcagaggtgaagaaacccggcgaatcactgaaaatcagctgtaaggggtccggatactcttttactagttattggattgcctgggtgcggcagatgcctggcaaagggctggagtacatggggctgatctatcccggagactctgatacaaagtactcacctagcttccagggccaagtgactattagcgtcgacaaatccgtgtctaccgcttatctgcagtggagctccctgaagcctagtgattcagctgtctacttttgcgcacgacacgacgtgggctattgcaccgatcggacatgtgccaagtggccagagtggctgggagtgtggggacagggaactctggtgaccgtctctagtggaggcgggggatcaagcggaggaggatctggaggaggaggcagccagtccgtcctgactcagccaccttctgtgagtgcagctccaggacagaaggtgaccatctcatgcagcggctcctctagtaacattgggaacaattacgtgagctggtatcagcagctgccaggaacagctcccaaactgctgatctacgaccatactaataggcctgcaggcgtgccagatcgcttctccggctctaagagtgggacatcagcaagcctggccatttccggctttagatctgaggacgaagccgattactattgtgctagttgggactatactctgtcagggtgggtcttcgggggaggcacaaagctgactgtgctgggatgaggatcc

SEQ ID NO: 262 - protein, 532 aa, synthetic construct
EFATMAVMAPRTLVLLLSGALALTQTWAGSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGSGQVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEWLGVWGQGTLVTVSSGGGGSSGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLG

SEQ ID NO: 263 - DNA, 1845 bp, synthetic construct
  source 1..1845: mol_type = other DNA
gaattcgcgaccatggctgtgatggctcctagaaccctggtgctgctgctgtccggcgctctggctctgactcagacctgggctggggatatcaaactgcagcagtctggagctgagctggcacgaccaggagccagtgtcaaaatgtcatgcaagactagcggctacaccttcaccagatataccatgcactgggtgaagcagaggccaggacagggactggaatggatcggatatattaaccccagccgcggctacacaaactataatcagaagttcaaagacaaggctacactgaccacagataagagctcctctactgcatacatgcagctgagttcactgaccagcgaggattccgccgtgtactattgcgctaggtactatgacgatcattactgtctggactattggggacagggcactaccctgactgtgagctccgtcgaaggagggtctggaggcagtggaggatcaggagggagcggaggagtggacgatatccagctgacccagagccccgccattatgagtgcttcacctggcgagaaggtcaccatgacatgccgcgcctctagttcagtgtcctacatgaattggtatcagcagaaatccggcacatctcctaagcggtggatctacgatacttccaaagtcgcctctggggtgccatatcggttcagcgggtccggatctggcaccagttactcactgacaattagctccatggaggctgaagacgccgctacctactattgtcagcagtggtctagtaaccctctgactttcggggccggaaccaaactggagctgaaggggggaagtgatgcacacaaatcagaagtcgcccataggttcaaggacctgggcgaggaaaattttaaagccctggtgctgatcgctttcgcacagtatctgcagcagtgcccatttgaggatcacgtgaagctggtcaacgaggtgacagagttcgccaaaacttgcgtcgcagacgagagcgccgaaaattgtgataagtccctgcataccctgtttggggataaactgtgtactgtggccaccctgagggagacatacggagaaatggctgactgctgtgcaaagcaggagcccgaacgcaacgagtgcttcctgcagcacaaagacgataaccccaatctgcctaggctggtgcgccctgaagtggacgtcatgtgtaccgctttccacgataatgaggaaacatttctgaagaaatacctgtatgagattgcccggagacatccttacttttatgctccagaactgctgttctttgcaaaacggtacaaggcagccttcacagagtgctgtcaggctgcagataaagccgcttgcctgctgccaaagctggacgagctgcgcgatgaaggaaaggcctcaagcgctaaacagcgactgaagtgtgcctccctgcagaaattcggcgagcgggcttttaaggcatgggctgtggcacgactgagccagcggttccccaaggcagagtttgccgaagtctccaaactggtgacagacctgactaaggtgcacaccgagtgctgtcatggggacctgctggaatgcgccgacgatagagctgatctggcaaagtatatctgtgagaaccaggactctatttcctctaaactgaaggagtgctgtgaaaaacctctgctggagaagagccactgcatcgcagaggtggaaaacgacgaaatgccagccgatctgccctctctggcagccgacttcgtcgagagtaaagatgtgtgtaagaattacgccgaagctaaggacgtgttcctgggcatgtttctgtacgagtatgcaagagcctgaggatcc

SEQ ID NO: 264 - protein, 612 aa, synthetic construct
EFATMAVMAPRTLVLLLSGALALTQTWAGDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARA

SEQ ID NO: 265 - DNA, 1590 bp, synthetic construct
  source 1..1590: mol_type = other DNA
gaattcgcgaccatggccgtgatggctccccgaacactggtgctgctgctgtccggcgctctggctctgactcagacttgggctggctccgtggtgctgctgctgcggctggccaagacctatgagaccacactggaaaaatgctgtgccgctgcagatccacacgagtgctacgctaaggtgttcgacgagttcaagcctctggtcgaggaaccacagaacctgatcaagcagaattgtgagctgttcgaacagctgggcgagtataaatttcagaacgccctgctggtgagatacacaaagaaagtgccccaggtctctacacctactctggtggaagtcagtcgaaatctgggaaaggtcggctcaaagtgctgtaaacacccagaggcaaaacggatgccctgcgccgaagactatctgagcgtggtcctgaaccagctgtgtgtgctgcatgagaagacacccgtgtccgatagggtcaccaaatgctgtacagaatctctggtgaaccggagaccctgcttcagtgccctggaggtggacgaaacttacgtccctaaggagtttaatgccgaaaccttcacatttcacgctgatatctgtactctgtccgagaaagaacgccagattaagaaacagaccgccctggtggagctggtcaagcataaacctaaggcaactaaggaacagctgaaagccgtgatggacgatttcgccgcttttgtcgagaagtgctgtaaagctgacgataaggaaacctgcttcgcagaggaaggcaagaaactggtggcagcctctcaggctgcactgggactgggagggagcggggacatccagctgacacagtcccctgcatctctggccgtgagcctgggacagcgagctactatttcctgtaaggcatcccagtctgtggactatgatggggacagctatctgaactggtaccagcagatcccaggacagccccctaagctgctgatctacgatgccagcaatctggtgtccggcatcccaccccgattcagtggatcaggcagcgggacagattttactctgaacattcacccagtggagaaggtcgacgccgctacctaccattgccagcagtctactgaggacccctggaccttcggaggcgggacaaagctggaaatcaaaggaggcgggggatcaggcggaggaggcagcggaggaggagggtcccaggtgcagctgcagcagagcggagcagagctggtgagacctggcagctccgtcaagatttcttgtaaagctagtggctatgcattttctagttactggatgaattgggtgaagcagaggcctggacagggactggagtggatcggacagatttggccaggggatggagacaccaactataatgggaaattcaagggaaaagccactctgaccgctgacgagtcaagctccacagcctatatgcagctgtctagtctggcaagtgaggattcagccgtgtacttttgcgctaggcgcgaaactaccacagtcggcaggtactattacgctatggactactggggccaggggactaccgtgaccgtctcaagctgaggatcc

SEQ ID NO: 266 - protein, 527 aa, synthetic construct
EFATMAVMAPRTLVLLLSGALALTQTWAGSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGSGDIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSS

SEQ ID NO: 267 - DNA, 3351 bp, synthetic construct
  source 1..3351: mol_type = other DNA
gaattcgcgaccatggccgtgatggcacctagaacactggtcctgctgctgagcggggcactggcactgacacagacttgggctggagacattaagctgcagcagagcggagctgagctggcacgaccaggagcaagtgtgaaaatgtcatgcaagacaagcggctatacttttaccaggtacactatgcactgggtgaagcagcgaccaggacagggactggagtggatcgggtacattaacccttccagaggatataccaactacaatcagaagttcaaagataaggccaccctgaccacagacaagagctcctctacagcctatatgcagctgagttcactgacttccgaggattctgccgtgtactattgcgctaggtactatgacgatcattattgtctggactactgggggcagggaactaccctgacagtgagctccgtcgagggagggagtggaggctcaggaggaagcggagggtccggaggagtggacgatatccagctgactcagtctcctgcaattatgtcagccagcccaggcgagaaggtcacaatgacttgccgcgcctctagttcagtgtcctatatgaattggtaccagcagaaatctggcaccagtcccaagagatggatctatgatacatccaaggtcgcctctggggtgccttacaggttttccggctctgggagtggaacttcatatagcctgaccattagctccatggaggctgaagacgccgctacctactattgccagcagtggtctagtaaccccctgacattcggcgccgggactaaactggagctgaaggggggatctgacgcacacaaaagtgaagtcgcccatcggttcaaggatctgggcgaggaaaattttaaagccctggtgctgatcgccttcgctcagtacctgcagcagtgcccttttgaggaccacgtgaagctggtcaacgaggtgaccgagttcgctaaaacatgcgtggctgatgagtctgcagaaaattgtgacaagagtctgcatacactgtttggggataaactgtgtaccgtggccacactgcgcgagacttacggagaaatggccgactgctgtgctaagcaggagcctgaacgaaacgagtgcttcctgcagcacaaagacgataaccctaatctgccacgcctggtgcgaccagaagtggatgtcatgtgtactgctttccacgacaatgaggaaacctttctgaagaaatatctgtacgagatcgcccggagacatccatatttttacgcacccgaactgctgttctttgccaaaagatacaaggcagccttcaccgagtgctgtcaggctgcagataaagccgcttgcctgctgcctaagctggatgagctgcgagacgaaggaaaggcctcaagcgctaaacagcggctgaagtgtgctagcctgcagaaattcggagagcgagccttcaaggcatgggcagtggctcggctgtctcagagattcccaaaggcagagtttgccgaagtcagcaaactggtgactgacctgaccaaggtgcacaccgagtgctgtcatggcgatctgctggaatgcgccgacgatagagctgacctggcaaagtacatctgtgagaaccaggatagcatttcctctaaactgaaggagtgctgtgaaaaacctctgctggagaagtcccactgcatcgccgaggtggaaaacgatgaaatgcccgctgacctgccttcactggcagccgatttcgtcgagagcaaagacgtgtgtaagaattacgcagaagccaaggatgtgttcctgggcatgtttctgtatgagtacgctaggcgacacccagactacagcgtggtcctgctgctgcggctggccaagacatatgagacaactctggaaaaatgctgtgctgcagccgaccctcatgagtgctatgccaaagtcttcgatgagttcaagcctctggtggaggaaccacagaacctgattaagcagaattgtgagctgtttgaacagctgggcgagtataaattccagaacgccctgctggtgagatacaccaagaaagtcccccaggtgtccacccctacactggtggaagtctctcggaatctgggaaaggtcggcagtaaatgctgtaagcacccagaggctaaaagaatgccctgcgcagaagattacctgtccgtggtcctgaaccagctgtgtgtgctgcatgagaagacaccagtctctgacagggtgaccaaatgctgtacagaatccctggtgaaccgacggccttgcttttctgccctggaggtcgatgaaacctatgtgccaaaggagttcaatgctgaaactttcacctttcacgcagacatctgtactctgagcgagaaagaacgccagattaagaaacagaccgccctggtcgagctggtgaaacataagcccaaagccacaaaagaacagctgaaggctgtcatggacgatttcgctgcatttgtggagaaatgctgtaaggcagacgataaggaaacctgcttcgccgaggaaggcaagaaactggtggccgctagccaggcagcactgggactgggagggtccggggatatccagctgacccagtcaccagccagcctggctgtctcactggggcagagggccacaattagttgtaaggcttcccagtctgtggactacgatggagactcctatctgaactggtaccagcagatcccaggacagccacctaaactgctgatctacgacgccagtaatctggtgtcaggcatcccaccccgctttagtggatcaggcagcgggacagacttcactctgaacattcacccagtcgagaaggtggatgctgcaacctaccattgccagcagagcactgaggacccctggacctttggaggcgggacaaaactggaaatcaagggaggcgggggatcaggcggaggaggcagcggaggaggagggtcccaggtgcagctgcagcagtccggagcagagctggtcaggccagggagttcagtgaaaattagctgtaaggcatccgggtatgccttcagctcctactggatgaattgggtcaaacagagacctggccagggcctggagtggatcggacagatttggcccggggatggagacactaactacaatgggaagtttaaaggaaaggctacactgactgcagatgaatctagttcaaccgcctacatgcagctgagctccctggcatccgaggactctgccgtctatttctgcgctagaagggaaaccacaactgtgggcaggtactattacgccatggactattggggccaggggaccacagtgaccgtctctagttgaggatcc

SEQ ID NO: 268 - protein, 1114 aa, synthetic construct
EFATMAVMAPRTLVLLLSGALALTQTWAGDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGSGDIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSS

SEQ ID NO: 269 - protein, 337 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARA

SEQ ID NO: 270 - protein, 244 aa, synthetic construct
SVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 271 - protein, 609 aa, Homo sapiens [sequence omitted]

SEQ ID NO: 272 - protein, 29 aa, synthetic construct
TCCCKSSCLRLITSHLKASQPTMRIRERK

SEQ ID NO: 273 - protein, 422 aa, synthetic construct
  VARIANT 1..20: note = Residues may be deleted
  VARIANT 22..41: note = Residues may be deleted
  VARIANT 43..62: note = Residues may be deleted
  VARIANT 64..83: note = Residues may be deleted
  VARIANT 85..104: note = Residues may be deleted
  VARIANT 106..125: note = Residues may be deleted
  VARIANT 127..146: note = Residues may be deleted
  VARIANT 148..167: note = Residues may be deleted
  VARIANT 169..188: note = Residues may be deleted
  VARIANT 190..209: note = Residues may be deleted
  VARIANT 211..230: note = Residues may be deleted
  VARIANT 232..251: note = Residues may be deleted
  VARIANT 253..272: note = Residues may be deleted
  VARIANT 274..293: note = Residues may be deleted
  VARIANT 295..314: note = Residues may be deleted
  VARIANT 316..335: note = Residues may be deleted
  VARIANT 337..356: note = Residues may be deleted
  VARIANT 358..377: note = Residues may be deleted
  VARIANT 379..398: note = Residues may be deleted
  VARIANT 400..419: note = Residues may be deleted
  VARIANT 1..420: note = GGGGGGGGGGGGGGGGGGGGS repeats may be deleted wherein some residues may be absent
GGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGG

SEQ ID NO: 274 - protein, 420 aa, synthetic construct
  VARIANT 2..21: note = Residues may be deleted
  VARIANT 23..42: note = Residues may be deleted
  VARIANT 44..63: note = Residues may be deleted
  VARIANT 65..84: note = Residues may be deleted
  VARIANT 86..105: note = Residues may be deleted
  VARIANT 107..126: note = Residues may be deleted
  VARIANT 128..147: note = Residues may be deleted
  VARIANT 149..168: note = Residues may be deleted
  VARIANT 170..189: note = Residues may be deleted
  VARIANT 191..210: note = Residues may be deleted
  VARIANT 212..231: note = Residues may be deleted
  VARIANT 233..252: note = Residues may be deleted
  VARIANT 254..273: note = Residues may be deleted
  VARIANT 275..294: note = Residues may be deleted
  VARIANT 296..315: note = Residues may be deleted
  VARIANT 317..336: note = Residues may be deleted
  VARIANT 338..357: note = Residues may be deleted
  VARIANT 359..378: note = Residues may be deleted
  VARIANT 380..399: note = Residues may be deleted
  VARIANT 401..420: note = Residues may be deleted
  VARIANT 1..420: note = SGGGGGGGGGGGGGGGGGGGG repeats may be deleted wherein some residues may be absent
SGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGG

SEQ ID NO: 275 - protein, 440 aa, synthetic construct
  VARIANT 3..22: note = Residues may be deleted
  VARIANT 25..44: note = Residues may be deleted
  VARIANT 47..66: note = Residues may be deleted
  VARIANT 69..88: note = Residues may be deleted
  VARIANT 91..110: note = Residues may be deleted
  VARIANT 113..132: note = Residues may be deleted
  VARIANT 135..154: note = Residues may be deleted
  VARIANT 157..176: note = Residues may be deleted
  VARIANT 179..198: note = Residues may be deleted
  VARIANT 201..220: note = Residues may be deleted
  VARIANT 223..242: note = Residues may be deleted
  VARIANT 245..264: note = Residues may be deleted
  VARIANT 267..286: note = Residues may be deleted
  VARIANT 289..308: note = Residues may be deleted
  VARIANT 311..330: note = Residues may be deleted
  VARIANT 333..352: note = Residues may be deleted
  VARIANT 355..374: note = Residues may be deleted
  VARIANT 377..396: note = Residues may be deleted
  VARIANT 399..418: note = Residues may be deleted
  VARIANT 421..440: note = Residues may be deleted
  VARIANT 1..440: note = SEGGGGGGGGGGGGGGGGGGGG repeats may be deleted wherein some residues may be absent
SEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGG

SEQ ID NO: 276 - protein, 17 aa, unidentified
  source 1..17: note = Stanniocalcin signal peptide
MLQNSAVLLLLVISASA

SEQ ID NO: 277 - protein, 22 aa, synthetic construct
MPTWAWWLFLVLLLALWAPARG

SEQ ID NO: 278 - protein, 4 aa, synthetic construct
GGGS

SEQ ID NO: 279 - protein, 18 aa, synthetic construct
GGSGGGSGGGSGGGSGGG

SEQ ID NO: 280 - protein, 4 aa, synthetic construct
GGSG

SEQ ID NO: 281 - protein, 15 aa, synthetic construct
GGGGSGGGGSGGGGS

SEQ ID NO: 282 - protein, 4 aa, synthetic construct
AAAL